(19) United States
(12) Patent Application Publication
(10) Pub. No.: US 2010/0272680 A1
(43) Pub. Date: Oct. 28, 2010

(54) Piperidinyl Cyclic Amido Antiviral Agents

(76) Inventors: Subramanian Baskaran, Durham, NC (US); Jack Maung, Durham, NC (US); Martin Neitzel, Pacifica, CA (US); Rai Roopa, Durham, NC (US); Christopher Roberts, Durham, NC (US); Vincent Tai, Durham, NC (US)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(21) Appl. No.: 12/702,702

(22) Filed: Feb. 9, 2010

Related U.S. Application Data
(60) Provisional application No. 61/151,180, filed on Feb. 9, 2009.

(51) Int. Cl. A61K 38/21; C07D 471/04; A61K 31/437; A61P 31/12; A61P 31/14

(52) U.S. Cl. 424/85.4; 546/121; 514/300

(57) ABSTRACT

Provided are compounds of Formula (I) and pharmaceutically acceptable salts thereof, their pharmaceutical compositions, their methods of preparation, and their use for treating viral infections mediated by a member of the Flaviviridae family of viruses such as hepatitis C virus (HCV).

### CROSS-REFERENCE TO RELATED APPLICATIONS

This application is related to and claims the priority benefit of U.S. Provisional Patent Application Ser. No. 61/151,180, filed Feb. 9, 2009, which is incorporated by reference herein in its entirety.

### FIELD OF THE INVENTION

Provided are compounds, pharmaceutical compositions, their methods of preparation, and their use for treating viral infections mediated by a member of the Flaviviridae family of viruses such as hepatitis C virus (HCV).

### BACKGROUND OF THE INVENTION

The Flaviviridae family of viruses is composed of three genera: pestivirus, flavivirus and hepacivirus. Within these genera, flaviviruses and hepaciviruses represent important pathogens of man and are prevalent throughout the world. There are 38 flaviviruses associated with human disease, including the dengue fever viruses, yellow fever virus, and Japanese encephalitis virus. Flaviviruses cause a range of acute febrile illnesses and encephalitic and hemorrhagic diseases. Hepaciviruses currently infect approximately 2 to 3% of the world population and cause persistent infections leading to chronic liver disease, cirrhosis, hepatocellular carcinoma and liver failure. Human pestiviruses have not been as extensively characterized as the animal pestiviruses. However, serological surveys indicate considerable pestivirus exposure in humans. Pestivirus infections in man have been implicated in several diseases including, but not limited to, congenital brain injury, infantile gastroenteritis and chronic diarrhea in human immunodeficiency virus (HIV).

Hepatitis C virus (HCV) is a hepacivirus and is a major causative agent for post-transfusion and for sporadic hepatitis. Infection by HCV is insidious in a high proportion of chronically infected (and infectious) carriers who may not experience clinical symptoms for many years.

At present, the only acceptable treatment for chronic HCV is interferon (IFN-alpha) and/or ribavirin and this requires at least six (6) months of treatment, which can reduce the viral load and also improve liver function in some people.

IFN-alpha belongs to a family of naturally occurring small proteins with characteristic biological effects such as antiviral, immunoregulatory and anti-tumoral activities. IFN-alpha is an important regulator of immunological control. Treatment of HCV with interferon, however, has limited long term efficacy with a response rate about 25%. In addition, treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction.

Ribavirin (1-beta-D-ribofuranosyl-1H-1,2,4-triazole-3-carboxamide), an inhibitor of inosine 5′-monophosphate dehydrogenase (IMPDH), enhances the efficacy of IFN-alpha in the treatment of HCV. Despite the introduction of ribavirin, up to 50% of the patients do not eliminate the virus with the current standard therapy of interferon-alpha (IFN) and ribavirin. Ribavirin causes significant hemolysis in 10-20% of patients treated at currently recommended doses, and the drug is both teratogenic and embryotoxic. By now, standard therapy of chronic hepatitis C has been changed to the combination of PEG-IFN (pegylated interferon) plus ribavirin which leads only to small improvement.

Other approaches are being taken to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among non-structural viral proteins, NS3/4a serine protease, NS5b RNA dependent RNA polymerase are considered as prime targets for new drugs.

There is a need for the development of new compounds that combat hepacivirus. There remains a need for agents with stronger response rates and fewer side effects in terms of relief of symptoms, safety, and patient mortality, both short-term and long-term and an improved therapeutic index.

### SUMMARY OF THE INVENTION

Provided is a compound of Formula (I):

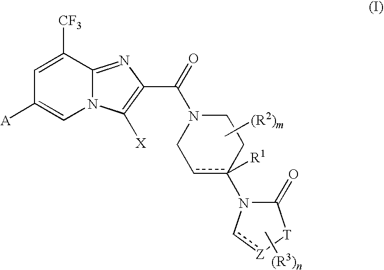

or a pharmaceutically acceptable salt thereof, wherein:
Z and T are independently selected from the group consisting of O, N, NR⁴, CH, CH₂, CH═CH, and CH₂═CH₂, optionally substituted with one to two R³;
R¹ is absent or is selected from the group consisting of hydrogen, halo, cyano, hydroxy, and alkyl;
R² and R³ are independently selected from the group consisting of hydrogen, deuterium, halo, cyano, oxo, —OR⁶, —NR⁶R⁶, —NR⁶S(O)₂(alkyl), —NR⁶C(O)OR⁶, —NR⁶C(O)R⁶, —C(O)NR⁶R⁶, —C(O)OR⁶, —C(O)R⁶, alkyl, and alkyl substituted with one to three R⁵;
R⁴ is hydrogen or alkyl;
R⁵ is independently selected from the group consisting of halo, cyano, —OR⁶, —C(O)OR⁶, —NR⁶R⁶, and alkoxy substituted with one to three R^{5a} wherein R^{5a} independently selected from the group consisting of halo, hydroxy, cyano, —OR⁶, —C(O)OR⁶, and —NR⁶R⁶;
m is 0, 1, 2, 3, 4, 5, or 6;
n is 0, 1, 2, 3, or 4;
[symbol] independently represents a single bond or a double bond, provided that when [symbol] is a double bond, then R¹ is absent;
X is selected from the group consisting of hydrogen, cyano, halo, —CH₂OH, hydroxy, alkyl, and cycloalkyl;
A is selected from the group consisting of hydrogen, halo, cyano, alkyl, alkoxy, alkenyl, alkynyl, cycloalkyl, aryl, heteroaryl, or heterocyclic, —OR⁶, —NR⁶R⁶, —NR⁶C(O)NR⁶R⁶, —NR⁶C(S)NR⁶R⁶, —NR⁶S(O)₂R⁶—NR⁶C(O)OR⁶, and —NR⁶C(O)R⁶; wherein said alkyl, alkoxy, alkenyl, alkynyl, cycloalkyl, aryl, heteroaryl, or heterocyclic is optionally substituted with one to three substituents independently selected from alkyl, alkenyl, alkynyl, halo, hydroxy, cyano, —OR⁶, —SR⁶, —S(O)R⁶, —S(O)₂R⁶, —S(O)₂NR⁶R⁶, —NR⁶R⁶, —NR⁶C(O)NR⁶R⁶, —NR⁶C(S)NR⁶R⁶, —NR⁶S(O)₂R⁶—NR⁶C(O)OR⁶, —NR⁶C(O)R⁶, —C(NR⁶)NR⁶R⁶, —C(O)NR⁶R⁶, —C(O)OR⁶, —CN, —C(O)R⁶, cycloalkyl, aryl, heteroaryl, and heterocyclic; and
R⁶ is independently hydrogen or alkyl.

Also provided is a pharmaceutical composition comprising a pharmaceutically acceptable diluent and a therapeutically effective amount of a compound of Formula (I) or a pharmaceutically acceptable salt thereof.

Also provided are synthetic intermediates, methods for preparing the compounds and compositions, and methods for treating a viral infection mediated at least in part by a virus in the Flaviviridae family of viruses, such as HCV.

These and further embodiments of invention are described in the detailed description that follows.

### DETAILED DESCRIPTION OF THE INVENTION

As used in the present specification, the following words and phrases are generally intended to have the meanings as set forth below, except to the extent that the context in which they are used indicates otherwise.

The following abbreviations and terms have the indicated meanings throughout:
HCV: hepacivirus
HIV: human immunodeficiency virus
IFN: interferon
IMPDH: inosine 5′-monophosphate dehydrogenase
MS: Mass Spectrometry
NMR: Nuclear Magnetic Resonance
ESI: Electrospray Ionization
mg: milligram
kg: kilogram
nm: nanometer
MDI: metered dose inhaler
DPI: dry powder inhaler
nM: nano-Molar
wt %: weight percent
μM: micro-Molar
EC₅₀: effective concentration of compound at 50% inhibition is observed
TC₅₀: toxic concentration of compound at which 50% inhibition is observed
b: Hill's coefficient
d: doublet
dd: doublet of doublets
m: multiplet
s: singlet
g: gram
Hz: Hertz
K: Kelvin
mL: milli-Liter
m/z: mass-to-charge ratio
1N: 1 Normal concentration
AIDS: Acquired Immunodeficiency syndrome
DMSO: Dimethylsulfoxide
NCS: N-Chlorosuccinimide
HBTU: 2(1H-Benzotriazole-1-yl)-1,1,3,3-tetramethyluronium hexafluorophosphate
RNA: Ribonucleic acid
TEA: Triethylamine
THF: Tetrahydrofuran

It is to be understood that the terminology used herein is for the purpose of describing particular embodiments only and is not intended to limit the scope of the present specification. In this specification and in the claims that follow, reference will be made to a number of terms that shall be defined to have the following meanings:

“Alkyl” refers to monovalent saturated aliphatic hydrocarbyl groups having from 1 to 10 carbon atoms and, in some embodiments, from 1 to 6 carbon atoms. “C_{x-y}alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). The term “alkyl” also refers to two alkyl groups substituted at the same carbon atom, for example, gem-dimethyl groups. Also, the term “alkyl” includes alkyl groups where one or more bonded hydrogen atoms have been replaced with one or more deuterium atoms.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, isopropenyl, 1,3-butadienyl, and the like.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Cyano” or “carbonitrile” refers to the group —CN.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups. Examples of cycloalkyl groups include, for instance, adamantyl, cyclopropyl, cyclobutyl, cyclopentyl, cyclooctyl, cyclopentenyl and cyclohexenyl. “Cᵤ₋ᵥcycloalkyl” refers to cycloalkyl groups having u to v carbon atoms.

“Halo” or “halogen” refers to fluoro, chloro, bromo, and iodo.

“Haloalkoxy” refers to substitution of alkoxy groups with 1 to 5 (e.g. when the alkoxy group has at least 2 carbon atoms) or in some embodiments 1 to 3 halo groups (e.g. trifluromethoxy).

“Hydroxy” or “hydroxyl” refers to the group —OH.

“Heteroaryl” refers to an aromatic group of from 1 to 14 carbon atoms and 1 to 6 heteroatoms selected from oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from nitrogen, sulfur, phosphorus or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen, phosphorus and/or sulfur atom(s) of the heterocyclic group are optionally oxidized to provide for the N-oxide, phosphinane oxide, sulfinyl, sulfonyl moieties. More specifically the heterocyclyl includes, but is not limited to, tetrahydropyranyl, piperidinyl, piperazinyl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

Examples of heterocycle and heteroaryl groups include, but are not limited to, azetidine, pyrrole, imidazole, pyrazole, pyridine, pyrazine, pyrimidine, pyridazine, pyridone, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholine, thiomorpholine (also referred to as thiamorpholine), piperidine, pyrrolidine, and tetrahydrofuran.

“Oxo” refers to the oxygen atom ═O.

“Compound”, “compounds”, “chemical entity”, and “chemical entities” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers.

“Solvate” or “solvates” of a compound refer to those compounds, as defined above, which are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

It is understood that the above definitions are not intended to include impermissible substitution patterns (e.g., methyl substituted with 5 fluoro groups). Such impermissible substitution patterns are well known to the skilled artisan.

Provided is a compound of Formula (I):

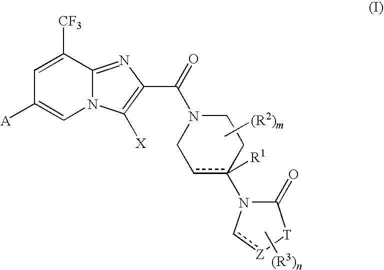

or a pharmaceutically acceptable salt thereof, wherein:
Z and T are independently selected from the group consisting of O, N, NR⁴, CH, CH₂, CH═CH, and CH₂═CH₂, optionally substituted with one to two R³;
R¹ is absent or is selected from the group consisting of hydrogen, halo, cyano, hydroxy, and alkyl;
R² and R³ are independently selected from the group consisting of hydrogen, deuterium, halo, cyano, oxo, —OR⁶, —NR⁶R⁶, —NR⁶S(O)₂(alkyl), —NR⁶C(O)OR⁶, —NR⁶C(O)R⁶, —C(O)NR⁶R⁶, —C(O)OR⁶, —C(O)R⁶, alkyl, and alkyl substituted with one to three R⁵;
R⁴ is hydrogen or alkyl;
R⁵ is independently selected from the group consisting of halo, cyano, —OR⁶, —C(O)OR⁶, —NR⁶R⁶, and alkoxy substituted with one to three R^{5a} wherein R^{5a} independently selected from the group consisting of halo, hydroxy, cyano, —OR⁶, —C(O)OR⁶, and —NR⁶R⁶;
m is 0, 1, 2, 3, 4, 5, or 6;
n is 0, 1, 2, 3, or 4;
[symbol] independently represents a single bond or a double bond, provided that when [symbol] is a double bond, then R¹ is absent;
X is selected from the group consisting of hydrogen, cyano, halo, —CH₂OH, hydroxy, alkyl, and cycloalkyl;
A is selected from the group consisting of hydrogen, halo, cyano, alkyl, alkoxy, alkenyl, alkynyl, cycloalkyl, aryl, heteroaryl, or heterocyclic, —OR⁶, —NR⁶R⁶, —NR⁶C(O)NR⁶R⁶, —NR⁶C(S)NR⁶R⁶, —NR⁶S(O)₂R⁶—NR⁶C(O)OR⁶, and —NR⁶C(O)R⁶; wherein said alkyl, alkoxy, alkenyl, alkynyl, cycloalkyl, aryl, heteroaryl, or heterocyclic is optionally substituted with one to three substituents independently selected from alkyl, alkenyl, alkynyl, halo, hydroxy, cyano, —OR⁶, —SR⁶, —S(O)R⁶, —S(O)₂R⁶, —S(O)₂NR⁶R⁶, —NR⁶R⁶, —NR⁶R⁶, —NR⁶C(O)NR⁶R⁶, —NR⁶C(S)NR⁶R⁶, —NR⁶S(O)₂R⁶—NR⁶C(O)OR⁶, —NR⁶C(O)R⁶, —C(NR⁶)NR⁶R⁶, —C(O)NR⁶R⁶, —C(O)OR⁶, —CN, —C(O)R⁶, cycloalkyl, aryl, heteroaryl, and heterocyclic; and
R⁶ is independently hydrogen or alkyl.

In another embodiment provided is a compound of Formula (I):

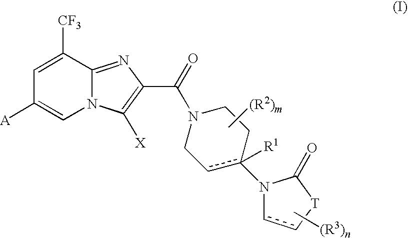

or a pharmaceutically acceptable salt thereof, wherein
T is selected from the group consisting of O, NR⁴, and CH₂ optionally substituted with one to two R³;
R¹ is absent or is selected from the group consisting of hydrogen, halo, cyano, hydroxy, and alkyl;
R² and R³ are independently selected from the group consisting of halo, cyano, oxo, —OR⁶, —NR⁶R⁶, —NR⁶S(O)₂(alkyl), —NR⁶C(O)OR⁶, —NR⁶C(O)R⁶, —C(O)NR⁶R⁶, —C(O)OR⁶, —C(O)R⁶, alkyl, and alkyl substituted with one to three R⁵;
R⁴ is hydrogen or alkyl;
R⁵ is independently selected from the group consisting of halo, cyano, —OR⁶, —C(O)OR⁶, —NR⁶R⁶, and alkoxy substituted with one to three R^{5a} wherein R^{5a} independently selected from the group consisting of halo, hydroxy, cyano, —OR⁶, —C(O)OR⁶, and —NR⁶R⁶;
m is 0, 1, 2, 3, 4, 5, or 6;
n is 0, 1, 2, 3, or 4;
[symbol] independently represents a single bond or a double bond, provided that when [symbol] is a double bond, then R¹ is absent;
X is selected from the group consisting of hydrogen, halo, —CH₂OH, hydroxy, alkyl and cycloalkyl;
A is selected from the group consisting of halo, alkyl, alkenyl, alkynyl, cycloalkyl, aryl, heteroaryl, heterocyclic, —OR⁶, —NR⁶R⁶, —NR⁶C(O)NR⁶R⁶, —NR⁶C(S)NR⁶R⁶, —NR⁶S(O)₂R⁶, —NR⁶C(O)OR⁶, and —NR⁶C(O)R⁶; wherein said alkyl, alkenyl, alkynyl, cycloalkyl, aryl, heteroaryl, or heterocyclic is optionally substituted with one to three substituents independently selected from alkyl, alkenyl, alkynyl, halo, hydroxy, cyano, —OR⁶, —SR⁶, —S(O)R⁶, —S(O)₂R⁶, —S(O)₂NR⁶R⁶, —NR⁶R⁶, —NR⁶C(O)NR⁶R⁶, —NR⁶C(S)NR⁶R⁶, —NR⁶S(O)₂R⁶, —NR⁶C(O)OR⁶, —NR⁶C(O)R⁶, —C(NR⁶)NR⁶R⁶, —C(O)NR⁶R⁶, —C(O)OR⁶, —CN, —C(O)R⁶, cycloalkyl, aryl, heteroaryl, and heterocyclic; and
R⁶ is independently hydrogen or alkyl.

In another embodiment, provided is a compound having the structure:

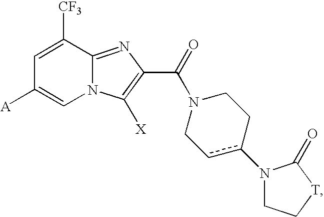

whererin A, X, T, and [symbol] are as defined herein.

In some embodiments, T is CH₂.

In some embodiments, T is O.

In some embodiments, R¹ is hydrogen.

In some embodiments, X is halo.

In some embodiments, X is chloro.

In some embodiments, X is cyano.

In some embodiments, X is chloro, and T is CH₂ or O.

In some embodiments, X is chloro, T is CH₂ or O, and R¹ is hydrogen.

In some embodiments, m is 0. In some embodiments, X is chloro, T is CH₂ or O, and m is 0.

In some embodiments, n is 0. In some embodiments, X is chloro, T is CH₂ or O, and n is 0.

In some embodiments, A is selected from the group consisting of halo, alkenyl, cycloalkyl, aryl, heteroaryl, and heterocyclic. In some embodiments, X is chloro, T is CH₂ or O, and A is selected from the group consisting of halo, alkenyl, cycloalkyl, aryl, heteroaryl, and heterocyclic.

In some embodiments, A is heteroaryl. In some embodiments, X is chloro, T is CH₂ or O, and A is heteroaryl.

In some embodiments, A is selected from furanyl and pyrazolyl.

In some embodiments, A is aryl. In some embodiments, X is chloro, T is CH₂ or O, and A is aryl.

In some embodiments, A is phenyl.

In some embodiments, A is cyano.

In some embodiments, A is cycloalkyl. In some embodiments, X is chloro, T is CH₂ or O, and A is cycloalkyl.

In some embodiments, A is cyclopentenyl.

In some embodiments, A is halo. In some embodiments, X is chloro, T is CH₂ or O, and A is halo.

In some embodiments, A is bromo.

In some embodiments, A is alkenyl. In some embodiments, X is chloro, T is CH₂ or O, and A is alkenyl.

In some embodiments, A is isopropenyl.

In another embodiment, the compound is selected from Table 1 or a pharmaceutically acceptable salt thereof. In even further embodiments, the compound is selected from the group consisting of:
1-(1-{[3-chloro-6-(3-furanyl)-8-(trifluoromethyl)imidazo[1,2-a]pyridin-2-yl]carbonyl}-4-piperidinyl)-2-pyrrolidinone,
1-(1-{[3-chloro-6-(1H-pyrazol-4-yl)-8-(trifluoromethyl)imidazo[ 1,2-a]pyridin-2-yl]carbonyl}-4-piperidinyl)-2-pyrrolidinone,
3-(1-{[3-chloro-6-(3-furanyl)-8-(trifluoromethyl)imidazo[1,2-a]pyridin-2-yl]carbonyl}-4-piperidinyl)-1,3-oxazolidin-2-one,
3-(1-{[3-chloro-6-phenyl-8-(trifluoromethyl)imidazo[1,2-a]pyridin-2-yl]carbonyl}-4-piperidinyl)-1,3-oxazolidin-2-one,
3-(1-{[6-bromo-3-chloro-8-(trifluoromethyl)imidazo[1,2-a]pyridin-2-yl]carbonyl}-4-piperidinyl)-1,3-oxazolidin-2-one,
3-(1-{[3-chloro-6-(1-methylethenyl)-8-(trifluoromethyl)imidazo[1,2-a]pyridin-2-yl]carbonyl}-4-piperidinyl)-1,3-oxazolidin-2-one,
3-(1-{[3-chloro-6-(1-cyclopenten-1-yl)-8-(trifluoromethyl)imidazo[1,2-a]pyridin-2-yl]carbonyl}-4-piperidinyl)-1,3-oxazolidin-2-one,
3-(1-{[3-chloro-6-(1H-pyrazol-4-yl)-8-(trifluoromethyl)imidazo[1,2-a]pyridin-2-yl]carbonyl}-4-piperidinyl)-1,3-oxazolidin-2-one,
1-(1-{[3-chloro-8-(trifluoromethyl)imidazo[1,2-a]pyridin-2-yl]carbonyl}-4-piperidinyl)-2-pyrrolidinone,
1-(1-{[3,6-dichloro-8-(trifluoromethyl)imidazo[1,2-a]pyridin-2-yl]carbonyl}-4-piperidinyl)-2-pyrrolidinone,
1-(1-{[3-chloro-8-(trifluoromethyl)imidazo[1,2-a]pyridin-2-yl]carbonyl}-4-piperidinyl)-2-pyrrolidinone,
3-(1-{[3,6-dichloro-8-(trifluoromethyl)imidazo[1,2-a]pyridin-2-yl]carbonyl}-4-piperidinyl)-1,3-oxazolidin-2-one,
3-(1-{[3-chloro-6-(4-pyridinyl)-8-(trifluoromethyl)imidazo[1,2-a]pyridin-2-yl]carbonyl}-4-piperidinyl)-1,3-oxazolidin-2-one,
3-(1-{[3-chloro-6-(3-fluorophenyl)-8-(trifluoromethyl)imidazo[1,2-a]pyridin-2-yl]carbonyl}-4-piperidinyl)-1,3-oxazolidin-2-one,
3-(1-{[3-chloro-6-(4-fluorophenyl)-8-(trifluoromethyl)imidazo[1,2-a]pyridin-2-yl]carbonyl}-4-piperidinyl)-1,3-oxazolidin-2-one,
3-(1-{[3-chloro-6-[2-(methyloxy)phenyl]-8-(trifluoromethyl)imidazo[1,2-a]pyridin-2-yl]carbonyl}-4-piperidinyl)-1,3-oxazolidin-2-one,
3-(1-{[3-chloro-6-[3-(methyloxy)phenyl]-8-(trifluoromethyl)imidazo[1,2-a]pyridin-2-yl]carbonyl}-4-piperidinyl)-1,3-oxazolidin-2-one,
3-(1-{[3-chloro-6-[4-(methyloxy)phenyl]-8-(trifluoromethyl)imidazo[1,2-a]pyridin-2-yl]carbonyl}-4-piperidinyl)-1,3-oxazolidin-2-one,
3-(1-{[3-chloro-6-(3-thienyl)-8-(trifluoromethyl)imidazo[1,2-a]pyridin-2-yl]carbonyl}-4-piperidinyl)-1,3-oxazolidin-2-one,
3-(1-{[3-chloro-6-(8-quinolinyl)-8-(trifluoromethyl)imidazo[1,2-a]pyridin-2-yl]carbonyl}-4-piperidinyl)-1,3-oxazolidin-2-one,
3-(1-{[3-chloro-6-(2-hydroxyphenyl)-8-(trifluoromethyl)imidazo[1,2-a]pyridin-2-yl]carbonyl}-4-piperidinyl)-1,3-oxazolidin-2-one,
3-(1-{[3-chloro-6-(3-hydroxyphenyl)-8-(trifluoromethyl)imidazo[1,2-a]pyridin-2-yl]carbonyl}-4-piperidinyl)-1,3-oxazolidin-2-one,
3-(1-{[3-chloro-6-(4-hydroxyphenyl)-8-(trifluoromethyl)imidazo[1,2-a]pyridin-2-yl]carbonyl}-4-piperidinyl)-1,3-oxazolidin-2-one,
3-(1-{[3-chloro-6-(1-methyl-1H-pyrazol-4-yl)-8-(trifluoromethyl)imidazo[1,2-a]pyridin-2-yl]carbonyl}-4-piperidinyl)-1,3-oxazolidin-2-one,
3-(1-{[3-chloro-6-(1-ethyl-1H-pyrazol-4-yl)-8-(trifluoromethyl)imidazo[1,2-a]pyridin-2-yl]carbonyl}-4-piperidinyl)-1,3-oxazolidin-2-one,
3-(1-{[3-chloro-6-[1-(2-methylpropyl)-1H-pyrazol-4-yl]-8-(trifluoromethyl)imidazo[1,2-a]pyridin-2-yl]carbonyl}-4-piperidinyl)-1,3-oxazolidin-2-one,
3-(1-{[3-chloro-6-cyclopentyl-8-(trifluoromethyl)imidazo[1,2-a]pyridin-2-yl]carbonyl}-4-piperidinyl)-1,3-oxazolidin-2-one,
3-(1-{[3-chloro-6-cyclobutyl-8-(trifluoromethyl)imidazo[1,2-a]pyridin-2-yl]carbonyl}-4-piperidinyl)-1,3-oxazolidin-2-one,
3-(1-{[3-chloro-6-propyl-8-(trifluoromethyl)imidazo[1,2-a]pyridin-2-yl]carbonyl}-4-piperidinyl)-1,3-oxazolidin-2-one,
3-(1-{[3-chloro-6-(2-methylpropyl)-8-(trifluoromethyl)imidazo[1,2-a]pyridin-2-yl]carbonyl}-4-piperidinyl)-1,3-oxazolidin-2-one,
3-(1-{[6-butyl-3-chloro-8-(trifluoromethyl)imidazo[1,2-a]pyridin-2-yl]carbonyl}-4-piperidinyl)-1,3-oxazolidin-2-one,
3-(1-{[3-chloro-6-(methyloxy)-8-(trifluoromethyl)imidazo[1,2-a]pyridin-2-yl]carbonyl}-4-piperidinyl)-1,3-oxazolidin-2-one,
3-(1-{[3-chloro-6-(5-methyl-2-furanyl)-8-(trifluoromethyl)imidazo[1,2-a]pyridin-2-yl]carbonyl}-4-piperidinyl)-1,3-oxazolidin-2-one,
3-(1-{[3-chloro-6-(tetrahydro-2-furanyl)-8-(trifluoromethyl)imidazo[1,2-a]pyridin-2-yl]carbonyl}-4-piperidinyl)-1,3-oxazolidin-2-one,
3-(1-{[3-chloro-6-(ethyloxy)-8-(trifluoromethyl)imidazo[1,2-a]pyridin-2-yl]carbonyl}-4-piperidinyl)-1,3-oxazolidin-2-one,
3-(1-{[3-chloro-6-(3-pyridinyl)-8-(trifluoromethyl)imidazo[1,2-a]pyridin-2-yl]carbonyl}-4-piperidinyl)-1,3-oxazolidin-2-one,
3-(1-{[3-chloro-6-(tetrahydro-3-furanyl)-8-(trifluoromethyl)imidazo[1,2-a]pyridin-2-yl]carbonyl}-4-piperidinyl)-1,3-oxazolidin-2-one,
3-(1-{[3-chloro-6-[(1-methylethyl)oxy]-8-(trifluoromethyl)imidazo[1,2-a]pyridin-2-yl]carbonyl}-4-piperidinyl)-1,3-oxazolidin-2-one,
3-(1-{[3-chloro-6-(1H-imidazol-1-yl)-8-(trifluoromethyl)imidazo[1,2-a]pyridin-2-yl]carbonyl}-4-piperidinyl)-1,3-oxazolidin-2-one,
3-(1-{[3-chloro-6-(2-methylpropyl)-8-(trifluoromethyl)imidazo[1,2-a]pyridin-2-yl]carbonyl}-4-piperidinyl)-1,3-oxazolidine-2,4-dione,
3-(1-{[3-chloro-6-(1-methylpropyl)-8-(trifluoromethyl)imidazo[1,2-a]pyridin-2-yl]carbonyl}-4-piperidinyl)-1,3-oxazolidin-2-one,
3-(1-{[6-bromo-3-chloro-8-(trifluoromethyl)imidazo[1,2-a]pyridin-2-yl]carbonyl}-4-piperidinyl)-1,3-oxazolidine-2,4-dione,
1-(1-{[6-bromo-3-chloro-8-(trifluoromethyl)imidazo[1,2-a]pyridin-2-yl]carbonyl}-4-piperidinyl)-2-pyrrolidinone,
3-(1-{[3-chloro-6-[(difluoromethyl)oxy]-8-(trifluoromethyl)imidazo[1,2-a]pyridin-2-yl]carbonyl}-4-piperidinyl)-1,3-oxazolidin-2-one,
3-(1-{[3-chloro-6-[(difluoromethyl)oxy]-8-(trifluoromethyl)imidazo[1,2-a]pyridin-2-yl]carbonyl}-4-piperidinyl)-1,3-oxazolidine-2,4-dione,
3-(1-{[3-chloro-6-(2-furanyl)-8-(trifluoromethyl)imidazo[1,2-a]pyridin-2-yl]carbonyl}-4-piperidinyl)-1,3-oxazolidin-2-one,
3-(1-{[3-chloro-6-(1H-pyrrol-3-yl)-8-(trifluoromethyl)imidazo[1,2-a]pyridin-2-yl]carbonyl}-4-piperidinyl)-1,3-oxazolidin-2-one,
3-(1-{[3-chloro-6-(2H-1,2,3-triazol-4-yl)-8-(trifluoromethyl)imidazo[1,2-a]pyridin-2-yl]carbonyl}-4-piperidinyl)-1,3-oxazolidin-2-one,
3-(1-{[3-chloro-6-(1H-imidazol-2-yl)-8-(trifluoromethyl)imidazo[1,2-a]pyridin-2-yl]carbonyl}-4-piperidinyl)-1,3-oxazolidin-2-one,
3-(1-{[3-chloro-6-(1H-pyrrol-2-yl)-8-(trifluoromethyl)imidazo[1,2-a]pyridin-2-yl]carbonyl}-4-piperidinyl)-1,3-oxazolidin-2-one,
3-(1-{[3-chloro-6-methyl-8-(trifluoromethyl)imidazo[1,2-a]pyridin-2-yl]carbonyl}-4-piperidinyl)-1,3-oxazolidin-2-one,
3-(1-{[3-chloro-6-ethyl-8-(trifluoromethyl)imidazo[1,2-a]pyridin-2-yl]carbonyl}-4-piperidinyl)-1,3-oxazolidin-2-one,
3-(1-{[3-chloro-6-ethenyl-8-(trifluoromethyl)imidazo[1,2-a]pyridin-2-yl]carbonyl}-4-piperidinyl)-1,3-oxazolidin-2-one,
3-(1-{[3-chloro-6-(1-methylethyl)-8-(trifluoromethyl)imidazo[1,2-a]pyridin-2-yl]carbonyl}-4-piperidinyl)-1,3-oxazolidin-2-one,
3-(1-{[3-chloro-6-(1-hydroxyethyl)-8-(trifluoromethyl)imidazo[1,2-a]pyridin-2-yl]carbonyl}-4-piperidinyl)-1,3-oxazolidin-2-one,
3-(1-{[3-chloro-6-cyclopropyl-8-(trifluoromethyl)imidazo[1,2-a]pyridin-2-yl]carbonyl}-4-piperidinyl)-1,3-oxazolidin-2-one,
3-(1-{[3-chloro-6-(difluoromethyl)-8-(trifluoromethyl)imidazo[1,2-a]pyridin-2-yl]carbonyl}-4-piperidinyl)-1,3-oxazolidin-2-one,
3-(1-{[3-bromo-6-(3-furanyl)-8-(trifluoromethyl)imidazo[1,2-a]pyridin-2-yl]carbonyl}-4-piperidinyl)-1,3-oxazolidin-2-one,
3-(1-{[3-bromo-6-(3-furanyl)-8-(trifluoromethyl)imidazo[1,2-a]pyridin-2-yl]carbonyl}-4-piperidinyl)-1,3-oxazolidine-2,4-dione,
6-(3-furanyl)-2-{[4-(2-oxo-1,3-oxazolidin-3-yl)-1-piperidinyl]carbonyl}-8-(trifluoromethyl)imidazo[1,2-a]pyridine-3-carbonitrile,
2-{[4-(2,4-dioxo-1,3-oxazolidin-3-yl)-1-piperidinyl]carbonyl}-6-(3-furanyl)-8-(trifluoromethyl)imidazo[1,2-a]pyridine-3-carbonitrile,
6-(3-furanyl)-2-{[4-(2-oxo-1,3-oxazolidin-3-yl)-1-piperidinyl]carbonyl}-8-(trifluoromethyl)imidazo[1,2-a]pyridine-3-carbonitrile,
1-(1-{[3-chloro-6-cyclopropyl-8-(trifluoromethyl)imidazo[1,2-a]pyridin-2-yl]carbonyl}-4-piperidinyl)-4-hydroxy-2-pyrrolidinone,
3-(1-{[6-(3-furanyl)-3-methyl-8-(trifluoromethyl)imidazo[1,2-a]pyridin-2-yl]carbonyl}-4-piperidinyl)-1,3-oxazolidin-2-one,
3-(1-{[6-(3-furanyl)-3-(1-methylethyl)-8-(trifluoromethyl)imidazo[1,2-a]pyridin-2-yl]carbonyl}-4-piperidinyl)-1,3-oxazolidin-2-one,
3-(1-{[6-(3-furanyl)-3-(hydroxymethyl)-8-(trifluoromethyl)imidazo[1,2-a]pyridin-2-yl]carbonyl}-4-piperidinyl)-1,3-oxazolidin-2-one,
1-(1-{[3-chloro-6-(1H-pyrazol-4-yl)-8-(trifluoromethyl)imidazo[1,2-a]pyridin-2-yl]carbonyl}-4-piperidinyl)-2,5-pyrrolidinedione,
1-(1-{[3-chloro-6-(3-furanyl)-8-(trifluoromethyl)imidazo[1,2-a]pyridin-2-yl]carbonyl}-4-piperidinyl)-2,5-pyrrolidinedione,
1-(1-{[3-chloro-6-(3-furanyl)-8-(trifluoromethyl)imidazo[1,2-a]pyridin-2-yl]carbonyl}-4-piperidinyl)-2-imidazolidinone,
1-(1-{[3-chloro-6-(1H-pyrazol-4-yl)-8-(trifluoromethyl)imidazo[1,2-a]pyridin-2-yl]carbonyl}-4-piperidinyl)-2-imidazolidinone,
1-(1-{[3-chloro-6-(3-furanyl)-8-(trifluoromethyl)imidazo[1,2-a]pyridin-2-yl]carbonyl}-4-piperidinyl)-2,4-imidazolidinedione,
1-(1-{[3-chloro-6-(1H-pyrazol-4-yl)-8-(trifluoromethyl)imidazo[1,2-a]pyridin-2-yl]carbonyl}-4-piperidinyl)-2,4-imidazolidinedione,
1-(1-{[3-chloro-6-(3-furanyl)-8-(trifluoromethyl)imidazo[1,2-a]pyridin-2-yl]carbonyl}-4-piperidinyl)-3-methyl-2-imidazolidinone,
3-(1-{[3-chloro-6-(1H-pyrazol-4-yl)-8-(trifluoromethyl)imidazo[1,2-a]pyridin-2-yl]carbonyl}-4-piperidinyl)-2,4-imidazolidinedione,
3-(1-{[3-chloro-6-(3-furanyl)-8-(trifluoromethyl)imidazo[1,2-a]pyridin-2-yl]carbonyl}-4-piperidinyl)-2,4-imidazolidinedione,
3-(1-{[3-chloro-6-(3-furanyl)-8-(trifluoromethyl)imidazo[1,2-a]pyridin-2-yl]carbonyl}-4-piperidinyl)-4,4-dimethyl-1,3-oxazolidin-2-one,
3-(1-{[3-chloro-6-(1H-pyrazol-4-yl)-8-(trifluoromethyl)imidazo[1,2-a]pyridin-2-yl]carbonyl}-4-piperidinyl)-4,4-dimethyl-1,3-oxazolidin-2-one,
3-(1-{[3-chloro-6-(3-furanyl)-8-(trifluoromethyl)imidazo[1,2-a]pyridin-2-yl]carbonyl}-4-piperidinyl)-1,3-oxazolidine-2,4-dione,
3-(1-{[3-chloro-6-(1H-pyrazol-4-yl)-8-(trifluoromethyl)imidazo[1,2-a]pyridin-2-yl]carbonyl}-4-piperidinyl)-1,3-oxazolidine-2,4-dione,
(4S)-3-(1-{[3-chloro-6-(3-furanyl)-8-(trifluoromethyl)imidazo[1,2-a]pyridin-2-yl]carbonyl}-4-piperidinyl)-4-methyl-1,3-oxazolidin-2-one,
3-(1-{[3-chloro-6-(3-furanyl)-8-(trifluoromethyl)imidazo[1,2-a]pyridin-2-yl]carbonyl}-4-piperidinyl)-5,5-dimethyl-1,3-oxazolidin-2-one,
(4R)-3-(1-{[3-chloro-6-(3-furanyl)-8-(trifluoromethyl)imidazo[1,2-a]pyridin-2-yl]carbonyl}-4-piperidinyl)-4-methyl-1,3-oxazolidin-2-one,
3-(1-{[3-chloro-6-(3-furanyl)-8-(trifluoromethyl)imidazo[1,2-a]pyridin-2-yl]carbonyl}-4-methyl-4-piperidinyl)-1,3-oxazolidin-2-one,
(5S)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidin-2-one,
(5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidin-2-one,
3-(1-{[3-chloro-6-(3-furanyl)-8-(trifluoromethyl)imidazo[1,2-a]pyridin-2-yl]carbonyl}-4-piperidinyl)-1,3-oxazol-2(3H)-one,
3-(1-{[6-amino-3-chloro-8-(trifluoromethyl)imidazo[1,2-a]pyridin-2-yl]carbonyl}-4-piperidinyl)-1,3-oxazolidin-2-one,
(5S)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione,
(5S)-3-(1-{[3-chloro-6-cyclopropyl-8-(trifluoromethyl)imidazo[1,2-a]pyridin-2-yl]carbonyl}-4-piperidinyl)-5-methyl-1,3-oxazolidine-2,4-dione,
(5S)-3-(1-{[6-bromo-3-chloro-8-(trifluoromethyl)imidazo[1,2-a]pyridin-2-yl]carbonyl}-4-piperidinyl)-5-methyl-1,3-oxazolidine-2,4-dione,
(5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione,
(5R)-3-(1-{[3-chloro-6-cyclopropyl-8-(trifluoromethyl)imidazo[1,2-a]pyridin-2-yl]carbonyl}-4-piperidinyl)-5-methyl-1,3-oxazolidine-2,4-dione,
(5R)-3-(1-{[6-bromo-3-chloro-8-(trifluoromethyl)imidazo[1,2-a]pyridin-2-yl]carbonyl}-4-piperidinyl)-5-methyl-1,3-oxazolidine-2,4-dione,
3-(1-{[3-bromo-6-iodo-8-(trifluoromethyl)imidazo[1,2-a]pyridin-2-yl]carbonyl}-4-piperidinyl)-1,3-oxazolidin-2-one,
3-(1-{[3-bromo-6-iodo-8-(trifluoromethyl)imidazo[1,2-a]pyridin-2-yl]carbonyl}-4-piperidinyl)-1,3-oxazolidine-2,4-dione,
3-(1-{[6-(1-methylethyl)-8-(trifluoromethyl)imidazo[1,2-a]pyridin-2-yl]carbonyl}-4-piperidinyl)-1,3-oxazolidin-2-one,
3-(1-{[3-bromo-6-cyclopropyl-8-(trifluoromethyl)imidazo[1,2-a]pyridin-2-yl]carbonyl}-4-piperidinyl)-1,3-oxazolidin-2-one,
N-[3-chloro-2-{[4-(2-oxo-1,3-oxazolidin-3-yl)-1-piperidinyl]carbonyl}-8-(trifluoromethyl)imidazo[1,2-a]pyridin-6-yl]acetamide,
4-(1-{[3-chloro-6-(3-furanyl)-8-(trifluoromethyl)imidazo[1,2-a]pyridin-2-yl]carbonyl}-4-piperidinyl)-2,4-dihydro-3H-1,2,4-triazol-3-one,
1-(1-{[3-chloro-6-cyclopropyl-8-(trifluoromethyl)imidazo[1,2-a]pyridin-2-yl]carbonyl}-4-piperidinyl)-2,5-pyrrolidinedione,
1-(1-{[3-chloro-6-cyclopropyl-8-(trifluoromethyl)imidazo[1,2-a]pyridin-2-yl]carbonyl}-4-piperidinyl)-2-pyrrolidinone,
3-(1-{[3-chloro-6-cyclopropyl-8-(trifluoromethyl)imidazo[1,2-a]pyridin-2-yl]carbonyl}-4-piperidinyl)-1,3-oxazolidine-2,4-dione,
3-(1-{[6-(3-furanyl)-8-(trifluoromethyl)imidazo[1,2-a]pyridin-2-yl]carbonyl}-4-piperidinyl)-1,3-oxazolidin-2-one,
3-(1-{[3-chloro-6-(3-furanyl)-8-(trifluoromethyl)imidazo[1,2-a]pyridin-2-yl]carbonyl}-3,3-dimethyl-4-piperidinyl)-1,3-oxazolidin-2-one,
(±) cis-3-(1-{[3-chloro-6-(3-furanyl)-8-(trifluoromethyl)imidazo[1,2-a]pyridin-2-yl]carbonyl}-2-methyl-4-piperidinyl)-1,3-oxazolidin-2-one,
(±) trans-3-(1-{[3-chloro-6-(3-furanyl)-8-(trifluoromethyl)imidazo[1,2-a]pyridin-2-yl]carbonyl}-2-methyl-4-piperidinyl)-1,3-oxazolidin-2-one,
3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 1),
3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 2),
3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 3),
3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 4),
3-(1-{[3-chloro-6-(3-furanyl)-8-(trifluoromethyl)imidazo[1,2-a]pyridin-2-yl]carbonyl}-2,2-dimethyl-4-piperidinyl)-1,3-oxazolidin-2-one,
(±)-trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one,
(±)-cis-3-(1-{[3-chloro-6-(3-furanyl)-8-(trifluoromethyl)imidazo[1,2-a]pyridin-2-yl]carbonyl}-3-fluoro-4-piperidinyl)-1,3-oxazolidin-2-one,
(±)-trans-3(1-{[3-chloro-6-(3-furanyl)-8-(trifluoromethyl)imidazo[1,2-a]pyridin-2-yl]carbonyl}-3-fluoro-4-piperidinyl)-1,3-oxazolidin-2-one,
(±) trans-3-(1-{[3-chloro-6-(2-methylpropyl)-8-(trifluoromethyl)imidazo[1,2-a]pyridin-2-yl]carbonyl}-3-hydroxy-4-piperidinyl)-1,3-oxazolidin-2-one,
(±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one,
3-((trans)-1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one (enantiomer 1),
3-((trans)-1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one (enantiomer 2),
(±) trans-3-(1-{[6-bromo-3-chloro-8-(trifluoromethyl)imidazo[1,2-a]pyridin-2-yl]carbonyl}-3-hydroxy-4-piperidinyl)-1,3-oxazolidin-2-one,
3-((trans)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 1),
3-((trans)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 2),
(±) trans-3-(1-{[6-bromo-3-chloro-8-(trifluoromethyl)imidazo[1,2-a]pyridin-2-yl]carbonyl}-3-hydroxy-4-piperidinyl)-1,3-oxazolidine-2,4-dione,
(±) trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidine-2,4-dione,
(±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidine-2,4-dione,
(±) trans-1-(1-{[3-chloro-6-cyclopropyl-8-(trifluoromethyl)imidazo[1,2-a]pyridin-2-yl]carbonyl}-3-hydroxy-4-piperidinyl)-2-pyrrolidinone,
(±) trans-1-(1-{[6-bromo-3-chloro-8-(trifluoromethyl)imidazo[1,2-a]pyridin-2-yl]carbonyl}-3-hydroxy-4-piperidinyl)-2-pyrrolidinone,
(±) trans-1-(1-{[3-chloro-6-(3-furanyl)-8-(trifluoromethyl)imidazo[1,2-a]pyridin-2-yl]carbonyl}-3-hydroxy-4-piperidinyl)-2-pyrrolidinone piperidinyl,
(±) cis-1-(1-{[3-chloro-6-cyclopropyl-8-(trifluoromethyl)imidazo[1,2-a]pyridin-2-yl]carbonyl}-3-hydroxy-4-)-2-pyrrolidinone,
(±) cis-1-(1-{[6-bromo-3-chloro-8-(trifluoromethyl)imidazo[1,2-a]pyridin-2-yl]carbonyl}-3-hydroxy-4-piperidinyl)-2-pyrrolidinone,
(±) cis-1-(1-{[3-chloro-6-(3-furanyl)-8-(trifluoromethyl)imidazo[1,2-a]pyridin-2-yl]carbonyl}-3-hydroxy-4-piperidinyl)-2-pyrrolidinone,
(±) cis-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one,
1-{[3-chloro-6-cyclopropyl-8-(trifluoromethyl)imidazo[1,2-a]pyridin-2-yl]carbonyl}-4-(2-oxo-1,3-oxazolidin-3-yl)-3-piperidinone,
(±) cis-3-(1-{[6-bromo-3-chloro-8-(trifluoromethyl)imidazo[1,2-a]pyridin-2-yl]carbonyl}-3-hydroxy-4-piperidinyl)-1,3-oxazolidin-2-one,
3-((cis)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 1),
3-((cis)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 2),
(±) trans-3-[1-{[3-chloro-6-(3-furanyl)-8-(trifluoromethyl)imidazo[1,2-a]pyridin-2-yl]carbonyl}-3-(methyloxy)-4-piperidinyl]-1,3-oxazolidin-2-one,
(±) trans-3-[1-{[6-bromo-3-chloro-8-(trifluoromethyl)imidazo[1,2-a]pyridin-2-yl]carbonyl}-3-(methyloxy)-4-piperidinyl]-1,3-oxazolidin-2-one,
(±) trans-3-[1-{[3-chloro-6-cyclopropyl-8-(trifluoromethyl)imidazo[1,2-a]pyridin-2-yl]carbonyl}-3-(methyloxy)-4-piperidinyl]-1,3-oxazolidin-2-one, and
3-chloro-2-{[4-(2-oxo-1,3-oxazolidin-3-yl)-1-piperidinyl]carbonyl}-8-(trifluoromethyl)imidazo[1,2-a]pyridine-6-carbonitrile, or a pharmaceutically acceptable salt thereof.

TABLE 1

| Ex | Compound | Structure | Name |
|---|---|---|---|
| 1 | 101 | 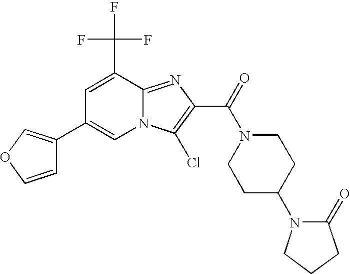 | 1-(1-{[3-chloro-6-(3-furanyl)-8- (trifluoromethyl)imidazo[1,2- a]pyridin-2-yl]carbonyl}-4- piperidinyl)-2-pyrrolidinone |
| 2 | 102 | 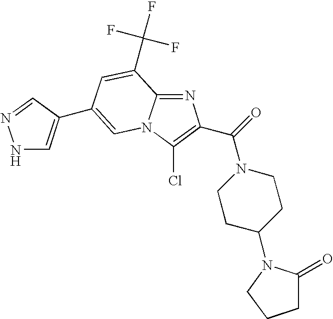 | 1-(1-{[3-chloro-6-(1H-pyrazol-4- yl)-8- (trifluoromethyl)imidazo[1,2- a]pyridin-2-yl]carbonyl}-4- piperidinyl)-2-pyrrolidinone |
| 3 | 103 | 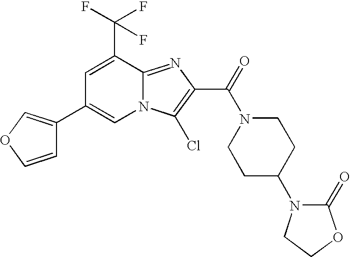 | 3-(1-{[3-chloro-6-(3-furanyl)-8- (trifluoromethyl)imidazo[1,2- a]pyridin-2-yl]carbonyl}-4- piperidinyl)-1,3-oxazolidin-2-one |
| 4 | 104 | 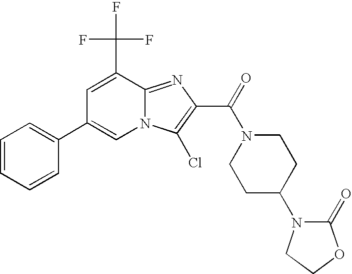 | 3-(1-{[3-chloro-6-phenyl-8- (trifluoromethyl)imidazo[1,2- a]pyridin-2-yl]carbonyl}-4- piperidinyl)-1,3-oxazolidin-2-one |
| 4 | 105 | 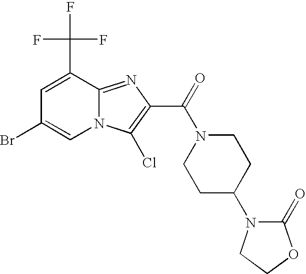 | 3-(1-{[6-bromo-3-chloro-8- (trifluoromethyl)imidazo[1,2- a]pyridin-2-yl]carbonyl}-4- piperidinyl)-1,3-oxazolidin-2-one |
| 5 | 106 | 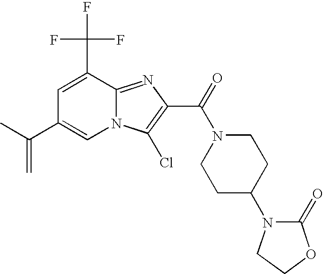 | 3-(1-{[3-chloro-6-(1- methylethenyl)-8- (trifluoromethyl)imidazo[1,2- a]pyridin-2-yl]carbonyl}-4- piperidinyl)-1,3-oxazolidin-2-one |
| 6 | 107 | 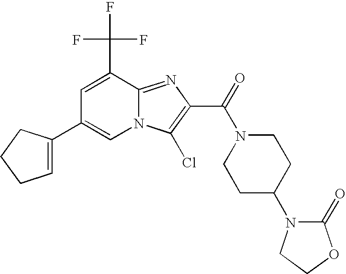 | 3-(1-{[3-chloro-6-(1-cyclopenten- 1-yl)-8- (trifluoromethyl)imidazo[1,2- a]pyridin-2-yl]carbonyl}-4- piperidinyl)-1,3-oxazolidin-2-one |
| 7 | 108 | 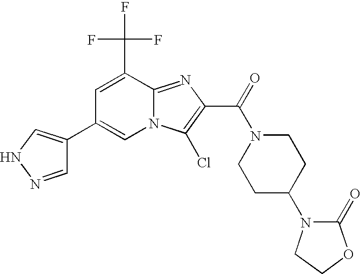 | 3-(1-{[3-chloro-6-(1H-pyrazol-4- yl)-8- (trifluoromethyl)imidazo[1,2- a]pyridin-2-yl]carbonyl}-4- piperidinyl)-1,3-oxazolidin-2-one |
| 8 | 109 | 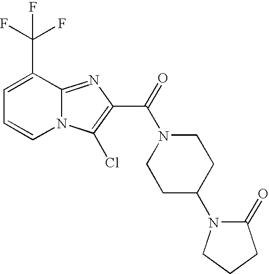 | 1-(1-{[3-chloro-8- (trifluoromethyl)imidazo[1,2- a]pyridin-2-yl]carbonyl}-4- piperidinyl)-2-pyrrolidinone |
| 9 | 110 | 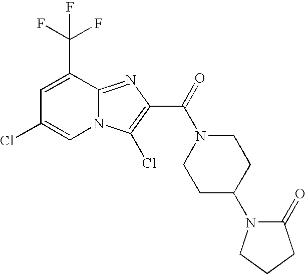 | 1-(1-{[3,6-dichloro-8- (trifluoromethyl)imidazo[1,2- a]pyridin-2-yl]carbonyl}-4- piperidinyl)-2-pyrrolidinone |
| 10 | 111 | 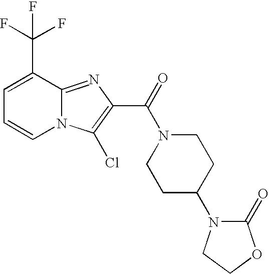 | 1-(1-{[3-chloro-8- (trifluoromethyl)imidazo[1,2- a]pyridin-2-yl]carbonyl}-4- piperidinyl)-2-pyrrolidinone |
| 11 | 112 | 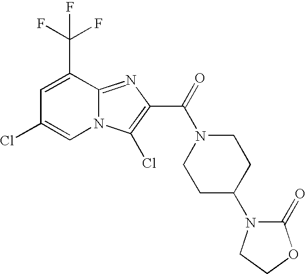 | 3-(1-{[3,6-dichloro-8- (trifluoromethyl)imidazo[1,2- a]pyridin-2-yl]carbonyl}-4- piperidinyl)-1,3-oxazolidin-2-one |
| 12 | 113 | 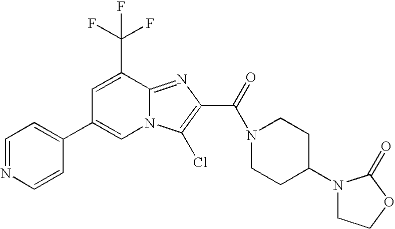 | 3-(1-{[3-chloro-6-(4-pyridinyl)-8- (trifluoromethyl)imidazo[1,2- a]pyridin-2-yl]carbonyl}-4- piperidinyl)-1,3-oxazolidin-2-one |
| 13 | 114 | 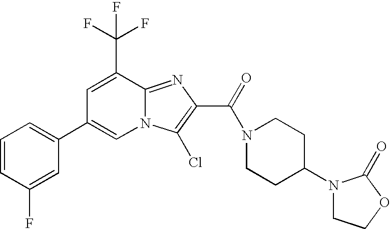 | 3-(1-{[3-chloro-6-(3- fluorophenyl)-8- (trifluoromethyl)imidazo[1,2- a]pyridin-2-yl]carbonyl}-4- piperidinyl)-1,3-oxazolidin-2-one |
| 14 | 115 | 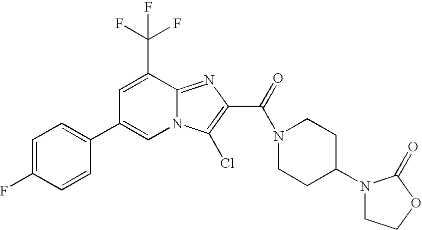 | 3-(1-{[3-chloro-6-(4- fluorophenyl)-8- (trifluoromethyl)imidazo[1,2- a]pyridin-2-yl]carbonyl}-4- piperidinyl)-1,3-oxazolidin-2-one |
| 15 | 116 | 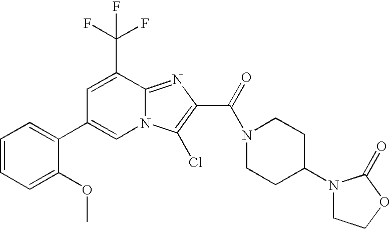 | 3-(1-{[3-chloro-6-[2- (methyloxy)phenyl]-8- (trifluoromethyl)imidazo[1,2- a]pyridin-2-yl]carbonyl}-4- piperidinyl)-1,3-oxazolidin-2-one |
| 16 | 117 | 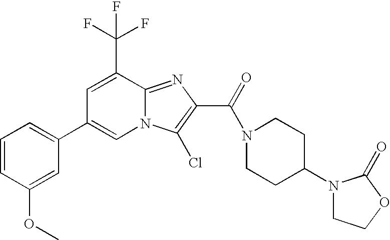 | 3-(1-{[3-chloro-6-[3- (methyloxy)phenyl]-8- (trifluoromethyl)imidazo[1,2- a]pyridin-2-yl]carbonyl}-4- piperidinyl)-1,3-oxazolidin-2-one |
| 17 | 118 | 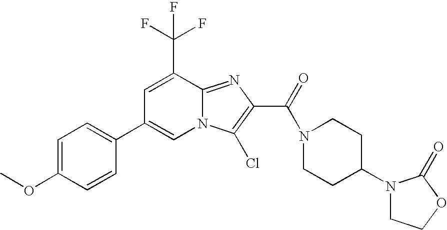 | 3-(1-{[3-chloro-6-[4- (methyloxy)phenyl]-8- (trifluoromethyl)imidazo[1,2- a]pyridin-2-yl]carbonyl}-4- piperidinyl)-1,3-oxazolidin-2-one |
| 18 | 119 | 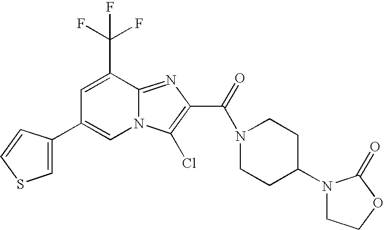 | 3-(1-{[3-chloro-6-(3-thienyl)-8- (trifluoromethyl)imidazo[1,2- a]pyridin-2-yl]carbonyl}-4- piperidinyl)-1,3-oxazolidin-2-one |
| 19 | 120 | 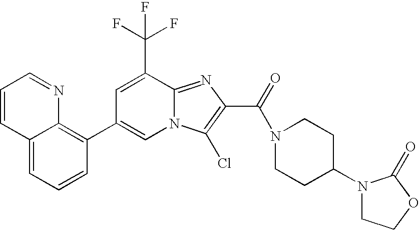 | 3-(1-{[3-chloro-6-(8-quinolinyl)-8- (trifluoromethyl)imidazo[1,2- a]pyridin-2-yl]carbonyl}-4- piperidinyl)-1,3-oxazolidin-2-one |
| 20 | 121 | 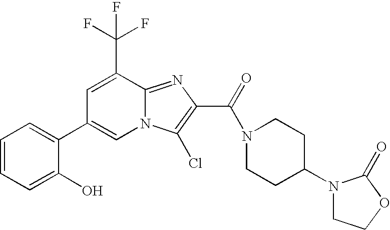 | 3-(1-{[3-chloro-6-(2- hydroxyphenyl)-8- (trifluoromethyl)imidazo[1,2- a]pyridin-2-yl]carbonyl}-4- piperidinyl)-1,3-oxazolidin-2-one |
| 21 | 122 | 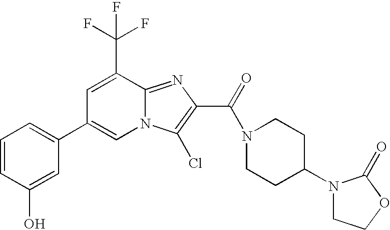 | 3-(1-{[3-chloro-6-(3- hydroxyphenyl)-8- (trifluoromethyl)imidazo[1,2- a]pyridin-2-yl]carbonyl}-4- piperidinyl)-1,3-oxazolidin-2-one |
| 22 | 123 | 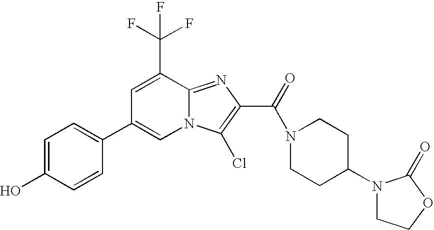 | 3-(1-{[3-chloro-6-(4- hydroxyphenyl)-8- (trifluoromethyl)imidazo[1,2- a]pyridin-2-yl]carbonyl}-4- piperidinyl)-1,3-oxazolidin-2-one |
| 23 | 124 | 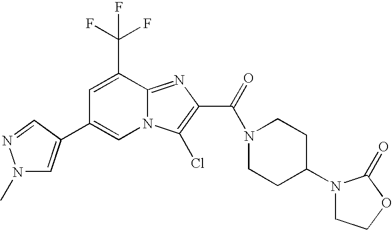 | 3-(1-{[3-chloro-6-(1-methyl-1H- pyrazol-4-yl)-8- (trifluoromethyl)imidazo[1,2- a]pyridin-2-yl]carbonyl}-4- piperidinyl)-1,3-oxazolidin-2-on |
| 24 | 125 | 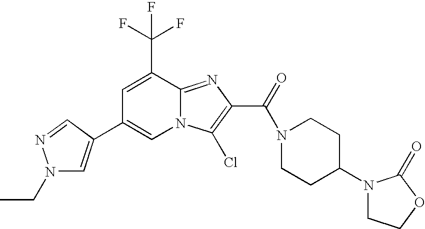 | 3-(1-{[3-chloro-6-(1-ethyl-1H- pyrazol-4-yl)-8- (trifluoromethyl)imidazo[1,2- a]pyridin-2-yl]carbonyl}-4- piperidinyl)-1,3-oxazolidin-2-one |
| 25 | 126 | 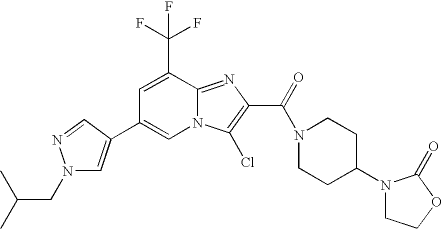 | 3-(1-{[3-chloro-6-[1-(2- methylpropyl-1H-pyrazol-4-yl]- 8-(trifluoromethyl)imidazo[1,2- a]pyridin-2-yl]carbonyl}-4- piperidinyl)-1,3-oxazolidin-2-one |
| 26 | 127 | 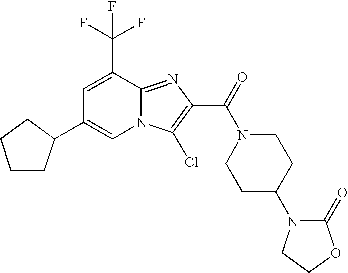 | 3-(1-{[3-chloro-6-cyclopentyl-8- (trifluoromethyl)imidazo[1,2- a]pyridin-2-yl]carbonyl}-4- piperidinyl)-1,3-oxazolidin-2-on |
| 27 | 128 | 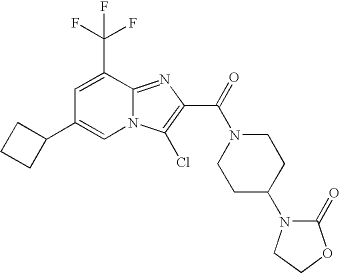 | 3-(1-{[3-chloro-6-cyclobutyl-8- (trifluoromethyl)imidazo[1,2- a]pyridin-2-yl]carbonyl}-4- piperidinyl)-1,3-oxazolidin-2-one |
| 28 | 129 | 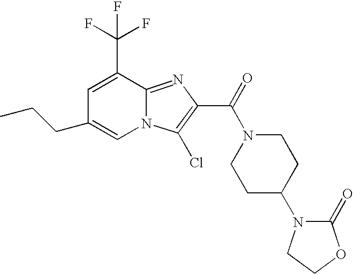 | 3-(1-{[3-chloro-6-propyl-8- (trifluoromethyl)imidazo[1,2- a]pyridin-2-yl]carbonyl}-4- piperidinyl)-1,3-oxazolidin-2-one |
| 29 | 130 | 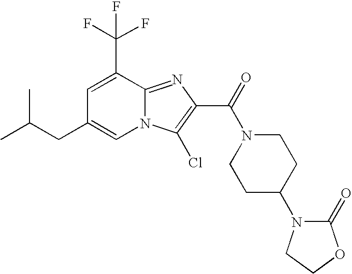 | 3-(1-{[3-chloro-6-(2- methylpropyl)-8- (trifluoromethyl)imidazo[1,2- a]pyridin-2-yl]carbonyl}-4- piperidinyl)-1,3-oxazolidin-2-one |
| 30 | 131 | 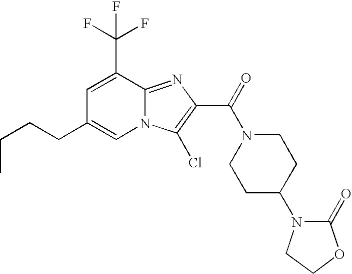 | 3-(1-{[6-butyl-3-chloro-8- (trifluoromethyl)imidazo[1,2- a]pyridin-2-yl]carbonyl}-4- piperidinyl)-1,3-oxazolidin-2-one |
| 31 | 132 | 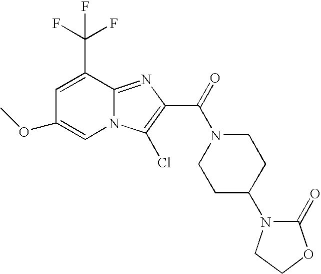 | 3-(1-{[3-chloro-6-(methyloxy)-8- (trifluoromethyl)imidazo[1,2- a]pyridin-2-yl]carbonyl}-4- piperidinyl)-1,3-oxazolidin-2-one |
| 32 | 133 | 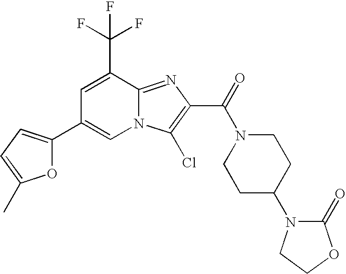 | 3-(1-{[3-chloro-6-(5-methyl-2- furanyl)-8- (trifluoromethyl)imidazo[1,2- a]pyridin-2-yl]carbonyl}-4- piperidinyl)-1,3-oxazolidin-2-one |
| 33 | 134 | 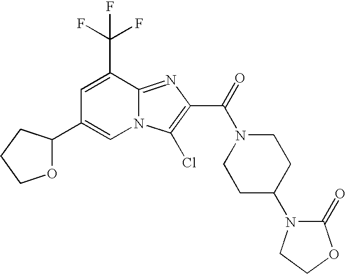 | 3-(1-{[3-chloro-6-(tetrahydro-2- furanyl)-8- (trifluoromethyl)imidazo[1,2- a]pyridin-2-yl]carbonyl}-4- piperidinyl)-1,3-oxazolidin-2-one |
| 34 | 135 | 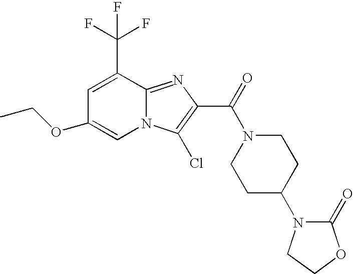 | 3-(1-{[3-chloro-6-(ethyloxy)-8- (trifluoromethyl)imidazo[1,2- a]pyridin-2-yl]carbonyl}-4- piperidinyl)-1,3-oxazolidin-2-one |
| 35 | 136 | 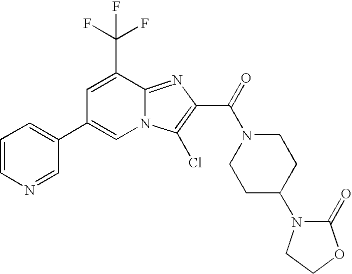 | 3-(1-{[3-chloro-6-(3-pyridinyl)-8- (trifluoromethyl)imidazo[1,2- a]pyridin-2-yl]carbonyl}-4- piperidinyl)-1,3-oxazolidin-2-one |
| 36 | 137 | 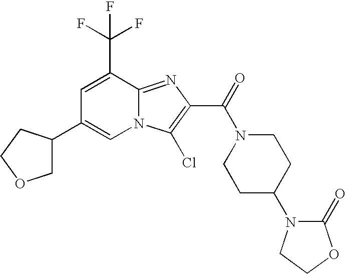 | 3-(1-{[3-chloro-6-(tetrahydro-3- furanyl)-8- (trifluoromethyl)imidazo[1,2- a]pyridin-2-yl]carbonyl}-4- piperidinyl)-1,3-oxazolidin-2-one |
| 37 | 138 | 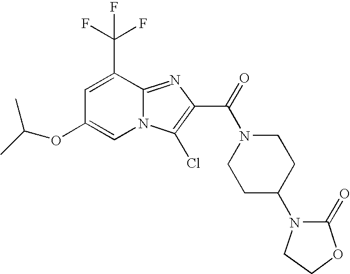 | 3-(1-{[3-chloro-6-[(1- methylethyl)oxy]-8- (trifluoromethyl)imidazo[1,2- a]pyridin-2-yl]carbonyl}-4- piperidinyl)-1,3-oxazolidin-2-one |
| 38 | 139 | 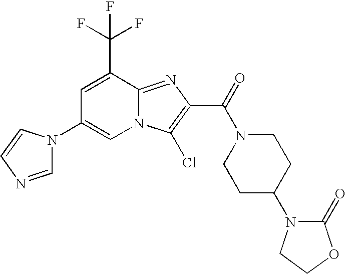 | 3-(1-{[3-chloro-6-(1H-imidazol-1- yl)-8- (trifluoromethyl)imidazo[1,2- a]pyridin-2-yl]carbonyl}-4- piperidinyl)-1,3-oxazolidin-2-one |
| 39 | 140 | 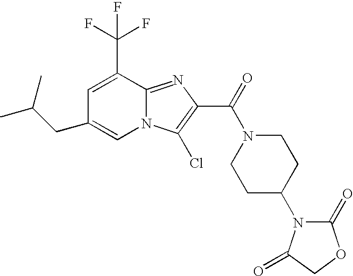 | 3-(1-{[3-chloro-6-(2- methylpropyl)-8- (trifluoromethyl)imidazo[1,2- a]pyridin-2-yl]carbonyl}-4- piperidinyl)-1,3-oxazolidine-2,4- dione |
| 40 | 141 | 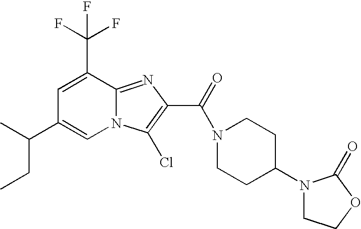 | 3-(1-{[3-chloro-6-(1- methylpropyl)-8- (trifluoromethyl)imidazo[1,2- a]pyridin-2-yl]carbonyl}-4- piperidinyl)-1,3-oxazolidin-2-one |
| 41 | 142 | 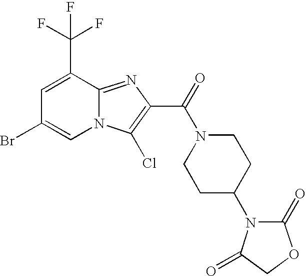 | 3-(1-{[6-bromo-3-chloro-8- (trifluoromethyl)imidazo[1,2- a]pyridin-2-yl]carbonyl}-4- piperidinyl)-1,3-oxazolidine-2,4- dione |
| 42 | 143 | 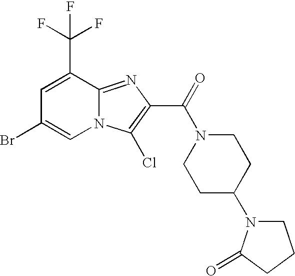 | 1-(1-{[6-bromo-3-chloro-8- (trifluoromethyl)imidazo[1,2- a]pyridin-2-yl]carbonyl}-4- piperidinyl)-2-pyrrolidinone |
| 43 | 144 | 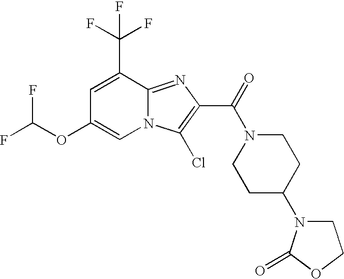 | 3-(1-{[3-chloro-6- [(difluoromethyl)oxy]-8- (trifluoromethyl)imidazo[1,2- a]pyridin-2-yl]carbonyl}-4- piperidinyl)-1,3-oxazolidin-2-one |
| 44 | 145 | 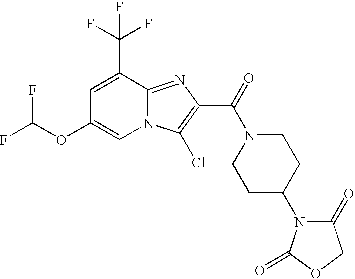 | 3-(1-{[3-chloro-6- [(difluoromethyl)oxy]-8- (trifluoromethyl)imidazo[1,2- a]pyridin-2-yl]carbonyl}-4- piperidinyl)-1,3-oxazolidine-2,4- dione |
| 45 | 146 | 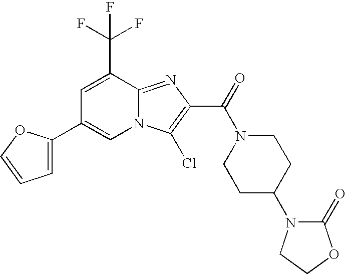 | 3-(1-{[3-chloro-6-(2-furanyl)-8- (trifluoromethyl)imidazo[1,2- a]pyridin-2-yl]carbonyl}-4- piperidinyl)-1,3-oxazolidin-2-one |
| 46 | 147 | 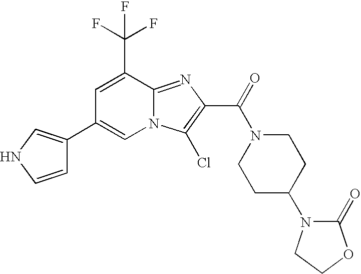 | 3-(1-{[3-chloro-6-(1H-pyrrol-3- yl)-8- (trifluoromethyl)imidazo[1,2- a]pyridin-2-yl]carbonyl}-4- piperidinyl)-1,3-oxazolidin-2-one |
| 47 | 148 | 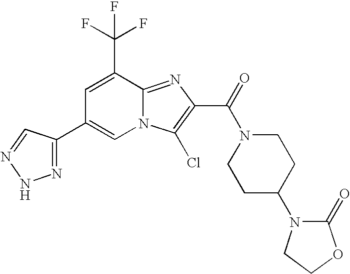 | 3-(1-{[3-chloro-6-(2H-1,2,3- triazol-4-yl)-8- (trifluoromethyl)imidazo[1,2- a]pyridin-2-yl]carbonyl}-4- piperidinyl)-1,3-oxazolidin-2-one |
| 48 | 149 | 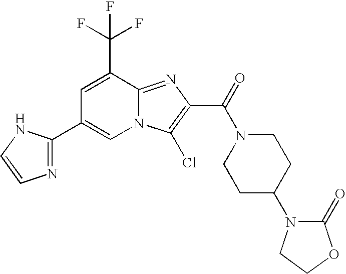 | 3-(1-{[3-chloro-6-(1H-imidazol-2- yl)-8- (trifluoromethyl)imidazo[1,2- a]pyridin-2-yl]carbonyl}-4- piperidinyl)-1,3-oxazolidin-2-one |
| 49 | 150 | 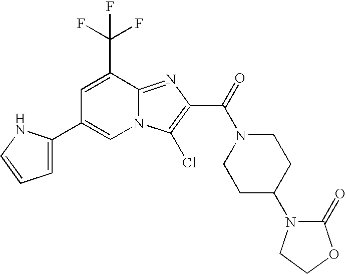 | 3-(1-{[3-chloro-6-(1H-pyrrol-3- yl)-8- (trifluoromethyl)imidazo[1,2- a]pyridin-2-yl]carbonyl}-4- piperidinyl)-1,3-oxazolidin-2-one |
| 50 | 151 | 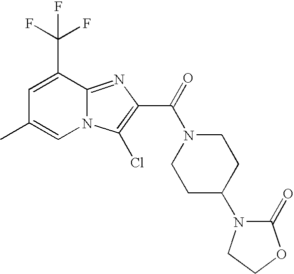 | 3-(1-{[3-chloro-6-methyl-8- (trifluoromethyl)imidazo[1,2- a]pyridin-2-yl]carbonyl}-4- piperidinyl)-1,3-oxazolidin-2-one |
| 51 | 152 | 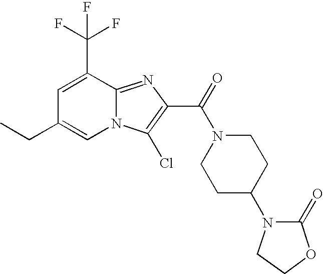 | 3-(1-{[3-chloro-6-ethyl-8- (trifluoromethyl)imidazo[1,2- a]pyridin-2-yl]carbonyl}-4- piperidinyl)-1,3-oxazolidin-2-one |
| 52 | 153 | 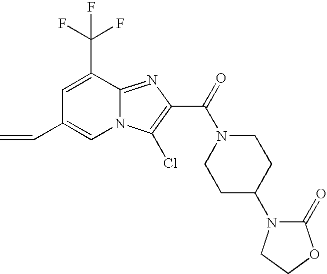 | 3-(1-{[3-chloro-6-ethenyl-8- (trifluoromethyl)imidazo[1,2- a]pyridin-2-yl]carbonyl}-4- piperidinyl)-1,3-oxazolidin-2-one |
| 53 | 154 | 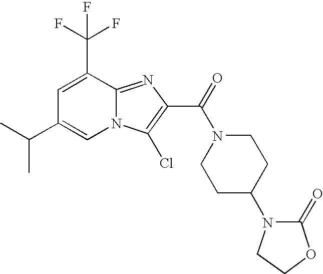 | 3-(1-{[3-chloro-6-(1-methylethyl)- 8-(trifluoromethyl)imidazo[1,2- a]pyridin-2-yl]carbonyl}-4- piperidinyl)-1,3-oxazolidin-2-one |
| 54 | 155 | 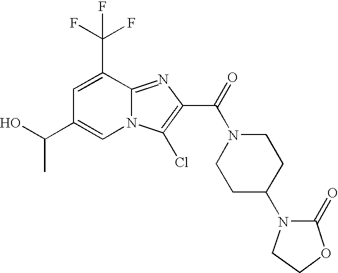 | 3-(1-{[3-chloro-6-(1- hydroxyethyl)-8- (trifluoromethyl)imidazo[1,2- a]pyridin-2-yl]carbonyl}-4- piperidinyl)-1,3-oxazolidin-2-one |
| 55 | 156 | 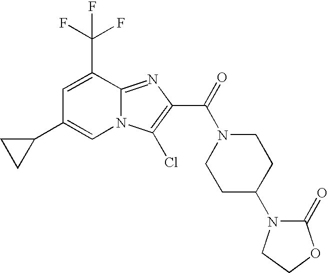 | 3-(1-{[3-chloro-6-cyclopropyl-8- (trifluoromethyl)imidazo[1,2- a]pyridin-2-yl]carbonyl}-4- piperidinyl)-1,3-oxazolidin-2-one |
| 56 | 157 | 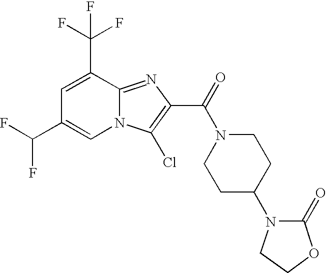 | 3-(1-{[3-chloro-6- (difluoromethyl)-8- (trifluoromethyl)imidazo[1,2- a]pyridin-2-yl]carbonyl}-4- piperidinyl)-1,3-oxazolidin-2-one |
| 57 | 158 | 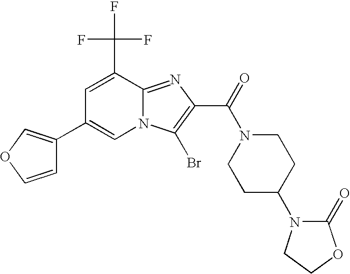 | 3-(1-{[3-bromo-6-(3-furanyl)-8- (trifluoromethyl)imidazo[1,2- a]pyridin-2-yl]carbonyl}-4- piperidinyl)-1,3-oxazolidin-2-one |
| 58 | 159 | 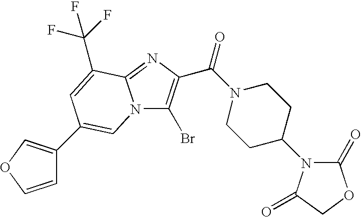 | 3-(1-{[3-bromo-6-(3-furanyl)-8- (trifluoromethyl)imidazo[1,2- a]pyridin-2-yl]carbonyl}-4- piperidinyl)-1,3-oxazolidine-2,4- dione |
| 59 | 160 | 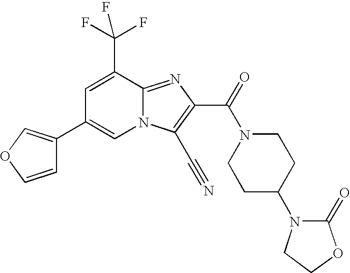 | 6-(3-furanyl)-2-{[4-(2-oxo-1,3- oxazolidin-3-yl)-1- piperidinyl]carbonyl}-8- (trifluoromethyl)imidazo[1,2- a]pyridine-3-carbonitrile |
| 60 | 161 | 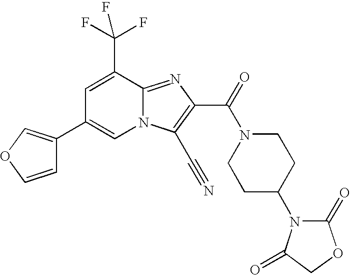 | 2-{[4-(2,4-dioxo-1,3-oxazolidin-3- yl)-1-piperidinyl]carbonyl}-6-(3- furanyl)-8- (trifluoromethyl)imidazo[1,2- a]pyridine-3-carbonitrile |
| 61 | 162 | 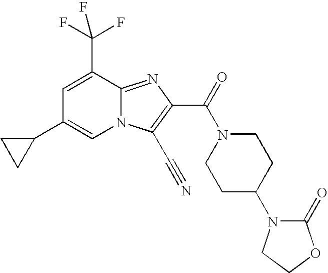 | 6-(3-furanyl)-2-{[4-(2-oxo-1,3- oxazolidin-3-yl)-1- piperidinyl]carbonyl}-8- (trifluoromethyl)imidazo[1,2- a]pyridine-3-carbonitrile |
| 62 | 163 | 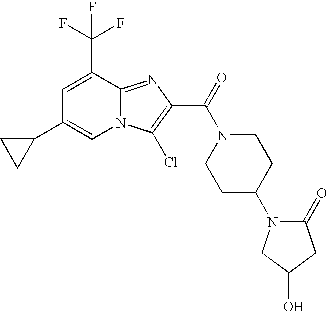 | 1-(1-{[3-chloro-6-cyclopropyl-8- (trifluoromethyl)imidazo[1,2- a]pyridin-2-yl]carbonyl}-4- piperidinyl)-4-hydroxy-2- pyrrolidinone |
| 63 | 164 | 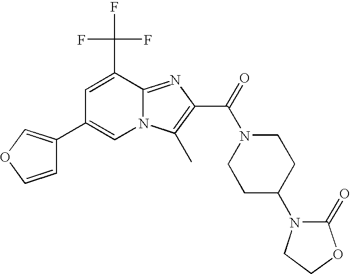 | 3-(1-{[6-(3-furanyl)-3-methyl-8- (trifluoromethyl)imidazo[1,2- a]pyridin-2-yl]carbonyl}-4- piperidinyl)-1,3-oxazolidin-2-one |
| 64 | 165 | 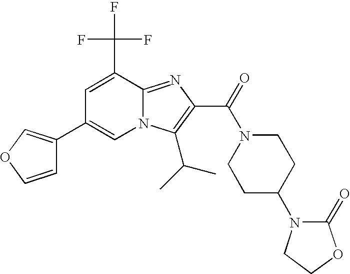 | 3-(1-{[6-(3-furanyl)-3-(1- methylethyl)-8- (trifluoromethyl)imidazo[1,2- a]pyridin-2-yl]carbonyl}-4- piperidinyl)-1,3-oxazolidin-2-one |
| 65 | 166 | 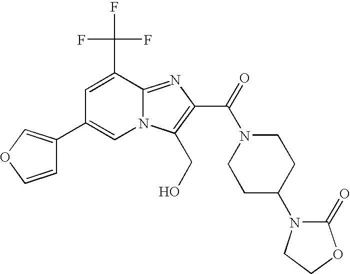 | 3-(1-{[6-(3-furanyl)-3- (hydroxymethyl)-8- (trifluoromethyl)imidazo[1,2- a]pyridin-2-yl]carbonyl}-4- piperidinyl)-1,3-oxazolidin-2-one |
| 66 | 167 | 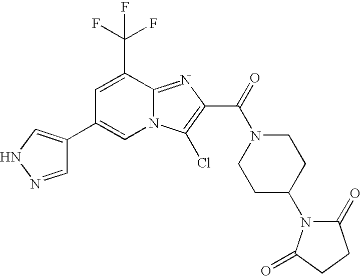 | 1-(1-{[3-chloro-6-(1H-pyrazol-4- yl)-8- (trifluoromethyl)imidazo[1,2- a]pyridin-2-yl]carbonyl}-4- piperidinyl)-2,5-pyrrolidinedione |
| 67 | 168 | 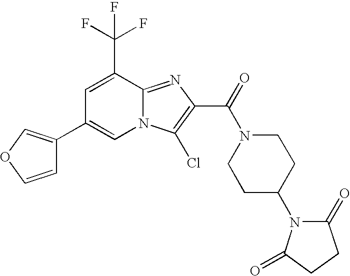 | 1-(1-{[3-chloro-6-(3-furanyl)-8- (trifluoromethyl)imidazo[1,2- a]pyridin-2-yl]carbonyl}-4- piperidinyl)-2,5-pyrrolidinedione |
| 68 | 169 | 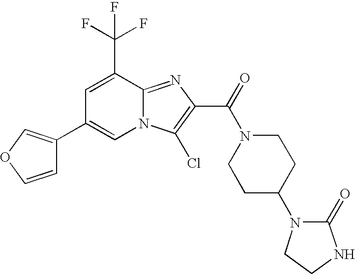 | 1-(1-{[3-chloro-6-(3-furanyl-8- (trifluoromethyl)imidazo[1,2- a]pyridin-2-yl]carbonyl}-4- piperidinyl)-2-imidazolidinone |
| 69 | 170 | 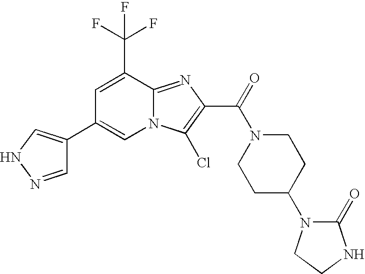 | 1-(1-{[3-chloro-6-(1H-pyrazol-4- yl)-8- (trifluoromethyl)imidazo[1,2- a]pyridin-2-yl]carbonyl}-4- piperidinyl)-2-imidazolidinone |
| 70 | 171 | 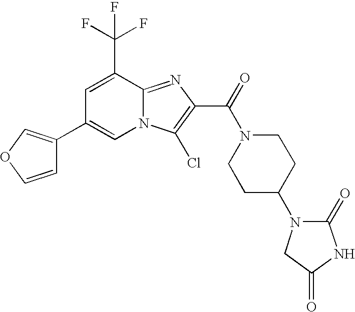 | 1-(1-{[3-chloro-6-(3-furanyl)-8- (trifluoromethyl)imidazo[1,2- a]pyridin-2-yl]carbonyl}-4- piperidinyl)-2,4- imidazolidinedione |
| 71 | 172 | 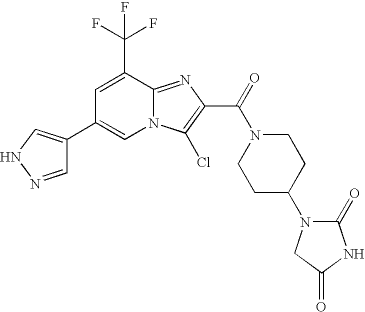 | 1-(1-{[3-chloro-6-(1H-pyrazol-4- yl)-8- (trifluoromethyl)imidazo[1,2- a]pyridin-2-yl]carbonyl}-4- piperidinyl)-2,4- imidazolidinedione |
| 72 | 173 | 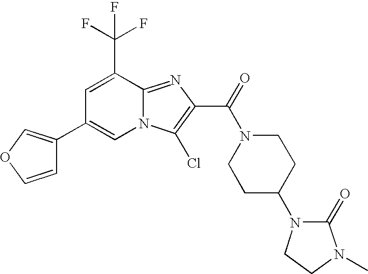 | 1-(1-{[3-chloro-6-(3-furanyl)-8- (trifluoromethyl)imidazo[1,2- a]pyridin-2-yl]carbonyl}-4- piperidinyl)-3-methyl-2- imidazolidinone |
| 73 | 174 | 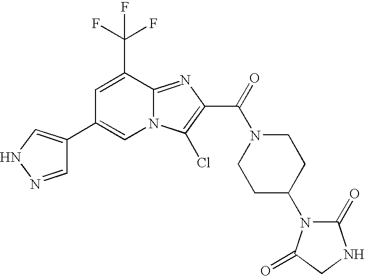 | 3-(1-{[3-chloro-6-(1H-pyrazol-4- yl)-8- (trifluoromethyl)imidazo[1,2- a]pyridin-2-yl]carbonyl}-4- piperidinyl)-2,4- imidazolidinedione |
| 74 | 175 | 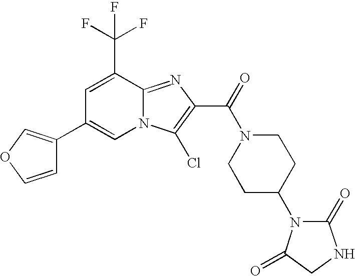 | 3-(1-{[3-chloro-6-(3-furanyl)-8- (trifluoromethyl)imidazo[1,2- a]pyridin-2-yl]carbonyl}-4- piperidinyl)-2,4- imidazolidinedione |
| 75 | 176 | 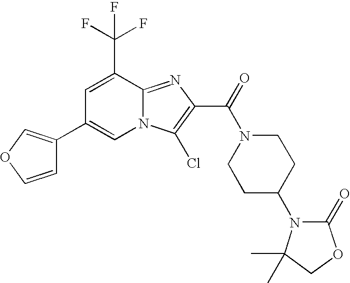 | 3-(1-{[3-chloro-6-(3-furanyl)-8- (trifluoromethyl)imidazo[1,2- a]pyridin-2-yl]carbonyl}-4- piperidinyl)-4,4-dimethyl-1,3- oxazolidin-2-one |
| 76 | 177 | 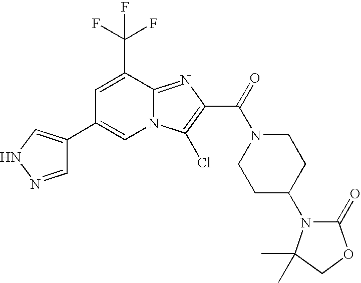 | 3-(1-{[3-chloro-6-(1H-pyrazol-4- yl)-8- (trifluoromethyl)imidazo[1,2- a]pyridin-2-yl]carbonyl}-4- piperidinyl)-4,4-dimethyl-1,3- oxazolidin-2-one |
| 77 | 178 | 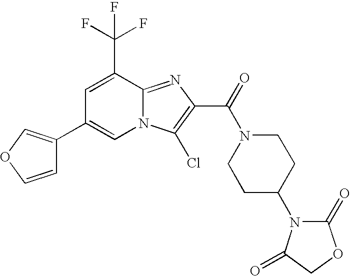 | 3-(1-{[3-chloro-6-(3-furanyl)-8- (trifluoromethyl)imidazo[1,2- a]pyridin-2-yl]carbonyl}-4- piperidinyl)-1,3-oxazolidine-2,4- dione |
| 78 | 179 | 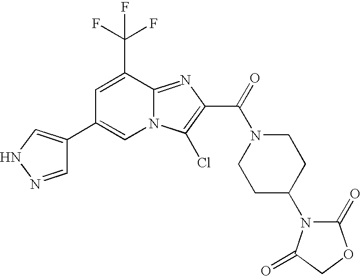 | 3-(1-{[3-chloro-6-(1H-pyrazol-4- yl)-8- (trifluoromethyl)imidazo[1,2- a]pyridin-2-yl]carbonyl}-4- piperidinyl)-1,3-oxazolidine-2,4- dione |
| 79 | 180 | 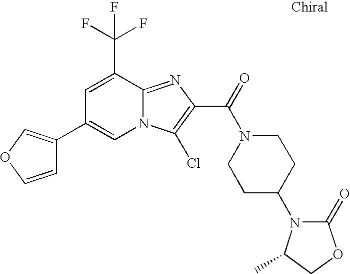 | (4S)-3-(1-{[3-chloro-6-(3- furanyl)-8- (trifluoromethyl)imidazo[1,2- a]pyridin-2-yl]carbonyl}-4- piperidinyl)-4-methyl-1,3- oxazolidin-2-one |
| 80 | 181 | 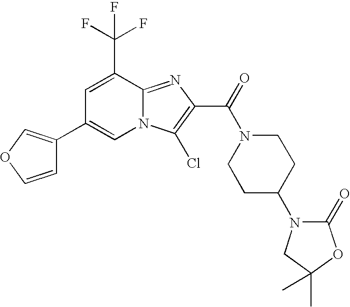 | 3-(1-{[3-chloro-6-(3-furanyl)-8- (trifluoromethyl)imidazo[1,2- a]pyridin-2-yl]carbonyl}-4- piperidinyl)-5,5-dimethyl-1,3- oxazolidin-2-one |
| 81 | 182 | 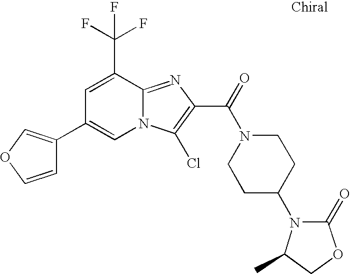 | (4R)-3-(1-{[3-chloro-6-(3- furanyl)-8- (trifluoromethyl)imidazo[1,2- a]pyridin-2-yl]carbonyl}-4- piperidinyl)-4-methyl-1,3- oxazolidin-2-one |
| 82 | 183 | 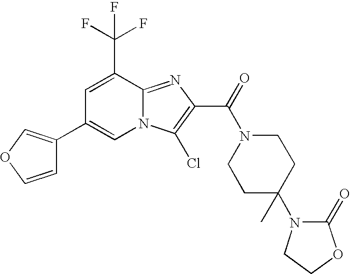 | 3-(1-{[3-chloro-6-(3-furanyl)-8- (trifluoromethyl)imidazo[1,2- a]pyridin-2-yl]carbonyl}-4- methyl-4-piperidinyl)-1,3- oxazolidin-2-one |
| 83 | 184 | 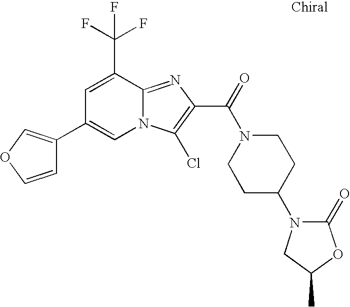 | (5S)-3-(1-{[3-chloro-6-(3- furanyl)-8- (trifluoromethyl)imidazo[1,2- a]pyridin-2-yl]carbonyl}-4- piperidinyl)-5-methyl-1,3- oxazolidin-2-one |
| 84 | 185 | 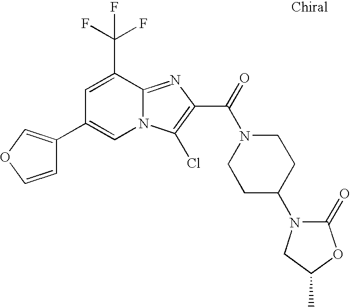 | (5R)-3-(1-{[3-chloro-6-(3- furanyl)-8- (trifluoromethyl)imidazo[1,2- a]pyridin-2-yl]carbonyl}-4- piperidinyl)-5-methyl-1,3- oxazolidin-2-one |
| 85 | 186 | 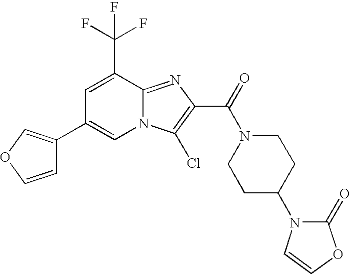 | 3-(1-{[3-chloro-6-(3-furanyl)-8- (trifluoromethyl)imidazo[1,2- a]pyridin-2-yl]carbonyl}-4- piperidinyl)-1,3-oxazol-2(3H)- one |
| 86 | 187 | 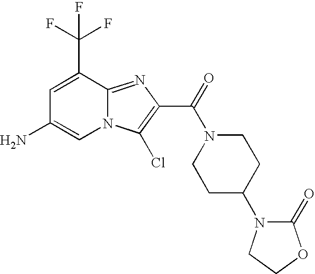 | 3-(1-{[6-amino-3-chloro-8- (trifluoromethyl)imidazo[1,2- a]pyridin-2-yl]carbonyl}-4- piperidinyl)-1,3-oxazolidin-2-one |
| 87 | 188 | 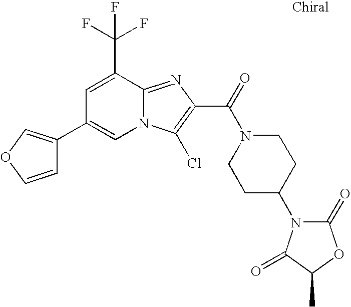 | (5S)-3-(1-{[3-chloro-6-(3- furanyl)-8- (trifluoromethyl)imidazo[1,2- a]pyridin-2-yl]carbonyl}-4- piperidinyl)-5-methyl-1,3- oxazolidine-2,4-dione |
| 88 | 189 | 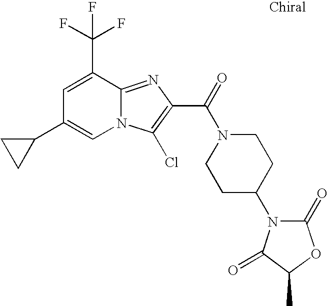 | (5S)-3-(1-{[3-chloro-6- cyclopropyl-8- (trifluoromethyl)imidazo[1,2- a]pyridin-2-yl]carbonyl}-4- piperidinyl)-5-methyl-1,3- oxazolidine-2,4-dione |
| 89 | 190 | 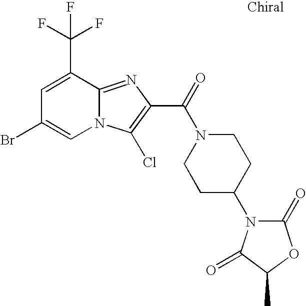 | (5S)-3-(1-{[6-bromo-3-chloro-8- (trifluoromethyl)imidazo[1,2- a]pyridin-2-yl]carbonyl}-4- piperidinyl)-5-methyl-1,3- oxazolidine-2,4-dione |
| 90 | 191 | 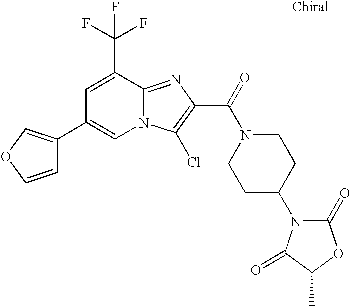 | (5R)-3-(1-{[3-chloro-6-(3- furanyl)-8- (trifluoromethyl)imidazo[1,2- a]pyridin-2-yl]carbonyl}-4- piperidinyl)-5-methyl-1,3- oxazolidine-2,4-dione |
| 91 | 192 | 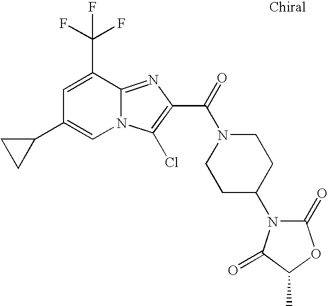 | (5R)-3-(1-{[3-chloro-6- cyclopropyl-8- (trifluoromethyl)imidazo[1,2- a]pyridin-2-yl]carbonyl}-4- piperidinyl)-5-methyl-1,3- oxazolidine-2,4-dione |
| 92 | 193 | 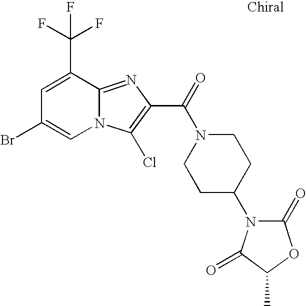 | (5R)-3-(1-{[6-bromo-3-chloro-8- (trifluoromethyl)imidazo[1,2- a]pyridin-2-yl]carbonyl}-4- piperidinyl)-5-methyl-1,3- oxazolidine-2,4-dione |
| 93 | 194 | 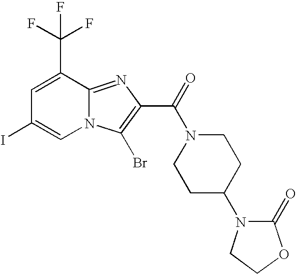 | 3-(1-{[3-bromo-6-iodo-8- (trifluoromethyl)imidazo[1,2- a]pyridin-2-yl]carbonyl}-4- piperidinyl)-1,3-oxazolidin-2-one |
| 94 | 195 | 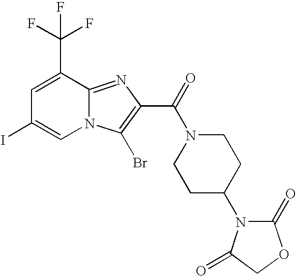 | 3-(1-{[3-bromo-6-iodo-8- (trifluoromethyl)imidazo[1,2- a]pyridin-2-yl]carbonyl}-4- piperidinyl)-1,3-oxazolidine-2,4- dione |
| 95 | 196 | 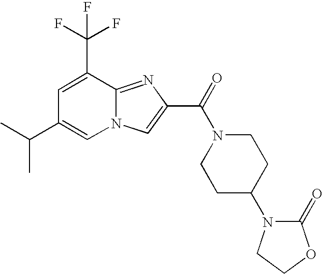 | 3-(1-{[6-(1-methylethyl)-8- (trifluoromethyl)imidazo[1,2- a]pyridin-2-yl]carbonyl}-4- piperidinyl)-1,3-oxazolidin-2-one |
| 96 | 197 | 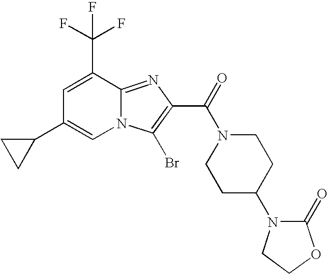 | 3-(1-{[3-bromo-6-cyclopropyl-8- (trifluoromethyl)imidazo[1,2- a]pyridin-2-yl]carbonyl}-4- piperidinyl)-1,3-oxazolidin-2-one |
| 97 | 198 | 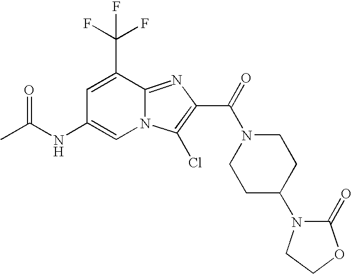 | N-[3-chloro-2-{[4-(2-oxo-1,3- oxazolidin-3-yl)-1- piperidinyl]carbonyl}-8- (trifluoromethyl)imidazo[1,2- a]pyridin-6-yl]acetamide |
| 98 | 199 | 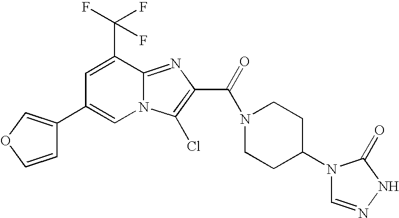 | 4-(1-{[3-chloro-6-(3-furanyl)-8- (trifluoromethyl)imidazo[1,2- a]pyridin-2-yl]carbonyl}-4- piperidinyl)-2,4-dihydro-3H- 1,2,4-triazol-3-one |
| 99 | 200 | 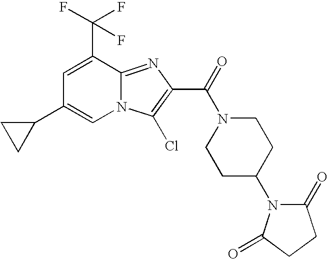 | 1-(1-{[3-chloro-6-cyclopropyl-8- (trifluoromethyl)imidazo[1,2- a]pyridin-2-yl]carbonyl}-4- piperidinyl)-2,5-pyrrolidinedione |
| 100 | 201 | 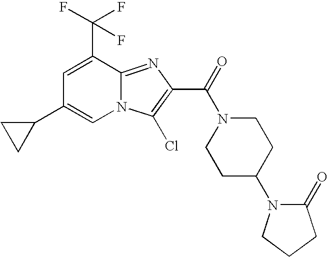 | 1-(1-{[3-chloro-6-cyclopropyl-8- (trifluoromethyl)imidazo[1,2- a]pyridin-2-yl]carbonyl}-4- piperidinyl)-2-pyrrolidinone |
| 101 | 202 | 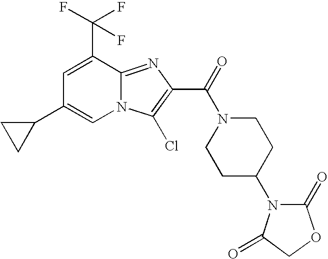 | 3-(1-{[3-chloro-6-cyclopropyl-8- (trifluoromethyl)imidazo[1,2- a]pyridin-2-yl]carbonyl}-4- piperidinyl)-1,3-oxazolidine-2,4- dione |
| 102 | 203 | 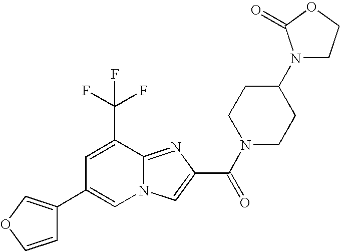 | 3-(1-{[6-(3-furanyl)-8- (trifluoromethyl)imidazo[1,2- a]pyridin-2-yl]carbonyl}-4- piperidinyl)-1,3-oxazolidin-2-one |
| 103 | 204 | 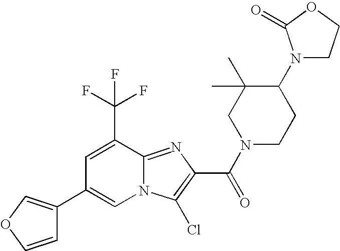 | 3-(1-{[3-chloro-6-(3-furanyl)-8- (trifluoromethyl)imidazo[1,2- a]pyridin-2-yl]carbonyl}-3,3- dimethyl-4-piperidinyl)-1,3- oxazolidin-2-one |
| 104 | 205 | 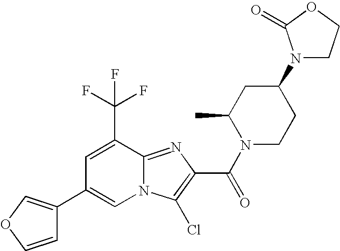 | (±) cis-3-(1-{[3-chloro-6-(3- furanyl)-8- (trifluoromethyl)imidazo[1,2- a]pyridin-2-yl]carbonyl}-2- methyl-4-piperidinyl)-1,3- oxazolidin-2-one |
| 105 | 206 | 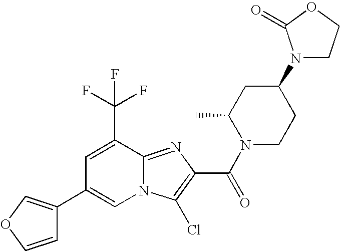 | (±) trans-3-(1-{[3-chloro-6-(3- furanyl)-8- (trifluoromethyl)imidazo[1,2- a]pyridin-2-yl]carbonyl}-2- methyl-4-piperidinyl)-1,3- oxazolidin-2-one |
| 106 | 207 | 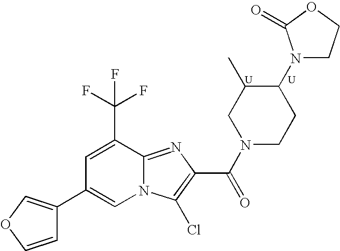 | 3-(1-{[3-chloro-6-(3-furanyl)-8- (trifluoromethyl)imidazo[1,2- a]pyridin-2-yl]carbonyl}-3- methyl-4-piperidinyl)-1,3- oxazolidin-2-one (Isomer 1) |
| 107 | 208 | 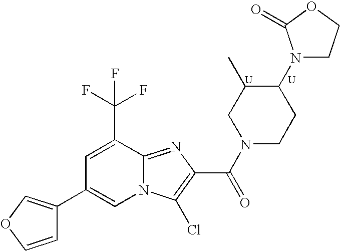 | 3-(1-{[3-chloro-6-(3-furanyl)-8- (trifluoromethyl)imidazo[1,2- a]pyridin-2-yl]carbonyl}-3- methyl-4-piperidinyl)-1,3- oxazolidin-2-one (Isomer 2) |
| 108 | 209 | 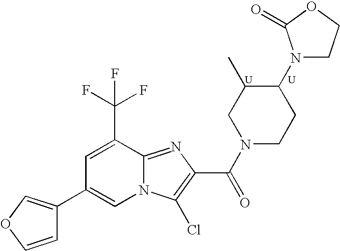 | 3-(1-{[3-chloro-6-(3-furanyl)-8- (trifluoromethyl)imidazo[1,2- a]pyridin-2-yl]carbonyl}-3- methyl-4-piperidinyl)-1,3- oxazolidin-2-one (Isomer 3) |
| 109 | 210 | 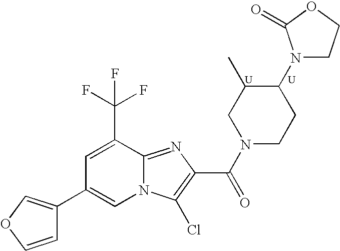 | 3-(1-{[3-chloro-6-(3-furanyl)-8- (trifluoromethyl)imidazo[1,2- a]pyridin-2-yl]carbonyl}-3- methyl-4-piperidinyl)-1,3- oxazolidin-2-one (Isomer 4) |
| 110 | 211 | 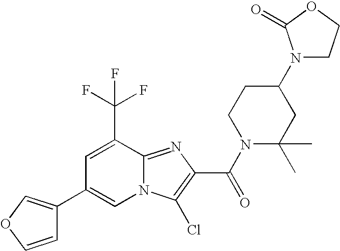 | 3-(1-{[3-chloro-6-(3-furanyl)-8- (trifluoromethyl)imidazo[1,2- a]pyridin-2-yl]carbonyl}-2,2- dimethyl-4-piperidinyl)-1,3- oxazolidin-2-one |
| 111 | 212 | 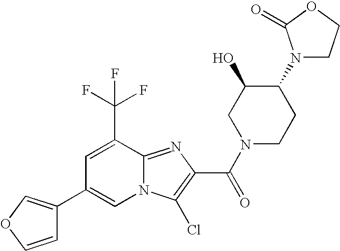 | (±) trans-3-(1-{[3-chloro-6-(3- furanyl)-8- (trifluoromethyl)imidazo[1,2- a]pyridin-2-yl]carbonyl}-3- hydroxy-4-piperidinyl)-1,3- oxazolidin-2-one |
| 112 | 213 | 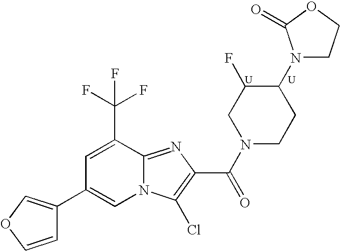 | (±) cis-3-(1-{[3-chloro-6-(3- furanyl)-8- (trifluoromethyl)imidazo[1,2- a]pyridin-2-yl]carbonyl}-3-fluoro- 4-piperidinyl)-1,3-oxazolidin-2- one |
| 113 | 214 | 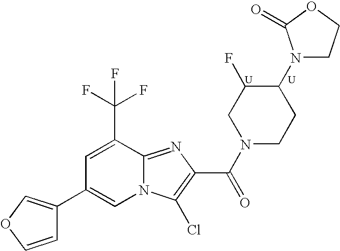 | (±) trans-3-(1-{[3-chloro-6-(3- furanyl)-8- (trifluoromethyl)imidazo[1,2- a]pyridin-2-yl]carbonyl}-3-fluoro- 4-piperidinyl)-1,3-oxazolidin-2- one |
| 114 | 215 | 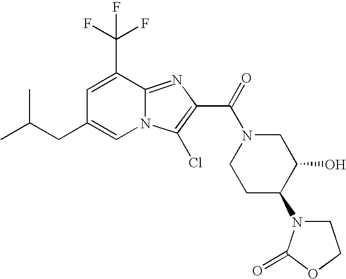 | (±) trans-3-(1-{[3-chloro-6-(2- methylpropyl)-8- (trifluoromethyl)imidazo[1,2- a]pyridin-2-yl]carbonyl}-3- hydroxy-4-piperidinyl)-1,3- oxazolidin-2-one |
| 115 | 216 | 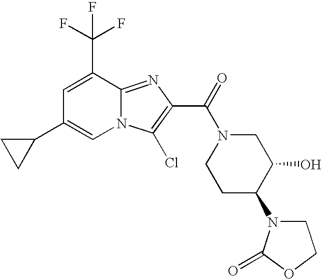 | (±) trans-3-(1-{[3-chloro-6- cyclopropyl)-8- (trifluoromethyl)imidazo[1,2- a]pyridin-2-yl]carbonyl}-3- hydroxy-4-piperidinyl)-1,3- oxazolidin-2-one |
| 116 | 217 | 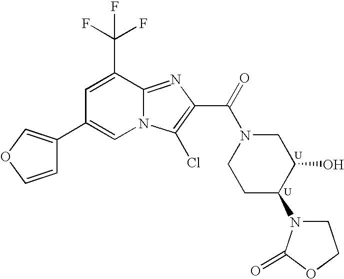 | 3-((trans)-1-{[3-chloro-6-(3- furanyl)-8- (trifluoromethyl)imidazo[1,2- a]pyridin-2-yl]carbonyl}-3- hydroxy-4-piperidinyl)-1,3- oxazolidin-2-one (enantiomer 1) |
| 117 | 218 | 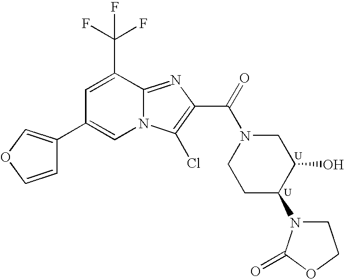 | 3-((trans)-1-{[3-chloro-6-(3- furanyl)-8- (trifluoromethyl)imidazo[1,2- a]pyridin-2-yl]carbonyl}-3- hydroxy-4-piperidinyl)-1,3- oxazolidin-2-one (enantiomer 2) |
| 118 | 219 | 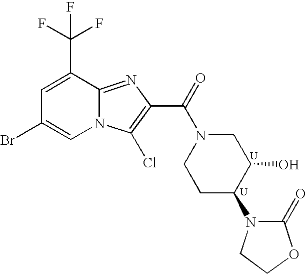 | (±) trans-3-(1-{[6-bromo-3- chloro-8- (trifluoromethyl)imidazo[1,2- a]pyridin-2-yl]carbonyl}-3- hydroxy-4-piperidinyl)-1,3- oxazolidin-2-one |
| 119 | 220 | 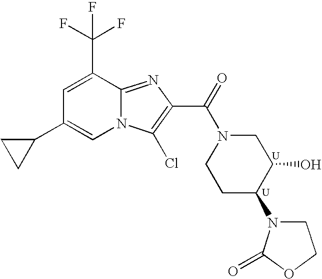 | 3-((trans)-1-{[3-chloro-6- cyclopropyl-8- (trifluoromethyl)imidazo[1,2- a]pyridin-2-yl]carbonyl}-3- hydroxy-4-piperidinyl)-1,3- oxazolidin-2-one (Enantiomer 1) |
| 120 | 221 | 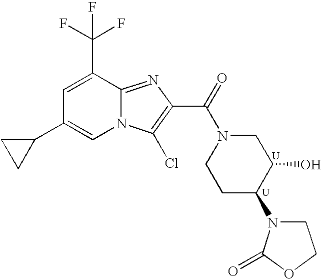 | 3-((trans)-1-{[3-chloro-6- cyclopropyl-8- (trifluoromethyl)imidazo[1,2- a]pyridin-2-yl]carbonyl}-3- hydroxy-4-piperidinyl)-1,3- oxazolidin-2-one (Enantiomer 2) |
| 121 | 222 | 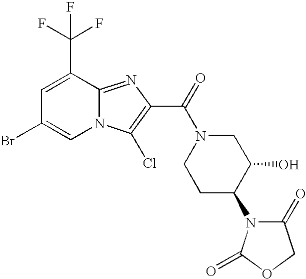 | (±) trans-3-(1-{[6-bromo-3- chloro-8- (trifluoromethyl)imidazo[1,2- a]pyridin-2-yl]carbonyl}-3- hydroxy-4-piperidinyl)-1,3- oxazolidine-2,4-dione |
| 122 | 223 | 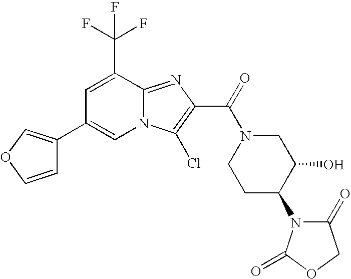 | (±) trans-3-(1-{[3-chloro-6-(3- furanyl)-8- (trifluoromethyl)imidazo[1,2- a]pyridin-2-yl]carbonyl}-3- hydroxy-4-piperidinyl)-1,3- oxazolidine-2,4-dione |
| 123 | 224 | 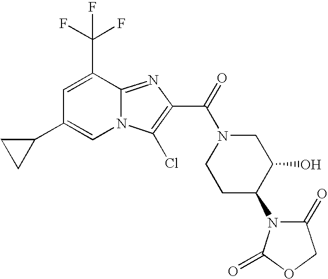 | (±) trans-3-(1-{[3-chloro-6- cyclopropyl-8- (trifluoromethyl)imidazo[1,2- a]pyridin-2-yl]carbonyl}-3- hydroxy-4-piperidinyl)-1,3- oxazolidine-2,4-dione |
| 124 | 225 | 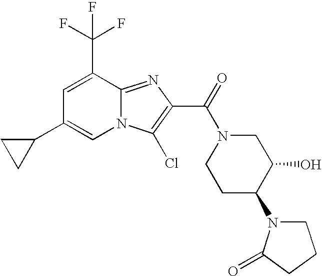 | (±) trans-1-(1-{[3-chloro-6- cyclopropyl-8- (trifluoromethyl)imidazo[1,2- a]pyridin-2-yl]carbonyl}-3- hydroxy-4-piperidinyl)-2- pyrrolidinone |
| 125 | 226 | 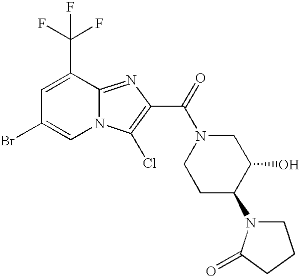 | (±) trans-1-(1-{[6-bromo-3- chloro-8- (trifluoromethyl)imidazo[1,2- a]pyridin-2-yl]carbonyl}-3- hydroxy-4-piperidinyl)-2- pyrrolidinone |
| 126 | 227 | 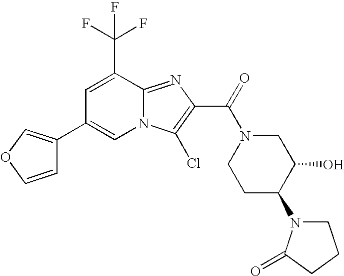 | (±) trans-1-(1-{[3-chloro-6-(3- furanyl)-8- (trifluoromethyl)imidazo[1,2- a]pyridin-2-yl]carbonyl}-3- hydroxy-4-piperidinyl)-2- pyrrolidinone |
| 127 | 228 | 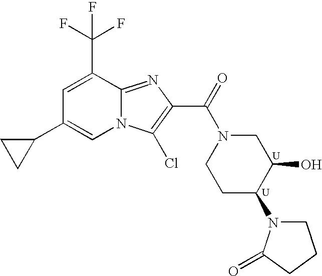 | (±) cis-1-(1-{[3-chloro-6- cyclopropyl-8- (trifluoromethyl)imidazo[1,2- a]pyridin-2-yl]carbonyl}-3- hydroxy-4-piperidinyl)-2- pyrrolidinone |
| 128 | 229 | 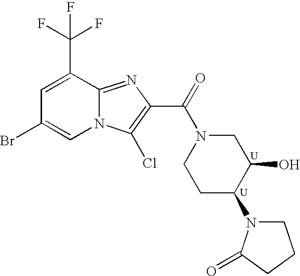 | (±) cis-1-(1-{[6-bromo-3-chloro- 8-(trifluoromethyl)imidazo[1,2- a]pyridin-2-yl]carbonyl}-3- hydroxy-4-piperidinyl)-2- pyrrolidinone |
| 129 | 230 | 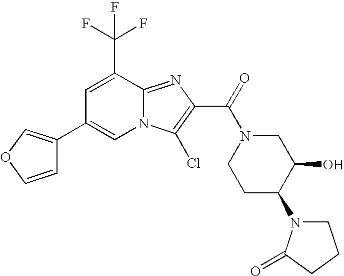 | (±) cis-1-(1-{[3-chloro-6-(3- furanyl)-8- (trifluoromethyl)imidazo[1,2- a]pyridin-2-yl]carbonyl}-3- hydroxy-4-piperidinyl)-2- pyrrolidinone |
| 130 | 231 | 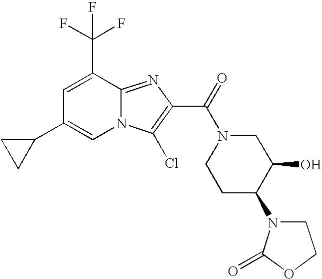 | (±) cis-3-(1-{[3-chloro-6- cyclopropyl-8- (trifluoromethyl)imidazo[1,2- a]pyridin-2-yl]carbonyl}-3- hydroxy-4-piperidinyl)-1,3- oxazolidin-2-one |
| 131 | 232 | 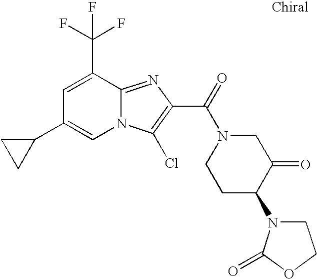 | 1-{[3-chloro-6-cyclopropyl-8- (trifluoromethyl)imidazo[1,2- a]pyridin-2-yl]carbonyl}-4-(2-oxo- 1,3-oxazolidin-3-yl)-3- piperidinone |
| 132 | 233 | 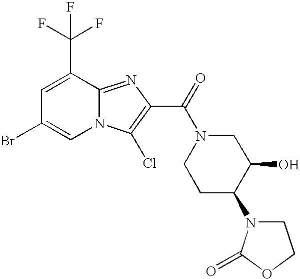 | (±) cis-3-(1-{[6-bromo-3-chloro- 8-(trifluoromethyl)imidazo[1,2- a]pyridin-2-yl]carbonyl}-3- hydroxy-4-piperidinyl)-1,3- oxazolidin-2-one |
| 133 | 234 | 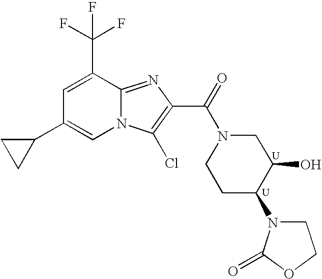 | 3-((cis)-1-{[3-chloro-6- cyclopropyl-8- (trifluoromethyl)imidazo[1,2- a]pyridin-2-yl]carbonyl}-3- hydroxy-4-piperidinyl)-1,3- oxazolidin-2-one (Enantiomer 1) |
| 134 | 235 | 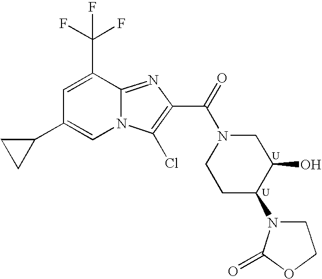 | 3-((cis)-1-{[3-chloro-6- cyclopropyl-8- (trifluoromethyl)imidazo[1,2- a]pyridin-2-yl]carbonyl}-3- hydroxy-4-piperidinyl)-1,3- oxazolidin-2-one (Enantiomer 2) |
| 135 | 236 | 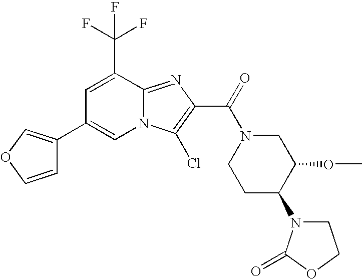 | (±) trans-3-[1-{[3-chloro-6-(3- furanyl)-8- (trifluoromethyl)imidazo[1,2- a]pyridin-2-yl]carbonyl}-3- (methyloxy)-4-piperidinyl]-1,3- oxazolidin-2-one |
| 136 | 237 | 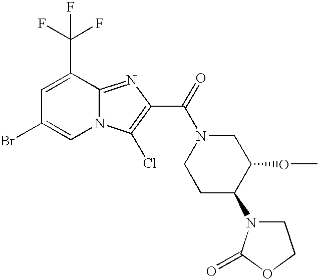 | (±) trans-3-[1-{[6-bromo-3- chloro-8- (trifluoromethyl)imidazo[1,2- a]pyridin-2-yl]carbonyl}-3- (methyloxy)-4-piperidinyl]-1,3- oxazolidin-2-one |
| 137 | 238 | 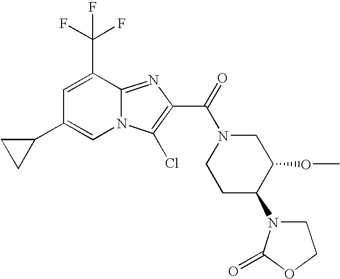 | (±) trans-3-[1-{[3-chloro-6- cyclopropyl-8- (trifluoromethyl)imidazo[1,2- a]pyridin-2-yl]carbonyl}-3- (methyloxy)-4-piperidinyl]-1,3- oxazolidin-2-one |
| 138 | 239 | 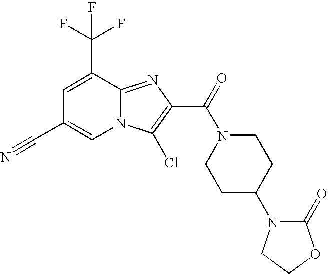 | 3-chloro-2-{[4-(2-oxo-1,3- oxazolidin-3-yl)-1- piperidinyl]carbonyl}-8- (trifluoromethyl)imidazo[1,2- a]pyridine-6-carbonitrile |
| | 240 | 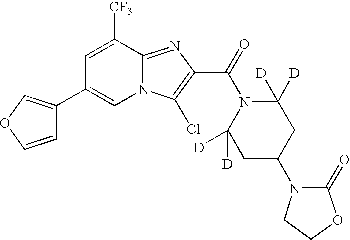 | |
| | 241 | 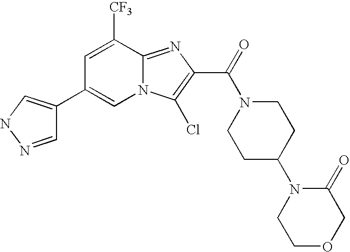 | |
| | 242 | 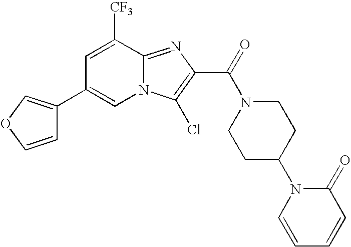 | |
| | 243 | 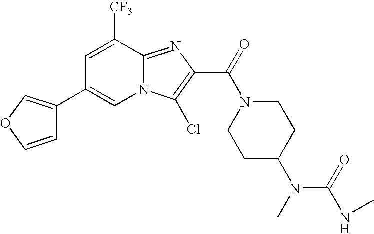 | |

In another embodiment, there is provided a pharmaceutical composition comprising a pharmaceutically acceptable diluent and a therapeutically effective amount of a compound of Formula (I) or a pharmaceutically acceptable salt thereof.

In another embodiment, there is provided a method for treating a viral infection in a mammal mediated at least in part by a virus in the Flaviviridae family of viruses, which method comprises administering to a mammal that has been diagnosed with said viral infection or is at risk of developing said viral infection a compound described herein. In another embodiment, the virus is hepatitis C virus.

In another embodiment, the method for treating a viral infection in a mammal mediated at least in part by a virus in the Flaviviridae family of viruses further comprises administration of a therapeutically effective amount of one or more agents active against hepatitis C virus. In another embodiment, the agent is an inhibitor of HCV protease, HCV polymerase, HCV helicase, HCV NS4B protein, HCV entry, HCV assembly, HCV egress, HCV replicase, HCV NS5A protein, or inosine 5′-monophosphate dehydrogenase. In another embodiment, the agent is interferon.

Other aspects and embodiments will be apparent to those skilled in the art from the following detailed description.

### Administration and Formulation

The chemical entities provided herein may inhibit viral replication by inhibiting the enzymes involved in replication, such as the non-structural proteins including RNA dependent RNA polymerase. They may also inhibit other enzymes utilized in the activity or proliferation of viruses in the Flaviviridae family, such as HCV. The chemical entities are administered at a therapeutically effective dosage, e.g., a dosage sufficient to provide treatment for the disease.

Administration of the chemical entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In addition, the chemical entities described herein can be co-administered with, and the pharmaceutical compositions can include, other medicinal agents, pharmaceutical agents, adjuvants, and the like. Suitable medicinal and pharmaceutical agents include therapeutically effective amounts of one or more agents active against HCV. In some embodiments, the agent active against HCV is an inhibitor of HCV protease, HCV polymerase, HCV helicase, HCV NS4B protein, HCV entry, HCV assembly, HCV egress, HCV replicase, HCV NS5A protein, or inosine 5′-monophosphate dehydrogenase.

Active agents against HCV include ribavirin, levovirin, viramidine, thymosin alpha-1, an inhibitor of NS3 serine protease, and inhibitor of inosine monophosphate dehydrogenase, interferon-alpha, either alone or in combination with ribavirin or levovirin. In some embodiments, the additional agent active against HCV is interferon-alpha or pegylated interferon-alpha alone or in combination with ribavirin or levovirin. In some embodiments, the agent active against hepatitis C virus is interferon.

The above other therapeutic agents, when employed in combination with the chemical entities described herein, may be used, for example, in those amounts indicated in the Physicians' Desk Reference (PDR) or as otherwise determined by one of ordinary skill in the art.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

The following examples serve to more fully describe the manner of using the above-described invention. It is understood that these examples in no way serve to limit the true scope of this invention, but rather are presented for illustrative purposes.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used, the route and form of administration, and other factors. The drug can be administered more than once a day, such as once or twice a day.

Therapeutically effective amounts of the chemical entities described herein may range from approximately 0.01 to 200 mg per kilogram body weight of the recipient per day; such as about 0.01-100 mg/kg/day, for example, from about 0.1 to 50 mg/kg/day. Thus, for administration to a 70 kg person, the dosage range may be about 7-3500 mg per day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %. Representative pharmaceutical compositions containing at least one chemical entity described herein are described below.

Additionally, the present specification is directed to a pharmaceutical composition comprising a therapeutically effective amount of at least one chemical entity described herein in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrogenase, interferon-alpha pegylated interferon-alpha (peginterferon-alpha), a combination of interferon-alpha and ribavirin, a combination of peginterferon-alpha and ribavirin, a combination of interferon-alpha and levovirin, and a combination of peginterferon-alpha and levovirin. Interferon-alpha includes, but is not limited to, recombinant interferon-alpha2a (such as ROFERON interferon available from Hoffman-LaRoche, Nutley, N.J.), interferon-alpha2b (such as Intron-A interferon available from Schering Corp., Kenilworth, N.J., USA), a consensus interferon, and a purified interferon-alpha product. For a discussion of ribavirin and its activity against HCV, see J. O. Saunders and S. A. Raybuck, “Inosine Monophosphate Dehydrogenase: Consideration of Structure, Kinetics and Therapeutic Potential,” Ann. Rep. Med. Chem., 2:201-210 (2000).

### Synthetic Methods

The methods of synthesis for the provided chemical entities employ readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, the methods of this specification employ protecting groups which are necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof For example, many of the starting materials are available from commercial suppliers such as Aldrich Chemical Co. (Milwaukee, Wis., USA), Bachem (Torrance, Calif., USA), Ernka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989). The synthesis of the compounds provided generally follows either a convergent or linear synthetic pathway as described below.

Unless specified to the contrary, the reactions described herein take place at atmospheric pressure, generally within a temperature range from −78° C. to 200° C. Further, except as employed in the Examples or as otherwise specified, reaction times and conditions are intended to be approximate, e.g., taking place at about atmospheric pressure within a temperature range of about −78° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, N-methylpyrrolidone (“NMP”), pyridine and the like].

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### Examples

The following examples serve to more fully describe the manner of making and using the above-described invention. It is understood that these examples in no way serve to limit the true scope of the invention, but rather are presented for illustrative purposes.

Scheme 1 shows a method of assembling the imidazopyridine scaffold with various substituents. 2-Amino-3-trifluromethyl pyridine 1.1 is brominated by treatment with N-bromosuccinimide (NBS) in a solvent such as dimethylformamide (DMF). Substituted 2-aminopyridine 1.2 is cyclized to the imidazopyridine 1.3 by heating it with ethyl bromopyruvate in a solvent like DMF. Treatment of intermediate 1.3 with a suitable halogenation reagent can afford the 3-substituted imidazopyridine 1.4. For example, treatment of intermediate 1.3 with N-chlorosuccinimide (NCS) in DMF can afford the 3-chlorosubstituted imidazopyridine (X═Cl), or treatment with N-bromosuccinimide (NBS) in DMF can afford the 3-bromosubstituted imidazopyridine (X═Br). Palladium mediated coupling reactions such as Suzuki couplings, Sonogashira couplings and Heck couplings can afford diversity at A in intermediates 1.5. Hydrolysis of the ester is effected by refluxing in 4N hydrochloric acid (HCl) and acetonitrile as co-solvent. The acid 1.6 is converted to amide 1.7 through standard amide coupling agents such as 1,1,3,3-tetramethylaminium hexafluorophosphate (HBTU) (not shown in the scheme) with pre-formed amine 1.8. In Scheme 1, A, R¹, R³, T, W, X and n are as defined herein.

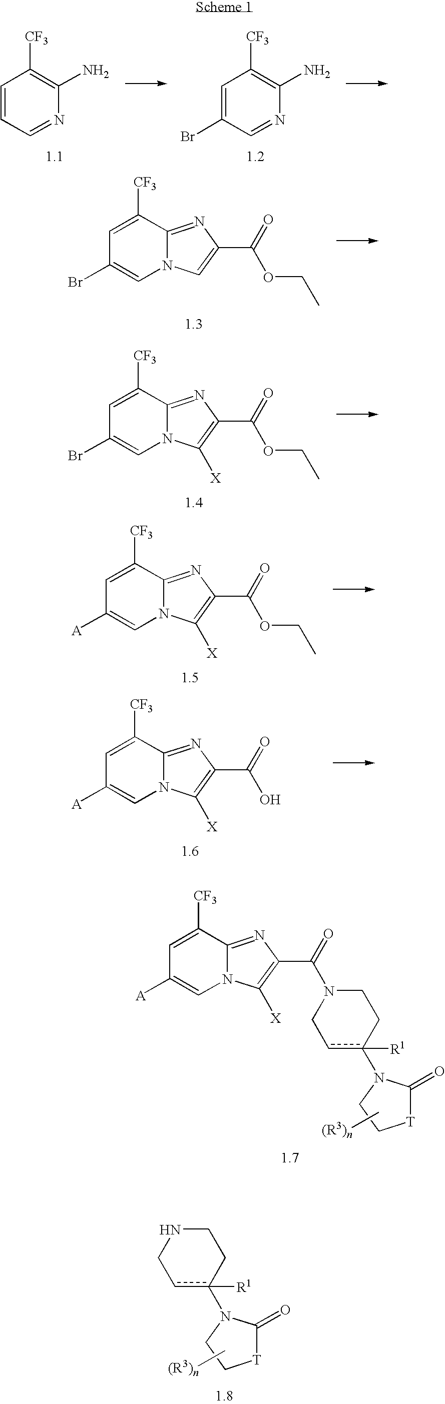

Additional synthetic protocols useful in making the synthetic intermediates may be found in U.S. Ser. No. 12/228,139, filed on Aug. 8, 2008 and entitled “Certain Nitrogen Containing Bicyclic Chemical Entities for Treating Viral Infections.”

### Example 1

### 1-[1-(3-Chloro-6-furan-3-yl-8-trifluoromethyl-imidazo[1,2-a]pyridine-2-carbonyl)-piperidin-4-yl]-pyrrolidin-2-one

### (Compound 101)

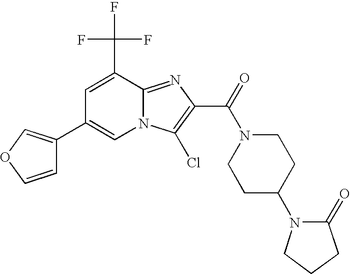

### Step 1

### 5-Bromo-3-trifluoromethyl-pyridin-2-ylamine

A solution of N-Bromosuccinimide (54.89 g, 308.41 mmol) in DMF (250 mL) was rapidly added dropwise via addition funnel to a stirring solution of 2-Amino-3-(trifluoromethyl)pyridine (50.00 g, 308.41 mmol) in DMF (250 mL) at room temperature. The mixture was stirred for 2 hours, concentrated to a volume of 200 mL and added slowly into a beaker containing a cold stirring mixture of 5% NaHSO₃ and water (1.5 L, 1:10 v/v). The precipitate was filtered, washed with water (3×500 mL) and dried under vacuum to afford 5-bromo-3-trifluoromethyl-pyridin-2-ylamine (62.89 g, 84.6%) as a light brown solid. MS 242.9 (M+H⁺).

### Step 2

### 6-Bromo-8-trifluoromethyl-imidazo[1,2-a]pyridine-2-carboxylic acid ethyl ester

A mixture of 2-amino-4-bromo-3-(trifluoromethyl)pyridine (21.78 g, 90.37 mmol) and ethyl bromopyruvate (90% pure, 25.3 mL, 180.74 mmol) was heated in DMF (180 mL) at 50° C. for 1 day. Upon cooling, the sovent was removed to half the volume under reduced pressure. The mixture was diluted with EtOAc (500 mL) and washed with water (3×150 mL), dried (Na₂SO₄), filtered and concentrated. The crude brown oil was dissolved in minimum amount of EtOAc and dripped slowly into n-hexanes (500 mL) with vigorous stirring. The suspension was allowed to stir overnight and filtered to give 6-bromo-8-trifluoromethyl-imidazo[1,2-a]pyridine-2-carboxylic acid ethyl ester (26.83 g, 89%) as a yellow solid. MS (ESI) m/z=337, 339 (MH⁺).

### Step 3

### 6-Bromo-3-chloro-8-trifluoromethyl-imidazo[1,2-a]pyridine-2-carboxylic acid ethyl ester

A mixture of the 6-bromo-8-trifluoromethyl-imidazo[1,2-a]pyridine-2-carboxylic acid ethyl ester (8.08 g, 23.97 mmol) and N-chlorosuccinimide (3.68 g, 27.56 mmol) was stirred in DMF (80 mL) at room temperature for 14.5 hours. The solvent was removed under reduced pressure to ˜20 mL and diluted with EtOAc (400 mL). The organic layer was washed successively with aqueous sodium thiosulfate (1M, 2×100 mL), saturated aqueous NaHCO₃ (100 mL) and brine (100 mL), filtered through a small pad of silica gel and concentrated to give 6-bromo-3-chloro-8-trifluoromethyl-imidazo[1,2-a]pyridine-2-carboxylic acid ethyl ester (7.64 g, 86%) as yellow solid. MS (ESI) m/z=370.9, 372.9, 374.9 (MH⁺).

### Step 4

### 3-Chloro-6-furan-3-yl-8-trifluoromethyl-imidazo[1,2-a]pyridine-2-carboxylic acid ethyl ester

A mixture of 6-bromo-3-chloro-8-trifluoromethyl-imidazo[1,2-a]pyridine-2-carboxylic acid ethyl ester (0.6 g, 1.61 mmol), 3-furanboronic acid (244 mg, 2.18 mmol) and tetrakis(triphenyl)phosphine palladium(0) (93.3 mg, 0.081mmol) in aqueous K₃PO₄ (1M, 4.5 mL) and 1,4-dioxane (13.5 mL) was heated at 100° C. under microwave conditions for 15 min. The reaction was repeated 8 times and combined. The combined reaction mixtures were concentrated and diluted with EtOAc (250 mL) and washed with saturated aqueous NaHCO₃ (2×150 mL), then brine (100 mL). The extracts were filtered through a pad of silica gel and the solvent concentrated under reduced pressure. The product was crystallized from EtOAc to give 3-chloro-6-furan-3-yl-8-trifluoromethyl-imidazo[1,2-a]pyridine-2-carboxylic acid ethyl ester (2.02 g) as white solid. The filtrate was column chromatographed [n-hex:EtOAc (5:1 v/v) to n-hex:EtOAc (3:1 v/v)]to give a second batch (0.78 g). MS (ESI) m/z=359 (MH⁺).

### Step 5

### 3-Chloro-6-furan-3-yl-8-trifluoromethyl-imidazo[1,2-a]pyridine-2-carboxylic acid

A mixture of 3-chloro-6-furan-3-yl-8-trifluoromethyl-imidazo[1,2-a]pyridine-2-carboxylic acid ethyl ester (1.16 g, 3.24 mmol) in 1,4-dioxane (50 mL) and 3N HCl (50 mL) was heated at 100° C. until reaction was done. Upon cooling, the solvent was removed under reduced pressure which after aqueous workup gave 3-chloro-6-furan-3-yl-8-trifluoromethyl-imidazo[1,2-a]pyridine-2-carboxylic acid (1.13 g) as white solid which was used for the next step without further purification. MS (ESI) m/z=331 (MH⁺).

### Step 6

### 1-[1-(3-Chloro-6-furan-3-yl-8-trifluoromethyl-imidazo[1,2-a]pyridine-2-carbonyl)-piperidin-4-yl]-pyrrolidin-2-one

### Compound 101

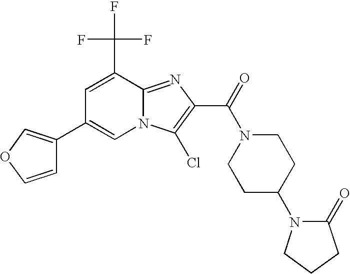

1-[1-(3-Chloro-6-furan-3-yl-8-trifluoromethyl-imidazo[1,2-a]pyridine-2-carbonyl)-piperidin-4-yl]-pyrrolidin-2-one was prepared by standard HATU coupling of 3-chloro-6-furan-3-yl-8-trifluoromethyl-imidazo[1,2-a]pyridine-2-carboxylic acid and 3-piperidin-4-yl-oxazolidin-2-one. ¹H NMR (d₆-DMSO, 300 MHz) δ 1.63 (m, 4H), 1.90 (t, 2H, J=6.90 Hz), 2.22 (t, 2H, J=8.40 Hz), 2.89 (m, 1H), 3.24 (m, 3H), 4.13 (m, 2H), 4.61 (m, 1H), 7.31 (m, 1H), 7.84 (t, 1H, J=1.50 Hz), 8.19 (s, 1H), 8.55 (s, 1H), 8.81 (s, 1H); MS (ESI) m/z=481.1 (MH⁺).

### Example 2

### 1-{1-[3-Chloro-6-(1H-pyrazol-4-yl)-8-trifluoromethyl-imidazo[1,2-a]pyridine-2-carbonyl]-piperidin-4-yl}-pyrrolidin-2-one

### Compound 102

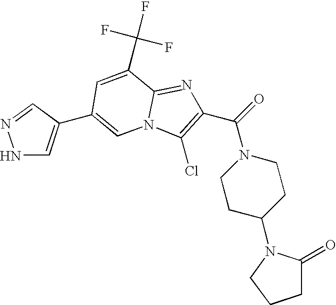

Prepared according to Example 1. ¹H NMR (d₆-DMSO, 300 MHz) δ 1.64 (m, 4H), 1.90 (m, 2H), 2.22 (m, 2H), 2.88 (m, 2H), 3.24 (m, 2H), 4.07 (m, 1H), 4.19 (m, 1H), 4.61 (m, 1H), 8.19 (s, 1H), 8.24 (s, 1H), 8.55 (s, 1H), 8.82 (s, 1H), 13.15 (s, 1H); MS (ESI) m/z=481.4 (MH⁺).

### Example 3

### 3-[1-(3-Chloro-6-furan-3-yl-8-trifluoromethyl-imidazo[1,2-a]pyridine-2-carbonyl)-piperidin-4-yl]-oxazolidin-2-one

### (Compound 103

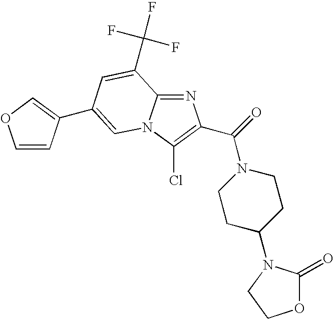

Prepared according to Example 1. ¹H NMR (d₆-DMSO, 300 MHz) δ 1.66 (m, 3H), 1.80 (m, 1H), 2.89 (m, 1H), 3.22 (m, 1H), 3.51 (m, 2H), 3.82 (m, 1H), 4.22 (m, 3H), 4.61 (bd, 1H, J=13.20 Hz), 7.31 (m, 1H), 7.84 (t, 1H, J=1.50 Hz), 8.19 (s, 1H), 8.55 (m, 1H), 8.81 (s, 1H); MS (ESI) m/z=483.1 (MH⁺).

### Example 4

### 3-[1-(3-Chloro-6-phenyl-8-trifluoromethyl-imidazo[1,2-a]pyridine-2-carbonyl)-piperidin-4-yl]-oxazolidin-2-one

### (Compound 104)

### Step 1

### 5-Bromo-3-chloro-7-trifluoromethyl-imidazo[1,2-a]pyridine-2-carboxylic acid

A mixture of 6-bromo-3-chloro-8-trifluoromethyl-imidazo[1,2-a]pyridine-2-carboxylic acid methyl ester (5 g, 14 mmol) and NaOH (2M, 21 mL, 42 mmol) was stirred at room temperature in THF/H₂O (3:1 v/v, 100 mL) for 2 hours. The reaction mixture was concentrated and the residue was acidified with 10% HCl and extracted with DCM (2×80 mL). The organic layer was washed with brine (50 mL), dried (Mg50₄), filtered and concentrated to afford 5-bromo-3-chloro-7-trifluoromethyl-imidazo[1,2-a]pyridine-2-carboxylic acid (4.42 g, 92%) as a light yellow powder, which was used without further purification. ¹H NMR (d₆-DMSO, 300 MHz) δ 8.09 (s, 1H), 8.98 (d, 1H, J=0.80 Hz), 13.5 (s, 1H); MS (ESI) m/z=344.9, 346.9 (MH)

### Step 2

### 3-[1-(6-Bromo-3-chloro-8-trifluoromethyl-imidazo[1,2-a]pyridine-2-carbonyl)-piperidin-4-yl]-oxazolidin-2-one

### (Compound 105)

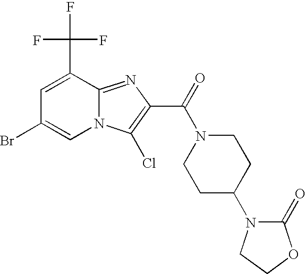

3-[1-(6-Bromo-3-chloro-8-trifluoromethyl-imidazo [1,2-a]pyridine-2-carbonyl)-piperidin-4-yl]-oxazolidin-2-one was prepared from standard amide bond coupling of 5-bromo-3-chloro-7-trifluoromethyl-imidazo[1,2-a]pyridine-2-carboxylic acid and 3-piperidin-4-yl-oxazolidin-2-one. ¹H NMR (d₆-DMSO, 300 MHz) δ1.65 (m, 3H), 1.80 (m, 1H), 2.90 (m, 1H), 3.19 (m, 1H), 3.52 (m, 2H), 3.81 (m, 1H), 4.12 (d, 1H, J=13.80 Hz), 4.25 (t, 2H, J=8.10 Hz), 4.59 (d, 1H, J=13.20 Hz), 8.07 (m, 1H), 8.99 (m, 1H); MS (ESI) m/z=496.4 (MH⁺).

### Step 3

### 3-[1-(3-Chloro-6-phenyl-8-trifluoromethyl-imidazo[1,2-a]pyridine-2-carbonyl)-piperidin-4-yl]-oxazolidin-2-one

### (Compound 104)

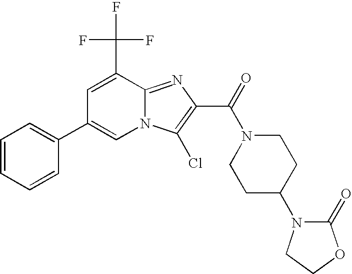

3-[1-(3-Chloro-6-phenyl-8-trifluoromethyl-imidazo [1,2-a]pyridine-2-carbonyl)-piperidin-4-yl]-oxazolidin-2-one was prepared from Suzuki reaction of 3-[1-(6-bromo-3-chloro-8-trifluoromethyl-imidazo[1,2-a]pyridine-2-carbonyl)-piperidin-4-yl]-oxazolidin-2-one and phenylboronic acid. ¹H NMR (d₆-DMSO, 300 MHz) δ 1.66 (m, 3H), 1.82 (m, 1H), 2.92 (m, 1H), 2.92 (m, 1H), 3.22 (m, 1H), 3.54 (m, 2H), 3.83 (m, 1H), 4.26 (m, 3H), 4.62 (d, 1H, J=13.20 Hz), 7.55 (m, 3H), 7.88 (m, 2H), 8.17 (s, 1H), 8.79 (s, 1H); MS (ESI) m/z=493.0 (MH⁺).

### Example 5

### 3-[1-(3-Chloro-6-isopropenyl-8-trifluoromethyl-imidazo[1,2-a]pyridine-2-carbonyl)-piperidin-4-yl]-oxazolidin-2-one

### (Compound 106)

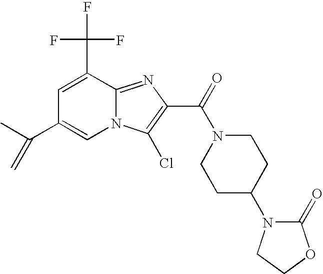

Prepared according to Example 4, Compound 104. ¹H NMR (d₆-DMSO, 300 MHz) δ 1.66 (m, 3H), 1.80 (m, 1H), 2.23 (s, 3H), 2.91 (m, 1H), 3.20 (m, 1H), 3.53 (m, 2H), 3.82 (m, 1H), 4.22 (m, 3H), 4.60 (d, 1H, J=13.20 Hz), 5.35 (s, 1H), 5.77 (s, 1H), 8.10 (s, 1H), 8.43 (s, 1H). MS (ESI) m/z=457.9 (MH⁺).

### Example 6

### 3-[1-(3-Chloro-6-cyclopent-1-enyl-8-trifluoromethyl-imidazo[1,2-a]pyridine-2-carbonyl)-piperidin-4-yl]-oxazolidin-2-one

### (Compound 107)

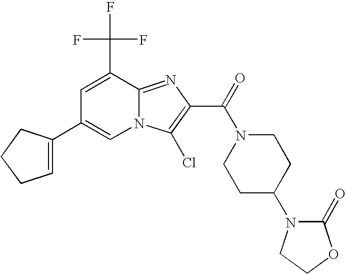

Prepared according to Example 4, Compound 104. ¹H NMR (d₆-DMSO, 300 MHz) δ 1.63 (m, 3H), 1.80 (m, 1H), 2.02 (m, 2H), 2.52 (m, 2H), 2.77 (m, 2H), 2.89 (m, 1H), 3.16 (m, 1H), 3.52 (m, 2H), 3.81 (m, 1H), 4.22 (m, 3H), 4.60 (d, 1H, J=14.10 Hz), 6.69 (s, 1H), 8.15 (s, 1H), 8.24 (s, 1H); MS (ESI) m/z=483 (MH).

### Example 7

### 3-{1-[3-Chloro-6-(1H-pyrazol-4-yl)-8-trifluoromethyl-imidazo [1,2-a]pyridine-2-carbonyl]-piperidin-4-yl}-oxazolidin-2-one

### (Compound 108)

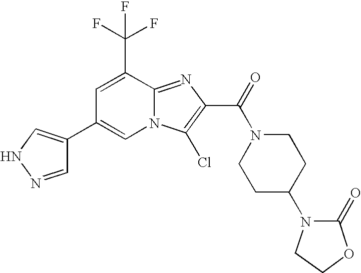

Prepared according to Example 3, Compound 103. ¹H NMR (d₆-DMSO, 300 MHz) δ 1.55-1.90 (m, 4H), 2.80-3.30 (m, 2H), 3.4-3.6 (m, 2H), 3.82 (m, 1H), 3.15-4.30 (m, 3H), 4.55-4.70 (m, 1H), 8.19 (s, 1H), 8.24 (s, 1H), 8.56 (s, 1H), 8.82 (s, 1H), 13.15 (s, 1H); MS (ESI) m/z =482.9 (MH⁺).

### Example 8

### 1-(1-{[3-chloro-8-(trifluoromethyl)imidazo[1,2-a]pyridin-2-yl]carbonyl}-4-piperidinyl)-2-pyrrolidinone

### (compound 109)

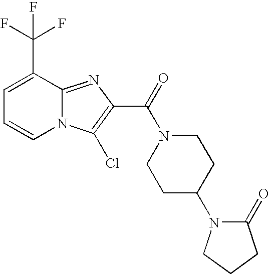

### Step A

### methyl 8-(trifluoromethyl)imidazo[1,2-a]pyridine-2-carboxylate

3-(trifluoromethyl)-2-pyridinamine (10 g, 61.7 mmol) and methyl bromopyruvate (16.54 mL, 123 mmol) in N,N-dimethylformamide (200 mL) were heated to 60° C. for 3 hours, and was then stirred at room temperature overnight. The mixture was concentrated to ˜50 mL. The residue was poured into stirring ice water. Solids precipitated, were collected by filtration, washed 2 times with water, air dried, and dried under vacuum overnight to give the title compound (10.8 g; 72%). LCMS: m/z 245 (M+1).

### Step B

### methyl 3-chloro-8-(trifluoromethyl)imidazo[1,2-a]pyridine-2-carboxylate

To a solution of methyl 8-(trifluoromethyl)imidazo[1,2-a]pyridine-2-carboxylate (2.6 g, 10.65 mmol) in N,N-dimethylformamide (40 mL) was added N-chlorosuccinimide (1.49 g, 11.2 mmol). The mixture was heated to 60° C., stirred for 2 hours, and then concentrated to ˜5 mL. The residue was taken up in ethyl acetate, washed with brine, washed with saturated sodium bisulfite, dried over sodium sulfate, and concentrated to give the title compound (3.1 g; 99%). LCMS: m/z 279, 281 (M+1).

### Step C

### 3-chloro-8-(trifluoromethyl)imidazo[1,2-a]pyridine-2-carboxylic acid

To a mixture of methyl 3-chloro-8-(trifluoromethyl)imidazo[1,2-a]pyridine-2-carboxylate (3.10 mL, 11.1 mmol) in tetrahydrofuran (20 mL) was added 1M sodium hydroxide (22.2 mL, 22.2 mmol). The mixture was stirred at room temperature for 2 hours and then was made acidic with 1N hydrochloric acid (27.8 mL, 27.8 mmol). The mixture was extracted with dichloromethane and the organic phase washed with brine, dried over sodium sulfate, and concentrated. The product was dried under high vacuum overnight to give the title compound (2.2 g; 75%). N10676-5-1=2.2 g; LCMS: m/z 265, 267 (M+1).

### Step D

### 1-(1-{[3-chloro-8-(trifluoromethyl)imidazo[1,2-a]pyridin-2-yl]carbonyl)-4-piperidinyl)-2-pyrrolidinone

To a mixture of 1-(4-piperidinyl)-2-pyrrolidinone (93 mg, 0.45 mmol), N-ethyl-N-(1-methylethyl)-2-propanamine (0.26 mL, 1.5 mmol), N-ethyl-N-(1-methylethyl)-2-propanamine (0.26 mL, 1.5 mmol), and 1-(4-piperidinyl)-2-pyrrolidinone (93 mg, 0.45 mmol) in N,N-dimethylformamide (5 mL) was added N-[(dimethylamino)(3H-[1,2,3]triazolo[4,5-b]pyridin-3-yloxy)methylidene]-N-methylmethanaminium hexafluorophosphate (HATU) (172 mg, 0.454 mmol). The mixture was stirred for 2 hours and was partitioned between ethyl acetate and water. The mixture was extracted 3 times with ethyl acetate and the combined organic layers washed with 5% lithium chloride, dried over sodium sulfate, and concentrated. The residue was purified by silica chromatography eluting with a 0% to 10% gradient of methanol in dichloromethane. Fractions were concentrated and the residue purified by reverse phase HPLC. A portion of the oily residue (54 mg) was slurried in ethyl ether and scratched with a metal spatula. Precipitation occurred and white solids were collected by filtration to give the title compound (27 mg; 29%). LCMS: m/z 415, 417 (M+1).

### Example 9

### 1-(1-{[3,6-dichloro-8-(trifluoromethl)imidazo[1,2-a]pyridin-2-yl]carbonyl}-4-piperidinyl)-2-pyrrolidinone

### (Compound 110)

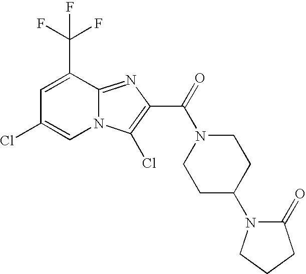

### Step A

### 5-chloro-3-(trifluoromethyl)-2-pyridinamine

To a solution of 3-(trifluoromethyl)-2-pyridinamine (12.2 g, 75 mmol) in N,N-dimethylformamide (100 mL) was added N-chlorosuccinimide (10.5 g, 79 mmol). The mixture was heated to 60° C. and stirred for 1 hour. The reaction was allowed to cool to room temperature, concentrated, and the residue purified with a large silica plug, eluting with dichloromethane to give the title compound (13.0 g; 88%). LCMS: m/z 197 (M+1).

### Step B

### ethyl 6-chloro-8-(trifluoromethyl)imidazo[1,2-a]pyridine-2-carboxylate

5-chloro-3-(trifluoromethyl)-2-pyridinamine (3.0 g, 15 mmol) and ethyl bromopyruvate (4.27 mL, 30.5 mmol) in N,N-dimethylformamide (50 mL) were stirred at 60° C. for 3 hours. The mixture was allowed to cool and then poured into stirring iced water. The precipitate was stirred for 5 minutes, collected by filtration, washed with water, and dried under vacuum to give the title compound (3.5 g; 78%). LCMS: m/z 293, 295 (M+1).

### Step C

### ethyl 3,6-dichloro-8-(trifluoromethyl)imidazo[1,2-a]pyridine-2-carboxylate

To a solution of ethyl 6-chloro-8-(trifluoromethyl)imidazo[1,2-a]pyridine-2-carboxylate (3.5 g, 12 mmol) in N,N-dimethylformamide (50 mL) was added N-chlorosuccinimide (1.68 g, 12.6 mmol). The mixture was heated to 60° C. After 2 hours, the mixture was cooled and concentrated to 10 mL. The mixture was partitioned between ethyl acetate and water, and then extracted 2 times with ethyl acetate. The combined organic layers were washed with saturated sodium sulfite, washed with saturated bicarbonate, washed with brine, dried over sodium sulfate, and concentrated. The product was dried under high vacuum to give the title compound (3.9 g; 99%). LCMS: m/z 327, 329 (M+1).

### Step D

### 1-(1-{[3,6-dichloro-8-(trifluoromethyl)imidazo[1,2-a]pyridin-2-yl]carbonyl}-4-piperidinyl)-2-pyrrolidinone

To a mixture of ethyl 3,6-dichloro-8-(trifluoromethyl)imidazo[1,2-a]pyridine-2-carboxylate (154 mg, 0.471 mmol) in tetrahydrofuran (4 mL) was added 1M sodium hydroxide (0.94 mL, 0.94 mmol). The mixture was stirred at room temperature for 4 hours and concentrated thoroughly. The residue was slurried in 15 mL of tetrahydrofuran before 1-(4-piperidinyl)-2-pyrrolidinone hydrochloride (96 mg, 0.471 mmol), N-ethyl-N-(1-methylethyl)-2-propanamine (0.33 mL, 1.9 mmol), and N-[(dimethylamino)(3H-[1,2,3]triazolo[4,5-b]pyridin-3-yloxy)methylidene]-N-methylmethanaminium hexafluorophosphate (HATU) (270 mg, 0.71 mmol) were added. The mixture was stirred at room temperature for 2 hours and was partitioned between ethyl acetate and water. The mixture was extracted 2 times with ethyl acetate. The combined organic layers were washed with brine, dried over sodium sulfate and concentrated. The residue was purified by reverse phase HPLC and appropriate fractions concentrated. The residue was slurried in ethyl ether and stirred for 10 minutes. White solids were collected by filtration and dried under vacuum to give the title compound (31 mg; 15%). LCMS: m/z 449, 451 (M+1).

### Example 10

### 1-(1-{[3-chloro-8-(trifluoromethyl)imidazo[1,2-a]pyridin-2-yl]carbonyl}-4-piperidinyl)-2-pyrrolidinone

### (Compound 111)

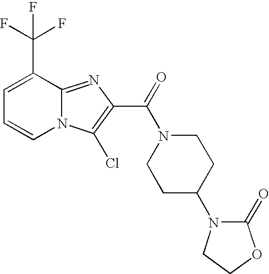

### 3-(1-{[3-chloro-8-(trifluoromethyl)imidazo[1,2-a]pyridin-2-yl]carbonyl}-4-piperidinyl)-1,3-oxazolidin-2-one

To 3-chloro-8-(trifluoromethyl)imidazo[1,2-a]pyridine-2-carboxylic acid (100 mg, 0.38 mmol) in N,N-dimethylformamide (3 mL) was added N-ethyl-N-(1-methylethyl)-2-propanamine (0.26 mL, 1.5 mmol). 3-(4-piperidinyl)-1,3-oxazolidin-2-one (86 mg: 0.42 mmol) was added to the reaction mixture before N-[(dimethylamino)(3H-[1,2,3]triazolo[4,5-b]pyridin-3-yloxy)methylidene]-N-methylmethanaminium hexafluorophosphate (HATU) (170 mg, 0.45 mmol) was added. The reaction was stirred at room temperature for 3 hours and partitioned between ethyl acetate (10 mL) and water (5 mL). The mixture was shaken and the water layer removed. The ethyl acetate layer was isolated, concentrated, and the residue purified by reverse phase HPLC. Appropriate fractions were concentrated and the residue slurried in ethyl ether and hexanes. The precipitate was collected by filtration to give the title compound (33 mg; 21%). LCMS: m/z 417, 419 (M+1).

### Example 11

### 3-(1-{[3,6-dichloro-8-(trifluoromethyl)imidazo[1,2-a]pyridin-2-yl]carbonyl)-4-piperidinyl)-1,3-oxazolidin-2-one

### (Compound 112)

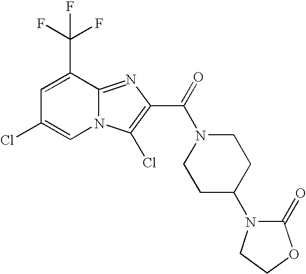

Ethyl 3,6-dichloro-8-(trifluoromethyl)imidazo[1,2-a]pyridine-2-carboxylate (0.13 g, 0.38 mmol) was dissolved in tetrahydrofuran (3 mL) before 1M sodium hydroxide (0.34 mL, 0.34 mmol) was added. The mixture was stirred at room temperature overnight. 1M sodium hydroxide (0.67 mL) was added and the mixture stirred for 2 hours. The mixture was concentrated and N,N-dimethylformamide (3 mL) added. To the mixture was added 3-(4-piperidinyl)-1,3-oxazolidin-2-one (95 mg; 0.46 mmol) followed by N-ethyl-N-(1-methylethyl)-2-propanamine (0.27 mL, 1.5 mmol), and N-[(dimethylamino)(3H-[1,2,3]triazolo[4,5-b]pyridin-3-yloxy)methylidene]-N-methylmethanaminium hexafluorophosphate (HATU) (0.22 g, 0.57 mmol). The mixture was stirred at room temperature overnight, diluted with 10 mL of 5% lithium chloride and 20 mL of ethyl acetate. The layers were separated and the organic phase concentrated. The residue was purified by reverse phase HPLC. Appropriate fractions were concentrated and the residue slurried in ethyl ether. Solids were collected by filtration to give the title compound (55 mg; 32%). LCMS: m/z 451, 453 (M+1).

### Example 12

### 3-(1-{[3-chloro-6-(4-pyridinyl)-8-(trifluoromethyl)imidazo[1,2-a]pyridin-2-yl carbonyl}-4-piperidinyl-1,3-oxazolidin-2-one

### (Compound 113)

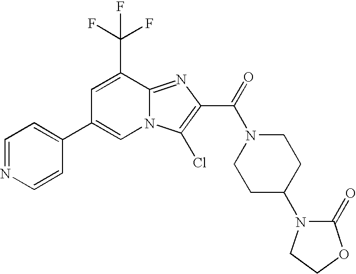

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (110 mg, 0.22 mmol), 4-pyridinylboronic acid (40.9 mg, 0.333 mmol), and 3M potassium phosphate tribasic (0.6 mL) in acetonitrile (2 mL) was added PdCl2(dppf)-CH2Cl2 adduct (36 mg, 0.044 mmol). The mixture was heated to 80° C. for 5 hours. The reaction was allowed to cool, quenched with water and extracted 2 times with dichloromethane. The combined organic layers were washed with brine, concentrated, and the residue subject to silica chromatography eluting with a gradient of 0% to 10% 2M ammonia/methanol in dichloromethane. Fractions were concentrated and the residue purified by reverse phase HPLC to give the title compound (15 mg; 14%). LCMS: m/z 494, 496 (M+1).

### Example 13

### 3-(1-{[3-chloro-6-(3-fluorophenyl)-8-(trifluoromethyl)imidazo[1,2-a]pyridin-2-yl[carbonyl}-4-piperidinyl)-1,3-oxazolidin-2-one

### (Compound 114)

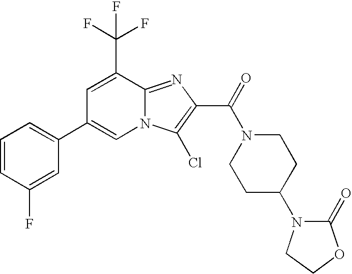

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), (3-fluorophenyl)boronic acid (0.30 mmol), and 3M potassium phosphate tribasic (0.6 mL, 1.800 mmol) in acetonitrile (2 mL) was added PdCl₂(dppf)-dichloromethane adduct (33.0 mg, 0.040 mmol) and the mixture heated to 85° C. for 4 hours. The reaction was quenched with water and extracted 2 times with dichloromethane. The combined organic layers were concentrated and the residue purified by silica chromatography eluting with a gradient of 0% to 5% 2M ammonia/methanol in dichloromethane.The material was dissolved in dimethylsulfoxide (˜0.5mL) and diluted with methanol. White solids were collected by filtration to give the title compound (6 mg; 6%). LCMS: m/z 511, 513 (M+1).

### Example 14

### 3-(1-{[3-chloro-6-(4-fluorophenyl)-8-(trifluoromethyl)imidazo[1,2-a]pyridin-2-yl]carbonyl}-4-piperidinyl)-1,3-oxazolidin-2-one

### (Compound 115)

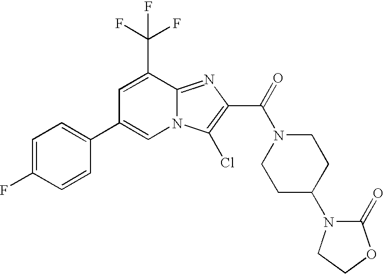

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), (4-fluorophenyl)boronic acid (0.303 mmol), and 3M potassium phosphate tribasic (0.6 mL, 1.800 mmol) in acetonitrile (2 mL) was added PdCl2(dppf)-dichloromethane adduct (33.0 mg, 0.040 mmol) and the mixture heated to 85° C. for 4 hours. The reaction was quenched with water and extracted 2 times with dichloromethane. The combined organic layers were concentrated. The residue was purified by silica chromatography eluting with a gradient of 0% to 5% 2M ammonia/methanol in dichloromethane. Fractions were concentrated and the residue was crystallized from ether and a small amount of methanol to give the title compound (32 mg; 26%). LCMS: m/z 511, 513 (M+1).

### Example 15

### 3-(1-{[3-chloro-6-[2-(methyloxy)phenyl]-8-(trifluoromethyl)imidazo[1,2-a]pyridin-2-yl]carbonyl)-4-piperidinyl-1,3-oxazolidin-2-one

### (Compound 116)

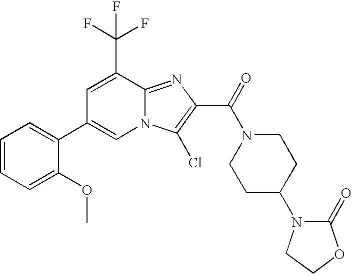

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), [2-(methyloxy)phenyl]boronic acid (0.303 mmol), and 3M potassium phosphate tribasic (0.60 mL, 1.8 mmol) in acetonitrile (2 mL) was added PdCl₂(dppf)-dichloromethane adduct (33.0 mg, 0.040 mmol). The mixture was heated to 85° C. for 4 hours, cooled to room temperature, quenched with water, extracted 2 times with dichloromethane, and concentrated. The residue was purified first by silica chromatography eluting with a gradient of 0% to 5% 2M ammonia/methanol in dichloromethane. This product was further purified by reverse phase HPLC to give the title compound (31 mg; 29%). LCMS: m/z 523, 525 (M+1).

### Example 16

### 3-(1-{[3-chloro-6-(3-(methyloxy)phenyl]-8-(trifluoromethyl)imidazo[1,2-a]pyridin-2-yl]carbonyl}-4-piperidinyl-1,3-oxazolidin-2-one

### (Compound 117)

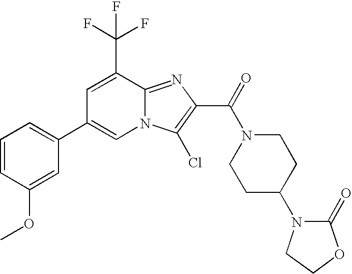

### Step A

### 3-(1-{[3-chloro-6-(3-(methyloxy)phenyl]-8-(trifluoromethyl)imidazo[1,2-a]pyridin-2-yl]carbonyl}-4-piperidinyl-1,3-oxazolidin-2-one

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), [3-(methyloxy)phenyl]boronic acid (0.303 mmol), and 3M potassium phosphate tribasic (0.6 mL, 1.8 mmol) in acetonitrile (2 mL) was added PdCl₂(dppf)-dichloromethane adduct (33.0 mg, 0.040 mmol). The mixture was heated to 85° C. for 4 hours, cooled to room temperature, quenched with water, extracted 2 times with dichloromethane, and concentrated. The residue was slurried in 3 mL of methanol, cooled to 0° C., and solids collected by filtration. The filter cake was washed with cold methanol and dried under vacuum to give the title compound (42 mg; 40%). LCMS: m/z 523, 525 (M+1).

### Example 17

### 3-(1-{[3-chloro-6-[4-(methyloxy)phenyl]-8-(trifluoromethyl)imidazo[1,2-a]pyridin-2-yl]carbonyl}-4-piperidinyl-1,3-oxazolidin-2-one

### (Compound 118)

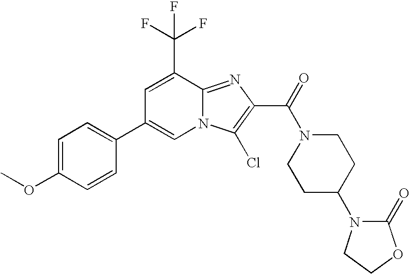

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), [4-(methyloxy)phenyl]boronic acid (0.303 mmol), and 3M potassium phosphate tribasic (0.6 mL, 1.8 mmol) in acetonitrile (2 mL) was added PdCl₂(dppf)-dichloromethane adduct (33.0 mg, 0.040 mmol). The mixture was heated to 85° C. for 4 hours, cooled to room temperature, quenched with water, extracted 2 times with dichloromethane, and concentrated. The residue was subject to reverse phase HPLC. Appropriate fractions were concentrated. The residue was stirred in cold methanol, white solids collected by filtration, and dried to give the title compound (26 mg; 25%). LCMS: m/z 523, 525 (M+1).

### Example 18

### 3-(1-{[3-chloro-6-(3-thienyl)-8-(trifluoromethyl)imidazo[1,2-a]pyridin-2-yl]carbonyl}-4-piperidinyl-1,3-oxazolidin-2-one

### (Compound 119)

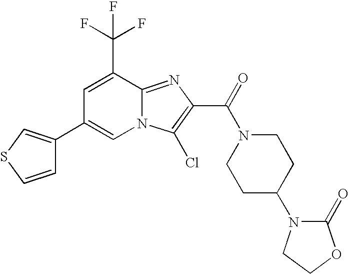

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (120 mg, 0.242 mmol), 3-thienylboronic acid (0.36 mmol), and 3M potassium phosphate tribasic (0.718 mL, 2.155 mmol) in acetonitrile (2 mL) was added PdCl₂(dppf)-dichloromethane adduct (40 mg, 0.048 mmol). The mixture was heated to 85° C. for 4 hours, cooled to room temperature, quenched with water, and extracted 2 times with dichloromethane. The combined organic layers were concentrated and the residue purified by reverse phase HPLC to give the title compound (36 mg; 30%). LCMS: m/z 499, 501 (M+1).

### Example 19

### 3-(1-{[3-chloro-6-(8-quinolinyl)-8-(trifluoromethyl)imidazo[1,2-a]pyridin-2-yl]carbonyl}-4-piperidinyl-1,3-oxazolidin-2-one

### (Compound 120)

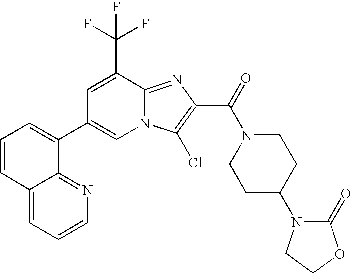

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (120 mg, 0.24 mmol), 8-quinolinylboronic acid (0.36 mmol), and 3M potassium phosphate tribasic (0.72 mL, 2.2 mmol) in acetonitrile (2 mL) was added PdCl₂(dppf)-dichloromethane adduct (40 mg, 0.048 mmol). The mixture was heated to 85° C. for 4 hours, cooled to room temperature, quenched with water, and extracted 2 times with dichloromethane. The combined organic layers were concentrated and the residue purified by reverse phase HPLC to give the title compound. LCMS: m/z 544, 546 (M+1).

### Example 20

### 3-(1-{[3-chloro-6-(2-hydroxyphenyl)-8-(trifluoromethyl)imidazo[1,2-a]pyridin-2-yl]carbonyl}-4-piperidinyl-1,3-oxazolidin-2-one

### (Compound 121)

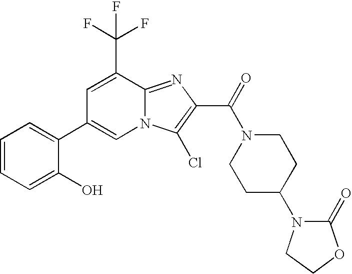

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), (2-hydroxyphenyl)boronic acid (0.30 mmol), and 3M potassium phosphate tribasic (0.60 mL, 1.8 mmol) in acetonitrile (2 mL) was added PdCl₂(dppf)-dichloromethane adduct (33 mg, 0.040 mmol). The mixture was heated to 85° C. for 4 hours, cooled to room temperature, quenched with water, extracted 2 times with dichloromethane, and concentrated. The residue was purified by silica chromatography eluting with a gradient of 0% to 5% 2M ammonia/methanol in dichloromethane. Fractions were concentrated and the residue crystallized from ethyl acetate and ethyl ether to give the title compound (10 mg; 10%). LCMS: m/z 509, 511 (M+1).

### Example 21

### 3-(1-{[3-chloro-6-(3-hydroxyphenyl-8-(trifluoromethyl)imidazo[1,2-a]pyridin-2-yl]carbonyl}-4-piperidinyl-1,3-oxazolidin-2-one

### (Compound 122)

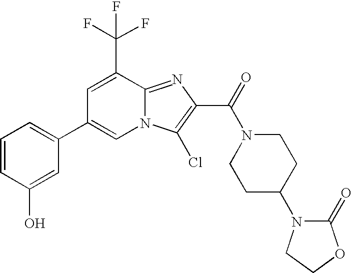

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), (3-hydroxyphenyl)boronic acid (0.30 mmol), and 3M potassium phosphate tribasic (0.60 mL, 1.8 mmol) in acetonitrile (2 mL) was added PdCl₂(dppf)-dichloromethane adduct (33 mg, 0.040 mmol). The mixture was heated to 85° C. for 4 hours, cooled to room temperature, quenched with water, extracted 2 times with dichloromethane, and concentrated. The residue was purified by silica chromatography eluting with a gradient of 0% to 5% 2M ammonia/methanol in dichloromethane. Fractions were concentrated and the residue crystallized from dichloromethane and hexanes to give the title compound (32 mg; 31%). LCMS: m/z 509, 511 (M+1).

### Example 22

### 3-(1-{[3-chloro-6-(4-hydroxyphenyl-8-(trifluoromethyl)imidazo[1,2-a]pyridin-2-yl]carbonyl}-4-piperidinyl-1,3-oxazolidin-2-one

### (Compound 123)

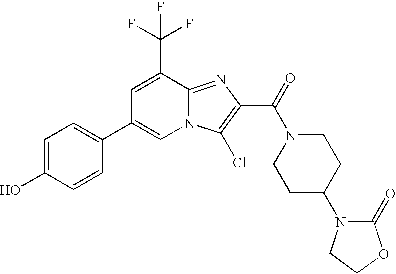

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), (4-hydroxyphenyl)boronic acid (0.30 mmol), and 3M potassium phosphate tribasic (0.6 mL, 1.800 mmol) in acetonitrile (2 mL) was added PdCl₂(dppf)-dichloromethane adduct (33 mg, 0.040 mmol). The mixture was heated to 85° C. for 4 hours, cooled to room temperature, quenched with water, extracted 2 times with dichloromethane, and concentrated. The residue was purified by silica chromatography eluting with a gradient of 0% to 5% 2M ammonia/methanol in dichloromethane. Fractions were concentrated and the residue crystallized from ethyl acetate and ether to give the title compound (14 mg; 14%). LCMS: m/z 509, 511 (M+1).

### Example 23

### 3-(1-{[3-chloro-6-(1-methyl-1H-pyrazol-4-yl)-8-(trifluoromethyl)imidazo[1,2-a]pyridin-2-yl]carbonyl}-4-piperidinyl-1,3-oxazolidin-2-one

### (Compound 124)

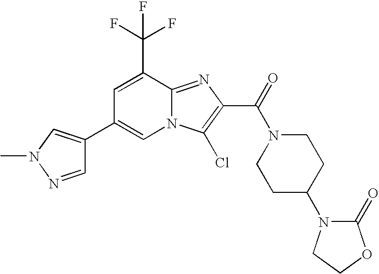

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), 1-methyl-4-(4,4,5,5-tetramethyl-1,3,2-dioxaborolan-2-yl)-1H-pyrazole (0.30 mmol), and 3M potassium phosphate tribasic (0.60 mL, 1.8 mmol) in acetonitrile (2 mL) was added PdCl₂(dppf)-dichloromethane adduct (33 mg, 0.040 mmol). The mixture was heated to 85° C. for 4 hours, cooled to room temperature, quenched with water, extracted 2 times with dichloromethane, and concentrated. The residue was purified by silica chromatography eluting with a gradient of 0% to 5% 2M ammonia/methanol in dichloromethane to give the title compound (20 mg; 20%). LCMS: m/z 497, 499 (M+1).

### Example 24

### 3-(1-{[3-chloro-6-(1-ethyl-1H-pyrazol-4-yl)-8-(trifluoromethyl)imidazo[1,2-a]pyridin-2-yl]carbonyl}-4-piperidinyl-1,3-oxazolidin-2-one

### (Compound 125)

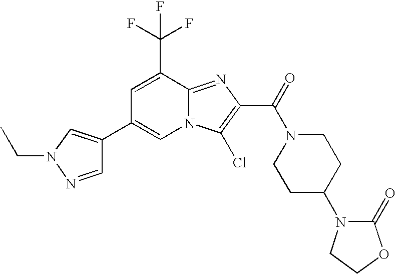

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.20 mmol), 1-ethyl-4-(4,45,5-tetramethyl-1,3,2-dioxaborolan-2-yl)-1H-pyrazole (0.30 mmol), and 3M potassium phosphate tribasic (0.60 mL, 1.8 mmol) in acetonitrile (2 mL) was added PdCl₂(dppf)-dichlromethane adduct (33 mg, 0.040 mmol). The mixture was heated to 85° C. for 4 hours, cooled to room temperature, quenched with water, extracted 2 times with dichloromethane, and concentrated. The residue was purified by silica chromatography eluting with a gradient of 0% to 5% 2M ammonia/methanol in dichloromethane to give the title compound (34 mg; 32%). LCMS: m/z 511, 513 (M+1).

### Example 25

### 3-(1-{[3-chloro-6-[1-(2-methylpropyl)-1H-pyrazol-4-yl]-8-(trifluoromethyl)imidazo[1,2-a]pyridin-2-yl]carbonyl}-4-piperidinyl)-1,3-oxazolidin-2-one

### (Compound 126)

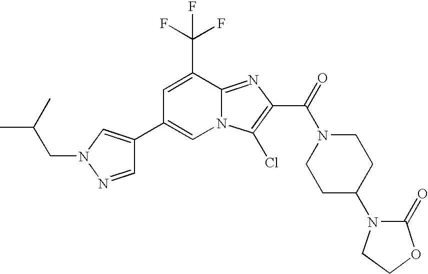

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (100 mg, 0.202 mmol), 1-(2-methylpropyl)-4-(4,4,5,5-tetramethyl-1,3,2-dioxaborolan-2-yl)-1H-pyrazole (0.303 mmol), and 3M potassium phosphate tribasic (0.6 mL, 1.800 mmol) in acetonitrile (2 mL) was added PdCl₂(dppf)-dichloromethane adduct (33 mg, 0.040 mmol). The mixture was heated to 85° C. for 4 hours, cooled to room temperature, quenched with water, extracted 2 times with dichloromethane, and concentrated. The residue was purified by silica chromatography eluting with a gradient of 0% to 5% 2M ammonia/methanol in dichloromethane. Fractions were concentrated and the residue further purified by reverse phase HPLC to give the title compound (9 mg; 8%). LCMS: m/z 539, 541 (M+1).

### Example 26

### 3-(1-{[3-chloro-6-cyclopentyl-8-(trifluoromethyl)imidazo[1,2-a]pyridin-2-yl]carbonyl)-4-piperidinyl)-1,3-oxazolidin-2-one

### (Compound 127)

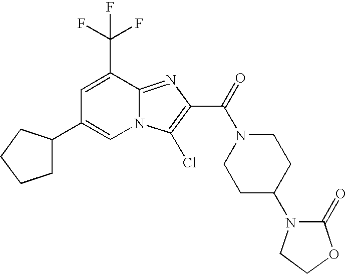

### Step A

### methyl 3-chloro-6-cyclopentyl-8-(trifluoromethyl)imidazo[1,2-a]pyridine-2-carboxylate

PdCl₂(dppf)-dichloromethane adduct (94 mg, 0.115 mmol) and copper(I) iodide (66 mg, 0.34 mmol) were added as one portion to methyl 6-bromo-3-chloro-8-(trifluoromethyl)imidazo[1,2-a]pyridine-2-carboxylate (410 mg, 1.14 mmol) in tetrahydrofuran (10 mL). Cyclopentylzinc bromide (0.5M in tetrahydrofuran) (3.44 mL, 1.72 mmol) was then added slowly dropwise (1 drop every 2-3 sec). The mixture was stirred at room temperature for 3 hours, concentrated, and the residue subject to silica chromatography eluting with a gradient of 0% to 3% ethyl acetate in dichloromethane. Fractions were concentrated and the residue further purified by reverse phase HPLC to give the title compound (80 mg; 20%). LCMS: m/z 347, 349 (M+1).

### Step B

### 3-chloro-6-cyclopentyl-8-(trifluoromethyl)imidazo[1,2-a]pyridine-2-carboxylic acid

Methyl 3-chloro-6-cyclopentyl-8-(trifluoromethyl)imidazo[1,2-a]pyridine-2-carboxylate (80 mg, 0.23 mmol) was mixed in tetrahydrofuran (1 mL) and water (1 mL) before sodium hydroxide (1M) (0.58 mL, 0.58 mmol) was added. The mixture was stirred for 1 hour before hydrochloric acid (1M) (1.15 mL, 1.15 mmol) was added. The mixture was extracted 3 times with ethyl acetate, washed with brine, dried over sodium sulfate, concentrated, and dried under vacuum to give the title compound (74 mg; 92%). LCMS: m/z 333, 335 (M+1).

### Step C

### 3-(1-{[3-chloro-6-cyclopentyl-8-(trifluoromethyl)imidazo[1,2-a]pyridin-2-yl]carbonyl}-4-piperidinyl)-1,3-oxazolidin-2-one

To a mixture of 3-chloro-6-cyclopentyl-8-(trifluoromethyl)imidazo[1,2-a]pyridine-2-carboxylic acid (75 mg, 0.23 mmol) and 3-(4-piperidinyl)-1,3-oxazolidin-2-one hydrochloride (49 mg, 0.24 mmol) in N,N-dimethylformamide (2 mL) was added N-ethyl-N-(1-methylethyl)-2-propanamine (0.12 mL, 0.68 mmol). N-[(dimethylamino)(3H[1,2,3]triazolo[4,5-b]pyridin-3-yloxy)methylidene]-N-methylmethanaminium hexafluorophosphate (HATU) (95 mg, 0.25 mmol) was added and the mixture stirred at room temperature for 1 hour. The mixture was diluted with water, extracted 2 times with ethyl acetate, washed with 5% lithium chloride, dried over sodium sulfate, and concentrated. The residue was triturated with ether and hexanes and solids collected by filtration to give the title compound (57 mg; 52%). LCMS: m/z 485, 487 (M+1).

### Example 27

### 3-(1-{[3-chloro-6-cyclobutyl-8-(trifluoromethyl)imidazo[1,2-a]pyridin-2-yl]carbonyl)-4-piperidinyl)-1,3-oxazolidin-2-one

### (Compound 128)

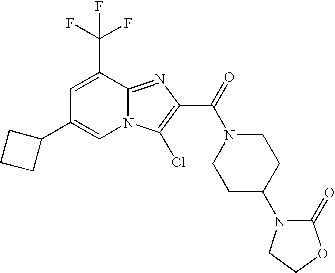

### Step A

### methyl 3-chloro-6-cyclobutyl-8-(trifluoromethyl)imidazo[1,2-a]pyridine-2-carboxylate

PdCl₂(dppf)-dichloromethane adduct (0.119 g, 0.145 mmol) and copper(I) iodide (0.083 g, 0.44 mmol) were added as one portion to methyl 6-bromo-3-chloro-8-(trifluoromethyl)imidazo[1,2-a]pyridine-2-carboxylate (0.52 g, 1.4 mmol) in tetrahydrofuran (10 mL). Cyclobutylzinc bromide (0.5M in tetrahydrofuran) (4.4 mL, 2.2 mmol) was then added slowly dropwise (1 drop every 2-3 sec). The mixture was stirred for 3 hours, concentrated, and the residue subject to subject to silica chromatography eluting with a gradient of 0% to 2.5% acetonitrile in dichloromethane. Fractions were concentrated and the residue was purified by reverse phase HPLC to give the title compound (60 mg; 12%). LCMS: m/z 333, 335 (M+1).

### Step B

### 3-chloro-6-cyclobutyl-8-(trifluoromethyl)imidazo[1,2-a]pyridine-2-carboxylic acid

Methyl 3-chloro-6-cyclobutyl-8-(trifluoromethyl)imidazo[1,2-a]pyridine-2-carboxylate (49 mg, 0.15 mmol) was dissolved in tetrahydrofuran (1 mL) before water (1 mL) was added. Sodium hydroxide (1 M) (0.37 mL, 0.37 mmol) was added and the mixture stirred at room temperature for 1 hour. The mixture was quenched with hydrochloric acid (1M) (0.59 mL, 0.59 mmol) and extracted 2 times with ethyl acetate. The combined ethyl acetate layers were washed with brine, dried over sodium sulfate, and concentrated to give the title compound (44 mg: 94%). LCMS: m/z 319, 321 (M+1).

### Step C

### 3-(1-{[3-chloro-6-cyclobutyl-8-(trifluoromethyl)imidazo[1,2-a]pyridin-2-yl]carbonyl}-4-piperidinyl)-1,3-oxazolidin-2-one

To a mixture of 3-chloro-6-cyclobutyl-8-(trifluoromethyl)imidazo[1,2-a]pyridine-2-carboxylic acid (44 mg, 0.14 mmol) and 3-(4-piperidinyl)-1,3-oxazolidin-2-one hydrochloride (31.4 mg, 0.152 mmol) in N,N-dimethylformamide (1 mL) was added N-ethyl-N-(1-methylethyl)-2-propanamine (0.096 mL, 0.55 mmol). N-[(dimethylamino)(3H-[1,2,3]triazolo[4,5-b]pyridin-3-yloxy)methylidene]-N-methylmethanaminium hexafluorophosphate (HATU) (63.0 mg, 0.166 mmol) was added and the mixture stirred at room temperature for 1 hour. The mixture was diluted with 200 mL of ethyl acetate and washed with water. The water layer was back extracted with ethyl acetate. The combined organic layers were washed 2 times with brine and 1 time with 5% lithium chloride, dried over sodium sulfate, concentrated, and the residue purified by silica chromatography eluting with a gradient of 0% to 4% 2M ammonia/methanol in dichloromethane. Appropriate fractions were concentrated and the residue dissolved in dichloromethane before hexane was added. The mixture was stirred for 5 minutes and the precipitate collected by filtration to give the title compound (42 mg; 65%). LCMS: m/z 471, 473 (M+1).

### Example 28

### 3-(1-{[3-chloro-6-propyl-8-(trifluoromethyl)imidazo[1,2-a]pyridin-2-yl]carbonyl}-4-piperidinyl-1,3-oxazolidin-2-one

### (Compound 129)

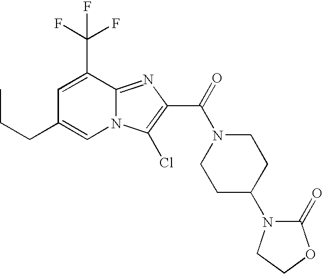

### Step A

### methyl 3-chloro-6-propyl-8-(trifluoromethyl)imidazo[1,2-a]pyridine-2-carboxylate

PdCl₂(dppf)-dichloromethane adduct (57 mg, 0.070 mmol) was added to methyl 6-bromo-3-chloro-8-(trifluoromethyl)imidazo[1,2-a]pyridine-2-carboxylate (250 mg, 0.699 mmol) in tetrahydrofuran (5 mL). Propylzinc bromide (0.5M in tetrahydrofuran) (2.10 mL, 1.05 mmol) was then added slowly dropwise (1 drop every 2-3 sec) at room temperature. The reaction appeared to be complete within 2 hours and was diluted with dichloromethane. The mixture was washed 1 time with water and 2 times with brine, dried over sodium sulfate, and concentrated. The residue was purified by reverse phase HPLC to give the title compound (96 mg; 43%). LCMS: m/z 321, 323 (M+1).

### Step B

### 3-chloro-6-propyl-8-(trifluoromethyl)imidazo[1,2-a]pyridine-2-carboxylic acid

Methyl 3-chloro-6-propyl-8-(trifluoromethyl)imidazo[1,2-a]pyridine-2-carboxylate (94 mg, 0.29 mmol) was dissolved in tetrahydrofuran (1.9 mL) and water (1.9 mL) added. Sodium hydroxide (1M) (0.733 mL, 0.733 mmol) was added and the mixture stirred at room temperature for 3 hours. The mixture was quenched with hydrochloric acid (1 M) (1.2 mL, 1.2 mmol), and extracted 3 times with ethyl acetate. The combined organic layers were washed with brine, dried over sodium sulfate, and concentrated to give the title compound (87 mg; 97%). LCMS: m/z 307, 309 (M+1).

### Step C

### 3-(1-{[3-chloro-6-propyl-8-(trifluoromethyl)imidazo[1,2-a]pyridin-2-yl]carbonyl}-4-piperidinyl)-1,3-oxazolidin-2-one

To a mixture of 3-chloro-6-propyl-8-(trifluoromethyl)imidazo[1,2-a]pyridine-2-carboxylic acid (45 mg, 0.15 mmol) and 3-(4-piperidinyl)-1,3-oxazolidin-2-one hydrochloride (33.4 mg, 0.161 mmol) was added N-ethyl-N-(1-methylethyl)-2-propanamine (0.10 mL, 0.59 mmol). N-[(dimethylamino)(3H-[1,2,3]triazolo[4,5-b]pyridin-3-yloxy)methylidene]-N-methylmethanaminium hexafluorophosphate (HATU) (67.0 mg, 0.176 mmol) was added and the mix stirred at room temperature for 1 hour. The mixture was diluted with ethyl acetate, washed with water, washed with brine, washed with 5% lithium chloride, dried over sodium sulfate, and concentrated. The residue was purified by silica chromatography eluting with a gradient of 0% to 4% 2M ammonia/methanol in dichloromethane. The residue was crystallized from dichloromethane and hexanes to give the title compound (45 mg; 67%). LCMS: m/z 459, 461 (M+1).

### Example 29

### 3-(1-{[3-chloro-6-(2-methylpropyl)-8-(trifluoromethyl)imidazo[1,2-a]pyridin-2-yl]carbonyl≡6-4-piperidinyl)-1,3-oxazolidin-2-one

### (Compound 130)

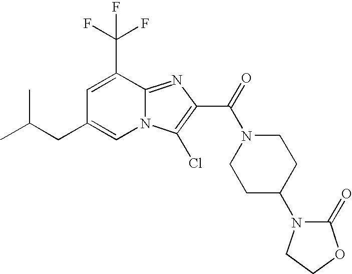

### Step A

### methyl 3-chloro-6-(2-methylpropyl)-8-(trifluoromethyl)imidazo[1,2-a]pyridine-2-carboxylate

PdCl₂(dppf)-dichloromethane adduct (96 mg, 0.12 mmol) was added to methyl 6-bromo-3-chloro-8-(trifluoromethyl)imidazo[1,2-a]pyridine-2-carboxylate (420 mg, 1.18 mmol) in tetrahydrofuran (5 ml). Isobutylzinc bromide (0.5M in tetrahydrofuran) (3.52 ml, 1.76 mmol) was then added slowly dropwise (1 drop every 2-3 sec) at room temperature. The mixture was stirred overnight, diluted with ethyl acetate, washed with water, washed 2 times with brine, dried over sodium sulfate, and concentrated. The residue was subject to reverse phase HPLC to give the title compound (95 mg; 24%). LCMS: m/z 335, 337 (M+1).

### Step B

### 3-(1-{[3-chloro-6-(2-methylpropyl)-8-(trifluoromethyl)imidazo[1,2-a]pyridin-2-yl]carbonyl}-4-piperidinyl)-1,3-oxazolidin-2-one

Methyl 3-chloro-6-(2-methylpropyl)-8-(trifluoromethyl)imidazo[1,2-a]pyridine-2-carboxylate (95 mg, 0.28 mmol) was dissolved in tetrahydrofuran (2 mL) and water (2 mL) added. Sodium hydroxide (1 M) (0.71 mL, 0.71 mmol) was added and the mixture stirred at room temperature for 1 hour. The mixture was quenched with hydrochloric acid (1 M) (1.13 mL, 1.13 mmol) and extracted 3 times with ethyl acetate. The combined organic layers were washed with brine, dried over sodium sulfate, and concentrated. The residue was dissolved in thionyl chloride (0.021 mL, 0.28 mmol) and refluxed for 1 hour. The mixture was concentrated and the residue co-evaporated 2 times with toluene, dried under vacuum, and dissolved in dichloromethane (4 mL). Two 2 mL aliquots were separated. One aliquot was added to a mixture of N-ethyl-N-(1-methylethyl)-2-propanamine (0.099 mL, 0.568 mmol) and 3-(4-piperidinyl)-1,3-oxazolidin-2-one hydrochloride (29.3 mg, 0.142 mmol) in dichloromethane. The reaction was complete within 1 hour and was quenched with saturated sodium bicarbonate. The mixture was extracted with dichloromethane, washed with brine, and concentrated. The residue was purified by silica chromatography eluting with a gradient of 0% to 4% 2M ammonia/methanol in dichloromethane to give the title compound (37 mg; 55%). LCMS: m/z 473, 475 (M+1).

### Example 30

### 3-(1-{[6-butyl-3-chloro-8-(trifluoromethyl)imidazo[1,2-a]pyridin-2-yl]carbonyl}-4-piperidinyl-1,3-oxazolidin-2-one

### (Compound 131)

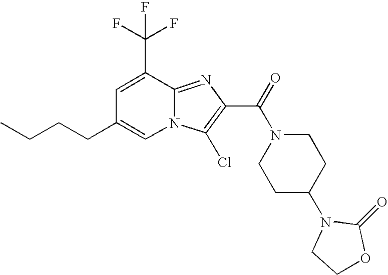

### Step A

### methyl 6-butyl-3-chloro-8-(trifluoromethyl)imidazo[1,2-a]pyridine-2-carboxylate

PdCl₂(dppf)-dichloromethane adduct (113 mg, 0.139 mmol) was added to methyl 6-bromo-3-chloro-8-(trifluoromethyl)imidazo[1,2-a]pyridine-2-carboxylate (496 mg, 1.39 mmol) in tetrahydrofuran (10 ml). Butylzinc bromide (0.5M in tetrahydrofuran) (4.16 ml, 2.08 mmol) was then added slowly dropwise (1 drop every 2-3 sec) at room temperature. The reaction appeared to be complete within 1 hour and was diluted with dichloromethane, washed 1 time with water, washed 2 times with brine, dried over sodium sulfate, and concentrated. The residue was purified by silica chromatography eluting with a gradient of 0% to 2% acetonitrile in dichloromethane to give the title compound (143 mg; 31%). LCMS: m/z 335, 337 (M+1).

### Step B

### 3-(1-{[6-butyl-3-chloro-8-(trifluoromethyl)imidazo[1,2-a]pyridin-2-yl]carbonyl}-4-piperidinyl-1,3-oxazolidin-2-one

Methyl 6-butyl-3-chloro-8-(trifluoromethyl)imidazo[1,2-a]pyridine-2-carboxylate (143 mg, 0.427 mmol) was dissolved in tetrahydrofuran (2 mL) and water (2 mL) added. Sodium hydroxide (1 M) (1.07 mL, 1.07 mmol) was added and the mixture stirred at room temperature for 1 hour. The mixture was quenched with hydrochloric acid (1 M) (1.71 mL, 1.71 mmol) and extracted 3 times with ethyl acetate. The combined organic layers were washed with brine, dried over sodium sulfate, and concentrated. The residue was dissolved in thionyl chloride (5 mL, 70 mmol), refluxed for 1 hour, and concentrated. The residue was co-evaporated 2 times with toluene and then dissolved in 3 mL of dichloromethane. Three 1 mL aliquots were separated. To one of the 1 mL aliquots was added N-ethyl-N-(1-methylethyl)-2-propanamine (0.097 mL, 0.56 mmol) and 3-(4-piperidinyl)-1,3-oxazolidin-2-one hydrochloride. The reaction was stirred for 1 hour and quenched with saturated sodium bicarbonate. The mixture was extracted with dichloromethane and the organic phase concentrated. The residue was triturated in a mixture of dichloromethane, ethyl ether, and hexanes. Solids were collected by filtration and washed with hexanes to give the title compound (50 mg; 75%). LCMS: m/z 473, 475 (M+1).

### Example 31

### 3-(1-{[3-chloro-6-(methyloxy)-8-(trifluoromethyl)imidazo[1,2-a]pyridin-2-yl]carbonyl}-4-piperidinyl)-1,3-oxazolidin-2-one

### (Compound 132)

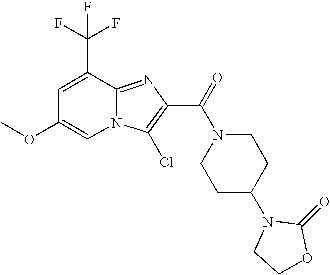

### Step A

### methyl 3-chloro-6-hydroxy-8-(trifluoromethyl)imidazo[1,2-a]pyridine-2-carboxylate

Methyl 6-bromo-3-chloro-8-(trifluoromethyl)imidazo[1,2-a]pyridine-2-carboxylate (280 mg, 0.783 mmol), potassium acetate (231 mg, 2.35 mmol), bis(pinacolato)diboron (447 mg, 1.76 mmol), and PdCl₂(dppf)-dichloromethane adduct (64 mg, 0.078 mmol) in 1,4-dioxane (20 mL) were heated to 100° C. for 2 hours. The mixture was cooled to room temperature before acetic acid (0.090 mL, 1.6 mmol) and 4 mL of water were added. The mixture was stirred for 1 hour and hydrogen peroxide (0.160 mL, 1.57 mmol) was added. The reaction was complete within 3 hours and was quenched with a sodium bisulfite solution. The mixture was extracted with ethyl acetate, washed with brine, and concentrated. The residue was slurried in dichloromethane and ethyl ether and the precipitate collected by filtration to give the title compound (107 mg; 46%). LCMS: m/z 295, 297 (M+1).

### Step B

### methyl 3-chloro-6-(methyloxy)-8-(trifluoromethyl)imidazo[1,2-a]pyridine-2-carboxylate

Methyl 3-chloro-6-hydroxy-8-(trifluoromethyl)imidazo[1,2-a]pyridine-2-carboxylate (107 mg, 0.363 mmol), potassium carbonate (100 mg, 0.726 mmol), and methyl iodide (0.027 mL, 0.44 mmol) in N,N-dimethylformamide (5 mL) were stirred at room temperature for 2 hours. The mixture was diluted with water and extracted with ethyl acetate. The organic phase was washed with 5% lithium chloride, washed with brine, dried over sodium sulfate, and concentrated. The residue was purified by silica chromatography eluting with a gradient of 0% to 5% ethyl acetate in dichloromethane to give the title compound (65 mg; 58%). LCMS: m/z 309, 311 (M+1).

### Step C

### 3-(1-{[3-chloro-6-(methyloxy)-8-(trifluoromethyl)imidazo[1,2-a]pyridin-2-yl]carbonyl}-4-piperidinyl)-1,3-oxazolidin-2-one

Methyl 3-chloro-6-(methyloxy)-8-(trifluoromethyl)imidazo[1,2-a]pyridine-2-carboxylate (59 mg, 0.19 mmol) was dissolved in tetrahydrofuran (1.2 mL). Water (1.2 mL) and sodium hydroxide (0.478 mL, 0.478 mmol) were added and the mixture stirred for 1 hour at room temperature. 2 mL of 1N hydrochloric acid was added and the mixture extracted 2 times with ethyl acetate. The combined organic layers were washed with brine, dried over sodium sulfate, and concentrated. The residue was dissolved in N,N-dimethylformamide (1 mL) before N-ethyl-N-(1-methylethyl)-2-propanamine (0.134 mL, 0.765 mmol), 3-(4-piperidinyl)-1,3-oxazolidin-2-one hydrochloride (44 mg; 0.21 mmol) and N-[(dimethylamino)(3H41,2,3]triazolo[4,5-b]pyridin-3-yloxy)methylidene]-N-methylmethanaminium hexafluorophosphate (HATU) (87 mg, 0.229 mmol) were added. The mixture was stirred for 1 hour, quenched with water, extracted 2 times with ethyl acetate. The combined organic layers were washed with brine, washed with 5% lithium chloride, and concentrated. The residue was purified by silica chromatography eluting with a gradient of 0% to 3% 2M ammonia/methanol in dichloromethane. Appropriate fractions were concentrated and the residue was re-crystallized from dichloromethane/ethyl ether/hexanes to give the slightly impure product (65 mg). The product was purified by reverse phase HPLC to give the title compound (11 mg; 13%). LCMS: m/z 447, 449 (M+1).

### Example 32

### 3-(1-{[3-chloro-6-(5-methyl-2-furanyl-8-(trifluoromethyl)imidazo[1,2-a]pyridin-2-yl]carbonyl}-4-piperidinyl-1,3-oxazolidin-2-one

### (Compound 133)

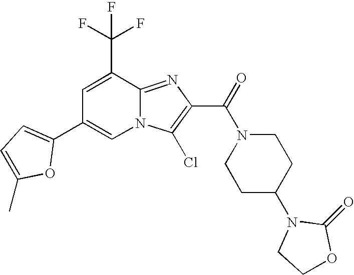

To a mixture of 3-(1-{[6-bromo-3-chloro-8-(trifluoromethyl)imidazo[1,2-a]pyridin-2-yl]carbonyl}-4-piperidinyl)-1,3-oxazolidin-2-one (277 mg, 0.559 mmol), 4,4,5,5-tetramethyl-2-(5-methyl-2-furanyl)-1,3,2-dioxaborolane (174 mg, 0.838 mmol), and 3M potassium phosphate tribasic (0.559 mL, 1.68 mmol) in N,N-dimethylformamide (3 mL) was added PdCl₂(dppf)-dichloromethane adduct (45.6 mg, 0.056 mmol) and the mixture heated to 80° C. for 2 hours. The mixture was allowed to cool to room temperature, diluted with water, and extracted with ethyl acetate. The organic phase was washed with 5% lithium chloride, washed with brine, dried over sodium sulfate, and concentrated. The residue was purified by silica chromatography eluting with a gradient of 0% to 5% ethyl acetate in dichloromethane to give the title compound (130 mg; 45%). LCMS: m/z 497, 499 (M+1).

### Example 33

### 3-(1-{[3-chloro-6-(tetrahydro-2-furanyl)-8-(trifluoromethyl)imidazo[1,2-a]pyridin-2-yl]carbonyl}-4-piperidinyl)-1,3-oxazolidin-2-one

### (Compound 134)

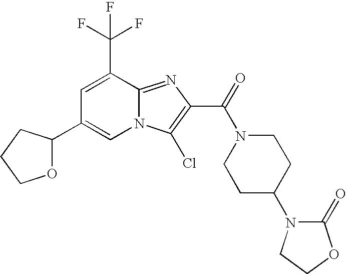

### Step A

### methyl 3-chloro-6-(2,5-dihydro-2-furanyl)-8-(trifluoromethyl)imidazo[1,2-a]pyridine-2-carboxylate

Methyl 6-bromo-3-chloro-8-(trifluoromethyl)imidazo[1,2-a]pyridine-2-carboxylate (2.0 g; 5.6 mmol), palladium(II) acetate (0.13 g; 0.56 mmol), potassium acetate (1.4 g; 14 mmol), 2,3-dihydrofuran (1.96 g; 28.0 mmol) and tetrabutylammonium bromide (1.8 g; 5.6 mmol) in N,N-dimethylformamide (30 mL) were stirred at 80° C. for 1 hour. The mixture was allowed to cool, diluted with water, and extracted with ethyl acetate. The organic phase was washed with 5% lithium chloride, washed with brine, dried over sodium sulfate, and concentrated. The residue was subject to silica chromatography eluting with a gradient of 0% to 10% ethyl acetate in dichloromethane to give the title compound (970 mg; 50%). LCMS: m/z 347, 349 (M+1).

### Step B

### methyl 3-chloro-6-(tetrahydro-2-furanyl)-8-(trifluoromethyl)imidazo[1,2-a]pyridine-2-carboxylate

Methyl 3-chloro-6-(2,5-dihydro-2-furanyl)-8-(trifluoromethyl)imidazo[1,2-a]pyridine-2-carboxylate (342 mg, 0.986 mmol) and diphenyl sulfide (0.033 mL, 0.197 mmol) in ethyl acetate (10 mL) were degassed with nitrogen before 10% Pd/C (157 mg, 0.148 mmol) was added. The mixture was stirred under 40psi hydrogen gas for 3 hours. The catalyst was filtered off over celite and the filtrate concentrated. The residue was purified by silica chromatography eluting with a gradient of 0% to 5% acetonitrile in dichloromethane to give the title compound (293 mg; 85%). LCMS: m/z 349, 351 (M+1).

### Step C

### 3-(1-{[3-chloro-6-(tetrahydro-2-furanyl)-8-(trifluoromethyl)imidazo[1,2-a]pyridin-2-yl]carbonyl}-4-piperidinyl)-1,3-oxazolidin-2-one

Methyl 3-chloro-6-(tetrahydro-2-furanyl)-8-(trifluoromethyl)imidazo[1,2-a]pyridine-2-carboxylate (293 mg, 0.840 mmol) was dissolved in tetrahydrofuran (11 mL). Water (11 mL) and sodium hydroxide (1M) (2.10 mL, 2.10 mmol) were added and the mixture stirred for 1 hour at room temperature. 1N hydrochloric acid (4 mL) was added and the mixture extracted 2 times with ethyl acetate. The combined organic layers were washed with brine, dried over sodium sulfate, and concentrated. The residue was slurried in thionyl chloride (1000 mg, 8.40 mmol) and heated to reflux. After 15 minutes, the mixture became homogeneous. The thionyl chloride was removed by rotovap and the residue co-evaporated 2 times with toluene. The residue was dissolved in dichloromethane (3 mL) and split into 3 equal 1 mL aliquots. One of the 1 mL aliquots of acid chloride was added a solution containing N-ethyl-N-(1-methylethyl)-2-propanamine (0.176 mL, 1.01 mmol) and 3-(4-piperidinyl)-1,3-oxazolidin-2-one hydrochloride (70 mg; 0.34 mmol) in dichloromethane. The mixture was stirred for 10 minutes, diluted with dichloromethane, washed with water, and the organic phase concentrated. The residue was triturated in ethyl ether and the precipitate collected by filtration to give the title compound (75 mg; 55%). LCMS: m/z 487, 489 (M+1).

### Example 34

### 3-(1-{[3-chloro-6-(ethyloxy)-8-(trifluoromethyl)imidazo[1,2-a]pyridin-2-yl]carbonyl}-4-piperidinyl)-1,3-oxazolidin-2-one

### (Compound 135)

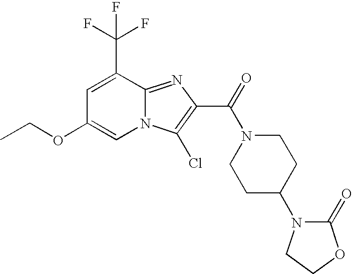

### Step A

### methyl 3-chloro-6-(ethyloxy)-8-(trifluoromethyl)imidazo[1,2-a]pyridine-2-carboxylate

Methyl 3-chloro-6-hydroxy-8-(trifluoromethyl)imidazo[1,2-a]pyridine-2-carboxylate (360 mg, 1.22 mmol), potassium carbonate (338 mg, 2.44 mmol), and ethyl iodide (0.099 mL, 1.22 mmol) in N,N-dimethylformamide (5 mL) were stirred at room temperature for 2 hours. The mixture was diluted with water and extracted with ethyl acetate. The organic phase was washed with 5% lithium chloride, washed with brine, dried over sodium sulfate, and concentrated. The residue was purified by silica chromatography eluting with a gradient of 0% to 5% ethyl acetate in dichloromethane to give the title compound (250 mg; 63%). LCMS: m/z 323, 325 (M+1).

### Step B

### 3-(1-{[3-chloro-6-(ethyloxy)-8-(trifluoromethyl)imidazo[1,2-a]pyridin-2-yl]carbonyl}-4-piperidinyl)-1,3-oxazolidin-2-one

Methyl 3-chloro-6-(ethyloxy)-8-(trifluoromethyl)imidazo[1,2-a]pyridine-2-carboxylate (250 mg, 0.775 mmol) was dissolved in tetrahydrofuran (10 mL). Water (10 mL) and sodium hydroxide (1.93 mL, 1.93 mmol) were added and the mixture stirred for 1 hour at room temperature. 4 mL of 1N hydrochloric acid was added and the mixture extracted 2 times with ethyl acetate. The combined organic layers were washed with brine, dried over sodium sulfate, and concentrated. The residue was slurried in thionyl chloride (1000 mg, 8.40 mmol) and the mixture heated to reflux for 15 minutes. The thionyl chloride was removed by rotovap and the residue co-evaporated 2 times with toluene. The residue was dissolved in 3 mL of dichloromethane and split into three equal 1 mL aliquots. To one of the 1 mL aliquots was added a solution of N-ethyl-N-(1-methylethyl)-2-propanamine (0.162 mL, 0.930 mmol) and 3-(4-piperidinyl)-1,3-oxazolidin-2-one hydrochloride (56 mg; 0.27 mmol) in dichloromethane (5mL). The reaction mixture was stirred for 10 minutes before being diluted with dichloromethane. The mixture was washed with saturated sodium bicarbonate, and the organic layer concentrated. The residue was purified by reverse phase HPLC. Fractions were concentrated and the residue re-crystallized from dichloromethane and hexanes to give the title compound (35 mg; 29%). LCMS: m/z 461, 463 (M+1).

### Example 35

### 3-(1-{[3-chloro-6-(3-pyridinyl)-8-(trifluoromethyl)imidazo[1,2-a]pyridin-2-yl]carbonyl}-4-piperidinyl)-1,3-oxazolidin-2-one

### (Compound 136)

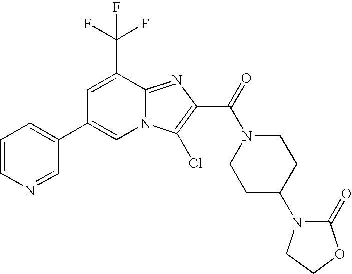

### Step A

### methyl 3-chloro-6-(3-pyridinyl)-8-(trifluoromethyl)imidazo[1,2-a]pyridine-2-carboxylate

To a mixture of methyl 6-bromo-3-chloro-8-(trifluoromethyl)imidazo[1,2-a]pyridine-2-carboxylate (600 mg, 1.68 mmol), 3-pyridinylboronic acid (309 mg, 2.52 mmol), and 3M potassium phosphate tribasic (1.68 mL, 5.03 mmol) in acetonitrile (10 mL) was added PdCl₂(dppf)-dichloromethane adduct (137 mg, 0.168 mmol). The mixture was heated to 80° C. for 2 hours, cooled to room temperature, diluted with water, and extracted with ethyl acetate. The organic phase was separated, washed with 5% lithium chloride, washed with brine, dried over sodium sulfate, and concentrated. The residue was purified by silica chromatography eluting with a gradient of 0% to 50% ethyl acetate in dichloromethane to give the title compound (260 mg; 44%). LCMS: m/z 356, 358 (M+1).

### Step B

### 3-(1-{[3-chloro-6-(3-pyridinyl)-8-(trifluoromethyl)imidazo[1,2-a]pyridin-2-yl]carbonyl}-4-piperidinyl)-1,3-oxazolidin-2-one

Methyl 3-chloro-6-(3-pyridinyl)-8-(trifluoromethyl)imidazo[1,2-a]pyridine-2-carboxylate (250 mg, 0.703 mmol) was dissolved in tetrahydrofuran (10 mL). Water (10.00 mL) and sodium hydroxide (1.757 mL, 1.757 mmol) were added and the mixture stirred for 1 hour at room temperature. 10 mL of 1N hydrochloric acid was added and the mixture concentrated. The residue was co-evaporated 2 times with toluene, dried under high vacuum, and split equally into 3 different reaction vials. To one of the reaction vials was added N,N-dimethylformamide (3 mL), 3-(4-piperidinyl)-1,3-oxazolidin-2-one hydrochloride (51 mg; 0.25 mmol), N-ethyl-N-(1-methylethyl)-2-propanamine (0.246 mL, 1.41 mmol), and N-[(dimethylamino)(3H-[1,2,3]triazolo[4,5-b]pyridin-3-yloxy)methylidene]-N-methylmethanaminium hexafluorophosphate (HATU) (94 mg, 0.246 mmol). The reaction was stirred for 1 hour and quenched with saturated sodium bicarbonate. The mixture was extracted with ethyl acetate and the organic phase washed with brine and concentrated. The residue was slurried in methanol and the precipitate collected by filtration to give the title compound (65 mg; 56%). LCMS: m/z 494, 496 (M+1).

### Example 36

### 3-(1-{[3-chloro-6-(tetrahydro-3-furanyl)-8-(trifluoromethyl)imidazo[1,2-a]pyridin-2-yl]carbonyl}-4-piperidinyl)-1,3-oxazolidin-2-one

### (Compound 137)

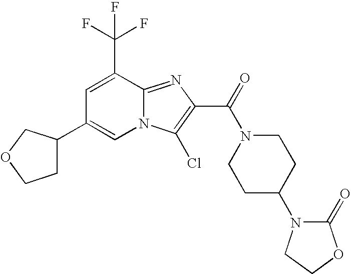

### Step A

### Methyl 3-chloro-6-(2,3-dihydro-3-furanyl)-8-(trifluoromethyl)imidazo[1,2-a]pyridine-2-carboxylate

Methyl 6-bromo-3-chloro-8-(trifluoromethyl)imidazo[1,2-a]pyridine-2-carboxylate (2.0, 5.6 mmol), palladium(II) acetate (0.13 g, 0.56 mmol), potassium acetate (1.4 g, 14 mmol), 2,5-dihydrofuran (1.96 g, 28 mmol), and tetrabutylammonium bromide (1.8 g, 5.6 mmol) in N,N-dimethylformamide (30 mL) were stirred at 80° C. for 1 hour. The mixture was allowed to cool, diluted with water, extracted with ethyl acetate, washed with 5% lithium chloride, washed with brine, dried over sodium sulfate, and concentrated. The residue was purified by silica chromatography eluting with a gradient of 0% to 10% ethyl acetate in dichloromethane to give the title compound (310 mg, 16%). ES-LCMS: m/z 347, 349 (M+1).

### Step B

### Methyl 3-chloro-6-(tetrahydro-3-furanyl)-8-(trifluoromethyl)imidazo[1,2-a]pyridine-2-carboxylate

Methyl 3-chloro-6-(2,3-dihydro-3-furanyl)-8-(trifluoromethyl)imidazo[1,2-a]pyridine-2-carboxylate (310 mg, 0.894 mmol) and diphenyl sulfide (0.030 mL, 0.179 mmol) were degassed with nitrogen before 10% palladium on carbon (143 mg, 0.134 mmol) was added. The mixture was stirred under 40 psi hydrogen for 3 hours. The catalyst was filtered off over celite and the filtrate concentrated. The residue was subject to silica chromatography eluting with a gradient of 0% to 20% ethyl acetate in dichloromethane to give the title compound (255 mg; 82%). ES-LCMS: m/z 349, 351 (M+1).

### Step C

### 3-(1-{[3-chloro-6-(tetrahydro-3-furanyl)-8-(trifluoromethyl)imidazo[1,2-a]pyridin-2-yl]carbonyl}-4-piperidinyl)-1,3-oxazolidin-2-one

Methyl 3-chloro-6-(tetrahydro-3-furanyl)-8-(trifluoromethyl)imidazo[1,2-a]pyridine-2-carboxylate (255 mg, 0.731 mmol) was dissolved in tetrahydrofuran (5 mL). Water (5 mL) and sodium hydroxide (1 M) (1.828 mL, 1.828 mmol) were added and the mix stirred for 1 hour at room temperature. 10 mL of 1N hydrochloric acid were added and the mixture and then extracted two times with ethyl acetate. The combined organic layers were washed with brine, dried over sodium sulfate, and concentrated. The residue was slurried in thionyl chloride (870 mg, 7.31 mmol) and the mixture heated to reflux. The mixture became homogeneous after 15 minutes. The thionyl chloride was removed by rotovap and the residue co-evaporated two times with toluene. The residue was dissolved in 3 mL of dichloromethane. A 1 mL aliquot of the acid chloride solution was added dropwise to a solution of N-ethyl-N-(1-methylethyl)-2-propanamine (0.153 mL, 0.878 mmol) and 3-(4-piperidinyl)-1,3-oxazolidin-2-one hydrochloride (59 mg, 0.29 mmol) in dichloromethane (2 mL). The mixture was stirred for 15 minutes, diluted with dichloromethane, and washed with saturated sodium bicarbonate. The organic layer was concentrated and the residue crystallized from dichloromethane and hexanes to give the title compound (85 mg; 71%). ES-LCMS: m/z 487, 489 (M+1).

### Example 37

### 3-(1-{[3-chloro-6-[(1-methylethyl)oxy]-8-(trifluoromethyl)imidazo[1,2-a]pyridin-2-yl]carbonyl}-4-piperidinyl)-1,3-oxazolidin-2-one

### (Compound 138)

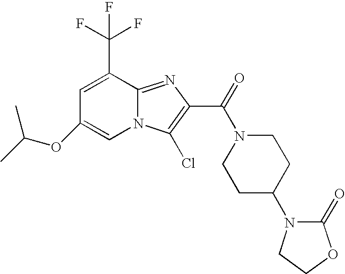

### Step A

### Methyl 3-chloro-6-[(1-methylethyl)oxy]-8-(trifluoromethyl)imidazo[1,2-a]pyridine-2-carboxylate

Methyl 3-chloro-6-hydroxy-8-(trifluoromethyl)imidazo[1,2-a]pyridine-2-carboxylate (495 mg, 1.680 mmol), potassium carbonate (464 mg, 3.36 mmol), and 2-iodopropane (0.185 mL, 1.85 mmol) in dimethylformamide (5 mL) were stirred at room temperature for 2 hours. The mixture was diluted with water and extracted with ethyl acetate. The organic phase was washed with 5% lithium chloride, washed with brine, dried over sodium sulfate, concentrated, and the residue purified by silica chromatography eluting with a gradient of 0% to 5% ethyl acetate in dichloromethane to give the title compound (325 mg, 57%). ES-LCMS: m/z 337, 339 (M+1).

### Step B

### 3-(1-{[3-chloro-6-[(1-methylethyl)oxy]-8-(trifluoromethyl)imidazo[1,2-a]pyridin-2-yl]carbonyl}-4-piperidinyl)-1,3-oxazolidin-2-one

Methyl 3-chloro-6-[(1-methylethyl)oxy]-8-(trifluoromethyl)imidazo[1,2-a]pyridine-2-carboxylate (325 mg, 0.965 mmol) was dissolved in tetrahydrofuran (5 mL). Water (5 mL) and sodium hydroxide (1M) (2.413 mL, 2.413 mmol) were added and the mixture stirred for 1 hour at room temperature. 10 mL of 1N hydrochloric acid were added and the mixture extracted two times with ethyl acetate. The combined organic layers were washed with brine, dried over sodium sulfate, and concentrated. The residue was slurried in thionyl chloride (1148 mg, 9.65 mmol) and the mixture heated to reflux. After 15 minutes, the mixture became homogeneous. The thionyl chloride was removed by rotovap and the residue co-evaporated two times with toluene. The residue was dissolved in 8 mL of dichloromethane. A 2 mL aliquot of the acid chloride solution was added dropwise to a solution of N-ethyl-N-(1-methylethyl)-2-propanamine (0.202 mL, 1.16 mmol) and 3-(4-piperidinyl)-1,3-oxazolidin-2-one hydrochloride (50 mg, 0.24 mmol) in dichloromethane (2 mL). After 10 minutes, the mixture was diluted with dichloromethane and washed with saturated sodium bicarbonate. The organic layer was concentrated and the residue re-crystallized from ethyl acetate to give the title compound (67 mg, 44%). ES-LCMS: mlz 475, 477 (M+1).

### Example 38

### 3-(1-{[3-chloro-6-(1H-imidazol-1-yl)-8-(trifluoromethyl)imidazo[1,2-a]pyridin-2-yl]carbonyl}-4-piperidinyl)-1,3-oxazolidin-2-one

### (Compound 139)

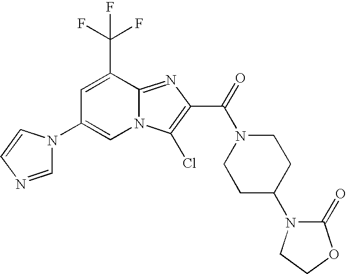

### Step A

### Methyl 3-chloro-6-(4,4,5,5-tetramethyl-1,3,2-dioxaborolan-2-yl)-8-(trifluoromethyl)imidazo[1,2-a]pyridine-2-carboxylate

A mixture of methyl 6-bromo-3-chloro-8-(trifluoromethyl)imidazo[1,2-a]pyridine-2-carboxylate (5 g, 13.99 mmol), Bis(pinacolato)diboron (8.88 g, 35.0 mmol), potassium acetate (4.12 g, 42.0 mmol) and [1,1′-bis(diphenylphosphino)ferrocene]dichloropalladium(II) dichloromethane adduct (0.512 g, 0.699 mmol) was heated at 100° C. in 1,4-Dioxane (186 ml). After 3.5 hours, the reaction was cooled to room temperature. The solid was filtered and the black filtrate absorbed onto silica gel before being chromatographed [ISCO 220 g column, 0-15% ethyl acetate in n-hexanes (15 min), 15% ethyl acetate in n-hexanes (10 min), (15-40% ethyl acetate in n-hexanes, 10 min). The compound eluted with 30% ethyl acetate in n-hexanes. Appropriate fractions were concentrated to give the title compound (4.54 g, 11.2 mmol, 80% yield) as a white solid. ES-LCMS: m/z 404, 407 (M+1).

### Step B

### Methyl 3-chloro-6-(1H-imidazol-1-yl)-8-(trifluoromethyl)imidazo[1,2-a]pyridine-2-carboxylate

Methyl 3-chloro-6-(4,4,5,5-tetramethyl-1,3,2-dioxaborolan-2-yl)-8-(trifluoromethyl)imidazo[1,2-a]pyridine-2-carboxylate (1.5 g, 3.7 mmol), copper (I) oxide (0.265 g, 1.85 mmol), and imidazole (0.303 g, 4.45 mmol) in methanol (10 ml) were stirred at room temperature for 1 day. LC-MS showed no conversion. Air was bubbled through the reaction and then heated to 35-40° C. No conversion was observed after 2 hours. The mixture was allowed to cool to room temperature. One drop of water was added and the mixture allowed to stir over the weekend. The reaction had gone dry over the weekend. Methanol (5 mL) was added and the mixture continued to stir for 2 hours. LC-MS showed a small amount of desired product had formed. An additional 0.4 equivalents of copper (I) oxide (0.21 g, 1.5 mmol) were added and the mixture continued to stir at room temperature overnight. LC-MS showed ˜50% conversion. An additional 0.5 equivalents of copper (I) oxide (265 mg, 1.85 mmol) was added and the mixture continued to stir at room temperature. After 24 hours, the reaction was filtered through a pad of celite, the pad of celite washed with methanol and dichloromethane, and the filtrate concentrated to give the title compound (1.6 g, <99%, 79% purity) as a solid. ES-LCMS: m/z 345, 347 (M+1).

### Step C

### 3-(1-{[3-chloro-6-(1H-imidazol-1-yl)-8-(trifluoromethyl)imidazo[1,2-a]pyridin-2-yl]carbonyl}-4-piperidinyl)-1,3-oxazolidin-2-one

Methyl 3-chloro-6-(1H-imidazol-1-yl)-8-(trifluoromethyl)imidazo[1,2-a]pyridine-2-carboxylate (40 mg, 0.12 mmol) was slurried in tetrahydrofuran (1 mL) before water (1 mL) and sodium hydroxide (1 M) (0.23 mL, 0.23 mmol) were added. The mixture was stirred at room temperature for 1 hour. The mixture was concentrated and the residue slurried in N,N-dimethylformamide (2 mL). N-ethyl-N-(1-methylethyl)-2-propanamine (0.081 mL, 0.46 mmol) and 3-(4-piperidinyl)-1,3-oxazolidin-2-one hydrochloride (29 mg, 0.14 mmol) were added followed by N-[(dimethylamino)(3H-[1,2,3]triazolo[4,5-b]pyridin-3-yloxy)methylidene]-N-methylmethanaminium hexafluorophosphate (HATU) (53 mg, 0.14 mmol). The mixture was stirred at room temperature for 1 hour and concentrated. The residue was purified by reverse phase HPLC to give the title compound (12 mg; 21%). ES-LCMS: m/z 483, 485 (M+1).

### Example 39

### 3-(1-{[3-chloro-6-(2-methylpropyl)-8-(trifluoromethyl)imidazo[1,2-a]pyridin-2-yl]carbonyl}-4-piperidinyl)-1,3-oxazolidine-2,4-dione (Compound 140)

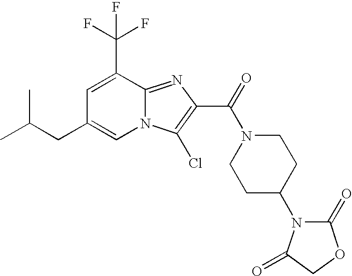

### Step A: 3-chloro-6-(2-methylpropyl)-8-(trifluoromethyl)imidazo[1,2-a]pyridine-2-carboxylic acid

Methyl 3-chloro-6-(2-methylpropyl)-8-(trifluoromethyl)imidazo[1,2-a]pyridine-2-carboxylate (1.7 g, 5.1 mmol) was dissolved in tetrahydrofuran (35 mL). Water (35 mL) and sodium hydroxide (1 M) (10.2 mL, 10.2 mmol) were added and the mixture stirred for 2 hours at room temperature. The mixture was quenched with 1N hydrochloric acid and extracted 2 times with ethyl acetate. The combined organic layers were washed with brine, dried over sodium sulfate, and concentrated to give the title compound (1.7 g; 99%). ES-LCMS: m/z 321, 323 (M+1).

### Step B: 3-(1-{[3-chloro-6-(2-methylpropyl)-8-(trifluoromethyl)imidazo[1,2-a]pyridin-2-yl]carbonyl}-4-piperidinyl)-1,3-oxazolidine-2,4-dione

To a mixture of 3-chloro-6-(2-methylpropyl)-8-(trifluoromethyl)imidazo[1,2-a]pyridine-2-carboxylic acid (50 mg, 0.16 mmol) and 3-(4-piperidinyl)-1,3-oxazolidine-2,4-dione hydrochloride (40 mg; 0.18 mmol) in N,N-dimethylformamide (1 mL) was added N-ethyl-N-(1-methylethyl)-2-propanamine (0.136 mL, 0.780 mmol). N-[(dimethylamino)(3H-[1,2,3]triazolo[4,5-b]pyridin-3-yloxy)methylidene]-N-methylmethanaminium hexafluorophosphate (HATU) (71.1 mg, 0.187 mmol) was added and the mixture stirred at room temperature for 30 minutes. The mixture was diluted with ethyl acetate and washed with water. The aqueous layer was extracted with ethyl acetate. The ethyl acetate layers were combined, washed with water, washed with 5% lithium chloride(aq), washed with brine, concentrated, and the residue purified by silica chromatography eluting with a gradient of 0% to 3% 2M ammonia/methanol in dichloromethane to give the title compound (67 mg; 88%). ES-LCMS: m/z 487, 489 (M+1).

### Example 40

### 3-(1-{[3-chloro-6-(1-methylpropyl)-8-(trifluoromethyl)imidazo[1,2-a]pyridin-2-yl]carbonyl}-4-piperidinyl)-1,3-oxazolidin-2-one (Compound 141)

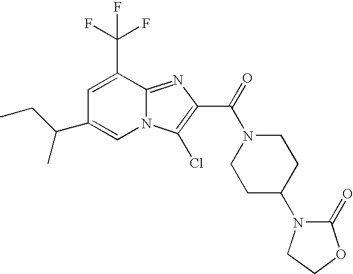

### Step A: tributyl[1-methyl-1-propen-1-yl]stannane

Tributyl(chloro)stannane (2.89 ml, 10.75 mmol) was added to bromo[(1E)-1-methyl-1-propen-1-yl]magnesium (0.5M in THF) (53.8 ml, 26.9 mmol) and the mix stirred at reflux for 5 hours. The mix was cooled to room temperature and quenched with saturated ammonium chloride. The organic phase separated, washed with saturated sodium bicarbonate, washed with brine, dried over sodium sulfate, concentrated, and the residue passed through a silica plug eluting with ethyl ether. The filtrate was concentrated to give the title compound as a mixture of E and Z isomers. ¹H NMR (CHLOROFORM-d) δ ppm 6.01-6.17 (m, 0.7H (major isomer)), 5.55-5.67 (m, 0.3H (minor isomer)), 1.78-1.93 (m, 3H), 1.60-1.72 (m, 3H), 1.37-1.56 (m, 6H), 1.20-1.38 (m, 7H), 0.77-0.96 (m, 14H).

### Step B: Methyl 3-chloro-6-[1-methyl-1-propen-1-yl]-8-(trifluoromethyl)imidazo[1,2-a]pyridine-2-carboxylate (mixture of E and Z isomers)

Methyl 6-bromo-3-chloro-8-(trifluoromethyl)imidazo[1,2-a]pyridine-2-carboxylate (0.50g, 1.4 mmol), tributyl[1-methyl-1-propen-1-yl]stannane (0.58g, 1.7 mmol), and tetrakis (0.16 g, 0.14 mmol) in N,N-dimethylformamide (10 mL) were purged with nitrogen for 5 minutes and then heated to 85° C. for 2 hours. LC-MS showed no reaction. The mix was heated to 110° C. No reaction after 1 hour. 80 mg of trans-benzyl(chloro)bis(triphenylphosphine)palladium (II) was added and the mixture continued to stir at 110° C. After 1 hr, LC-MS showed ˜5:1 SM/desired product. The mixture was heated for 2 hours. Starting material was consumed and the product was observed by LC-MS, along with several other components. The mixture was allowed to cool to room temperature and stirred overnight. The mixture was diluted with ethyl acetate, washed with brine, washed with 5% lithium chloride, dried over sodium sulfate, concentrated, and the residue purified by silica chromatography eluting with 25% ethyl acetate in hexanes to give the title compound (214 mg, 46%) as a mixture (˜7:3) of isomers. ES-LCMS: m/z 333, 335 (M+1).

### Step C: Methyl 3-chloro-6-(1-methylpropyl)-8-(trifluoromethyl)imidazo[1,2-a]pyridine-2-carboxylate

Methyl 3-chloro-6-[1-methyl-1-propen-1-yl]-8-(trifluoromethyl)imidazo[1,2-a]pyridine-2-carboxylate (184 mg, 0.553 mmol) was dissolved in ethanol (10 mL) and tetrahydrofuran (5 mL). 3% platinum on carbon (90 mg, 0.014 mmol) was added under nitrogen. The mixture was purged with nitrogen and then stirred under hydrogen (balloon pressure) for 3 hours. The mixture was then stirred under 30psi of hydrogen overnight. The catalyst was filtered off over celite and the wash concentrated. The residue was purified by silica chromatography eluting with 25% ethyl acetate in hexanes to give the title compound (128 mg, 69%). ES-LCMS: m/z 335, 337 (M+1).

### Step D: 3-(1-{[3-chloro-6-(1-methylpropyl)-8-(trifluoromethyl)imidazo[1,2-a]pyridin-2-yl]carbonyl}-4-piperidinyl)-1,3-oxazolidin-2-one

Methyl 3-chloro-6-(1-methylpropyl)-8-(trifluoromethyl)imidazo[1,2-a]pyridine-2-carboxylate (63 mg, 0.19 mmol) was dissolved in tetrahydrofuran (2.5 mL) before sodium hydroxide (1M) (0.376 mL, 0.376 mmol) and water (2.5 mL) were added. The mixture was stirred for 1 hour, quenched with 1N hydrochloric acid, and extracted two times with ethyl acetate. The combined organic layers were washed with brine, dried over sodium sulfate, and concentrated. The residue was dissolved in dichloromethane (3 mL) before oxalyl chloride (2M in DCM) (0.188 mL, 0.376 mmol) was added dropwise. The mixture was stirred for 1 hour, concentrated, and co-evaporated with hexanes. The acid chloride solution was dissolved in 2 mL of dichloromethane. A 1 mL aliquot of the acid chloride solution was added dropwise to a solution of N-ethyl-N-(1-methylethyl)-2-propanamine (0.053 mL, 0.30 mmol) and 3-(4-piperidinyl)-1,3-oxazolidin-2-one hydrochloride (21 mg, 0.10 mmol) in dichloromethane (2 mL). The mixture was stirred for 1 hour, concentrated, and the residue purified by silica chromatography eluting with a gradient of 0% to 3% 2M ammonium/methanol in dichloromethane to give the title compound (32 mg, 72%). ES-LCMS: m/z 473, 475 (M+1).

### Example 41

### 3-(1-{[6-bromo-3-chloro-8-(trifluoromethyl)imidazo[1,2-a]pyridin-2-yl]carbonyl}-4-piperidinyl)-1,3-oxazolidine-2,4-dione (Compound 142)

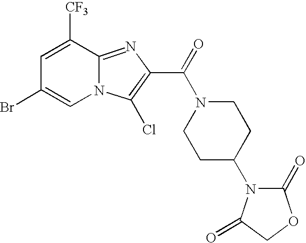

A solution of 6-bromo-3-chloro-8-(trifluoromethyl)imidazo[1,2-a]pyridine-2-carboxylic acid (0.111 g, 0.32 mmol) and 3-(4-piperidinyl)-1,3-oxazolidine-2,4-dione HCl (.065 g, 0.30 mmol) in DMF (1.47 ml) was treated by the addition of DIEA (0.129 ml, 0.736 mmol) and HATU (0.123 g, 0.32 mmol). The reaction was stirred at for 4 hours and diluted with EtOAc and washed with 5% LiCl (×2), brine, dried MgSO₄, filtered, and concentrated. The crude residue was purified with silica gel chromatography (1-5% MeOH/DCM) to give 3-(1-{[6-bromo-3-chloro-8-(trifluoromethyl)imidazo[1,2-a]pyridin-2-yl]carbonyl}-4-piperidinyl)-1,3-oxazolidine-2,4-dione (0.119 g. 79%). ES-LCMS: 511.0 (M+1).

### Example 42

### 1-(1-{[6-bromo-3-chloro-8-(trifluoromethyl)imidazo[1,2-a]pyridin-2-yl]carbonyl}-4-piperidinyl)-2-pyrrolidinone (Compound 143)

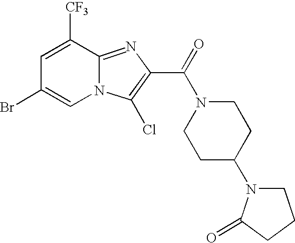

A solution of 1-(4-piperidinyl)-2-pyrrolidinone HCl (.050 g, 0.24 mmol) and 6-bromo-3-chloro-8-(trifluoromethyl)imidazo[1,2-a]pyridine-2-carboxylic acid (0.092 g, 0.27 mmol) in DMF (1.22 ml) was treated with DIEA (0.128 ml, 0.73 mmol) and HATU (0.102 g, 0.27 mmol) and the reaction was stirred at room temperature overnight. The reaction was diluted with EtOAc and washed with 5% LiCl, saturated NaHCO₃, and then brine. The organics were dried MgSO₄, filtered, and concentrated. The crude residue was purified by silica gel chromatography (1-4% MeOH/DCM) to give 1-(1-{[6-bromo-3-chloro-8-(trifluoromethyl)imidazo[1,2-a]pyridin-2-yl]carbonyl}-4-piperidinyl)-2-pyrrolidinone (0.093 g, 77%). ES-LCMS: 495.1 (M+1).

### Example 43

### 3-(1-{[3-chloro-6-[(difluoromethyl)oxy]-8-(trifluoromethyl)imidazo[1,2-a]pyridin-2-yl]carbonyl}-4-piperidinyl)-1,3-oxazolidin-2-one (Compound 144)

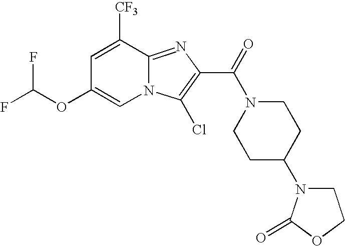

### Step A: methyl 3-chloro-6-[(difluoromethyl)oxy]-8-(trifluoromethyl)imidazo[1,2-a]pyridine-2-carboxylate

A solution of methyl 3-chloro-6-hydroxy-8-(trifluoromethyl)imidazo[1,2-a]pyridine-2-carboxylate (0.25 g, 0.84 mmol), chlorodifluoroacetic acid sodium salt (0.27 g, 1.94 mmol), and Cs₂CO₃ (0.38 g, 1.18 mmol) in DMF (23.4 ml) was stirred at 90° C. for 4 hours. The reaction was then diluted with EtOAc, the combined organic phase was washed with 5% LiCl (2×), brine, dried (MgSO₄), filtered, evaporated and purified by column chromatography (0-3% MeOH/DCM) to give methyl 3-chloro-6-[(difluoromethyl)oxy]-8-(trifluoromethyl)imidazo[1,2-a]pyridine-2-carboxylate (0.12 g, 42%). ES-LCMS: 345.3 (M+1).

### Step B: 3-chloro-6-[(difluoromethyl)oxy]-8-(trifluoromethyl)imidazo[1,2-a]pyridine-2-carboxylic acid

A solution of methyl 3-chloro-6-[(difluoromethyl)oxy]-8-(trifluoromethyl)imidazo[1,2-a]pyridine-2-carboxylate (0.12 g, 0.35 mmol) in THF (2.35 ml) and water (2.35 ml) was treated with 1M NaOH (0.70 ml, 0.70 mmol). The reaction was stirred at room temperature for 30 minutes. The reaction was then quenched by the addition of 1M HCl (0.70 ml, 0.70 mmol). The reaction was diluted with EtOAc and the layers were separated. The combined organics phase was washed with brine, dried (MgSO₄), filtered, and the solvent was evaporated to give 3-chloro-6-[(difluoromethyl)oxy]-8-(trifluoromethyl)imidazo[1,2-a]pyridine-2-carboxylic acid which was used without further purification (0.117 g, 100%). ES-LCMS: 331.0 (M+1).

### Step C: 3-(1-{[3-chloro-6-[(difluoromethyl)oxy]-8-(trifluoromethyl)imidazo[1,2-a]pyridin-2-yl]carbonyl}-4-piperidinyl)-1,3-oxazolidin-2-one

A solution of 3-(4-piperidinyl)-1,3-oxazolidin-2-one (0.022 g, 0.13 mmol) in DMF (0.726 ml) was treated by the addition of DIEA (0.038 ml, 0.29 mmol), 3-chloro-6-[(difluoromethyl)oxy]-8-(trifluoromethyl)imidazo[1,2-a]pyridine-2-carboxylic acid (0.048 g, 0.15 mmol), and HATU (0.061 g, 0.16 mmol). The reaction was stirred at room temperature overnight. The mixture was diluted with EtOAc, washed with 5% LiCl (2×), saturated NaHCO₃, brine, dried MgSO₄, filtered, and concentrated. The crude residue was purified by silica gel chromatography (0-3% MeOH/DCM) to give 3-(1-{[3-chloro-6-[(difluoromethyl)oxy]-8-(trifluoromethyl)imidazo[1,2-a]pyridin-2-yl]carbonyl}-4-piperidinyl)-1,3-oxazolidin-2-one (0.032 g, 46%). ES-LCMS: 483.2 (M+1).

### Example 44

### 3-(1-{[3-chloro-6-[(difluoromethyl)oxy]-8-(trifluoromethyl)imidazo[1,2-a]pyridin-2-yl]carbonyl}-4-piperidinyl)-1,3-oxazolidine-2,4-dione (Compound 145)

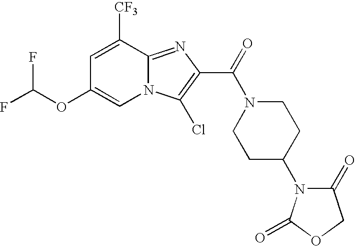

A solution of 3-(4-piperidinyl)-1,3-oxazolidine-2,4-dione HCl (0.029 g, 0.13 mmol) and 3-chloro-6-[(difluoromethyl)oxy]-8-(trifluoromethyl)imidazo[1,2-a]pyridine-2-carboxylic acid (0.048 g, 0.16 mmol) in DMF (0.726 ml) was treated with DIEA (0.063 ml, 0.36 mmol) and HATU (0.061 g, 0.16 mmol). The reaction was stirred at room temperature for 2.5 hours. The reaction was then diluted with EtOAc and washed with 5% LiCl (×2) and then brine. The organics were dried MgSO₄, filtered, and concentrated. The crude residue was purified with silica gel chromatography (0-3% MeOH/DCM) to give the 3-(1-{[3-chloro-6-[(difluoromethypoxy]-8-(trifluoromethyl)imidazo[1,2-a]pyridin-2-yl]carbonyl}-4-piperidinyl)-1,3-oxazolidine-2,4-dione (0.058 g, 80%). ES-LCMS: 497.1 (M+1).

### Example 45

### 3-(1-{[3-chloro-6-(2-furanyl)-8-(trifluoromethyl)imidazo[1,2-a]pyridin-2-yl]carbonyl}-4-piperidinyl)-1,3-oxazolidin-2-one (Compound 146)

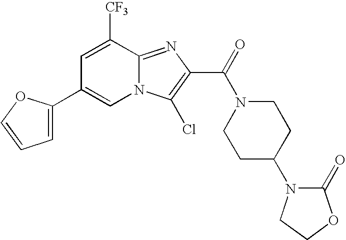

### Step A: methyl 3-chloro-6-(2-furanyl)-8-(trifluoromethyl)imidazo[1,2-a]pyridine-2-carboxylate

A solution of methyl 6-bromo-3-chloro-8-(trifluoromethyl)imidazo[1,2-a]pyridine-2-carboxylate (0.60 g, 1.68 mmol), 2-furanylboronic acid (0.28 g, 2.52 mmol), and 3M tripotassium phosphate (1.68 ml, 5.03 mmol) in ACN (13.99 ml) was treated with PdCl₂(dppf)-CH₂Cl₂ adduct (0.14 g, 0.17 mmol). The reaction was heated to 85° C. for 1 hour. The reaction was cooled to room temperature and diluted with water. The mixture was extracted with DCM, the combined organic phase was washed with brine, dried (MgSO₄), filtered, evaporated and purified by silica gel chromatography (0-1% MeOH/DCM) to give methyl 3-chloro-6-(2-furanyl)-8-(trifluoromethyl)imidazo[1,2-a]pyridine-2-carboxylate (0.46 g, 79%). ES-LCMS: 345.4, 347.1 (M+1).

### Step B: 3-chloro-6-(2-furanyl)-8-(trifluoromethyl)imidazo[1,2-a]pyridine-2-carboxylic acid

A solution of methyl 3-chloro-6-(2-furanyl)-8-(trifluoromethyl)imidazo[1,2-a]pyridine-2-carboxylate (0.46 g, 1.32 mmol) in THF (8.80 ml) and water (8.80 ml) was treated by the addition of 1N NaOH (2.64 ml, 2.64 mmol). The reaction was stirred at room temperature for 7 hours and then acidified with 1N HCl (3.96 ml, 3.96 mmol). The reaction was extracted with EtOAc, the combined organic phase was washed with brine, dried (MgSO₄), filtered, and the solvent was evaporated to give 3-chloro-6-(2-furanyl)-8-(trifluoromethyl)imidazo[1,2-a]pyridine-2-carboxylic acid which was used without purification (0.42 g, 97%). ES-LCMS: 330.8, 333.1 (M+1).

### Step C: 3-(1-{[3-chloro-6-(2-furanyl)-8-(trifluoromethyl)imidazo[1,2-a]pyridin-2-yl]carbonyl}-4-piperidinyl)-1,3-oxazolidin-2-one

A solution of 3-chloro-6-(2-furanyl)-8-(trifluoromethyl)imidazo[1,2-a]pyridine-2-carboxylic acid (0.072 g, 0.218 mmol) and 3-(4-piperidinyl)-1,3-oxazolidin-2-one HCl (.041 g, 0.198 mmol) in DMF (0.992 ml) was treated by the addition of DIEA (0.052 ml, 0.298 mmol) and HATU (0.083 g, 0.218 mmol). The reaction was stirred at room temperature for 2 hours and diluted with EtOAc. The reaction was washed with 5% LiCl, saturated NaHCO₃, brine, dried MgSO₄, filtered, and concentrated. The crude residue was purified by silica gel chromatography (1-4% MeOH/DCM) to give the product as a yellow solid. The residue was then triturated with Et2O give 3-(1-{[3-chloro-6-(2-furanyl)-8-(trifluoromethyl)imidazo[1,2-a]pyridin-2-yl]carbonyl}-4-piperidinyl)-1,3-oxazolidin-2-one (0.065 g, 98%) as a pale yellow solid. ES-LCMS: 485.2 (M+1).

### Example 46

### 3-(1-{[3-chloro-6-(1H-pyrrol-3-yl)-8-(trifluoromethyl)imidazo[1,2-a]pyridin-2-yl]carbonyl}-4-piperidinyl)-1,3-oxazolidin-2-one (Compound 147)

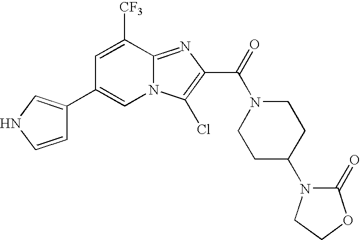

### Step A: methyl 3-chloro-6-(1-{[(1,1-dimethylethyl)oxy]carbonyl}-1H-pyrrol-3-yl)-8-(trifluoromethyl)imidazo[1,2-a]pyridine-2-carboxylate

A solution of methyl 6-bromo-3-chloro-8-(trifluoromethyl)imidazo[1,2-a]pyridine-2-carboxylate (0.60 g, 1.68 mmol), 1,1-dimethylethyl 3-(4,4,5,5-tetramethyl-1,3,2-dioxaborolan-2-yl)-1H-pyrrole-1-carboxylate (0.54 g, 1.85 mmol), and 3M tripotassium phosphate (1.68 ml, 5.03 mmol) in ACN (13.99 ml) was treated with PdCl₂(dppf)-CH₂Cl₂ adduct (0.14 g, 0.17 mmol). The reaction was heated to 85° C. for 1 hour. The reaction was cooled to room temperature and diluted with water. The mixture was extracted with DCM, the combined organic phase was washed with brine, dried (MgSO₄), filtered, evaporated and purified by silica gel chromatography (0-5% MeOH/DCM) to give methyl 3-chloro-6-(1-{[(1,1-dimethylethypoxy]carbonyl}-1H-pyrrol-3-yl)-8-(trifluoromethyl)imidazo[1,2-a]pyridine-2-carboxylate (0.78 g, 100%). ES-LCMS: 444.2 (M+1).

### Step B: 3-chloro-6-(1-{[(1,1-dimethylethyl)oxy]carbonyl}-1H-pyrrol-3-yl)-8-(trifluoromethyl)imidazo[1,2-a]pyridine-2-carboxylic acid

A solution of methyl 3-chloro-6-(1-{[(1,1-dimethylethyl)oxy]carbonyl}-1H-pyrrol-3-yl)-8-(trifluoromethyl)imidazo[1,2-a]pyridine-2-carboxylate (0.78 g, 1.76 mmol) in THF (11.70 ml) and water (11.70 ml) was treated by the addition of 1N NaOH (2.63 ml, 2.63 mmol). The reaction was stirred at room temperature overnight. The reaction was extracted with EtOAc and set aside. The aqueous phase was then acidified with 1N HCl (5.27 ml, 5.27 mmol) and then was extracted with EtOAc, the combined organic phase was washed with brine, dried (MgSO₄), filtered, and the solvent was evaporated to give 3-chloro-6-(1-{[(1,1-dimethylethyl)oxy]carbonyl}-1H-pyrrol-3-yl)-8-(trifluoromethyl)imidazo[1,2-a]pyridine-2-carboxylic acid which was used without purification (0.470 g, 62.3%). ES-LCMS: 430.1 (M+1).

### Step C: 1,1-dimethylethyl 3-[3-chloro-2-{[4-(2-oxo-1,3-oxazolidin-3-yl)-1-piperidinyl]carbonyl}-8-(trifluoromethyl)imidazo[1,2-a]pyridin-6-yl]-1H-pyrrole-1-carboxylate

A solution of 3-chloro-6-(1-{[(1,1-dimethylethyl)oxy]carbonyl}-1H-pyrrol-3-yl)-8-(trifluoromethyl)imidazo[1,2-a]pyridine-2-carboxylic acid (0.091 g, 0.21 mmol)and 3-(4-piperidinyl)-1,3-oxazolidin-2-one HCl (.040 g, 0.19 mmol) in DMF (0.97 ml) was treated by the addition of DIEA (0.101 ml, 0.58 mmol) and HATU (0.081 g, 0.21 mmol). The reaction was stirred at room temperature for 3 hours and diluted with EtOAc. The reaction was washed with 5% LiCl, saturated NaHCO₃, brine, dried MgSO₄, filtered, and concentrated. The crude residue was purified by silica gel chromatography (1-5% MeOH/DCM) to give 1,1-dimethylethyl 3-[3-chloro-2-{[4-(2-oxo-1,3-oxazolidin-3-yl)-1-piperidinyl]carbonyl}-8-(trifluoromethyl)imidazo[1,2-a]pyridin-6-yl]-1H-pyrrole-1-carboxylate (0.089 g, 79%). ES-LCMS: 582.3 (M+1).

### Step D: 3-(1-{[3-chloro-6-(1H-pyrrol-3-yl)-8-(trifluoromethyl)imidazo[1,2-a]pyridin-2-yl]carbonyl}-4-piperidinyl)-1,3-oxazolidin-2-one

A solution of 1,1-dimethylethyl 3-[3-chloro-2-{[4-(2-oxo-1,3-oxazolidin-3-yl)-1-piperidinyl]carbonyl}-8-(trifluoromethyl)imidazo[1,2-a]pyridin-6-yl]-1H-pyrrole-1-carboxylate (0.089 g, 0.15 mmol) in DCM (2.0 ml) was treated by the addition of 4M in dioxanes HCl (0.5 ml, 16.46 mmol). The reaction was stirred at room temperature overnight and then the red liquid concentrated under reduced pressure. The solid was dissolved in MeOH and treated by the addition of 2M in MeOH ammonia (0.3 ml, 13.86 mmol). The mixture was concentrated and the solid was taken up DCM and purified by silica gel chromatography (0-3% MeOH/DCM). The isolated product triturated with Et₂O to give 3-(1-{[3-chloro-6-(1H-pyrrol-3-yl)-8-(trifluoromethyl)imidazo[1,2-a]pyridin-2-yl]carbonyl}-4-piperidinyl)-1,3-oxazolidin-2-one (0.044 g, 60%). ES-LCMS: 482.4 (M+1).

### Example 47

### 3-(1-{[3-chloro-6-(2H-1,2,3-triazol-4-yl)-8-(trifluoromethyl)imidazo[1,2-a]pyridin-2-yl]carbonyl}-4-piperidinyl)-1,3-oxazolidin-2-one (Compound 148)

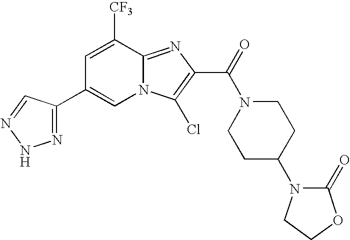

### Step A: methyl 3-chloro-8-(trifluoromethyl)-6-[(trimethylsilyl)ethynyl]imidazo[1,2-a]pyridine-2-carboxylate

A solution of methyl 6-bromo-3-chloro-8-(trifluoromethyl)imidazo[1,2-a]pyridine-2-carboxylate (0.80 g, 2.24 mmol), trimethylsilyethyne (0.35 ml, 2.46 mmol), copper(I) iodide (10.65 mg, 0.056 mmol), TEA (1.72 ml, 12.31 mmol), bis(triphenylphosphine)palladium(II) chloride (0.039 g, 0.056 mmol) in benzene (11.19 ml) was purged with nitrogen and was stirred at room temperature for 3 hours and then concentrated. The crude dark brown residue was purified by silica gel chromatography (100% DCM) to give methyl 3-chloro-8-(trifluoromethyl)-6-[(trimethylsilypethynyl]imidazo[1,2-a]pyridine-2-carboxylate as a light brown solid (0.789 g, 94%). ES-LCMS: 376.7 (M+1).

### Step B: methyl 3-chloro-6-ethynyl-8-(trifluoromethyl)imidazo[1,2-a]pyridine-2-carboxylate

A solution of methyl 3-chloro-8-(trifluoromethyl)-6-[(trimethylsilyl)ethynyl]imidazo[1,2-a]pyridine-2-carboxylate (.789 g, 2.10 mmol) in THF (21.05 ml) was treated by the addition of 1.0M in THF TBAF (2.10 ml, 2.10 mmol). After stirring at room temperature for 20 minutes, the reaction was diluted with EtOAc and the combined organic phase was washed with water (3×), brine, dried (MgSO₄), filtered, and concentrated. The crude black residue was purified by silica gel chromatography (0-50% EtOAc/Hexanes) to give mostly pure product. The residue was then repurified by silica gel chromatography (0-30% EtOAc/Hexanes) to give methyl 3-chloro-6-ethynyl-8-(trifluoromethyl)imidazo[1,2-a]pyridine-2-carboxylate as a white solid (0.183 g, 29%). ES-LCMS: 303.1 (M+1).

### Step C: methyl 3-chloro-6-[2-(hydroxymethyl)-2H-1,2,3-triazol-4-yl]-8-(trifluoromethyl)imidazo[1,2-a]pyridine-2-carboxylate

A 0° C. mixture of 37% aqueous formaldehyde (0.34 ml, 4.56 mmol), glacial acetic acid (0.039 ml, 0.68 mmol), and 1,4-Dioxane (0.34 ml) was stirred for 15 minutes and then was treated with sodium azide (0.044 g, 0.68 mmol), followed by methyl 3-chloro-6-ethynyl-8-(trifluoromethyl)imidazo[1,2-a]pyridine-2-carboxylate (0.138 g, 0.46 mmol). After an additional 10 minutes of stirring, sodium ascorbate (0.018 g, 0.091 mmol) was added, followed by copper(II) sulfate (3.64 mg, 0.023 mmol) in water (0.1 mL). The mixture (suspension) was stirred with warming to room temperature overnight. The suspension was diluted with water and extracted with DCM. The combined organic phase was washed with brine, dried (MgSO₄), filtered, evaporated to give methyl 3-chloro-6-[2-(hydroxymethyl)-2H-1,2,3-triazol-4-yl]-8-(trifluoromethyl)imidazo[1,2-a]pyridine-2-carboxylate which was used without further purification (0.147 g, 86%). ES-LCMS: 376.1 (M+1).

### Step D: 3-chloro-6-(2H-1,2,3-triazol-4-yl)-8-(trifluoromethyl)imidazo[1,2-a]pyridine-2-carboxylic acid

A suspension of methyl 3-chloro-6-[2-(hydroxymethyl)-2H-1,2,3-triazol-4-yl]-8-(trifluoromethyl)imidazo[1,2-a]pyridine-2-carboxylate (0.147 g, 0.39 mmol) in THF (2.61 ml) and water (2.61 ml) was treated by the addition of 1M NaOH (1.17 ml, 1.17 mmol). The reaction became clear and was stirred at room temperature for 1.5 hours and then treated by the addition of 1N HCl (1.17 ml, 1.17 mmol). The reaction was diluted with EtOAc, the combine organic phase was washed with brine, dried (MgSO₄), filtered, and concentrated to give 3-chloro-6-(2H-1,2,3-triazol-4-yl)-8-(trifluoromethyl)imidazo[1,2-a]pyridine-2-carboxylic acid which was used without purification (0.098 g, 76%). ES-LCMS: 332.1 (M+1).

### Step E: 3-(1-{[3-chloro-6-(2H-1,2,3-triazol-4-yl)-8-(trifluoromethyl)imidazo[1,2-a]pyridin-2-yl]carbonyl}-4-piperidinyl)-1,3-oxazolidin-2-one

A solution of 3-chloro-6-(2H-1,2,3-triazol-4-yl)-8-(trifluoromethyl)imidazo[1,2-a]pyridine-2-carboxylic acid (0.024 g, 0.072 mmol) and 3-(4-piperidinyl)-1,3-oxazolidin-2-one HCl (0.018 g, 0.087 mmol) in DMF (0.362 ml) was treated by the addition of DIEA (0.032 ml, 0.181 mmol) and HATU (0.030 g, 0.080 mmol). The reaction was stirred at room temperature for 3 hours and diluted with EtOAc. The reaction was washed with 5% LiCl, saturated NaHCO₃, brine, dried MgSO₄, filtered, and concentrated. The crude residue was triturated with Et₂O to give 3-(1-{[3-chloro-6-(2H-1,2,3-triazol-4-yl)-8-(trifluoromethyl)imidazo[1,2-a]pyridin-2-yl]carbonyl}-4-piperidinyl)-1,3-oxazolidin-2-one (0.017 g, 49%). ES-LCMS: 482.4 (M+1).

### Example 48

### 3-(1-{[3-chloro-6-(1H-imidazol-2-yl)-8-(trifluoromethyl)imidazo[1,2-a]pyridin-2-yl]carbonyl}-4-piperidinyl)-1,3-oxazolidin-2-one (Compound 149)

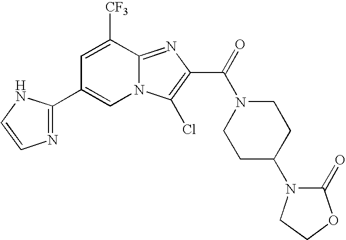

### Step A: methyl 3-chloro-6-ethenyl-8-(trifluoromethyl)imidazo[1,2-a]pyridine-2-carboxylate

A solution of methyl 6-bromo-3-chloro-8-(trifluoromethyl)imidazo[1,2-a]pyridine-2-carboxylate (2.5 g, 6.99 mmol), potassium vinyl triflouroborate (0.937 g, 6.99 mmol), TEA (0.975 ml, 6.99 mmol), and PdCl₂(dppf)-CH₂Cl₂ adduct (5.71 g, 6.99 mmol) in n-propanol (35.0 ml) was purged with nitrogen. The reaction was then heated to 90° C. for 60 minutes. The reaction was then allowed to cool to room temperature and diluted with EtOAc and washed with water, brine, dried MgSO₄, filtered, and concentrated to give methyl 3-chloro-6-ethenyl-8-(trifluoromethyl)imidazo[1,2-a]pyridine-2-carboxylate (2.13 g, 100%) which was used without further purification. ES-LCMS: 305.5 (M+1).

### Step B: methyl 3-chloro-6-formyl-8-(trifluoromethyl)imidazo[1,2-a]pyridine-2-carboxylate

A solution of methyl 3-chloro-6-ethenyl-8-(trifluoromethyl)imidazo[1,2-a]pyridine-2-carboxylate (2.13 g, 6.99 mmol) in THF (35.0 ml) and water (35.0 ml) was treated by the addition of 2.5% in H2O osmium tetroxide (1.756 ml, 0.140 mmol) and sodium periodate (3.74 g, 17.48 mmol). The mixture was stirred at room temperature overnight. The reaction mixture was treated by the addition of 0.5 equivalent of sodium periodate (0.734 g, 3.47 mmol). The reaction was stirred at room temperature for an additional 3 hours. The reaction was diluted with EtOAc and washed with water, brine, dried MgSO₄, filtered, and concentrated. The crude residue was purified by column chromatography (EtOAc/Hexanes) to give methyl 3-chloro-6-formyl-8-(trifluoromethyl)imidazo[1,2-a]pyridine-2-carboxylate (1.68 g, 78%). ES-LCMS: 307.1 (M+1).

### Step C: methyl 3-chloro-6-(1H-imidazol-2-yl)-8-(trifluoromethyl)imidazo[1,2-a]pyridine-2-carboxylate

A solution of methyl 3-chloro-6-formyl-8-(trifluoromethyl)imidazo[1,2-a]pyridine-2-carboxylate (0.300 g, 0.978 mmol) in EtOH (4.89 ml) was cooled to 0° C. and treated a solution of 40% in water glyoxal (0.224 ml, 1.957 mmol) and concentrated ammonium hydroxide (0.337 ml, 4.89 mmol). The reaction was stirred with warming to room temperature over the weekend. The reaction was concentrated and purified by silica gel chromatography (0-5% MeOH/DCM) to give methyl 3-chloro-6-(1H-imidazol-2-yl)-8-(trifluoromethyl)imidazo[1,2-a]pyridine-2-carboxylate (0.137 g, 41%). ES-LCMS: 345.3 (M+1).

### Step D: 3-chloro-6-(1H-imidazol-2-yl)-8-(trifluoromethyl)imidazo[1,2-a]pyridine-2-carboxylic acid

A solution of methyl 3-chloro-6-(1H-imidazol-2-yl)-8-(trifluoromethyl)imidazo[1,2-a]pyridine-2-carboxylate (0.137 g, 0.40 mmol) in THF (2.65 ml) and Water (2.65 ml) was treated by the addition of 1N NaOH (0.795 ml, 0.795 mmol). The reaction was stirred at room temperature for 2 hours and then acidified with 1N HCl (1.19 ml, 1.19 mmol). The reaction was extracted with EtOAc, washed with brine, dried MgSO₄, filtered and concentrated to give 3-chloro-6-(1H-imidazol-2-yl)-8-(trifluoromethyl)imidazo[1,2-a]pyridine-2-carboxylic acid (0.049 g, 37%) which was used without further purification. ES-LCMS: 331.0 (M+1).

### Step E: 3-(1-{[3-chloro-6-(1H-imidazol-2-yl)-8-(trifluoromethyl)imidazo[1,2-a]pyridin-2-yl]carbonyl}-4-piperidinyl)-1,3-oxazolidin-2-one

A solution of 3-chloro-6-(1H-imidazol-2-yl)-8-(trifluoromethyl)imidazo[1,2-a]pyridine-2-carboxylic acid (0.049 g, 0.15 mmol), 3-(4-piperidinyl)-1,3-oxazolidin-2-one HCl (0.034 g, 0.16 mmol), DIEA (0.065 ml, 0.37 mmol), and HATU (0.056 g, 0.19 mmol) in DMF (0.741 ml) was stirred at room temperature for 1.5 hours. The reaction was diluted with EtOAc, washed with 5% LiCl, saturated NaHCO₃, brine, dried MgSO₄, filtered, and concentrated. The crude residue was purified by silica gel chromatography (0-5% MeOH/DCM) to give 3-(1-{[3-chloro-6-(1H-imidazol-2-yl)-8-(trifluoromethyl)imidazo[1,2-a]pyridin-2-yl]carbonyl}-4-piperidinyl)-1,3-oxazolidin-2-one (0.012 g, 17%). ES-LCMS: 483.2 (M+1).

### Example 49

### 3-(1-{[3-chloro-6-(1H-pyrrol-2-yl)-8-(trifluoromethyl)imidazo[1,2-a]pyridin-2-yl]carbonyl}-4-piperidinyl)-1,3-oxazolidin-2-one (Compound 150)

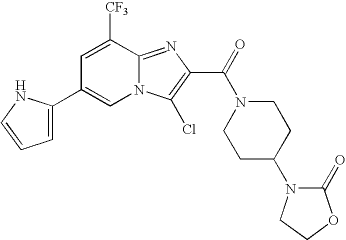

### Step A: methyl 3-chloro-6-(1-{[(1,1-dimethylethyl)oxy]carbonyl}-1H-pyrrol-2-yl)-8-(trifluoromethyl)imidazo[1,2-a]pyridine-2-carboxylate

A solution of 6-bromo-3-chloro-8-(trifluoromethyl)imidazo[1,2-a]pyridine-2-carboxylic acid (0.60 g, 1.68 mmol), (1-{[(1,1-dimethylethyl)oxy]carbonyl}-1H-pyrrol-2-yl)boronic acid (0.43 g, 2.01 mmol), and 3M tripotassium phosphate (1.07 g, 5.03 mmol) in ACN (13.99 ml) was treated with PdCl₂(dppf)-CH₂Cl₂ adduct (0.14 g, 0.17 mmol). The reaction was heated to 85° C. for 1 hour. The reaction was cooled to room temperature and diluted with water and extracted with DCM. The combined organic phase was washed with brine, dried (MgSO₄), filtered and concentrated to yield a reddish-brown residue that was purified by silica gel chromatography (0-1% MeOH/DCM) to yield mostly pure product. The mostly pure product was repurified by silica gel chromatography (10-30% EtOAc/Hexanes) to give methyl 3-chloro-6-(1-{[(1,1-dimethylethyl)oxy]carbonyl}-1H-pyrrol-2-yl)-8-(trifluoromethyl)imidazo[1,2-a]pyridine-2-carboxylate (0.504 g, 68%). ES-LCMS: 444.5, 446.2 (M+1).

### Step B: 3-chloro-6-(1-{[(1,1-dimethylethyl)oxy]carbonyl}-1H-pyrrol-2-yl)-8-(trifluoromethyl)imidazo[1,2-a]pyridine-2-carboxylic acid

A suspension of methyl 3-chloro-6-(1-{[(1,1-dimethylethyl)oxy]carbonyl}-1H-pyrrol-2-yl)-8-(trifluoromethyl)imidazo[1,2-a]pyridine-2-carboxylate (0.504 g, 1.14 mmol) in H2O (7.57 ml) and THF (7.57 ml) was treated by the addition of 1M NaOH (2.27 ml, 2.27 mmol). The reaction became clear and was stirred at room temperature for 1.5 hours and treated by the addition of 1N HCl (3.41 ml, 3.41 mmol). The reaction was diluted with EtOAc and the combine organic phase was washed with brine, dried (MgSO₄), filtered and concentrated to 3-chloro-6-(1-{[(1,1-dimethylethyl)oxy]carbonyl}-1H-pyrrol-2-yl)-8-(trifluoromethyl)imidazo[1,2-a]pyridine-2-carboxylic acid which was used without purification (0.506 g, 100%). ES-LCMS: 430.2 (M+1).

### Step C: 1,1-dimethylethyl 2-≢3-chloro-2-{[4-(2-oxo-1,3-oxazolidin-3-yl)-1-piperidinyl]carbonyl}-8-(trifluoromethyl)imidazo[1,2-a]pyridin-6-yl]-1H-pyrrole-1-carboxylate

A solution of 3-chloro-6-(1-{[(1,1-dimethylethyl)oxy]carbonyl}-1H-pyrrol-2-yl)-8-(trifluoromethyl)imidazo[1,2-a]pyridine-2-carboxylic acid (0.080 g, 0.19 mmol) and 3-(4-piperidinyl)-1,3-oxazolidin-2-one HCl (.035 g, 0.17 mmol) in DMF (0.847 ml) was treated by the addition of DIEA (0.089 ml, 0.51 mmol) and HATU (0.071 g, 0.19 mmol). The reaction was stirred at room temperature for 3 hours and diluted with EtOAc. The reaction was washed with 5% LiCl, saturated NaHCO₃, brine, dried MgSO₄, filtered, and concentrated to give 1,1-dimethylethyl 2-[3-chloro-2-{[4-(2-oxo-1,3-oxazolidin-3-yl)-1-piperidinyl]carbonyl}-8-(trifluoromethyl)imidazo[1,2-a]pyridin-6-yl]-1H-pyrrole-1-carboxylate (0.097 g, 98%) which was used without further purification. ES-LCMS: 582.3 (M+1).

### Step D: 3-(1-{[3-chloro-6-(1H-pyrrol-2-yl)-8-(trifluoromethyl)imidazo[1,2-a]pyridin-2-yl]carbonyl}-4-piperidinyl)-1,3-oxazolidin-2-one

A solution of 1,1-dimethylethyl 2-[3-chloro-2-{[4-(2-oxo-1,3-oxazolidin-3-yl)-1-piperidinyl]carbonyl}-8-(trifluoromethyl)imidazo[1,2-a]pyridin-6-yl]-1H-pyrrole-1-carboxylate (0.097 g, 0.17 mmol) in DCM (2.0 mL) was treated by the addition of 4M in dioxanes HCl (0.042 mL, 0.17 mmol). The reaction was stirred at room temperature for 48 hours and concentrated. The residue was taken up in MeOH and basified with 2M in MeOH ammonia (3.61 μL, 0.167 mmol). The reaction was concentrated and the crude residue was purified by silica gel chromatography (1-4% MeOH/DCM) to give 3-(1-{[3-chloro-6-(1H-pyrrol-2-yl)-8-(trifluoromethyl)imidazo[1,2-a]pyridin-2-yl]carbonyl}-4-piperidinyl)-1,3-oxazolidin-2-one (0.028 g, 35%). ES-LCMS: 482.2 (M+1).

### Example 50

### 3-(1-{[3-chloro-6-methyl-8-(trifluoromethyl)imidazo[1,2-a]pyridin-2-yl]carbonyl}-4-piperidinyl)-1,3-oxazolidin-2-one (Compound 151)

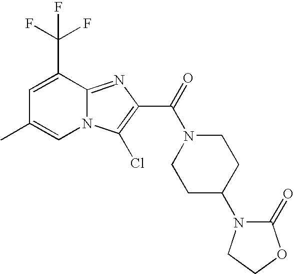

A solution of 3-(1-{[6-bromo-3-chloro-8-(trifluoromethyl)imidazo[1,2-a]pyridin-2-yl]carbonyl}-4-piperidinyl)-1,3-oxazolidin-2-one (50 mg, 0.101 mmol), dimethylzinc (1.0 M solution in heptane) (0.151 mL, 0.151 mmol) and Pd(Ph₃P)₄ (5.83 mg, 5.04 μmol) in THF (6 mL) was stirred with heating at 60° C. The reaction mixture was diluted with EtOAc and washed with water. The solvent was removed and the crude material was purified via reverse phase HPLC to give3-(1-{[3-chloro-6-methyl-8-(trifluoromethyl)imidazo[1,2-a]pyridin-2-yl]carbonyl}-4-piperidinyl)-1,3-oxazolidin-2-one (27 mg, 0.056 mmol, 56% yield). ES-LCMS 430.7 (M+1)

### Example 51

### 3-(1-{[3-chloro-6-ethyl-8-(trifluoromethyl)imidazo[1,2-a]pyridin-2-yl]carbonyl}-4-piperidinyl)-1,3-oxazolidin-2-one (compound 152)

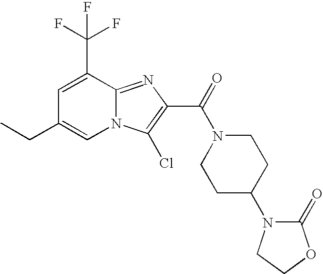

A solution of 3-(1-{[6-bromo-3-chloro-8-(trifluoromethyl)imidazo[1,2-a]pyridin-2-yl]carbonyl}-4-piperidinyl)-1,3-oxazolidin-2-one (200 mg, 0.403 mmol), diethylzinc (49.8 mg, 0.403 mmol) and Pd(Ph₃P)₄ (46.6 mg, 0.040 mmol) in THF (5 mL) was stirred at 60° C. for 30 minutes. The reaction mixture was diluted with EtOAc and washed with water. The solvent was removed and the crude material was purified via silica gel chromatography to give 3-(1-{[3-chloro-6-ethyl-8-(trifluoromethyl)imidazo[1,2-a]pyridin-2-yl]carbonyl}-4-piperidinyl)-1,3-oxazolidin-2-one (27 mg, 0.055 mmol, 14% yield). ES-LCMS : 444.7 (M+1)

### Example 52

### 3-(1-{[3-chloro-6-ethenyl-8-(trifluoromethyl)imidazo[1,2-a]pyridin-2-yl]carbonyl}-4-piperidinyl)-1,3-oxazolidin-2-one (Compound 153)

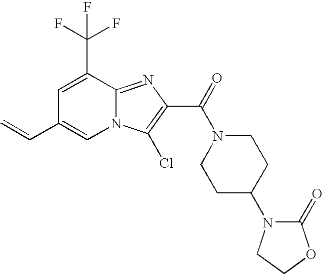

A solution of 3-(1-{[6-bromo-3-chloro-8-(trifluoromethyl)imidazo[1,2-a]pyridin-2-yl]carbonyl}-4-piperidinyl)-1,3-oxazolidin-2-one (3.5 g, 7.06 mmol), potassium vinyl trifluoroborate (1.906 g, 14.12 mmol), Hunig's base (6.17 mL, 35.3 mmol) and PdCl₂(dppf)-CH₂Cl₂ adduct (0.288 g, 0.353 mmol) in propanol (60 mL) was stirred at 100° C. for 40 minutes. The reaction mixture was diluted with EtOAc and washed with water. The solvent was removed and the crude material was purified via silica gel chromatography to give 3-(1-{[3-chloro-6-ethenyl-8-(trifluoromethyl)imidazo[1,2-a]pyridin-2-yl]carbonyl}-4-piperidinyl)-1,3-oxazolidin-2-one (2.32 g, 4.72 mmol, 67% yield). ES-LCMS : 443.1 (M+1)

### Example 53

### 3-(1-{[3-chloro-6-(1-methylethyl)-8-(trifluoromethyl)imidazo[1,2-a]pyridin-2-yl]carbonyl}-4-piperidinyl)-1,3-oxazolidin-2-one (Compound 154)

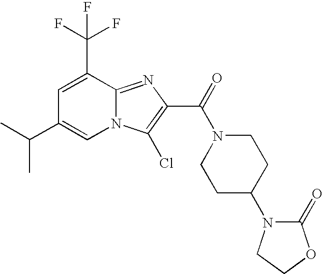

### Step A: 3-(1-{[3-chloro-6-(1-methylethenyl)-8-(trifluoromethyl)imidazo[1,2-a]pyridin-2-yl]carbonyl}-4-piperidinyl)-1,3-oxazolidin-2-one

A solution of 3-(1-{[6-bromo-3-chloro-8-(trifluoromethyl)imidazo[1,2-a]pyridin-2-yl]carbonyl}-4-piperidinyl)-1,3-oxazolidin-2-one (200 mg, 0.403 mmol), potassium isopropenyl trifluoroborate (90 mg, 0.605 mmol), triethylamine (0.281 mL, 2.02 mmol) and PdCl₂(dppf)-CH₂Cl₂ adduct (6.59 mg, 8.07 μmol) in propanol (5 mL) was stirred at 100° C. for 45 minutes. The reaction mixture was diluted with EtOAc and washed with water. The solvent was removed and the crude material was purified via silica gel chromatography to give 3-(1-{[3-chloro-6-(1-methylethenyl)-8-(trifluoromethyl)imidazo[1,2-a]pyridin-2-yl]carbonyl}-4-piperidinyl)-1,3-oxazolidin-2-one (169 mg, 0.333 mmol, 83% yield). ES-LCMS : 457.1 (M+1)

### Step B: 3-(1-{[3-chloro-6-(1-methylethyl)-8-(trifluoromethyl)imidazo[1,2-a]pyridin-2-yl]carbonyl}-4-piperidinyl)-1,3-oxazolidin-2-one

A suspension of 3-(1-{[3-chloro-6-(1-methylethenyl)-8-(trifluoromethyl)imidazo[1,2-a]pyridin-2-yl]carbonyl}-4-piperidinyl)-1,3-oxazolidin-2-one (80 mg, 0.175 mmol), diphenylsulfide (3.26 mg, 0.018 mmol), and Pd/C (37.3 mg, 0.018 mmol) in EtOAc (6 mL) was hydrogenated under 40 psi hydrogen for 5 hours and the solid was filtered off. The solvent was removed and the crude material was purified via reverse phase HPLC to give 3-(1-{[3-chloro-6-(1-methylethyl)-8-(trifluoromethyl)imidazo[1,2-a]pyridin-2-yl]carbonyl}-4-piperidinyl)-1,3-oxazolidin-2-one (38 mg, 0.075 mmol, 43% yield). ES-LCMS : 461.2 (M+1)

### Example 54

### 3-(1-{[3-chloro-6-(1-hydroxyethyl)-8-(trifluoromethyl)imidazo[1,2-a]pyridin-2-yl]carbonyl}-4-piperidinyl)-1,3-oxazolidin-2-one (Compound 155)

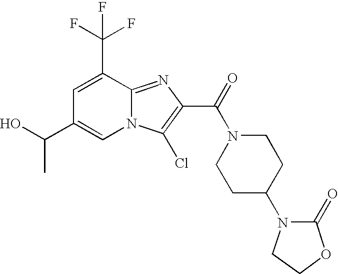

### Step A: 3-(1-{[6-acetyl-3-chloro-8-(trifluoromethyl)imidazo[1,2-a]pyridin-2-yl]carbonyl}-4-piperidinyl)-1,3-oxazolidin-2-one

To a solution of 3-(1-{[6-bromo-3-chloro-8-(trifluoromethyl)imidazo[1,2-a]pyridin-2-yl]carbonyl}-4-piperidinyl)-1,3-oxazolidin-2-one (1.13 g, 2.280 mmol), tetraethylammoniumchloride (1.147 g, 6.84 mmol) and PdCl₂(PPh₃)₂ (0.080 g, 0.114 mmol) in acetonitrile (7 ml) was added tributyl[1-(ethyloxy)ethenyl]stannane (0.847 ml, 2.508 mmol) and the reaction mixture was stirred at 80° C. for 2 hours. The reaction mixture was cooled to RT, diluted with EtOAc and washed with water. The solvent was evaporated and the residue was dissolved in THF (7.00 ml) and HCl (1.0 M) (2.508 ml, 2.508 mmol) was added. The resulting mixture was stirred for 4 hours at RT. EtOAc was added and the organics were separated. The solvent was removed and the crude material was purified via silica gel chromatography to give 3-(1-{[6-acetyl-3-chloro-8-(trifluoromethyl)imidazo[1,2-a]pyridin-2-yl]carbonyl}-4-piperidinyl)-1,3-oxazolidin-2-one (645 mg, 1.406 mmol, 62% yield). ES-LCMS : 459.1 (M+1)

### Step B: 3-(1-{[3-chloro-6-(1-hydroxyethyl)-8-(trifluoromethyl)imidazo[1,2-a]pyridin-2-yl]carbonyl}-4-piperidinyl)-1,3-oxazolidin-2-one

A solution of 3-(1-{[6-acetyl-3-chloro-8-(trifluoromethyl)imidazo[1,2-a]pyridin-2-yl]carbonyl}-4-piperidinyl)-1,3-oxazolidin-2-one (90 mg, 0.196 mmol) and sodium borohydride (7.42 mg, 0.196 mmol) in methanol (5 mL) was stirred at 0° C. for 30 minutes. The reaction mixture was diluted with EtOAc and washed with 1N HCl. The solvent was removed and the crude material was purified via reverse phase HPLC to give 3-(1-{[3-chloro-6-(1-hydroxyethyl)-8-(trifluoromethyl)imidazo[1,2-a]pyridin-2-yl]carbonyl}-4-piperidinyl)-1,3-oxazolidin-2-one (63 mg, 0.123 mmol, 62.7% yield). ES-LCMS: 444.7 (M+1)

### Example 55

### 3-(1-{[3-chloro-6-cyclopropyl-8-(trifluoromethyl)imidazo[1,2-a]pyridin-2-yl]carbonyl}-4-piperidinyl)-1,3-oxazolidin-2-one (Compound 156)

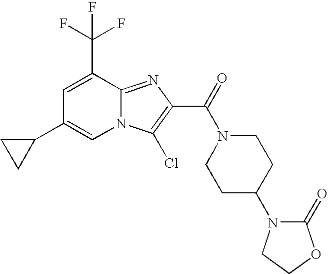

A solution of 3-(1-{[6-bromo-3-chloro-8-(trifluoromethyl)imidazo[1,2-a]pyridin-2-yl]carbonyl}-4-piperidinyl)-1,3-oxazolidin-2-one (300 mg, 0.605 mmol), potassium phosphate (3.0 M) (1.210 mL, 3.63 mmol), 2-cyclopropyl-4,4,5,5-tetramethyl-1,3,2-dioxaborolane (509 mg, 3.03 mmol) and PdCl₂(dppf)-CH₂Cl₂ adduct (49.4 mg, 0.061 mmol) in acetonitrile (5 mL) was stirred at 90° C. overnight. The reaction mixture was diluted with EtOAc and washed with water. The solvent was removed and the crude material was purified via reverse phase HPLC to give 3-(1-{[3-chloro-6-cyclopropyl-8-(trifluoromethyl)imidazo[1,2-a]pyridin-2-yl]carbonyl}-4-piperidinyl)-1,3-oxazolidin-2-one (71 mg, 0.148 mmol, 24% yield). ES-LCMS : 457.3 (M+1)

### Example 56

### 3-(1-{[3-chloro-6-(difluoromethyl)-8-(trifluoromethyl)imidazo[1,2-a]pyridin-2-yl]carbonyl}-4-piperidinyl)-1,3-oxazolidin-2-one (Compound 157)

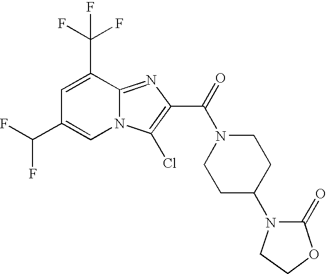

### Step A: 3-chloro-2-{[4-(2-oxo-1,3-oxazolidin-3-yl)-1-piperidinyl]carbonyl}-8-(trifluoromethyl)imidazo[1,2-a]pyridine-6-carbaldehyde

A solution of 3-(1-{[6-bromo-3-chloro-8-(trifluoromethyl)imidazo[1,2-a]pyridin-2-yl]carbonyl}-4-piperidinyl)-1,3-oxazolidin-2-one (500 mg, 1.009 mmol), potassium vinyl trifluoroborate (270 mg, 2.017 mmol), Et3N (0.703 mL, 5.04 mmol) and PdCl₂(dppf)-CH₂Cl₂ adduct (82 mg, 0.101 mmol) in propanol (5 mL) was stirred at 90° C. for 30 minutes. The reaction mixture was diluted with EtOAc and washed with water. The solvent was removed and the crude material was diluted with THF (5.00 mL) and water (5.00 mL). Osmium tetroxide (4.0% in water) (0.791 mL, 0.101 mmol) and sodium periodate (647 mg, 3.03 mmol) was added and the mixture was stirred at RT for 3 hours. The reaction mixture was diluted with EtOAc and washed with water. The solvent was removed and the crude material was purified via silica gel chromatography to give 3-chloro-2-{[4-(2-oxo-1,3-oxazolidin-3-yl)-1-piperidinyl]carbonyl}-8-(trifluoromethyl)imidazo[1,2-a]pyridine-6-carbaldehyde (412 mg, 0.926 mmol, 92% yield). ES-LCMS: 445.5 (M+1)

### Step B: 3-(1-{[3-chloro-6-(difluoromethyl)-8-(trifluoromethyl)imidazo[1,2-a]pyridin-2-yl]carbonyl}-4-piperidinyl)-1,3-oxazolidin-2-one

A solution of 3-chloro-2-{[4-(2-oxo-1,3-oxazolidin-3-yl)-1-piperidinyl]carbonyl}-8-(trifluoromethyl)imidazo[1,2-a]pyridine-6-carbaldehyde (150 mg, 0.337 mmol) and DAST (0.134 mL, 1.012 mmol) in DCM (5 mL) was stirred for 1 hour at RT. The reaction mixture was diluted with EtOAc and washed with water. The solvent was removed and the crude material was purified via reverse phase HPLC to give 3-(1-{[3-chloro-6-(difluoromethyl)-8-(trifluoromethyl)imidazo[1,2-a]pyridin-2-yl]carbonyl}-4-piperidinyl)-1,3-oxazolidin-2-one (94 mg, 0.191 mmol, 57% yield). ES-LCMS : 467.5 (M+1)

### Example 57

### 3-(1-{[3-bromo-6-(3-furanyl)-8-(trifluoromethyl)imidazo[1,2-a]pyridin-2-yl]carbonyl}-4-piperidinyl)-1,3-oxazolidin-2-one (Compound 158)

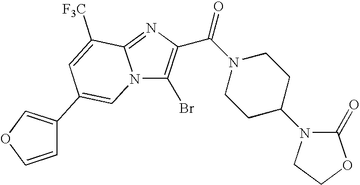

### Step A: methyl 3-bromo-6-(3-furanyl)-8-(trifluoromethyl)imidazo[1,2-a]pyridine-2-carboxylate

An aqueous solution of 3M K₃PO₄ (6.68 mL, 20.05 mmol) was added to a mixture of methyl 3-bromo-6-iodo-8-(trifluoromethyl)imidazo[1,2-a]pyridine-2-carboxylate (3.0 g, 6.68 mmol) and 3-furanylboronic acid (0.785 g, 7.02 mmol) in Acetonitrile (30 mL) under N₂ at room temperature. PdCl₂(dppf)-CH₂Cl₂ adduct (0.273 g, 0.334 mmol) was then added. The reaction mixture was stirred at room temperature for 1 hour, then heated at 50° C. for 1 hour. Cooled to room temperature, the solvent was evaporated. The residue was dissolved in CH₂Cl₂, washed with water and brine, dried over Na₂SO₄, filtered, concentrated and purified by silica gel chromatography (0-1% MeOH/CH₂C1₂) to give the title compound (2.06 g, 79% yield) as an off-white solid. ¹H NMR (400 MHz, CHLOROFORM-d) δ ppm 8.39 (s, 1H) 7.85 (s, 1H), 7.80 (s, 1H), 7.58 (t, J=1.56 Hz, 1H), 6.74 (d, J=0.78 Hz, 1H), 4.01 (s, 3H). ES-LCMS m/z: 389, 391(M+1).

### Step B: 3-bromo-6-(3-furanyl)-8-(trifluoromethyl)imidazo[1,2-a]pyridine-2-carboxylic acid

1N NaOH (8.0 mL, 8.00 mmol) was added to a suspension of methyl 3-bromo-6-(3-furanyl)-8-(trifluoromethyl)imidazo[1,2-a]pyridine-2-carboxylate (1.0 g, 2.57 mmol) in Tetrahydrofuran (THF) (20 mL) and Water (20 mL) at room temperature. The reaction mixture was stirred for 3 h, acidified to pH ˜2 with 1N HCl, extracted with EtOAc three times. The combined organic layer was washed with brine, dried over Na₂SO₄, filtered and concentrated to dryness to give 3-bromo-6-(3-furanyl)-8-(trifluoromethyl)imidazo[1,2-a]pyridine-2-carboxylic acid (0.96 g, 100% yield) as a light yellow solid. ¹H NMR (400 MHz, DMSO-d₆) δ ppm 13.37 (br. s, 1H), 8.74 (s, 1 H), 8.58 (s, 1H), 8.22 (s, 1H), 7.86 (t, J=1.56 Hz, 1H), 7.31 (d, 1H). ES-LCMS m/z: 375, 377 (M+1).

### Step C: 3-(1-{[3-bromo-6-(3-furanyl)-8-(trifluoromethyl)imidazo[1,2-a]pyridin-2-yl]carbonyl}-4-piperidinyl)-1,3-oxazolidin-2-one

Et₃N (1.486 mL, 10.66 mmol) was added to a mixture of 3-bromo-6-(3-furanyl)-8-(trifluoromethyl)imidazo[1,2-a]pyridine-2-carboxylic acid (1.0 g, 2.67 mmol), 3-(4-piperidinyl)-1,3-oxazolidin-2-one hydrochloride (0.551 g, 2.67 mmol), EDC (0.613 g, 3.20 mmol) and HOBT (0.490 g, 3.20 mmol) in N,N-Dimethylformamide (DMF) (25 mL) at room temperature. The mixture was stirred overnight. The solvent was evaporated, and the residue was dissolved in 10% of MeOH in DCM, washed with aqueous NaHCO₃, dried over Na₂SO₄, filtered, concentrated and purified by silica gel chromatography (0-5% MeOH/DCM) to give 3-(1-{[3-bromo-6-(3-furanyl)-8-(trifluoromethyl)imidazo[1,2-a]pyridin-2-yl]carbonyl}-4-piperidinyl)-1,3-oxazolidin-2-one (1.26 g, 90% yield) as a white solid. ES-LCMS m/z: 527, 529 (M+1).

### Example 58

### 3-(1-{[3-bromo-6-(3-furanyl)-8-(trifluoromethyl)imidazo[1,2-a]pyridin-2-yl]carbonyl}-4-piperidinyl)-1,3-oxazolidine-2,4-dione (Compound 159)

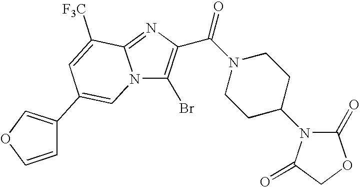

A solution of 3-bromo-6-(3-furanyl)-8-(trifluoromethyl)imidazo[1,2-a]pyridine-2-carboxylic acid (140 mg, 0.373 mmol), 3-(4-piperidinyl)-1,3-oxazolidine-2,4-dione hydrochloride (86 mg, 0.392 mmol), HATU (170 mg, 0.448 mmol) and DIPEA (0.196 mL, 1.120 mmol) in N,N-Dimethylformamide (DMF) (4 mL) was stirred at room temperature for 3 h. The reaction mixture was poured into ethyl acetate and washed sequentially with 5% LiCl (twice). The organic layer was dried over Na₂SO₄, filtered, concentrated and purified by silica gel chromatography (0-60% EtOAc/Hexanes) to yield a white solid, which was stirred with 1:1 Hexanes:Et₂O containing 1% of MeOH for 1 h. The solid was collected and washed with 1:1 Hexanes:Et₂O containing 1% of MeOH and air dried to give 3-(1-{[3-bromo-6-(3-furanyl)-8-(trifluoromethyl)imidazo[1,2-a]pyridin-2-yl]carbonyl}-4-piperidinyl)-1,3-oxazolidine-2,4-dione (188 mg, 93% yield) as a white solid. LCMS m/z: 541, 543 (M+1).

### Example 59

### 6-(3-furanyl)-2-{[4-(2-oxo-1,3-oxazolidin-3-yl)-1-piperidinyl]carbonyl}-8-(trifluoromethyl)imidazo[1,2-a]pyridine-3-carbonitrile (Compound 160)

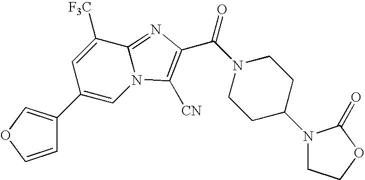

A mixture of 3-(1-{[3-bromo-6-(3-furanyl)-8-(trifluoromethyl)imidazo[1,2-a]pyridin-2-yl]carbonyl}-4-piperidinyl)-1,3-oxazolidin-2-one (100 mg, 0.190 mmol), copper(I) cyanide (67.9 mg, 0.759 mmol) in N,N-Dimethylformamide (DMF) (1.5 mL) was heated in microwave oven at 160° C. for 30 minutes. Cooled to room temperature, the mixture was diluted with EtOAc, washed with 5% LiCl (twice), brine, and dried over Na₂SO₄, filtered, concentrated and purified by silica gel chromatography (0-15% Acetone/CH₂Cl₂) to give 6-(3-furanyl)-2-{[4-(2-oxo-3-yl)-1-piperidinyl]carbonyl}-8-(trifluoromethyl)imidazo[1,2-a]pyridine-3-carbonitrile (44 mg, 49% yield) as a white solid. ES-LCMS m/z: 474 (M+1).

### Example 60

### 2-{[4-(2,4-dioxo-1,3-oxazolidin-3-yl)-1-piperidinyl]carbonyl}-6-(3-furanyl)-8-(trifluoromethyl)imidazo[1,2-a]pyridine-3-carbonitrile (Compound 161)

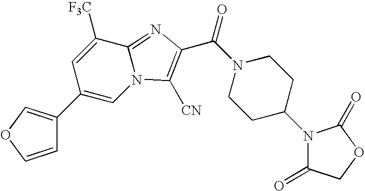

A mixture of 3-(1-{[3-bromo-6-(3-furanyl)-8-(trifluoromethyl)imidazo[1,2-a]pyridin-2-yl]carbonyl}-4-piperidinyl)-1,3-oxazolidine-2,4-dione (100 mg, 0.185 mmol), copper(I) cyanide (66.2 mg, 0.739 mmol) in N,N-Dimethylformamide (DMF) (1.5 mL) was heated in microwave oven at 160° C. for 25 minutes. Cooled to room temperature, the mixture was diluted with EtOAc, washed with 5% LiCl (twice), brine, and dried over Na₂SO₄, filtered, concentrated and purified by silica gel chromatography (0-55% EtOAc/Hexanes) to give 2-{[4-(2,4-dioxo-1,3-oxazolidin-3-yl)-1-piperidinyl]carbonyl}-6-(3-furanyl)-8-(trifluoromethyl)imidazo[1,2-a]pyridine-3-carbonitrile (47.5 mg, 52.8% yield) as a white solid. ES-LCMS m/z: 488 (M+1).

### Example 61

### 6-(3-furanyl)-2-{[4-(2-oxo-1,3-oxazolidin-3-yl)-1-piperidinyl]carbonyl}-8-(trifluoromethyl)imidazo[1,2-a]pyridine-3-carbonitrile (Compound 162)

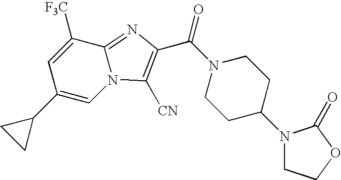

### Step A: methyl 6-cyclopropyl-8-(trifluoromethyl)imidazo[1,2-a]pyridine-2-carboxylate

A mixture of methyl 6-bromo-8-(trifluoromethyl)imidazo[1,2-a]pyridine-2-carboxylate (150 mg, 0.464 mmol), cyclopropylboronic acid (80 mg, 0.929 mmol), potassium phosphate (305 mg, 1.393 mmol), PdCl₂(dppf)-CH₂Cl₂ adduct (18.96 mg, 0.023 mmol) in 1,4-Dioxane (4 mL) was degassed with N₂ and heated at 90° C. for 3 h. Cooled to room temperature, the mixture was diluted with EtOAc, washed with brine and the organic layer was dried over Na₂SO₄, filtered, concentrated and purified by silica gel chromatography (0-45% EtOAc/Hexanes) to give methyl 6-cyclopropyl-8-(trifluoromethyl)imidazo[1,2-a]pyridine-2-carboxylate (104 mg, 79% yield) as a white solid. ES-LCMS m/z: 488 (M+1).

### Step B: methyl 3-bromo-6-cyclopropyl-8-(trifluoromethyl)imidazo[1,2-a]pyridine-2-carboxylate

NBS (66.4 mg, 0.369 mmol) was added to a mixture of methyl 6-cyclopropyl-8-(trifluoromethyl)imidazo[1,2-a]pyridine-2-carboxylate (100 mg, 0.352 mmol) in 1,2-Dichloroethane (DCE) (3 mL). The reaction mixture was stirred at room temperature for 3 h, diluted with CH₂Cl₂, and washed with 5% aqueous Na₂S₂O₃, aqueous saturated NaHCO₃ and brine. The organic layer was separated and dried over Na₂SO₄, filtered and concentrated to give methyl 3-bromo-6-cyclopropyl-8-(trifluoromethyl)imidazo[1,2-a]pyridine-2-carboxylate (129 mg, 100% yield) as an off-white solid. ES-LCMS m/z: 363, 365 (M+1).

### Step C: 3-bromo-6-cyclopropyl-8-(trifluoromethyl)imidazo[1,2-a]pyridine-2-carboxylic acid

1N NaOH (1.1 mL, 1.100 mmol) was added to a solution of methyl 3-bromo-6-cyclopropyl-8-(trifluoromethyl)imidazo[1,2-a]pyridine-2-carboxylate (129 mg, 0.355 mmol) in Tetrahydrofuran (THF) (4 mL) and Water (3 mL). The reaction mixture was stirred at room temperature for 3 h, and acidified to pH ˜2 with 1N HCl, extracted with EtOAc twice. The combined organic extract was washed with brine, dried over Na₂SO₄, filtered and concentrated to give 3-bromo-6-cyclopropyl-8-(trifluoromethyl)imidazo[1,2-a]pyridine-2-carboxylic acid (124 mg, 100% yield) as a light yellow solid. ES-LCMS m/z: 349, 351 (M+1).

### Step D: 3-(1-{[3-bromo-6-cyclopropyl-8-(trifluoromethyl)imidazo[1,2-a]pyridin-2-yl]carbonyl}-4-piperidinyl)-1,3-oxazolidin-2-one

A solution of 3-bromo-6-cyclopropyl-8-(trifluoromethyl)imidazo[1,2-a]pyridine-2-carboxylic acid (122 mg, 0.349 mmol), 3-(4-piperidinyl)-1,3-oxazolidin-2-one hydrochloride (76 mg, 0.367 mmol), HATU (159 mg, 0.419 mmol) and DIPEA (0.183 mL, 1.048 mmol) in N,N-Dimethylformamide (DMF) (4 mL) was maintained with stirring at room temperature overnight. The reaction mixture was poured into ethyl acetate and washed sequentially with 5% LiCl (twice). The organic layer was dried over Na₂SO₄, filtered, concentrated and purified by silica gel chromatography (0-3% MeOH/CH₂Cl₂) to yield a light brown solid, which was stirred with 1:1 Hexanes:Et₂O containing 1% of MeOH for 1 h. The solid was collected and washed with 1:1 Hexanes:Et₂O containing 1% of MeOH and air dried to give 3-(1-{[3-bromo-6-cyclopropyl-8-(trifluoromethyl)imidazo[1,2-a]pyridin-2-yl]carbonyl}-4-piperidinyl)-1,3-oxazolidin-2-one (153 mg, 87% yield) as a white solid. ES-LCMS m/z: 501, 503 (M+1).

### Step E: 6-cyclopropyl-2-{[4-(2-oxo-1,3-oxazolidin-3-yl)-1-piperidinyl]carbonyl}-8-(trifluoromethyl)imidazo[1,2-a]pyridine-3-carbonitrile

A mixture of 3-(1-{[3-bromo-6-cyclopropyl-8-(trifluoromethyl)imidazo[1,2-a]pyridin-2-yl]carbonyl}-4-piperidinyl)-1,3-oxazolidin-2-one (119 mg, 0.237 mmol), copper(I) cyanide (85 mg, 0.950 mmol) in N,N-Dimethylformamide (DMF) (2 mL) was heated in microwave oven at 140° C. for 20 minutes. Cooled to room temperature, the mixture was diluted with EtOAc, filtered through a pad of Celite, washed with water/brine twice, brine, and dried over Na₂SO₄, filtered, concentrated and purified by silica gel chromatography (0-40% EtOAc/CH₂Cl₂) to give 6-cyclopropyl-2-{[4-(2-oxo-1,3-oxazolidin-3-yl)-1-piperidinyl]carbonyl}-8-(trifluoromethyl)imidazo[1,2-a]pyridine-3-carbonitrile (90 mg, 85% yield) as a white solid. ES-LCMS m/z: 448 (M+1).

### Example 62

### 1-(1-{[3-chloro-6-cyclopropyl-8-(trifluoromethyl)imidazo[1,2-a]pyridin-2-yl]carbonyl}-4-piperidinyl)-4-hydroxy-2-pyrrolidinone (Compound 163)

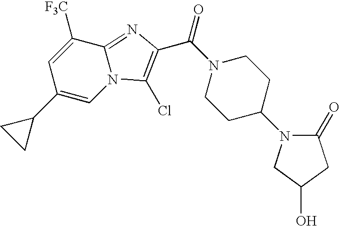

To a mixture of 3-chloro-6-cyclopropyl-8-(trifluoromethyl)imidazo[1,2-a]pyridine-2-carboxylic acid (150 mg, 0.492 mmol), 4-hydroxy-1-(4-piperidinyl)-2-pyrrolidinone hydrochloride (146 mg, 0.640 mmol) in N,N-Dimethylformamide (DMF) (3 mL) was added dropwise DIPEA (0.344 mL, 1.969 mmol). The mixture was stirred at room temperature for 30 minutes, cooled to 0° C. in an ice bath, and 1-propanephosphoric acid cyclic anhydride (50% wt.% in EtOAc) (0.440 mL, 0.739 mmol) was added dropwise. The mixture was stirred for 1.5 h and added to 20 mL of water with stirring, extracted with EtOAc. The combined organic extract was washed with 5% LiCl, aqueous saturated NaHCO₃ and brine, dried over Na₂SO₄, filtered, concentrated and purified by silica gel chromatography (0-8% MeOH/CH₂Cl₂) to give 1-(1-{[3-chloro-6-cyclopropyl-8-(trifluoromethyl)imidazo[1,2-a]pyridin-2-yl]carbonyl}-4-piperidinyl)-4-hydroxy-2-pyrrolidinone (170 mg, 73.3% yield) as a white solid. ES-LCMS m/z: 471 (M+1).

### Example 63

### 3-(1-{[6-(3-furanyl)-3-methyl-8-(trifluoromethyl)imidazo[1,2-a]pyridin-2-yl]carbonyl}-4-piperidinyl)-1,3-oxazolidin-2-one (Compound 164)

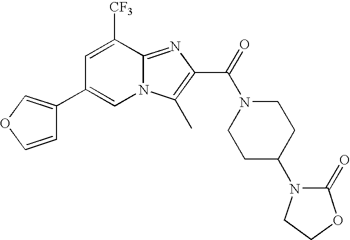

Dimethylzinc (0.095 mL, 0.095 mmol) was added to a solution of tetrakis(triphenylphosphine)palladium(0) (21.92 mg, 0.019 mmol) and 3-(1-{[3-bromo-6-(3-furanyl)-8-(trifluoromethyl)imidazo[1,2-a]pyridin-2-yl]carbonyl}-4-piperidinyl)-1,3-oxazolidin-2-one (50 mg, 0.095 mmol) in tetrahydrofuran (2.0 mL) in a seal tube at room temperature under N₂. The reaction mixture was heated to 60° C. for 2 hours. Cooled down and removal off the solvent, the residue was redissolved in dichloromethane, aqueous NaHCO3 was added, extracted with 10% MeOH in CH₂Cl₂. The combined extracts were dried over Na₂SO₄, filtered and concentrated. The crude residue was purified by reverse phase HPLC to afford the title compound as an off-white solid (21 mg, 48%). 1H NMR (400 MHz, DMSO-d₆) δ ppm 8.79 (s, 1H) 8.49 (s, 1H) 8.08 (s, 1H) 7.84 (t, J=1.66 Hz, 1H) 7.27 (d, J=0.98 Hz, 1H) 4.63 (d, J=12.68 Hz, 1H) 4.52 (d, J=12.88 Hz, 1H) 4.26 (t, J=8.00 Hz, 2H) 3.74-3.87 (m, 1H) 3.47-3.59 (m, 2H) 3.11-3.24 (m, 1H) 2.87 (t, J=12.00 Hz, 1H) 2.66 (s, 3H) 1.80 (d, J=10.93 Hz, 1H) 1.58-1.72 (m, 3H). ES-LCMS: m/z 463.2 (M+1).

### Example 64

### 3-(1-{[6-(3-furanyl)-3-(1-methylethyl)-8-(trifluoromethyl)imidazo[1,2-a]pyridin-2-yl]carbonyl}-4-piperidinyl)-1,3-oxazolidin-2-one (Compound 165)

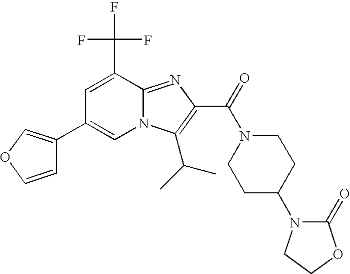

### Step A: 3-(1-{[6-(3-furanyl)-3-(1-methylethenyl)-8-(trifluoromethyl)imidazo[1,2-a]pyridin-2-yl]carbonyl}-4-piperidinyl)-1,3-oxazolidin-2-one

Et₃N (0.159 mL, 1.138 mmol) was added to a mixture of 3-(1-{[3-bromo-6-(3-furanyl)-8-(trifluoromethyl)imidazo[1,2-a]pyridin-2-yl]carbonyl}-4-piperidinyl)-1,3-oxazolidin-2-one (120 mg, 0.228 mmol), potassium isopropenyltrifluoroborate (50.5 mg, 0.341 mmol) and PdCl₂(dppf)-CH₂Cl₂ adduct (3.72 mg, 4.55 μmol) in n-propanol (3.0 mL) under N₂. The mixture was heated to 100° C. and stirred for 2 h. Cooled down to room temperature and the reaction mixture was filtered through celite and concentrated to dryness. The residue was dissolved in EtOAc, washed with saturated NaHCO3, dried over Na₂SO₄, filtered and concentrated. The crude residue was purified by chromatography on silica gel eluted with 0-5% 2 M NH₃ solution of MeOH in CH₂Cl₂ to give the product as a light-yellow solid (99 mg, 89%). 1H NMR (400 MHz, CHLOROFORM-d) δ ppm 8.33 (s, 1H) 7.77 (s, 1H) 7.66 (s, 1H) 7.52-7.56 (m, 1H) 6.66 (d, J=0.78 Hz, 1H) 5.67 (s, 1H) 5.34 (s, 1H) 4.80-4.90 (m, 1H) 4.53 (dd, J=13.27, 1.76 Hz, 1H) 4.29-4.38 (m, 2H) 3.95-4.08 (m, 1H) 3.54 (t, J=8.00 Hz, 2H) 3.07-3.18 (m, 1H) 2.82 (td, J=12.93, 2.44 Hz, 1H) 2.20 (s, 3H) 1.67-1.94 (m, 4H). ES-LCMS: m/z 489.2 (M+1).

### Step B: 3-(1-{[6-(3-furanyl)-3-(1-methylethyl)-8-(trifluoromethyl)imidazo[1,2-a]pyridin-2-yl]carbonyl}-4-piperidinyl)-1,3-oxazolidin-2-one

A mixture of 3-(1-{[6-(3-furanyl)-3-(1-methylethenyl)-8-(trifluoromethyl)imidazo[1,2-a]pyridin-2-yl]carbonyl}-4-piperidinyl)-1,3-oxazolidin-2-one (100 mg, 0.205 mmol), diphenyl sulfide (3.44 μL, 0.020 mmol) and Pd/C (21.79 mg, 0.020 mmol) in methanol (8 mL) and ethyl acetate (8.00 mL) was reacted under H₂ (40 psi). After completing, filtered off the catalyst and the filtrate was concentrated. The crude residue was purified by reverse phase HPLC to give the title compound as a white solid (70 mg, 70%). 1H NMR (400 MHz, DMSO-d₆) δ ppm 8.85 (s, 1H) 8.49 (s, 1H) 8.04 (s, 1H) 7.83 (t, J=1.66 Hz, 1H) 7.31 (d, J=0.98 Hz, 1H) 4.64 (d, J=13.27 Hz, 1H) 4.26 (t, J=7.90 Hz, 2H) 3.63-3.88 (m, 3H) 3.42-3.58 (m, 2H) 3.08 (m, 1H) 2.89 (td, J=12.73, 2.24 Hz, 1H) 1.81 (d, J=10.73 Hz, 1H) 1.49-1.69 (m, 3H) 1.36 (dd, 6H). ES-LCMS: m/z 492.5 (M+1).

### Example 65

### 3-(1-{[6-(3-furanyl)-3-(hydroxymethyl)-8-(trifluoromethyl)imidazo[1,2-a]pyridin-2-yl]carbonyl}-4-piperidinyl)-1,3-oxazolidin-2-one (Compound 166)

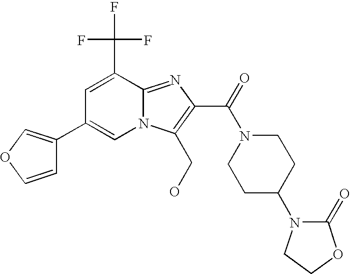

### Step A: methyl 6-bromo-3-formyl-8-(trifluoromethyl)imidazo[1,2-a]pyridine-2-carboxylate

POCl₃ (6.0 mL, 65.5 mmol) was added to a solution of methyl 6-bromo-8-(trifluoromethyl)imidazo[1,2-a]pyridine-2-carboxylate (2.0 g, 6.19 mmol) in N,N-dimethylformamide (5.0 mL) and the mixture was stirred for 1 h at 120° C. Cooled down and the mixture was poured into concentrate HCl and stirred for 15 min. Basified with 5 N NaOH and then extracted with EtOAc. The combined extracts were dried over Na₂SO₄, filtered and concentrated. The crude residue was purified by chromatography on silica gel eluted with 0-30% EtOAc in hexane to give the product as a light-yellow solid (1.3 g, 60%). ES-LCMS: m/z 351.0 (M+1).

### Step B: 3-formyl-6-(3-furanyl)-8-(trifluoromethyl)imidazo[1,2-a]pyridine-2-carboxylic acid

K₃PO₄ (3.25 mL, 3.0 M, 9.75 mmol) was added to a mixture of methyl 6-bromo-3-formyl-8-(trifluoromethyl)imidazo[1,2-a]pyridine-2-carboxylate (380 mg, 1.082 mmol) and 3-furanylboronic acid (157 mg, 1.407 mmol) in 1,4-dioxane (8.0 mL) at room temperature under N₂, PdCl2(dppf)-CH2Cl2 adduct (53.0 mg, 0.065 mmol) was then added. The mixture was microwaved at 110° C. for 10 minutes. Crude LCMS shown that the product is methyl ester. Concentrated to removal off 1,4-dioxane and MeCN (8.0 mL) was added, the mixture was heated to reflux overnight. Concentrated and the residue was redissolved in EtOAc and water. Acidified to PH ˜2 with 1N HCl and then extracted with EtOAc. The combined extracts were washed with brine, dried over Na₂SO₄. Filtered and concentrated and dried in vacuo to give the desired product as a light-yellow solid (340 mg, 97%) which was used for the next step without further purification. ES-LCMS: m/z 325.1 (M+1).

### Step C: 6-(3-furanyl)-2-{[4-(2-oxo-1,3-oxazolidin-3-yl)-1-piperidinyl]carbonyl}-8-(trifluoromethyl)imidazo[1,2-a]pyridine-3-carbaldehyde

A mixture of 3-formyl-6-(3-furanyl)-8-(trifluoromethyl)imidazo[1,2-a]pyridine-2-carboxylic acid (85 mg, 0.262 mmol), 3-(4-piperidinyl)-1,3-oxazolidin-2-one (59.6 mg, 0.288 mmol), EDC (60.3 mg, 0.315 mmol), HOBt (48.2 mg, 0.315 mmol) and Et₃N (0.292 mL, 2.097 mmol) in N,N-dimethylformamide (3 mL) at room temperature was stirred overnight. It was evaporated under reduced pressure, the residue was dissolved in EtOAc, washed with aqueous NaHCO₃, dried over Na₂SO₄. Filtered and concentrated, the crude residue was purified by chromatography on silica gel eluted with 30-80% EtOAc in hexane to give the desired product as a light-yellow solid (40 mg, 32%). ES-LCMS: m/z 477.2 (M+1).

### Step D: 3-(1-{[6-(3-furanyl)-3-(hydroxymethyl)-8-(trifluoromethyl)imidazo[1,2-a]pyridin-2-yl]carbonyl}-4-piperidinyl)-1,3-oxazolidin-2-one

NaBH4 (9.53 mg, 0.252 mmol) was added to a suspension of 6-(3-furanyl)-2-{[4-(2-oxo-1,3-oxazolidin-3-yl)-1-piperidinyl]carbonyl}-8-(trifluoromethyl)imidazo[1,2-a]pyridine-3-carbaldehyde (40 mg, 0.084 mmol) in methanol (5.0 mL) at 0° C. under N₂. It was warmed up to room temperature and stirred for 30 minutes. Starting material was consumed up, it was then concentrated. The residue was purified by reverse phase HPLC to give the title compound as a white solid (32 mg, 80%). 1H NMR (400 MHz, DMSO-d₆) δ ppm 8.90 (s, 1H) 8.48 (s, 1H) 8.14 (s, 1H) 7.86 (t, J=1.56 Hz, 1H) 7.17 (d, J=0.98 Hz, 1H) 5.46 (t, J=5.56 Hz, 1H), 5.01 (d, J=2.15 Hz, 2H) 4.63 (d, J=13.07 Hz, 1H) 4.39 (d, J=13.07 Hz, 1H) 4.27 (t, J=8.00 Hz, 2H) 3.76-3.89 (m, 1H) 3.44-3.60 (m, 2H) 3.11-3.24 (m, 1H) 2.88 (t, J=11.90 Hz, 1H) 1.75-1.86 (m, 1H) 1.59-1.73 (m, 3H). ES-LCMS: m/z 480.0 (M+1).

### Example 66

### 1-(1-{[3-chloro-6-(1H-pyrazol-4-yl)-8-(trifluoromethyl)imidazo[1,2-a]pyridin-2-yl]carbonyl}-4-piperidinyl)-2,5-pyrrolidinedione (Compound 167)

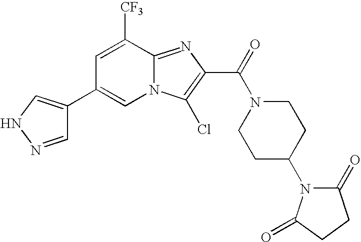

### Step A: 1-(4-piperidinyl)-2,5-pyrrolidinedione

A solution of 1-[1-(phenylmethyl)-4-piperidinyl]-2,5-pyrrolidinedione (prepared according to the procedure of Verschueren et al. J. Med. Chem. 2005, 48, 1931) (0.473 g, 1.74 mmol) and Pd(OH)₂ (catalytic) in MeOH (30 mL) was stirred under a hydrogen atmosphere (50 psi) for 1 hour. The solution was filtered through celite, washing with MeOH and evaporated to dryness. The white solid was azeotroped with EtOAc (3×) to afford 1-(4-piperidinyl)-2,5-pyrrolidinedione, which was used without further purification (assumed quantitative).

### Step B: 1-(1-{[3-chloro-6-(1H-pyrazol-4-yl)-8-(trifluoromethyl)imidazo[1,2-a]pyridin-2-yl]carbonyl}-4-piperidinyl)-2,5-pyrrolidinedione

A mixture of 3-chloro-6-(1H-pyrazol-4-yl)-8-(trifluoromethyl)imidazo[1,2-a]pyridine-2-carboxylic acid (0.100 g, 0.30 mmol), 1-(4-piperidinyl)-2,5-pyrrolidinedione (0.083 g, 0.45 mmol), DIPEA (0.21 mL, 1.2 mmol) and HATU (0.15 g, 0.39 mmol) in DMF (1.5 mL) was stirred at room temperature for 1 hour. The reaction mixture was diluted with methylene chloride and washed with saturated sodium bicarbonate. The organic layer was dried over sodium sulfate and the solvent was evaporated. The residue was purified by silica gel chromatography (0-5% MeOH in CH₂Cl₂) to afford the title compound (0.11 g, 72%) as a white solid.

### Example 67

### 1-(1-{[3-chloro-6-(3-furanyl)-8-(trifluoromethyl)imidazo[1,2-a]pyridin-2-yl]carbonyl}-4-piperidinyl)-2,5-pyrrolidinedione (Compound 168)

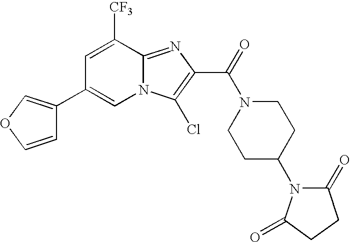

A mixture of 3-chloro-6-(3-furanyl)-8-(trifluoromethyl)imidazo[1,2-a]pyridine-2-carboxylic acid (0.100 g, 0.30 mmol), 1-(4-piperidinyl)-2,5-pyrrolidinedione (0.083 g, 0.45 mmol), DIPEA (0.21 mL, 1.2 mmol) and HATU (0.15 g, 0.39 mmol) in DMF (1.5 mL) was stirred at room temperature for 1 hour. The reaction mixture was diluted with methylene chloride and washed with saturated sodium bicarbonate. The organic layer was dried over sodium sulfate and the solvent was evaporated. The residue was purified by reverse phase HPLC (acetonitrile/water with 0.1% formic acid) to afford the title compound (0.073 g, 49%) as a white solid.

### Example 68

### 1-(1-{[3-chloro-6-(3-furanyl)-8-(trifluoromethyl)imidazo[1,2-a]pyridin-2-yl]carbonyl}-4-piperidinyl)-2-imidazolidinone (Compound 169)

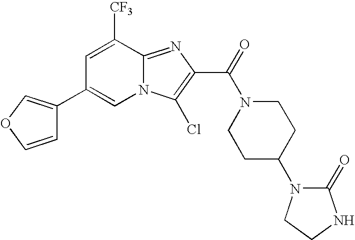

### Step A: 1-[1-(phenylmethyl)-4-piperidinyl]-2-imidazolidinone

2-chloroethylisocyanate (1.055 g, 10 mmol) was added to a solution of 1-(phenylmethyl)-4-piperidinamine (1.903 g, 10 mmol) in THF (50.0 ml) and the mixture was stirred for 1 hour at room temperature. Lithium bis(trimethylsilyl)amide in THF (10.00 ml, 10.00 mmol) was added and stirring was continued overnight. The reaction mixture was evaporated to dryness, 1M HCl was added and the solution was extracted with CH₂Cl₂. The aqueous layer was neutralized to pH 7 and extracted with CH₂Cl₂. The organic layers were pooled, dried (Na₂SO₄), and evaporated to give a light yellow residue. The residue was dissolved in THF (20 mL) and NaH (0.800g 60% dispersion in mineral oil, 20.00 mmol). The reaction mixture was stirred at room temperature for 1 hour and DMF (5 mL) was added. After another 2 hours, water was added and the solution was extracted with EtOAc The organic layer was dried (Na₂SO₄), filtered, evaporated and purified by silica gel chromatography(0-5% MeOH (2M NH₃) in CH₂Cl₂) to afford the title compound (0.8507 g, 3.28 mmol, 32.8% yield) as a white solid.

### Step B: 1-(4-piperidinyl)-2-imidazolidinone

The title compound (quant.) was obtained from 1-[1-(phenylmethyl)-4-piperidinyl]-2-imidazolidinone (0.351 g, 1.35 mmol) as described previously.

### Step C: 1-(1-{[3-chloro-6-(3-furanyl)-8-(trifluoromethyl)imidazo[1,2-a]pyridin-2-yl]carbonyl}-4-piperidinyl)-2-imidazolidinone

A mixture of 3-chloro-6-(3-furanyl)-8-(trifluoromethyl)imidazo[1,2-a]pyridine-2-carboxylic acid (0.100 g, 0.30 mmol), 1-(4-piperidinyl)-2-imidazolidinone (0.077 g, 0.45 mmol), DIPEA (0.21 mL, 1.2 mmol) and HATU (0.15 g, 0.39 mmol) in DMF (1.5 mL) was stirred at room temperature for 1 hour. The reaction mixture was diluted with methylene chloride and washed with saturated sodium bicarbonate. The organic layer was dried over sodium sulfate and the solvent was evaporated. The residue was purified by reverse phase HPLC (acetonitrile/water with 0.1% formic acid) to afford the title compound (0.034 g, 24%) as a white solid.

### Example 69

### 1-(1-{[3-chloro-6-(1H-pyrazol-4-yl)-8-(trifluoromethyl)imidazo[1,2-a]pyridin-2-yl]carbonyl}-4-piperidinyl)-2-imidazolidinone (Compound 170)

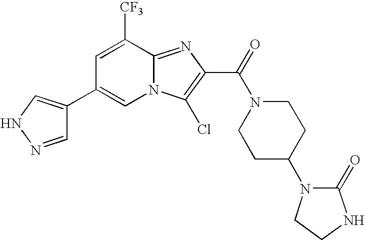

A mixture of 3-chloro-6-(1H-pyrazol-4-yl)-8-(trifluoromethyl)imidazo[1,2-a]pyridine-2-carboxylic acid (0.100 g, 0.30 mmol), 1-(4-piperidinyl)-2-imidazolidinone (0.077 g, 0.45 mmol), DIPEA (0.21 mL, 1.2 mmol) and HATU (0.15 g, 0.39 mmol) in DMF (1.5 mL) was stirred at room temperature for 1 hour. The reaction mixture was diluted with methylene chloride and washed with saturated sodium bicarbonate. The organic layer was dried over sodium sulfate and the solvent was evaporated. The residue was purified by reverse phase HPLC (acetonitrile/water with 0.1% formic acid) to afford the title compound (0.075 g, 51%) as a white solid.

### Example 70

### 1-(1-{[3-chloro-6-(3-furanyl)-8-(trifluoromethyl)imidazo[1,2-a]pyridin-2-yl]carbonyl}-4-piperidinyl)-2,4-imidazolidinedione (Compound 171)

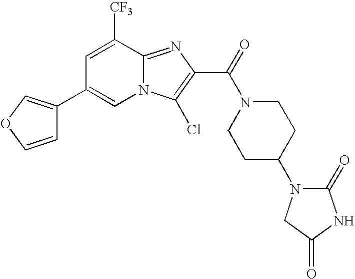

A mixture of 3-chloro-6-(3-furanyl)-8-(trifluoromethyl)imidazo[1,2-a]pyridine-2-carboxylic acid (0.070 g, 0.21 mmol), 1-(4-piperidinyl)-2,4-imidazolidinedione (0.056 g, 0.25 mmol), DIPEA (0.15 mL, 0.85 mmol) and HATU (0.12 g, 0.32 mmol) in DMF (1 mL) was stirred at room temperature for 1 hour. The reaction mixture was diluted with methylene chloride and washed with saturated sodium bicarbonate. The organic layer was dried over sodium sulfate and the solvent was evaporated. The residue was purified by reverse phase HPLC (acetonitrile/water with 0.1% formic acid) to afford the title compound (0.017 g, 16%) as a white solid.

### Example 71

### 1-(1-{[3-chloro-6-(1H-pyrazol-4-yl)-8-(trifluoromethyl)imidazo[1,2-a]pyridin-2-yl]carbonyl}-4-piperidinyl)-2,4-imidazolidinedione (compound 172)

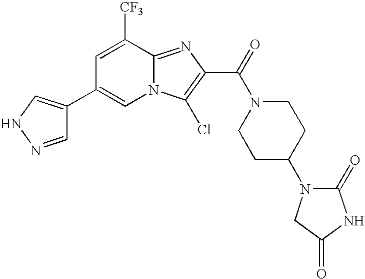

A mixture of 3-chloro-6-(1H-pyrazol-4-yl)-8-(trifluoromethyl)imidazo[1,2-a]pyridine-2-carboxylic acid (0.070 g, 0.21 mmol), 1-(4-piperidinyl)-2,4-imidazolidinedione (0.056 g, 0.25 mmol), DIPEA (0.15 mL, 0.85 mmol) and HATU (0.12 g, 0.32 mmol) in DMF (1 mL) was stirred at room temperature for 1 hour. The reaction mixture was diluted with methylene chloride and washed with saturated sodium bicarbonate. The organic layer was dried over sodium sulfate and the solvent was evaporated. The residue was purified by reverse phase HPLC (acetonitrile/water with 0.1% formic acid) and silica gel (0-8% MeOH in CH2Cl2) to afford the title compound (0.008 g, 8%) as a white solid.

### Example 72

### 1-(1-{[3-chloro-6-(3-furanyl)-8-(trifluoromethyl)imidazo[1,2-a]pyridin-2-yl]carbonyl}-4-piperidinyl)-3-methyl-2-imidazolidinone (Compound 173)

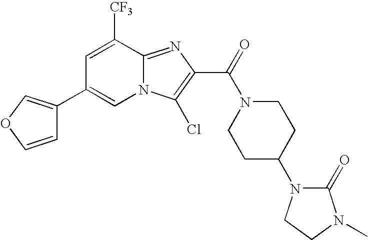

### Step A: 1-methyl-3-[1-(phenylmethyl)-4-piperidinyl]-2-imidazolidinone

NaH (0.109 g of 60% dispersion in mineral oil, 2.73 mmol) was added to a solution of 1-[1-(phenylmethyl)-4-piperidinyl]-2-imidazolidinone (0.472 g, 1.82 mmol) in DMF (9.1 ml). Gas was evolved. After 1.5 hours, iodomethane (0.102 ml, 1.64 mmol) was added and the reaction was stirred for another 3 hours at room temperature. Water was added and the reaction mixture was extracted with CH₂Cl₂. The organic layer was dried (Na₂SO₄), filtered, evaporated and purified by silica gel chromatography (0-4% MeOH in CH₂Cl₂) to afford the title compound (0.34 g, 48%) which was contaminated. The material was used without further purification.

### Step B: 1-methyl-3-(4-piperidinyl)-2-imidazolidinone

The title compound (quant.) was obtained from 1-methyl-3-[1-(phenylmethyl)-4-piperidinyl]-2-imidazolidinone (0.34 g, 0.87 mmol) as described previously.

### Step C. 1-(1-{[3-chloro-6-(3-furanyl)-8-(trifluoromethyl)imidazo[1,2-a]pyridin-2-yl]carbonyl}-4-piperidinyl)-3-methyl-2-imidazolidinone

A mixture of 3-chloro-6-(3-furanyl)-8-(trifluoromethyl)imidazo[1,2-a]pyridine-2-carboxylic acid (0.070 g, 0.21 mmol), 1-methyl-3-(4-piperidinyl)-2-imidazolidinone (0.080 g, 0.44 mmol), (0.15 mL, 0.85 mmol) and HATU (0.12 g, 0.32 mmol) in DMF (1 mL) was stirred at room temperature for 1 hour. The reaction mixture was diluted with methylene chloride and washed with saturated sodium bicarbonate. The organic layer was dried over sodium sulfate and the solvent was evaporated. The residue was purified by reverse phase HPLC (acetonitrile/water with 0.1% formic acid) and silica gel (0-8% MeOH in CH₂Cl₂) to afford the title compound (0.004 g, 4%) as a white solid.

### Example 73

### 3-(1-{[3-chloro-6-(1H-pyrazol-4-yl)-8-(trifluoromethyl)imidazo[1,2-a]pyridin-2-yl]carbonyl}-4-piperidinyl)-2,4-imidazolidinedione (Compound 174)

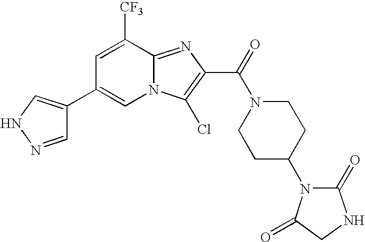

### Step A: 3-[1-(phenylmethyl)-4-piperidinyl]-2,4-imidazolidinedione

A solution of glycine ethyl ester isocyanate (1.27 ml, 11.14 mmol) in 40 mL CHCl₃ was stirred at room temperature. A solution of 1-(phenylmethyl)-4-piperidinamine (2.12 g, 11.14 mmol) in 24 mL CHCl₃ was added over 15 mins. The reaction mixture was then evaporated to dryness to give a yellow solid. The residue was taken up in 1:1 EtOH:10M HCl (70 mL) and heated under reflux for 3 h. The EtOH was evaporated, and the solution was neutralized with 5N NaOH first, then NaHCO₃. The mixture was extracted with CH₂Cl₂, dried (Na₂SO₄), filtered, evaporated and purified by silica gel chromatography (0-100% EtOAc/CH₂Cl₂) to afford the title compound (2.12 g, 70%) as a white crystalline solid.

### Step B: 3-(4-piperidinyl)-2,4-imidazolidinedione

The title compound (quant.) was obtained from 3-[1-(phenylmethyl)-4-piperidinyl]-2,4-imidazolidinedione (1.73 g, 6.33 mmol) as described previously.

### Step C. 3-(1-{[3-chloro-6-(1H-pyrazol-4-yl)-8-(trifluoromethyl)imidazo[1,2-a]pyridin-2-yl]carbonyl}-4-piperidinyl)-2,4-imidazolidinedione

A mixture of 3-chloro-6-(1H-pyrazol-4-yl)-8-(trifluoromethyl)imidazo[1,2-a]pyridine-2-carboxylic acid (0.080 g, 0.24 mmol), 3-(4-piperidinyl)-2,4-imidazolidinedione (0.058 g, 0.32 mmol), hydroxybenzotriazole (0.056 g, 0.36 mmol) and 1-ethyl-3-(3-dimethyllaminopropyl)carbodiimide hydrochloride (0.060 g, 0.32 mmol) in DMF (1 mL) was stirred at room temperature for 1 hour. The reaction mixture was diluted with methylene chloride and washed with saturated sodium bicarbonate. The organic layer was dried over sodium sulfate and the solvent was evaporated. The residue was purified by silica gel chromatography (0-10% MeOH in CH₂Cl₂) to afford the title compound (0.020 g, 17%) as a white solid.

### Example 74

### 3-(1-{[3-chloro-6-(3-furanyl)-8-(trifluoromethyl)imidazo[1,2-a]pyridin-2-yl]carbonyl}-4-piperidinyl)-2,4-imidazolidinedione (Compound 175)

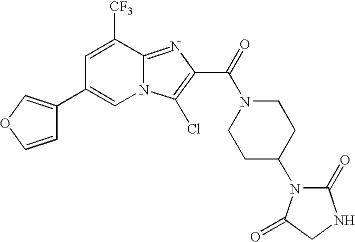

A mixture of 3-chloro-6-(3-furanyl)-8-(trifluoromethyl)imidazo[1,2-a]pyridine-2-carboxylic acid (0.17 g, 0.50 mmol), 3-(4-piperidinyl)-2,4-imidazolidinedione (0.101 g, 0.55 mmol), hydroxybenzotriazole (0.115 g, 0.75 mmol) and 1-ethyl-3-(3-dimethyllaminopropyl)carbodiimide hydrochloride (0.125 g, 0.65 mmol) in DMF (2.5 mL) was stirred at room temperature for 1 hour. The reaction mixture was diluted with methylene chloride and washed with saturated sodium bicarbonate. The organic layer was dried over sodium sulfate and the solvent was evaporated. The residue was purified by silica gel chromatography (0-8% MeOH in CH2Cl2) to afford the title compound (0.152 g, 61%) as a white solid.

### Example 75

### 3-(1-{[3-chloro-6-(3-furanyl)-8-(trifluoromethyl)imidazo[1,2-a]pyridin-2-yl]carbonyl}-4-piperidinyl)-4,4-dimethyl-1,3-oxazolidin-2-one (compound 176)

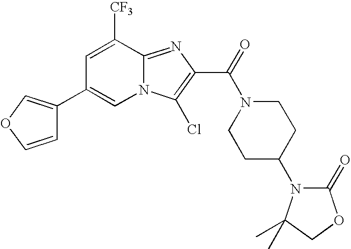

### Step A: phenylmethyl 4-[(2-hydroxy-1,1-dimethylethyl)amino]-1-piperidinecarboxylate

A solution of phenylmethyl 4-oxo-1-piperidinecarboxylate (2.5 g, 10.7 mmol) in 7 mL MeOH was added to a solution of 2-amino-2-methyl-1-propanol (1.91 g, 21.4 mmol) and AcOH (1.23 mL, 21.4 mmol) in MeOH (20 mL). After stirring for 15 mins, sodium cyanoborohydride (1.35 g, 21.4 mmol) was added in one portion and the reaction mixture was heated at 50° C. overnight. Sat'd NaHCO₃ was added and the solution was stirred for 1 h and extracted with CH₂Cl₂ (3×). The organic layers were evaporated, dissolved in 1N HCl and extracted with CH2Cl2. The aqueous layer was isolated, basified with 1N NaOH and extracted with CH₂Cl₂ (3×). The organic layers were dried (Na₂SO4), filtered, and evaporated to afford the title compound as a clear oil (1.89 g, 58%), which was contaminated. The material was used without further purification.

### Step B: phenylmethyl 4-(4,4-dimethyl-2-oxo-1,3-oxazolidin-3-yl)-1-piperidinecarboxylate

A solution of phenylmethyl 4-[(2-hydroxy-1,1-dimethylethyl)amino]-1-piperidinecarboxylate (1.89 g, 6.2 mmol), CDI (4 g, 24.7 mmol) and DBU (7 mL, 1.56 mmol) in CH₃CN (31 mL) was heated under reflux overnight. The reaction mixture was evaporated to dryness, sat'd NaHCO₃ was added, and the solution was extracted with CH₂Cl₂ (3×). The organic layers were dried (Na₂SO₄), filtered, evaporated and purified by silica gel chromatography (0-100% EtOAc/CH₂Cl₂) to afford the title compound (0.52 g, 25% as a yellow oil.

### Step C: 4,4-dimethyl-3-(4-piperidinyl)-1,3-oxazolidin-2-one

A solution of phenylmethyl 4-(4,4-dimethyl-2-oxo-1,3-oxazolidin-3-yl)-1-piperidinecarboxylate (0.52 g, 1.56 mmol) and 10% Pd/C (catalytic) in MeOH (25 mL) was stirred under a hydrogen atmosphere (60 psi) for 2 hours. The solution was filtered through celite, washing with MeOH and evaporated to dryness to afford the title compound as a white solid. The material was used without further purification (assumed quantitative).

### Step D: 3-(1-{[3-chloro-6-(3-furanyl)-8-(trifluoromethyl)imidazo[1,2-a]pyridin-2-yl]carbonyl}-4-piperidinyl)-4,4-dimethyl-1,3-oxazolidin-2-one

A mixture of 3-chloro-6-(3-furanyl)-8-(trifluoromethyl)imidazo[1,2-a]pyridine-2-carboxylic acid (0.099 g, 0.30 mmol), 4,4-dimethyl-3-(4-piperidinyl)-1,3-oxazolidin-2-one (0.085 g, 0.30 mmol), hydroxybenzotriazole (0.060 g, 0.39 mmol) and 1-ethyl-3-(3-dimethyllaminopropyl)carbodiimide hydrochloride (0.069 g, 0.36 mmol) in DMF (3 mL) was stirred at room temperature for 1 hour. The reaction mixture was diluted with methylene chloride and washed with saturated sodium bicarbonate. The organic layer was dried over sodium sulfate and the solvent was evaporated. The residue was purified by silica gel chromatography (0-8% MeOH in CH2Cl2) and reverse phase HPLC (acetonitrile/water with 0.1% formic acid) to afford the title compound (0.022 g, 14%) as an off-white solid.

### Example 76

### 3-(1-{[3-chloro-6-(1H-pyrazol-4-yl)-8-(trifluoromethyl)imidazo[1,2-a]pyridin-2-yl]carbonyl}-4-piperidinyl)-4,4-dimethyl-1,3-oxazolidin-2-one (Compound 177)

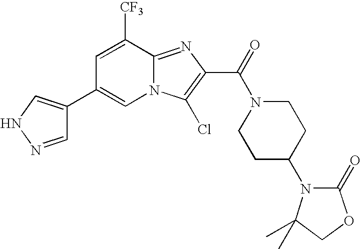

A mixture of 3-chloro-6-(1H-pyrazol-4-yl)-8-(trifluoromethyl)imidazo[1,2-a]pyridine-2-carboxylic acid (0.070 g, 0.21 mmol), 4,4-dimethyl-3-(4-piperidinyl)-1,3-oxazolidin-2-one (0.042 g, 0.21 mmol), hydroxybenzotriazole (0.042 g, 0.28 mmol) and 1-ethyl-3-(3-dimethyllaminopropyl)carbodiimide hydrochloride (0.049 g, 0.25 mmol) in DMF (1 mL) was stirred at room temperature for 1 hour. The reaction mixture was diluted with methylene chloride and washed with saturated sodium bicarbonate. The organic layer was dried over sodium sulfate and the solvent was evaporated. The residue was purified by silica gel chromatography (0-10% MeOH in CH₂Cl₂) to afford the title compound (0.058 g, 53%) as a white solid.

### Example 77

### 3-(1-{[3-chloro-6-(3-furanyl)-8-(trifluoromethyl)imidazo[1,2-a]pyridin-2-yl]carbonyl}-4-piperidinyl)-1,3-oxazolidine-2,4-dione (Compound 178)

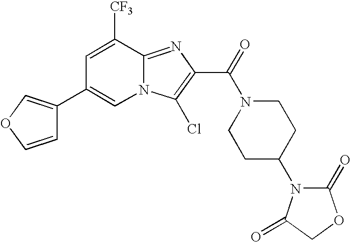

### Step A: 2-hydroxy-N-[1-(phenylmethyl)-4-piperidinyl]acetamide

A solution of 1-(phenylmethyl)-4-piperidinamine (2.0 g, 10.5 mmol), glycolic acid (0.8 g, 10.5 mmol), hydroxybenzotriazole (2.09 g, 13.7 mmol) and 1-ethyl-3-(3-dimethyllaminopropyl)carbodiimide hydrochloride (2.42 g, 12.6 mmol) in DMF (20 mL) and CH₃CN (20 mL) was stirred at room temperature for 3 days. The reaction mixture was evaporated, sat'd NaHCO₃ was added and the solution was extracted with CH₂Cl₂ (3×). The organic layers were dried (Na₂SO4), filtered, evaporated and purified by silica gel chromatography (0-10% MeOH/CH₂Cl₂) to afford the title compound (0.86 g, 33%) as a light yellow foam.

### Step B: 341-(phenylmethyl)-4-piperidinyl]-1,3-oxazolidine-2,4-dione

CDI (0.85 g, 5.2 mmol) was added to a solution of 2-hydroxy-N-[1-(phenylmethyl)-4-piperidinyl]acetamide (0.86 g, 3.5 mmol) in benzene (17 mL) and the reaction mixture was stirred at room temperature for 1 hour and heated to reflux overnight. The solution was evaporated and the residue was purified by silica gel chromatography (0-10% MeOH/CH₂Cl₂) to afford the title compound (0.84 g, 88%) as a clear oil.

### Step C: 3-(4-piperidinyl)-1,3-oxazolidine-2,4-dione

The title compound (quant.) was obtained from 3-[1-(phenylmethyl)-4-piperidinyl]-1,3-oxazolidine-2,4-dione (0.84 g, 3.1 mmol) as described previously.

### Step D. 3-(1-{[3-chloro-6-(3-furanyl)-8-(trifluoromethyl)imidazo[1,2-a]pyridin-2-yl]carbonyl}-4-piperidinyl)-1,3-oxazolidine-2,4-dione

A mixture of 3-chloro-6-(3-furanyl)-8-(trifluoromethyl)imidazo[1,2-a]pyridine-2-carboxylic acid (0.099 g, 0.30 mmol), 3-(4-piperidinyl)-1,3-oxazolidine-2,4-dione (0.055 g, 0.30 mmol), hydroxybenzotriazole (0.060 g, 0.39 mmol) and 1-ethyl-3-(3-dimethyllaminopropyl)carbodiimide hydrochloride (0.069 g, 0.36 mmol) in DMF (3 mL) was stirred at room temperature overnight. The reaction mixture was diluted with methylene chloride and washed with saturated sodium bicarbonate. The organic layer was dried over sodium sulfate and the solvent was evaporated. The residue was purified by silica gel chromatography (20-100% EtOAc/hexanes) to afford the title compound (0.067 g, 45%) as an off-white solid.

### Example 78

### 3-(1-{[3-chloro-6-(1H-pyrazol-4-yl)-8-(trifluoromethyl)imidazo[1,2-a]pyridin-2-yl]carbonyl}-4-piperidinyl)-1,3-oxazolidine-2,4-dione (compound 179)

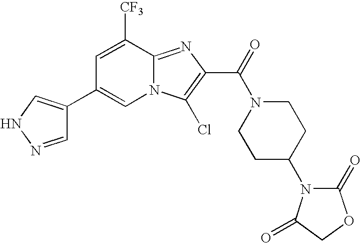

A mixture of 3-chloro-6-(1H-pyrazol-4-yl)-8-(trifluoromethyl)imidazo[1,2-a]pyridine-2-carboxylic acid (0.099 g, 0.30 mmol), 3-(4-piperidinyl)-1,3-oxazolidine-2,4-dione (0.055 g, 0.30 mmol), hydroxybenzotriazole (0.060g, 0.39 mmol) and 1-ethyl-3-(3-dimethyllaminopropyl)carbodiimide hydrochloride (0.069 g, 0.36 mmol) in DMF (3 mL) was stirred at room temperature overnight. The reaction mixture was diluted with methylene chloride and washed with saturated sodium bicarbonate. The organic layer was dried over sodium sulfate and the solvent was evaporated. The residue was purified by silica gel chromatography (20-100% EtOAc/hexanes) to afford the title compound (0.071 g, 48%) as an off-white solid.

### Example 79

### (4S)-3-(1-{[3-chloro-6-(3-furanyl)-8-(trifluoromethyl)imidazo[1,2-a]pyridin-2-yl]carbonyl}-4-piperidinyl)-4-methyl -1,3-oxazolidin-2-one (Compound 180)

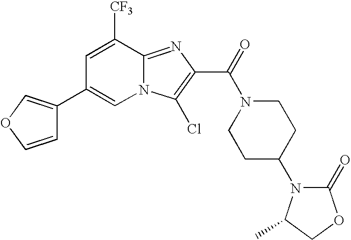

### Step A: phenylmethyl 4-{[(1S)-2-hydroxy-1-methylethyl]amino}-1-piperidinecarboxylate

A solution of phenylmethyl 4-oxo-1-pipeidinecarboxylate (2.0 g, 8.6 mmol) in 5.6 mL MeOH was added to a solution of L-alaninol (1.29 g, 17.2 mmol) and AcOH (0.98 ml, 17.2 mmol) in MeOH (16 mL). After stirring for 15 mins, sodium cyanoborohydride (1.08 g, 17.2 mmol) was added in one portion. The reaction mixture was stirred for 3 days. Sat'd NaHCO3 was added and the solution was stirred for 1 h and extracted with CH₂Cl₂ (3×). The organic layer evaporated and the residue was dissolved in 1N HCl. The solution was extracted with CH₂Cl₂. The aqueous layer was isolated, basified with 1N NaOH, and extracted with CH₂Cl₂ (3×). The organic layers were dried (Na2SO4), filtered and evaporated to provide the title compound (2.5 g, quant.) as a clear oil.

### Step B: phenylmethyl 4-[(4S)-4-methyl-2-oxo-1,3-oxazolidin-3-yl]-1-piperidinecarboxylate

The title compound (1.8 g, 65%)was obtained from phenylmethyl 4-{[(1S)-2-hydroxy-1-methylethyl]amino}-1-piperidinecarboxylate (2.52 g, 8.6 mmol) as described previously.

### Step C: (4S)-4-methyl-3-(4-piperidinyl)-1,3-oxazolidin-2-one

The title compound (quant.) was obtained from phenylmethyl 4-[(4S)-4-methyl-2-oxo-1,3-oxazolidin-3-yl]-1-piperidinecarboxylate (1.8 g, 5.6 mmol) as described previously.

### Step D: (4S)-3-(1-{[3-chloro-6-(3-furanyl)-8-(trifluoromethyl)imidazo[1,2-a]pyridin-2-yl]carbonyl}-4-piperidinyl)-4-methyl-1,3-oxazolidin-2-one

A mixture of 3-chloro-6-(3-furanyl)-8-(trifluoromethyl)imidazo[1,2-a]pyridine-2-carboxylic acid (0.215 g, 0.65 mmol), (4S)-4-methyl-3-(4-piperidinyl)-1,3-oxazolidin-2-one (0.120 g DIPEA (0.46 mL, 2.6 mmol) and HATU (0.30 g, 0.78 mmol) in DMF (4.3 mL) was stirred at room temperature overnight. The reaction mixture was diluted with methylene chloride and washed with saturated sodium bicarbonate. The organic layer was dried over sodium sulfate and the solvent was evaporated. The residue was purified by silica gel chromatography (20-100% EtOAc/hexs) to afford the title compound (0.23 g, 72%) as an off-white solid.

### Example 80

### 3-(1-{[3-chloro-6-(3-furanyl)-8-(trifluoromethyl)imidazo[1,2-a]pyridin-2-yl]carbonyl}-4-piperidinyl)-

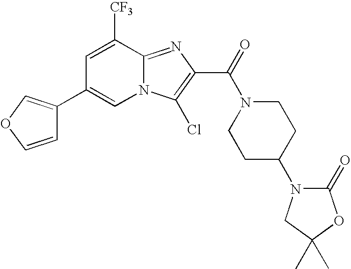

### Step A: phenylmethyl 4-[(2-hydroxy-2-methylpropyl)amino]-1-piperidinecarboxylate

The title compound (2.61 g, 99%) was obtained from phenylmethyl 4-oxo-1-piperidinecarboxylate (2.0 g, 8.6 mmol) and 1-amino-2-methyl-2-propanol (1.5 g, 17.2 mmol) as previously described.

### Step B. phenylmethyl 4-(5,5-dimethyl-2-oxo-1,3-oxazolidin-3-yl)-1-piperidinecarboxylate

The title compound (2.3 g, 81%) was obtained from phenylmethyl 44(2-hydroxy-2-methylpropyl)amino]-1-piperidinecarboxylate (2.6 g, 8.5 mmol) as previously described.

### Step C. 5,5-dimethyl-3-(4-piperidinyl)-1,3-oxazolidin-2-one

The title compound (quant.) was obtained from phenylmethyl 4-(5,5-dimethyl-2-oxo-1,3-oxazolidin-3-yl)-1-piperidinecarboxylate (2.3 g, 6.9 mmol) as previously described.

### Step D: 3-(1-{[3-chloro-6-(3-furanyl)-8-(trifluoromethyl)imidazo[1,2-a]pyridin-2-yl]carbonyl}-4-piperidinyl)-5,5-dimethyl-1,3-oxazolidin-2-one

A mixture of 3-chloro-6-(3-furanyl)-8-(trifluoromethyl)imidazo[1,2-a]pyridine-2-carboxylic acid (0.083 g, 0.25 mmol), 5,5-dimethyl-3-(4-piperidinyl)-1,3-oxazolidin-2-one (0.05 g, 0.25 mmol), DIPEA (0.18 mL, 1.0 mmol) and HATU (0.114 g, 0.30 mmol) in DMF (1.7 mL) was stirred at room temperature for 1 hour. The reaction mixture was evaporated and the residue was purified by reverse phase HPLC (acetonitrile/water with 0.1% formic acid) to afford the title compound (0.104 g, 82%) as a white solid.

### Example 81

### (4R)-3-(1-{[3-chloro-6-(3-furanyl)-8-(trifluoromethyl)imidazo[1,2-a]pyridin-2-yl]carbonyl}-4-piperidinyl)-4-methyl-1,3-oxazolidin-2-one (compound 182)

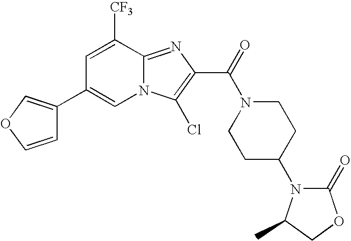

### Step A: phenylmethyl 4-{[(1R)-2-hydroxy-1-methylethyl]amino}-1-piperidinecarboxylate

The title compound (2.5 g, quant.) was obtained from phenylmethyl 4-oxo-1-piperidinecarboxylate (2.0 g, 8.6 mmol) and (R)-alaninol (1.3 g, 17.2 mmol) as previously described.

### Step B: phenylmethyl 4-[(4R)-4-methyl-2-oxo-1,3-oxazolidin-3-yl]-1-piperidinecarboxylate

The title compound (2.0 g, 74%) was obtained from phenylmethyl 4-{[(1R)-2-hydroxy-1-methylethyl]amino}-1-piperidinecarboxylate (2.51g, 8.6 mmol) as previously described.

### Step C: (4R)-4-methyl-3-(4-piperidinyl)-1,3-oxazolidin-2-one

The title compound (quant.) was obtained from phenylmethyl 4-[(4R)-4-methyl-2-oxo-1,3-oxazolidin-3-yl]-1-piperidinecarboxylate (2.0 g, 6.3 mmol) as previously described.

### Step D: (4R)-3-(1-{[3-chloro-6-(3-furanyl)-8-(trifluoromethyl)imidazo[1,2-a]pyridin-2-yl]carbonyl}-4-piperidinyl)-4-methyl-1,3-oxazolidin-2-one

A mixture of 3-chloro-6-(3-furanyl)-8-(trifluoromethyl)imidazo[1,2-a]pyridine-2-carboxylic acid (0.083 g, 0.25 mmol), (4R)-4-methyl-3-(4-piperidinyl)-1,3-oxazolidin-2-one (0.046 g, 0.25 mmol), DIPEA (0.18 mL, 1.0 mmol) and HATU (0.114 g, 0.30 mmol) in DMF (1.7 mL) was stirred at room temperature for 2 hours. The reaction mixture was diluted with methylene chloride and washed with saturated sodium bicarbonate. The organic layer was dried over sodium sulfate and the solvent was evaporated. The residue was purified by reverse phase HPLC (acetonitrile/water with 0.1% formic acid) to afford the title compound (0.105 g, 84%) as a white solid.

### Example 82

### 3-(1-{[3-chloro-6-(3-furanyl)-8-(trifluoromethyl)imidazo[1,2-a]pyridin-2-yl]carbonyl}-4-methyl-4-piperidinyl)-1,3-oxazolidin-2-one (compound 183)

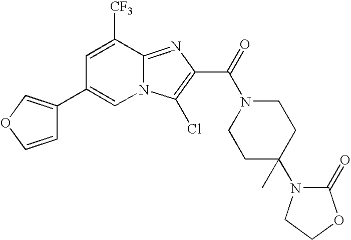

### Step A: 1,1-dimethylethyl 4-({[(2-chloroethyl)oxy]carbonyl}amino)-4-methyl -1-piperidinecarboxylate

Et₃N (1.7 mL, 12.3 mmol) and DPPA (2.4 mL, 11.1 mmol) was added to a solution of 1-{[(1,1-dimethylethyl)oxy]carbonyl}-4-methyl-4-piperidinecarboxylic acid (2.0 g, 8.22 mmol) in toluene (16 mL). The reaction mixture was stirred at room temperature for 1 hour. 2-chloroethanol (3.3 g, 41 mmol) was added and the solution was heated at 80° C. overnight. The solution was evaporated and purified by silica gel chromatography (0-50% EtOAc/hexs) to afford the title compound (2.2 g, 83%) as an oil.

### Step B: 1,1-dimethylethyl 4-methyl-4-(2-oxo-1,3-oxazolidin-3-yl)-1-piperidinecarboxylate

NaH (0.36 g of 60% dispersion in mineral oil, 8.9 mmol) was added to a solution of 1,1-dimethylethyl 4-({[(2-chloroethyl)oxy]carbonyl}amino)-4-methyl-1-piperidinecarboxylate (2.2 g, 6.9 mmol) in DMF (23 mL) and the reaction mixture was heated overnight. The reaction mixture was diluted with methylene chloride and washed with saturated sodium bicarbonate. The organic layer was dried over sodium sulfate and the solvent was evaporated. The residue was purified by silica gel chromatography (0-70% EtOAc/hexs) to afford the title compound (1.0 g, 51%) as a clear oil.

### Step C: 3-(4-methyl-4-piperidinyl)-1,3-oxazolidin-2-one

The title compound (0.32 g, 49%) was obtained from 1,1-dimethylethyl 4-methyl-4-(2-oxo-1,3-oxazolidin-3-yl)-1-piperidinecarboxylate (1.0 g, 3.5 mmol) as previously described.

### Step D: 3-(1-{[3-chloro-6-(3-furanyl)-8-(trifluoromethyl)imidazo[1,2-a]pyridin-2-yl]carbonyl}-4-methyl-4-piperidinyl)-1,3-oxazolidin-2-one

A mixture of 3-chloro-6-(3-furanyl)-8-(trifluoromethyl)imidazo[1,2-a]pyridine-2-carboxylic acid (0.083 g, 0.25 mmol), 3-(4-methyl-4-piperidinyl)-1,3-oxazolidin-2-one (0.046 g, 0.25 mmol), DIPEA (0.18 mL, 1.0 mmol) and HATU (0.114 g, 0.30 mmol) in DMF (1.7 mL) was stirred at room temperature for 2 hours. The reaction mixture was diluted with methylene chloride and washed with saturated sodium bicarbonate. The organic layer was dried over sodium sulfate and the solvent was evaporated. The residue was purified by reverse phase HPLC (acetonitrile/water with 0.1% formic acid) to afford the title compound (0.112 g, 90%) as a white solid.

### Example 83

### (5S)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidin-2-one (compound 184)

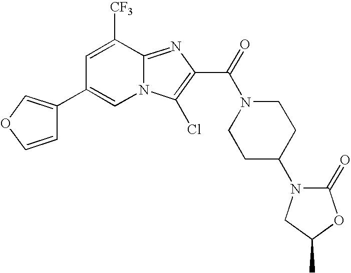

### Step A: phenylmethyl 4-[(5S)-5-methyl-2-oxo-1,3-oxazolidin-3-yl]-1-piperidinecarboxylate

The title compound (2.0 g, 73%) was obtained in two steps from phenylmethyl 4-oxo-1-piperidinecarboxylate (2.0 g, 8.57 mmol) as previously described.

### Step B: (5S)-5-methyl-3-(4-piperidinyl)-1,3-oxazolidin-2-one

The title compound (quant.) was obtained from phenylmethyl 4-[(5S)-5-methyl-2-oxo-1,3-oxazolidin-3-yl]-1-piperidinecarboxylate (2.0g, 6.3 mmol) as previously described.

### Step C: (5S)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidin-2-one

A mixture of 3-chloro-6-(3-furanyl)-8-(trifluoromethyl)imidazo[1,2-a]pyridine-2-carboxylic acid (0.083 g, 0.25 mmol), (5S)-5-methyl-3-(4-piperidinyl)-1,3-oxazolidin-2-one (0.046 g, 0.25 mmol), DIPEA (0.18 mL, 1.0 mmol) and HATU (0.114 g, 0.30 mmol) in DMF (1.7 mL) was stirred at room temperature for 2 hours. The reaction mixture was evaporated and the residue was purified by reverse phase HPLC (acetonitrile/water with 0.1% formic acid) to afford the title compound (0.094 g, 76%) as a white solid.

### Example 84

### (5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidin-2-one (Compound 185)

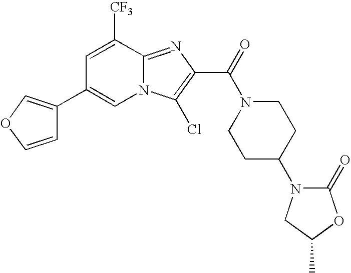

### Step A: phenylmethyl 4-[(5R)-5-methyl-2-oxo-1,3-oxazolidin-3-yl]-1-piperidinecarboxylate

The title compound (2.4 g, 86%) was obtained in two steps from phenylmethyl 4-oxo-1-piperidinecarboxylate (2.0 g, 8.57 mmol) as previously described.

### Step B: (5R)-5-methyl-3-(4-piperidinyl)-1,3-oxazolidin-2-one

The title compound (quant.) was obtained from phenylmethyl 4-[(5R)-5-methyl-2-oxo-1,3-oxazolidin-3-yl]-1-piperidinecarboxylate (2.4 g, 7.4 mmol) as previously described.

### Step C: (5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidin-2-one

### Step C: (5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidin-2-one

A mixture of 3-chloro-6-(3-furanyl)-8-(trifluoromethyl)imidazo[1,2-a]pyridine-2-carboxylic acid (0.083 g, 0.25 mmol), (5R)-5-methyl-3-(4-piperidinyl)-1,3-oxazolidin-2-one (0.046 g, 0.25 mmol), DIPEA (0.18 mL, 1.0 mmol) and HATU (0.114 g, 0.30 mmol) in DMF (1.7 mL) was stirred at room temperature for 2 hours. The reaction mixture was evaporated and the residue was purified by reverse phase HPLC (acetonitrile/water with 0.1% formic acid) to afford the title compound (0.088 g, 71%) as a white solid.

### Example 85

### 3-(1-{[3-chloro-6-(3-furanyl)-8-(trifluoromethyl)imidazo[1,2-a]pyridin-2-yl]carbonyl}-4-piperidinyl)-1,3-oxazol-2(3H)-one (Compound 186)

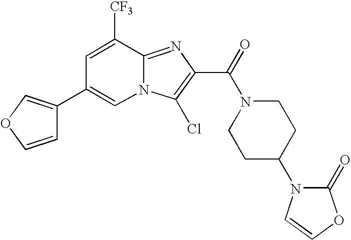

Sodium borohydride (0.008 mg, 0.22 mmol) was added to a solution of 3-(1-{[3-chloro-6-(3-furanyl)-8-(trifluoromethyl)imidazo[1,2-a]pyridin-2-yl]carbonyl}-4-piperidinyl)-1,3-oxazolidine-2,4-dione (0.036 g, 0.072 mmol) in MeOH (1 mL). The reaction mixture was stirred at room temperature for 2 hours, then heated to 50° C. overnight. Saturated sodium bicarbonate was added and the solution was extracted with CH₂Cl₂. The organic layers were dried (Na₂SO₄), filtered, and evaporated. The residue was taken up in CH₂Cl₂ (1 mL) and Et₃N (0.1 mL, 0.73 mmol) and MsCl (0.02 mL, 0.22 mmol) was added. The reaction mixture was stirred at room temperature overnight. The reaction mixture was diluted with methylene chloride and washed with saturated sodium bicarbonate. The organic layer was dried over sodium sulfate and the solvent was evaporated. The residue was purified by reverse phase HPLC (acetonitrile/water with 0.1% formic acid) to afford the title compound (0.002 g, 6%) as a white solid.

### Example 86

### 3-(1-{[6-amino-3-chloro-8-(trifluoromethyl)imidazo[1,2-a]pyridin-2-yl]carbonyl}-4-piperidinyl)-1,3-oxazolidin-2-one (Compound 187)

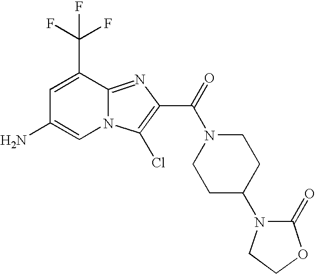

To a solution of 6-amino-3-chloro-8-(trifluoromethyl)imidazo[1,2-a]pyridine-2-carboxylic acid (671.4 mg, 2.401 mmol), 3-(4-piperidinyl)-1,3-oxazolidin-2-one (450 mg, 2.64 mmol), N,N-diisopropylethylamine (2.1 mL, 12.01 mmol) in DMF (12.0 mL) was added PyBrOP (1.175 g, 2.52 mmol) at room temperature. After 2 hours, the mixture was diluted with EtOAc (120 mL) and washed with saturated aqueous NH₄Cl (20 mL), saturated NaHCO₃ (20 mL), then brine (20 mL), dried (Na₂SO₄), filtered and concentrated. The crude was absorbed on silica gel and purified by silica gel chromatography [0-100% EtOAc in hexanes] to give the desired product (239.3 mg, 23%). 45 mg was further purified by reverse phase HPLC (10-70% ACN gradient) to give the title compound (29.1 mg). MS(ESI: 432.1 (M+1).

### Example 87

### (5S)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione (Compound 188)

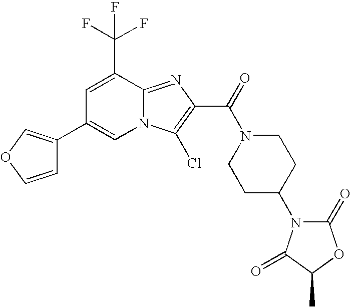

### Step1: 1,1-dimethylethyl 4-{[(2S)-2-hydroxypropanoyl]amino}-1-piperidinecarboxylate

To a solution of 4-aminopiperidine N-1 Boc (1 g, 4.99 mmol), L-(+)-lactic acid (0.529 g, 4.99 mmol), N,N-diisopropylethylamine (3.49 mL, 19.97 mmol) in DMF (24.97 mL) was added HATU (1.993 g, 5.24 mmol). After 1.5 hours, the mixture was combined with another batch (1.248 mmol scale) and the solvent was removed under reduced pressure. The crude oil was diluted with EtOAc (150 mL), and washed with water (2×50 mL), saturated NaHCO₃ (30 mL), then brine (30 mL), dried (Na₂SO₄), filtered and concentrated to give crude product (2.0105 g) as a foam (turned to yellow powder). This was used for the next step without further purification. MS(ESI): 217.3 (M+1−^{t}Bu).

### Step 2: 1,1-dimethylethyl 4-[(5S)-5-methyl-2,4-dioxo-1,3-oxazolidin-3-yl]-1-piperidinecarboxylate

To a solution of crude 1,1-dimethylethyl 4-{[(2S)-2-hydroxypropanoyl]amino}-1-piperidinecarboxylate (1.7563 g, 5.29 mmol) in DMF (35.3 mL) was added 1,1′-carbonyldiimidazole (0.900 g, 5.55 mmol) in one portion. After 1 hour, the reaction was heated to 80° C. and stirred overnight. The mixture was cooled and the solvent removed under reduced pressure. The crude brown oil was diluted with EtOAc (200 mL), and washed with water (2×100 mL), brine (100 mL), dried (Na₂SO₄), filtered and concentrated. The crude was absorbed on silica gel and purified by silica gel chromatography (0-35% EtOAc in hexanes, 80 g ISCO column) to give 1,1-dimethylethyl 4-[(5S)-5-methyl-2,4-dioxo-1,3-oxazolidin-3-yl]-1-piperidinecarboxylate (719.5 mg, 2.412 mmol, 45.6% yield) as a white solid. MS(ESI): 284.2 (M+1-Me); 243.2 (M+1-^{t}Bu).

### Step 3: (5S)-5-methyl-3-(4-piperidinyl)-1,3-oxazolidine-2,4-dione hydrochloride

To a solution of 1,1-dimethylethyl 4-[(5S)-5-methyl-2,4-dioxo-1,3-oxazolidin-3-yl]-1-piperidinecarboxylate (800.4 mg, 2.68 mmol) in DCM (6.7 mL) at room temperature was added a solution of 4 N hydrogen chloride in 1,4-dioxane (6.71 mL, 26.8 mmol). After 1.5 hours, the solvent was removed under reduced pressure to give (5S)-5-methyl-3-(4-piperidinyl)-1,3-oxazolidine-2,4-dione hydrochloride (651.5 mg, 2.78 mmol, quantitative yield) as white solid. MS(ESI): 199.3 (M+1).

### Step 4: (5S)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione

To a suspension of 3-chloro-6-(3-furanyl)-8-(trifluoromethyl)imidazo[1,2-a]pyridine-2-carboxylic acid (99 mg, 0.3 mmol), (55)-5-methyl-3-(4-piperidinyl)-1,3-oxazolidine-2,4-dione (77 mg, 0.330 mmol), N,N-diisopropylethylamine (262 μl, 1.500 mmol) in DMF (1.5 mL) was added HATU (120 mg, 0.315 mmol) at room temperature. After 1.75 hours, the mixture was diluted with EtOAc (25 mL) and washed with saturated aqueous NaHCO₃ (10 mL), brine (10 mL), dried (Na₂SO₄), filtered and concentrated. The crude was absorbed on silica gel and purified by silica gel chromatography (0-70% EtOAc in hexanes) to give (55)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione (139.3 mg, 0.267 mmol, 89% yield) as a white powder. MS(ESI): 511.3 (M+1).

### Example 88

### (5S)-3-(1-{[3-chloro-6-cyclopropyl-8-(trifluoromethyl)imidazo[1,2-a]pyridin-2-yl]carbonyl}-4-piperidinyl)-5-methyl-1,3-oxazolidine-2,4-dione (Compound 189)

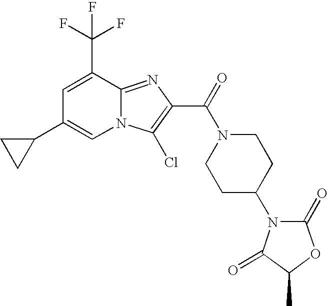

To a suspension of 3-chloro-6-cyclopropyl-8-(trifluoromethyl)imidazo[1,2-a]pyridine-2-carboxylic acid (91 mg, 0.3 mmol), (5S)-5-methyl-3-(4-piperidinyl)-1,3-oxazolidine-2,4-dione (77 mg, 0.330 mmol), N,N-diisopropylethylamine (262 μl, 1.500 mmol) in DMF (1500 μl) was added HATU (120 mg, 0.315 mmol) at room temperature. After 1.75 hours, the mixture was diluted with EtOAc (25 mL) and washed with saturated aqueous NaHCO₃ (10 mL), brine (10 mL), dried (Na₂SO₄), filtered and concentrated. The crude was absorbed on silica gel and purified by silica gel chromatography (0-70% EtOAc in hexanes) to give (5S)-3-(1-{[3-chloro-6-cyclopropyl-8-(trifluoromethyl)imidazo[1,2-a]pyridin-2-yl]carbonyl}-4-piperidinyl)-5-methyl-1,3-oxazolidine-2,4-dione (135.4 mg, 0.274 mmol, 91% yield) as a white powder. MS(ESI): 485.3 (M+1).

### Example 89

### (5S)-3-(1-{[6-bromo-3-chloro-8-(trifluoromethyl)imidazo[1,2-a]pyridin-2-yl]carbonyl}-4-piperidinyl)-5-methyl-1,3-oxazolidine-2,4-dione (Compound 190)

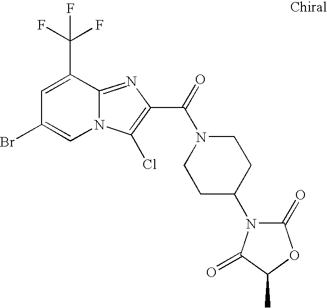

To a suspension of 6-bromo-3-chloro-8-(trifluoromethyl)imidazo[1,2-a]pyridine-2-carboxylic acid (103 mg, 0.3 mmol), (5S)-5-methyl-3-(4-piperidinyl)-1,3-oxazolidine-2,4-dione (77 mg, 0.330 mmol), N,N-diisopropylethylamine (262 μl, 1.500 mmol) in DMF (1.5 mL) was added HATU (120 mg, 0.315 mmol) at room temperature. After 1.75 hours, the mixture was diluted with EtOAc (25 mL) and washed with saturated aqueous NaHCO₃ (10 mL), brine (10 mL), dried (Na₂SO₄), filtered and concentrated. The crude was absorbed on silica gel and purified by silica gel chromatography (0-70% EtOAc in hexanes) to give (5S)-3-(1-{[6-bromo-3-chloro-8-(trifluoromethyl)imidazo[1,2-a]pyridin-2-yl]carbonyl}-4-piperidinyl)-5-methyl-1,3-oxazolidine-2,4-dione (143.4 mg, 0.268 mmol, 89% yield) as a white powder. MS(ESI): 525.2 (M+1).

### Example 91

### (5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione (Compound 191)

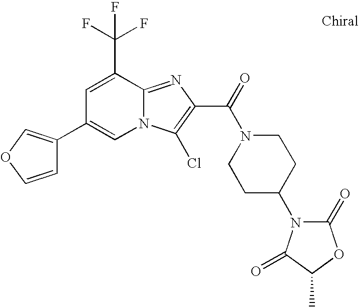

### Step 1: 1,1-dimethylethyl 4-{[(2R)-2-hydroxypropanoyl]amino}-1-piperidinecarboxylate

To a solution of 4-aminopiperidine N-1 Boc (1 g, 4.99 mmol), lactic acid (0.529 g, 4.99 mmol), N,N-diisopropylethylamine (3.49 mL, 19.97 mmol) in DMF (24.97 mL) was added HATU (1.993 g, 5.24 mmol). After 1.5 hours, the solvent was removed under reduced pressure. The crude oil was diluted with EtOAc (150 mL), and washed with water (2×50 mL), saturated NaHCO₃ (30 mL), then brine (30 mL), dried (Na₂SO₄), filtered and concentrated to give 1,1-dimethylethyl 4-{[(2R)-2-hydroxypropanoyl]amino}-1-piperidinecarboxylate (1.5361 g, 4.96 mmol, 99% yield) as a foam which was used for the next step without further purification. MS(ESI): 217.3 (M+1-^{t}Bu)

### Step 2: 1,1-dimethylethyl 4-[(5R)-5-methyl-2,4-dioxo-1,3-oxazolidin-3-yl]-1-piperidinecarboxylate

To a solution of crude 1,1-dimethylethyl 4-{[(2R)-2-hydroxypropanoyl]amino}-1-piperidinecarboxylate (1.5361 g, 4.96 mmol) in DMF (33.1 mL) was added 1,1′-carbonyldiimidazole (0.845 g, 5.21 mmol) in one portion. After 1 hour, the reaction was heated to 80° C. and stirred overnight. Upon cooling, the solvent was removed under reduced pressure. The crude brown oil was diluted with EtOAc (200 mL), and washed with water (2×100 mL), brine (100 mL), dried (Na₂SO₄), filtered and concentrated. The crude was absorbed on silica gel and purified by silica gel chromatography(0-35% EtOAc in hexanes, 80 g ISCO column) to give 1,1-dimethylethyl 4-[(5R)-5-methyl-2,4-dioxo-1,3-oxazolidin-3-yl]-1-piperidinecarboxylate (694 mg, 2.326 mmol, 46.9% yield) as a white solid. MS(ESI): 284.2 (M+1-Me); 243.2 (M+1-^{t}Bu).

### Step 3: (5R)-5-methyl-3-(4-piperidinyl)-1,3-oxazolidine-2,4-dione hydrochloride

To a solution of 1,1-dimethylethyl 4-[(5R)-5-methyl-2,4-dioxo-1,3-oxazolidin-3-yl]-1-piperidinecarboxylate (690 mg, 2.313 mmol) in DCM (5.8 mL) at room temperature was added a solution of 4 N hydrogen chloride in 1,4-dioxane (5.78 mL, 23.13 mmol). After 1.5 hours, the solvent was removed under reduced pressure to give (5R)-5-methyl-3-(4-piperidinyl)-1,3-oxazolidine-2,4-dione hydrochloride (558.6 mg, 2.380 mmol, quantitative yield) as white solid. MS(ESI): 199.3 (M+1).

### Step 4: (5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione

To a suspension of 3-chloro-6-(3-furanyl)-8-(trifluoromethyl)imidazo[1,2-a]pyridine-2-carboxylic acid (99 mg, 0.3 mmol), (5R)-5-methyl-3-(4-piperidinyl)-1,3-oxazolidine-2,4-dione (70.4 mg, 0.300 mmol), N,N-diisopropylethylamine (262 μl, 1.500 mmol) in DMF (1.5 mL) was added HATU (120 mg, 0.315 mmol) at room temperature. After 1.5 hours, the mixture was diluted with EtOAc (25 mL) and washed with saturated aqueous NaHCO₃ (10 mL), brine (10 mL), dried (Na₂SO₄), filtered and concentrated. The crude was absorbed on silica gel and purified by silica gel chromatography (0-70% EtOAc in hexanes) to give (5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione (139.7 mg, 0.268 mmol, 89% yield) as a white powder. MS(ESI): 511.3 (M+1).

### Example 91

### (5R)-3-(1-{[3-chloro-6-cyclopropyl-8-(trifluoromethyl)imidazo[1,2-a]pyridin-2-yl]carbonyl}-4-piperidinyl)-5-methyl-1,3-oxazolidine-2,4-dione (Compound 192)

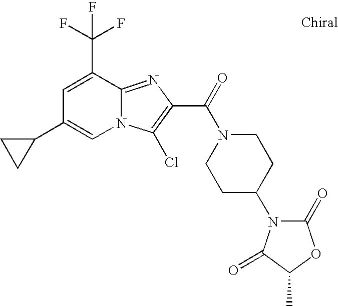

To a suspension of 3-chloro-6-cyclopropyl-8-(trifluoromethyl)imidazo[1,2-a]pyridine-2-carboxylic acid (0.091 g, 0.3 mmol), (5R)-5-methyl-3-(4-piperidinyl)-1,3-oxazolidine-2,4-dione (0.059 g, 0.251 mmol), N,N-diisopropylethylamine (0.262 mL, 1.500 mmol) in DMF (1.5 mL) was added HATU (0.120 g, 0.315 mmol) at room temperature. After 1.5 hours, the mixture was diluted with EtOAc (25 mL) and washed with saturated aqueous NaHCO₃ (10 mL), brine (10 mL), dried (Na₂SO₄), filtered and concentrated. The crude was absorbed on silica gel and purified by silica gel chromatography (0-70% EtOAc in hexanes) to give (5R)-3-(1-{[3-chloro-6-cyclopropyl-8-(trifluoromethyl)imidazo[1,2-a]pyridin-2-yl]carbonyl}-4-piperidinyl)-5-methyl-1,3-oxazolidine-2,4-dione (129.1 mg, 0.261 mmol, 87% yield) as a white powder. MS(ESI): 485.4 (M+1).

### Example 92

### (5R)-3-(1-{[6-bromo-3-chloro-8-(trifluoromethyl)imidazo[1,2-a]pyridin-2-yl]carbonyl}-4-piperidinyl)-5-methyl-1,3-oxazolidine-2,4-dione (Compound 193)

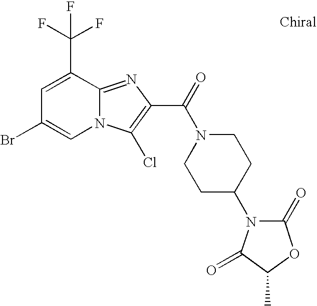

To a suspension of 6-bromo-3-chloro-8-(trifluoromethyl)imidazo[1,2-a]pyridine-2-carboxylic acid (103 mg, 0.3 mmol), (5R)-5-methyl-3-(4-piperidinyl)-1,3-oxazolidine-2,4-dione (70.4 mg, 0.300 mmol), N,N-diisopropylethylamine (262 μL, 1.500 mmol) in DMF (1.5 mL) was added HATU (120 mg, 0.315 mmol) at room temperature. After 1.5 hours, the mixture was diluted with EtOAc (25 mL) and washed with saturated aqueous NaHCO₃ (10 mL), brine (10 mL), dried (Na₂SO₄), filtered and concentrated. The crude was absorbed on silica gel and purified by silica gel chromatography (0-70% EtOAc in hexanes) to give (5R)-3-(1-{[6-bromo-3-chloro-8-(trifluoromethyl)imidazo[1,2-a]pyridin-2-yl]carbonyl}-4-piperidinyl)-5-methyl-1,3-oxazolidine-2,4-dione (137.3 mg, 0.257 mmol, 86% yield) as a white powder. MS(ESI): 525.2 (M+1).

### Example 93

### 3-(1-{[3-bromo-6-iodo-8-(trifluoromethyl)imidazo[1,2-a]pyridin-2-yl]carbonyl}-4-piperidinyl)-1,3-oxazolidin-2-one (Compound 194)

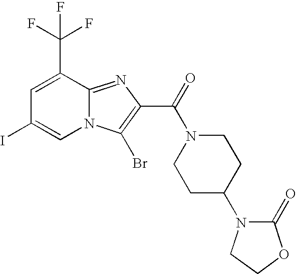

### Step 1: 5-iodo-3-(trifluoromethyl)-2-pyridinamine

A solution of N-iodosuccinimide (61 g, 0.272 mol) in DMF (200 mL) was rapidly added dropwise via an additional funnel to a stirred solution of 3-(trifluoromethyl)-2-pyridinamine (40 g, 0.247 mol) in N,N-dimethylformamide (200 mL) at room temperature. The mixture was allowed to stir at room temperature overnight. Part of the DMF (300 mL) was removed under reduced pressure and the residue was poured into 10% aqueous Na₂S₂O₃ and extracted with EtOAc. The organic phase was separated and washed with brine, dried and concentrated to give 5-iodo-3-(trifluoromethyl)-2-pyridinamine (50 g, 70%) as yellow solid.

### Step 2: methyl 6-iodo-8-(trifluoromethyl)imidazo[1,2-a]pyridine-2-carboxylate

A solution of 5-iodo-3-(trifluoromethyl)-2-pyridinamine (45 g, 0.156 mol) and methyl bromopyruvate (70.6 g, 0.39 mol) in N,N-dimethylformamide (600 mL) was heated at 70° C. overnight. The reaction mixture was cooled to room temperature and poured into ice water. The mixture was stirred for 1 hour and filtered to give the title compound (37 g) as brown solid. This material was used for next step without further purification.

### Step 3: methyl 3-bromo-6-iodo-8-(trifluoromethyl)imidazo[1,2-a]pyridine-2-carboxylate

To a solution of methyl 6-iodo-8-(trifluoromethyl)imidazo[1,2-a]pyridine-2-carboxylate (37 g, 0.1 mol) in N,N-dimethylformamide (350 mL) was added N-bromosuccinimide (18.7 g, 0.105 mol). The mixture was heated at 50° C. for 3 hours. Upon cooling, the mixture was diluted with water and extracted with EtOAc. The organic layer was washed with brine, dried and concentrated. The crude was purified by silica gel chromatography [petroleum ether: EtOAc (10:1 v/v)] to give the title compound (32 g, 71%) as white solid.

### Step 4: 3-bromo-6-iodo-8-(trifluoromethyl)imidazo[1,2-a]pyridine-2-carboxylic acid

To a solution of methyl 3-bromo-6-iodo-8-(trifluoromethyl)imidazo[1,2-a]pyridine-2-carboxylate (1 g, 2.227 mmol) in THF (11.93 mL) and water (3.98 mL) at room temperature was added a chilled solution of 1N sodium hydroxide (3.90 mL, 3.90 mmol). After 35 minutes, the mixture was poured into chilled 1 N hydrochloric acid (5.57 mL, 5.57 mmol). The mixture was stirred for 1 hour and the precipitate was filtered and dried under high vacuum to give 3-bromo-6-iodo-8-(trifluoromethyl)imidazo[1,2-a]pyridine-2-carboxylic acid (834.7 mg, 1.823 mmol, 82% yield) as an off-white solid. The material was used for the next step without further purification. MS(ESI): 437.1 (M+1).

### Step 5: 3-(1-{[3-bromo-6-iodo-8-(trifluoromethyl)imidazo[1,2-a]pyridin-2-yl]carbonyl}-4-piperidinyl)-1,3-oxazolidin-2-one

To a solution of 3-bromo-6-iodo-8-(trifluoromethyl)imidazo[1,2-a]pyridine-2-carboxylic acid (180 mg, 0.414 mmol), 3-(4-piperidinyl)-1,3-oxazolidin-2-one (77 mg, 0.455 mmol), N,N-diisopropylethylamine (0.361 mL, 2.069 mmol) in DMF (2.069 mL) was added HATU (165 mg, 0.435 mmol) at room temperature. After 30 minutes, the mixture was diluted with EtOAc (50 mL) and washed with saturated aqueous NaHCO₃ (25 mL), then brine (25 mL), dried (Na₂SO₄), filtered and concentrated. The crude was absorbed on silica gel and purified by silica gel chromatography [0-100% EtOAc in hexanes] to give 3-(1-{[3-bromo-6-iodo-8-(trifluoromethyl)imidazo[1,2-a]pyridin-2-yl]carbonyl}-4-piperidinyl)-1,3-oxazolidin-2-one (15.6 mg, 0.250 mmol, 60.5% yield) as white powder. MS(ESI): 587.2, 589.2 (M+1).

### Example 94

### 3-(1-{[3-bromo-6-iodo-8-(trifluoromethyl)imidazo[1,2-a]pyridin-2-yl]carbonyl}-4-piperidinyl)-1,3-oxazolidine-2,4-dione (Compound 195)

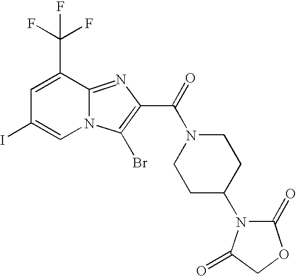

To a solution of 3-bromo-6-iodo-8-(trifluoromethyl)imidazo[1,2-a]pyridine-2-carboxylic acid (180 mg, 0.414 mmol), 3-(4-piperidinyl)-1,3-oxazolidine-2,4-dione (91 mg, 0.414 mmol), N,N-diisopropylethylamine (0.361 mL, 2.069 mmol) in DMF (2.069 mL) was added HATU (165 mg, 0.435 mmol) at room temperature. After 30 minutes, the mixture was diluted with EtOAc (50 mL), washed with saturated aqueous NaHCO₃ (25 mL), then brine (25 mL), dried (Na₂SO₄), filtered and concentrated. The crude was absorbed on silica gel and purified by silica gel chromatography [0-60% EtOAc in hexanes] to give 3-(1-{[3-bromo-6-iodo-8-(trifluoromethyl)imidazo[1,2-a]pyridin-2-yl]carbonyl}-4-piperidinyl)-1,3-oxazolidine-2,4-dione (228.7 mg, 0.373 mmol, 90% yield) as white powder. MS(ESI): 601.1, 603.1 (M+1).

### Example 95

### 3-(1-{[6-(1-methylethyl)-8-(trifluoromethyl)imidazo[1,2-a]pyridin-2-yl]carbonyl}-4-piperidinyl)-1,3-oxazolidin-2-one (Compound 196)

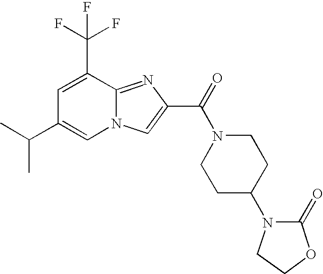

### Step 1: 3-(1-{[3-chloro-6-(1-methylethenyl)-8-(trifluoromethyl)imidazo[1,2-a]pyridin-2-yl]carbonyl}-4-piperidinyl)-1,3-oxazolidin-2-one

To a suspension of 3-(1-{[6-bromo-3-chloro-8-(trifluoromethyl)imidazo[1,2-a]pyridin-2-yl]carbonyl}-4-piperidinyl)-1,3-oxazolidin-2-one (400 mg, 0.807 mmol), potassium isopropenyl trifluoborate (167 mg, 1.130 mmol), potassium isopropenyl trifluoborate (167 mg, 1.130 mmol), and PdCl₂(dppf)-CH₂Cl₂ adduct (33.0 mg, 0.040 mmol) in n-propanol (4.03 mL) at 95° C. A 1 hour, the mixture was cooled, diluted with EtOAc (80 mL), washed with brine (20 mL), dried (Na₂SO₄), filtered and concentrated. The crude was absorbed on silica gel and purified by silica gel chromatography (80% EtOAc in hexanes) to give 320.9 mg of product. This was crystallized from EtOH/hexanes to give 3-(1-{[3-chloro-6-(1-methylethenyl)-8-(trifluoromethyl)imidazo[1,2-a]pyridin-2-yl]carbonyl}-4-piperidinyl)-1,3-oxazolidin-2-one (215.3 mg, 0.462 mmol, 57.2% yield) as white solid. MS(ESI): 457.4 (M+1).

### Step 2: 3-(1-{[6-(1-methylethyl)-8-(trifluoromethyl)imidazo[1,2-a]pyridin-2-yl]carbonyl}-4-piperidinyl)-1,3-oxazolidin-2-one

A suspension of 3-(1-{[3-chloro-6-(1-methylethenyl)-8-(trifluoromethyl)imidazo[1,2-a]pyridin-2-yl]carbonyl}-4-piperidinyl)-1,3-oxazolidin-2-one (26.7 mg, 0.058 mmol), 10% palladium on carbon (13 mg, 0.012 mmol), phenyl sulfide (0.146 μL, 0.877 μmol) was stirred under hydrogen (1 atm) in methanol (2.9 mL). After 5.5 hours, the mixture was filtered through 45 um disc and the solvent was removed under reduced pressure. The crude was subjected to reverse phase HPLC (30-90% ACN gradient) to give 3-(1-{[6-(1-methylethyl)-8-(trifluoromethyl)imidazo[1,2-a]pyridin-2-yl]carbonyl}-4-piperidinyl)-1,3-oxazolidin-2-one (5.3 mg, 0.012 mmol, 20.9% yield) [MS(ESI): 425.4 (M+1)] and 3-(1-{[3-chloro-6-(1-methylethyl)-8-(trifluoromethyl)imidazo[1,2-a]pyridin-2-yl]carbonyl}-4-piperidinyl)-1,3-oxazolidin-2-one (12.1 mg, 0.026 mmol, 44.2% yield) [MS(ESI): 459.3 (M+1)] both as white powders.

### Example 96

### 3-(1-{[3-bromo-6-cyclopropyl-8-(trifluoromethyl)imidazo[1,2-a]pyridin-2-yl]carbonyl}-4-piperidinyl)-1,3-oxazolidin-2-one (Compound 197)

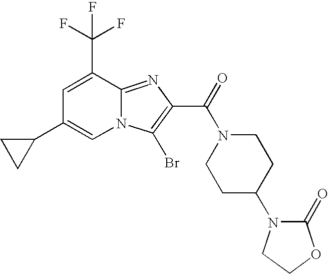

To a degassed solution of 3-(1-{[3-bromo-6-iodo-8-(trifluoromethyl)imidazo[1,2-a]pyridin-2-yl]carbonyl}-4-piperidinyl)-1,3-oxazolidin-2-one (99 mg, 0.169 mmol), cyclopropylboronic acid (29.0 mg, 0.337 mmol), PdCl₂(dppf)-CH₂Cl₂ adduct (6.88 mg, 8.43 μmol) in 1 M tripotassium phosphate (0.674 mL, 0.674 mmol) and 1,4-dioxane (2 mL) was heated at 80° C. After 1 day, the mixture was cooled, diluted with EtOAc (25 mL) and washed with brine (10 mL), dried (Na₂SO₄), filtered and concentrated. The crude was purified by preparative HPLC [10-65% ACN gradient] to give 3-(1-{[3-bromo-6-cyclopropyl-8-(trifluoromethyl)imidazo[1,2-a]pyridin-2-yl]carbonyl}-4-piperidinyl)-1,3-oxazolidin-2-one (9.4 mg, 0.018 mmol, 10.56% yield) as white powder. MS(ESI): 501.1, 503.3 (M+1).

### Example 97

### N-[3-chloro-2-{[4-(2-oxo-1,3-oxazolidin-3-yl)-1-piperidinyl]carbonyl}-8-(trifluoromethyl)imidazo[1,2-a]pyridin-6-yl]acetamide (Compound 198)

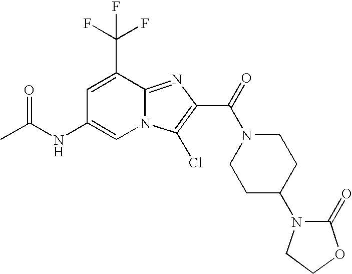

### Step 1: 3-(1-{[6-amino-3-chloro-8-(trifluoromethyl)imidazo[1,2-a]pyridin-2-yl]carbonyl}-4-piperidinyl)-1,3-oxazolidin-2-one

To a solution of 6-amino-3-chloro-8-(trifluoromethyl)imidazo[1,2-a]pyridine-2-carboxylic acid (prepared according to WO09023179A2) (220 mg, 0.787 mmol), 3-(4-piperidinyl)-1,3-oxazolidin-2-one (147 mg, 0.866 mmol), N,N-diisopropylethylamine (0.687 mL, 3.93 mmol) in DMF was added PyBrOP (385 mg, 0.826 mmol) at room temperature. After 25 minutes, the mixture was diluted with EtOAc (50 mL) and washed with saturated aqueous NaHCO₃ (20 mL), then brine (20 mL), dried (Na₂SO₄), filtered and concentrated. The crude material was absorbed on silica gel and purified by silica gel chromatography [25-100% EtOAc in hexanes] to give a yellow syrup. This was lyophilized to give 3-(1-{[6-amino-3-chloro-8-(trifluoromethyl)imidazo[1,2-a]pyridin-2-yl]carbonyl}-4-piperidinyl)-1,3-oxazolidin-2-one (135.8 mg, 0.314 mmol, 40% yield) as a light green powder. MS(ESI): 432.1 (M+1).

### Step 2: N-[3-chloro-2-{[4-(2-oxo-1,3-oxazolidin-3-yl)-1-piperidinyl]carbonyl}-8-(trifluoromethyl)imidazo[1,2-a]pyridin-6-yl]acetamide

To a solution of 3-(1-{[6-amino-3-chloro-8-(trifluoromethyl)imidazo[1,2-a]pyridin-2-yl]carbonyl}-4-piperidinyl)-1,3-oxazolidin-2-one (35 mg, 0.081 mmol) and triethylamine (0.034 mL, 0.243 mmol) in DMF (1 mL) was added acetyl chloride (6.92 μL, 0.097 mmol). After 30 minutes, extra TEA (30 μL) and acetyl chloride (20 μL) were added. After 1 hour, the mixture was diluted with EtOAc (20 mL) and washed with saturated NH₄Cl (10 mL), brine (10 mL), dried (Na₂SO₄), filtered and concentrated. The crude was purified by preparative TLC [DCM/MeOH (96:4 v/v)] to give N-[3-chloro-2-{[4-(2-oxo-1,3-oxazolidin-3-yl)-1-piperidinyl]carbonyl}-8-(trifluoromethyl)imidazo[1,2-a]pyridin-6-yl]acetamide (14.9 mg, 0.031 mmol, 38.8% yield) as a white powder. MS(ESI): 474.1 (M+1).

### Example 98

### 4-(1-{[3-chloro-6-(3-furanyl)-8-(trifluoromethyl)imidazo[1,2-a]pyridin-2-yl]carbonyl}-4-piperidinyl)-2,4-dihydro-3H-1,2,4-triazol-3-one (Compound 199)

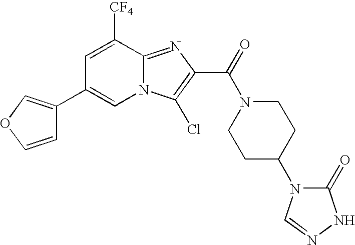

### Step A: methyl (2E)-2-[(ethyloxy)methylidene]hydrazinecarboxylate

A mixture of methyl hydrazinecarboxylate (1.5 g, 16.65 mmol) and triethyl orthoformate (41.6 mL, 250 mmol) were heated to 88° C. with stirring under nitrogen. After 16 h, the reaction was cooled to RT and concentrated to dryness to give a yellow solid. The crude material was dissolved in hot ethyl acetate and allowed to cool to RT. No crystals formed so solvent was removed under reduced pressure to give methyl (2E)-2-[(ethyloxy)methylidene]hydrazinecarboxylate (2.4 g, 15.60 mmol, 94% yield) as a yellow powder. Used as is without further purification.

### Step B: 4-[1-(phenylmethyl)-4-piperidinyl]-2,4-dihydro-3H-1,2,4-triazol-3-one

A mixture of methyl (2E)-2-[(ethyloxy)methylidene]hydrazinecarboxylate (1.920 g, 13.14 mmol) and 1-(phenylmethyl)-4-piperidinamine (0.5 g, 2.63 mmol) in methanol (8 mL) was heated to 50° C. with stirring for 16 h under nitrogen. The red solution was diluted with MeOH (5 mL) then a 25% wt. solution of NaOMe in MeOH (5.68 mL, 26.3 mmol) was added. The solution was heated to 75° C. for 6 h then cooled to RT and concentrated under vacuum. The material was diluted with ethyl acetate and washed with saturated NH₄Cl solution. The organic layer was separated, washed with water and brine, dried over Na₂SO₄, filtered and concentrated. 1H NMR & LC/MS show desired product. The crude material was recrystallized from hot ethyl acetate to give 4-[1-(phenylmethyl)-4-piperidinyl]-2,4-dihydro-3H-1,2,4-triazol-3-one (0.46 g, 1.692 mmol, 64.4% yield) as a beige solid. 1H NMR (400 MHz, DMSO-d₆) δ ppm 11.63 (br. s., 1H) 8.00 (s, 1H) 7.08-7.48 (m, 5H) 3.58-3.82 (m, 1H) 3.48 (s, 2H) 2.87 (d, 2H) 2.03 (td, 2H) 1.58-1.91 (m, 4H).

### Step C: 4-(4-piperidinyl)-2,4-dihydro-3H-1,2,4-triazol-3-one

A mixture of 4-[1-(phenylmethyl)-4-piperidinyl]-2,4-dihydro-3H-1,2,4-triazol-3-one (0.35 g, 1.355 mmol) and 20% palladium hydroxide on carbon (0.095 g, 0.135 mmol) in cyclohexadiene (1.923 mL, 20.32 mmol) was heated to 100° C. for 30 min in a microwave reactor. Ethanol (30 mL) and water (30 mL) were added and the mixture was filtered thru Celite and concentrated to dryness to give a grey solid. Used as is without further purification.

### Step D: 4-(1-{[3-chloro-6-(3-furanyl)-8-(trifluoromethyl)imidazo[1,2-a]pyridin-2-yl]carbonyl}-4-piperidinyl)-2,4-dihydro-3H-1,2,4-triazol-3-one

A solution of 4-(4-piperidinyl)-2,4-dihydro-3H-1,2,4-triazol-3-one (142 mg, 0.844 mmol), 3-chloro-6-(3-furanyl)-8-(trifluoromethyl)imidazo[1,2-a]pyridine-2-carboxylic acid (279 mg, 0.844 mmol), HATU (353 mg, 0.929 mmol) and DIPEA (0.295 mL, 1.689 mmol) in N,N-Dimethylformamide (DMF) (8 mL) was stirred at RT for 1 h then 1N HCl (20 mL) and ethyl acetate (25 mL) were added. The organic layer was separated, washed with saturated NaHCO₃ solution (50 mL), water (50 mL), brine (50 mL), dried over MgSO₄, filtered and concentrated to give a brown solid. The crude material was purified by Reverse Phase HPLC (MeCN/water+0.1% TFA) to give 4-(1-{[3-chloro-6-(3-furanyl)-8-(trifluoromethyl)imidazo[1,2-a]pyridin-2-yl]carbonyl}-4-piperidinyl)-2,4-dihydro-3H-1,2,4-triazol-3-one (35 mg, 0.069 mmol, 8.19% yield) as a white powder. 1H NMR (400 MHz, DMSO-d₆) δ ppm 11.62 (br. s., 1H) 8.77 (s, 1H) 8.51 (s, 1H) 8.16 (s, 1H) 8.02 (s, 1H) 7.79 (s, 1H) 7.28 (s, 1H) 4.59 (br. s., 1H) 4.21 (br. s., 1H) 3.90-4.09 (m, 1H) 3.14-3.32 (m, 1H) 2.81-3.03 (m, 1H) 1.55-2.07 (m, 4H), LCMS m/z 481.0 (M+1).

### Example 99

### 1-(1-{[3-chloro-6-cyclopropyl-8-(trifluoromethyl)imidazo[1,2-a]pyridin-2-yl]carbonyl}-4-piperidinyl)-2,5-pyrrolidinedione (Compound 200)

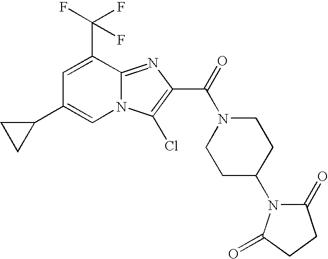

HATU (105 mg, 0.276 mmol) was added to a solution of 3-chloro-6-cyclopropyl-8-(trifluoromethyl)imidazo[1,2-a]pyridine-2-carboxylic acid (70 mg, 0.230 mmol), 1-(4-piperidinyl)-2,5-pyrrolidinedione and DIPEA (0.080 mL, 0.460 mmol) in N,N-Dimethylformamide (DMF) (2 mL) at RT under nitrogen. After 48 h, the clear solution was poured slowly into an aqueous 1N HCl solution (˜15 mL) and stirred for 10 min at RT then DCM (20 mL) was added. The organic layer was separated, washed with saturated NaHCO₃ solution, water, brine, dried over MgSO₄, filtered and concentrated. The crude material was purified by Reverse Phase HPLC (MeCN/water+0.1% formic acid) to give 1-(1-{[3-chloro-6-cyclopropyl-8-(trifluoromethyl)imidazo[1,2-a]pyridin-2-yl]carbonyl}-4-piperidinyl)-2,5-pyrrolidinedione (45 mg 0.091 mmol, 39.7% yield) as a white powder. 1H NMR (400 MHz, DMSO-d₆) δ ppm 8.43 (s, 1H) 7.58 (s, 1H) 4.61 (d, 1H) 4.05-4.28 (m, 2H) 3.15 (t, 1H) 2.79-2.93 (m, 1H) 2.59 (s, 4H) 2.10-2.32 (m, 3H) 1.68 (br. s., 1H) 1.52 (d, 1H) 0.97-1.11 (m, 2H) 0.86-0.95 (m, 2H). LCMS m/z 469.1 (M+H).

### Example 100

### 1-(1-{[3-chloro-6-cyclopropyl-8-(trifluoromethyl)imidazo[1,2-a]pyridin-2-yl]carbonyl}-4-piperidinyl)-2-pyrrolidinone (Compound 201)

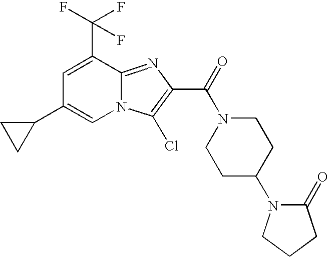

HATU (150 mg, 0.394 mmol) was added to a solution of 3-chloro-6-cyclopropyl-8-(trifluoromethyl)imidazo[1,2-a]pyridine-2-carboxylic acid (100 mg, 0.328 mmol), 1-(4-piperidinyl)-2-pyrrolidinone and DIPEA (0.115 mL, 0.656 mmol) in N,N-Dimethylformamide (DMF) (2 mL) at RT under nitrogen. After 48 h, the clear solution was poured slowly into an aqueous 1N HCl solution (˜15 mL) and stirred for 10 min at RT then DCM (20 mL) was added. The organic layer was separated, washed with saturated NaHCO₃ solution, water, brine, dried over MgSO₄, filtered and concentrated. The crude material was purified by Reverse Phase HPLC (MeCN/water+0.1% formic acid) to give 1-(1-{[3-chloro-6-cyclopropyl-8-(trifluoromethyl)imidazo[1,2-a]pyridin-2-yl]carbonyl}-4-piperidinyl)-2-pyrrolidinone (95 mg, 0.198 mmol, 60.4% yield) as a white powder. 1H NMR (400 MHz, DMSO-d₆) δ ppm 8.37 (s, 1H) 7.53 (s, 1H) 4.55 (d, 1H) 4.11 (d, 1H) 3.92-4.06 (m, 1H) 3.19-3.37 (m, 2H) 3.02-3.19 (m, 1H) 2.82 (td, 1H) 2.09-2.25 (m, 3H) 1.85 (qd, 2H) 1.39-1.73 (m, 4H) 0.90-1.02 (m, 2H) 0.78-0.90 (m, 2H). LCMS m/z 455.1 (M+H).

### Example 101

### 3-(1-{[3-chloro-6-cyclopropyl-8-(trifluoromethyl)imidazo[1,2-a]pyridin-2-yl]carbonyl}-4-piperidinyl)-1,3-oxazolidine-2,4-dione (Compound 202)

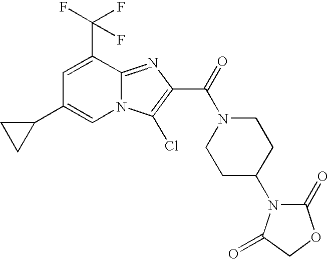

HATU (150 mg, 0.394 mmol) was added to a solution of 3-chloro-6-cyclopropyl-8-(trifluoromethyl)imidazo[1,2-a]pyridine-2-carboxylic acid (100 mg, 0.328 mmol), 3-(4-piperidinyl)-1,3-oxazolidine-2,4-dione HCl (80 mg, 0.361 mmol) and DIPEA (0.115 mL, 0.656 mmol) in N,N-Dimethylformamide (DMF) (2 mL) at RT under nitrogen. After 48 h, the clear solution was poured slowly into an aqueous 1N HCl solution (˜15 mL) and stirred for 10 min at RT then DCM (20 mL) was added. The organic layer was separated, washed with saturated NaHCO₃ solution, water, brine, dried over MgSO₄, filtered and concentrated. The crude material was purified by Reverse Phase HPLC (MeCN/water+0.1% formic acid) to give 3-(1-{[3-chloro-6-cyclopropyl-8-(trifluoromethyl)imidazo[1,2-a]pyridin-2-yl]carbonyl}-4-piperidinyl)-1,3-oxazolidine-2,4-dione (100 mg, 0.202 mmol, 61.5% yield) as a white powder. 1H NMR (400 MHz, DMSO-d₆) δ ppm 8.38 (s, 1H) 7.53 (s, 1H) 4.72 (s, 2H) 4.55 (br. s., 1H) 3.97-4.28 (m, 2H) 3.14 (t, 1H) 2.77-2.96 (m, 1H) 1.99-2.29 (m, 3H) 1.80 (br. s., 1H) 1.65 (br. s., 1H) 0.91-1.04 (m, 2H), 0.70-0.91 (m, 2H). LCMS m/z 471.07 (M+H).

### Example 102

### 3-(1-{[6-(3-furanyl)-8-(trifluoromethyl)imidazo[1,2-a]pyridin-2-yl]carbonyl}-4-piperidinyl)-1,3-oxazolidin-2-one (Compound 203)

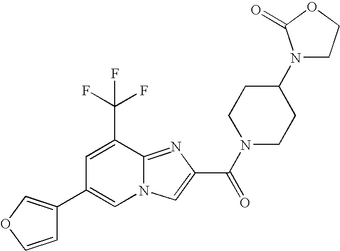

A solution of 6-(3-furanyl)-8-(trifluoromethyl)imidazo[1,2-a]pyridine-2-carboxylic acid (125 mg 0.422 mmol—see Schmitz, Franz Ulrich; Tai, Vincent W-F.; Rai, Roopa; Roberts, Christopher Don; Abadi, Ali Dehghani Mohammad; Baskaran, Subramanian; Slobodov, Irina; Maung, Jack; Neitzel, Martin Leon. Preparation of substituted imidazopyridine derivatives and analogs for use as antiviral agents. PCT Int. Appl. (2009), 434 pp. WO 2009023179), 3-(4-piperidinyl)-1,3-oxazolidin-2-one (90 mg, 0.528 mmol), HATU (201 mg, 0.528 mmol), and DIPEA (0.295 mL, 1.688 mmol) in N,N-dimethylformamide (5 mL) was maintained with stirring at room temperature for 2 hours. The solution was poured into ethyl acetate and washed twice with 5% LiCl (aq). The organic layer was dried over sodium sulfate, filtered, taken to a residue under reduced pressure, and purified by column chromatography to afford 3-(1-{[6-(3-furanyl)-8-(trifluoromethyl)imidazo[1,2-a]pyridin-2-yl]carbonyl}-4-piperidinyl)-1,3-oxazolidin-2-one (105 mg, 0.234 mmol, 55.5% yield) as a white solid. LCMS (m/z, ES⁺)=449 (M+H).

### Example 103

### 3-(1-{[3-chloro-6-(3-furanyl)-8-(trifluoromethyl)imidazo[1,2-a]pyridin-2-yl]carbonyl}-3,3-dimethyl-4-piperidinyl)-1,3-oxazolidin-2-one (Compound 204)

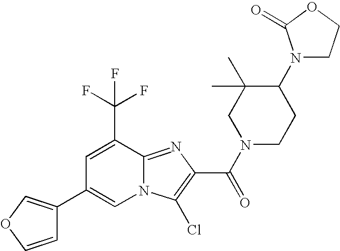

### Step A: 1,1-dimethylethyl 4-[(2-hydroxyethyl)amino]-3,3-dimethyl-1-piperidinecarboxylate

A solution of 1,1-dimethylethyl 3,3-dimethyl-4-oxo-1-piperidinecarboxylate (1000 mg, 4.40 mmol, ARK PHARMA), ethanolamine (0.798 mL, 13.20 mmol), and acetic acid (0.756 mL, 13.20 mmol) in methanol (20 mL) was maintained with stirring at room temperature for one hour. sodium cyanoborohydride (829 mg, 13.20 mmol) was added in one portion and the mixture was stirred overnight. The solution was poured into dichloromethane and washed with saturated aqueous sodium bicarbonate. The organic layer was dried over sodium sulfate, filtered, and taken to a residue under reduced pressure to afford 1,1-dimethylethyl 4-[(2-hydroxyethyl)amino]-3,3-dimethyl-1-piperidinecarboxylate (1.24 g, 4.55 mmol, 103% yield) as a yellow oil.

### Step B: 1,1-dimethylethyl 3,3-dimethyl-4-(2-oxo-1,3-oxazolidin-3-yl)-1-piperidinecarboxylate

A solution of 1,1-dimethylethyl 4-[(2-hydroxyethyl)amino]-3,3-dimethyl-1-piperidinecarboxylate (1.24 g, 4.55 mmol) and CDI (1.476 g, 9.10 mmol) was stirred at room temperature for 1 hour and then warmed to reflux and maintained for 16 hours. The mixture was cooled, diluted with ethyl acetate, and washed sequentially with saturated ammonium chloride (aq) and saturated sodium chloride (aq). The organic layer was dried over sodium sulfate, filtered, concentrated to a residue under reduced pressure, and purified by column chromatography to afford 1,1-dimethylethyl 3,3-dimethyl-4-(2-oxo-1,3-oxazolidin-3-yl)-1-piperidinecarboxylate (1.30 g, 4.36 mmol, 96% yield) as a clear oil.

### Step C: 3-(1-{[3-chloro-6-(3-furanyl)-8-(trifluoromethyl)imidazo[1,2-a]pyridin-2-yl]carbonyl}-3,3-dimethyl-4-piperidinyl)-1,3-oxazolidin-2-one

A mixture of 1,1-dimethylethyl 3,3-dimethyl-4-(2-oxo-1,3-oxazolidin-3-yl)-1-piperidinecarboxylate (50 mg, 0.168 mmol) and TFA (0.129 mL, 1.676 mmol) in dichloromethane (2 mL) was maintained with stirring for 2 hours. The volatiles were removed under reduced pressure to afford a crude residue containing 3-(3,3-dimethyl-4-piperidinyl)-1,3-oxazolidin-2-one (probably 50-60% pure). A solution of 3-chloro-6-(3-furanyl)-8-(trifluoromethyl)imidazo[1,2-a]pyridine-2-carboxylic acid (40 mg, 0.121 mmol), 3-(3,3-dimethyl-4-piperidinyl)-1,3-oxazolidin-2-one (30.0 mg, 0.151 mmol, crude residue from above), HATU (46.0 mg, 0.121 mmol), and DIPEA (0.021 mL, 0.121 mmol) in N,N-dimethylformamide (5 mL) was maintained with stirring at room temperature for 1 hour. The mixture was poured into ethyl acetate and extracted with aqueous LiCl and NaCl (sat). The organic layer was dried over sodium sulfate, filtered, taken to a residue under reduced pressure, and purified by reverse phase hplc to afford 3-(1-{[3-chloro-6-(3-furanyl)-8-(trifluoromethyl)imidazo[1,2-a]pyridin-2-yl]carbonyl}-3,3-dimethyl-4-piperidinyl)-1,3-oxazolidin-2-one (5.2 mg, 10.18 μmol, 8.41% yield) as a white solid. LCMS (m/z, ES⁺)=511 (M+H).

### Examples 104

### (±) cis-3-(1-{[3-chloro-6-(3-furanyl)-8-(trifluoromethyl)imidazo[1,2-a]pyridin-2-yl]carbonyl}-2-methyl-4-piperidinyl)-1,3-oxazolidin-2-one (Compound 205)

### Example 105

### (±) trans-3-(1-{[3-chloro-6-(3-furanyl)-8-(trifluoromethyl)imidazo[1,2-a]pyridin-2-yl]carbonyl}-2-methyl-4-piperidinyl)-1,3-oxazolidin-2-one (Compound 206)

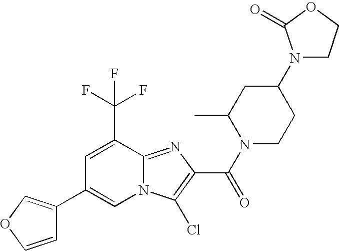

### Step A: 1,1-dimethylethyl 4-[(2-hydroxyethyl)amino]-2-methyl-1-piperidinecarboxylate

To a stirred solution of 1,1-dimethylethyl 2-methyl-4-oxo-1-piperidinecarboxylate (0.22 g, 1.0 mmol) and ethanolamine (0.092 g, 1.5 mmol) in methanol (20 mL) was added acetic acid (0.09 g). The mixture was stirred for three hours at room temperature, and then sodium triacetoxyborohydride (0.320 g, 1.5 mmol) was added and the mixture was maintained at room temperature with stirring overnight. The mixture was diluted with water and extracted with ethyl acetate and the resulting organic phase was dried and concentrated under reduced pressure to afford 1,1-dimethylethyl 4-[(2-hydroxyethyl)amino]-2-methyl-1-piperidinecarboxylate (0.358 g, 1.4 mmol) suitable for direct use in the subsequent transformation.

### Step B: 1,1-dimethylethyl 2-methyl-4-(2-oxo-1,3-oxazolidin-3-yl)-1-piperidinecarboxylate

A solution of 1,1-dimethylethyl 4-[(2-hydroxyethyl)amino]-2-methyl-1-piperidinecarboxylate (0.35 g, 1.4 mmol), dimethyl carbonate (0.13 g, 1.43 mmol), and sodium methoxide (0.036g, 0.08 mmol) in methanol was heated at reflux overnight. The mixture was diluted with water and mixed with ethyl acetate. The organic layer was concentrated to dryness and the residue purified by column chromatography to afford 1,1-dimethylethyl 2-methyl-4-(2-oxo-1,3-oxazolidin-3-yl)-1-piperidinecarboxylate (0.386 g, 1.36 mmol) as a solid.

### Step C: 3-(2-methyl-4-piperidinyl)-1,3-oxazolidin-2-one

A solution of 1,1-dimethylethyl 2-methyl-4-(2-oxo-1,3-oxazolidin-3-yl)-1-piperidinecarboxylate (0.86 g, 3 mmol) in 1.0M HCl in dioxane (10 mL) was maintained with stirring for two hours. The solvent was removed under reduced pressure and the residue was triturated with diethyl ether to afford 3-(2-methyl-4-piperidinyl)-1,3-oxazolidin-2-one as the hydrochloride salt (0.66g, 3 mmol) as a white solid.

Step D: A solution of 3-chloro-6-(3-furanyl)-8-(trifluoromethyl)imidazo[1,2-a]pyridine-2-carboxylic acid (550 mg, 1.663 mmol), 3-(2-methyl-4-piperidinyl)-1,3-oxazolidin-2-one (404 mg, 1.830 mmol), HATU (727 mg, 1.913 mmol), and DIPEA (0.872 mL, 4.99 mmol) in N,N-dimethylformamide (5 mL) was maintained with stirring at room temperature for 45 minutes. The mixture was poured into ethyl acetate and washed twice with 5% LiCl (aq) and once with saturated sodium chloride (aq). The organic layer was dried over sodium sulfate and taken to a residue under reduced pressure. The two diastereomers were purified (and isolated as racemates with single relative configurations) using a Phenomenex Gemini C18 column using 20 mL/min and 40% MeCN (isocratic) method to afford (±) cis-3-(1-{[3-chloro-6-(3-furanyl)-8-(trifluoromethyl)imidazo[1,2-a]pyridin-2-yl]carbonyl}-2-methyl-4-piperidinyl)-1,3-oxazolidin-2-one (200 mg) and (±) trans-3-(1-{[3-chloro-6-(3-furanyl)-8-(trifluoromethyl)imidazo[1,2-a]pyridin-2-yl]carbonyl}-2-methyl-4-piperidinyl)-1,3-oxazolidin-2-one (43 mg) as white solids. Both compounds show LCMS (m/z, ES⁺)=497 (M+H). Relative configuration assigned based on NOESY correlations.

### Example 106

### 3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 1) (Compound 207)

### Example 107

### 3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 2) (Compound 208)

### Example 108

### 3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 3) (Compound 209)

### Example 109

### 3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 4) (Compound 210)

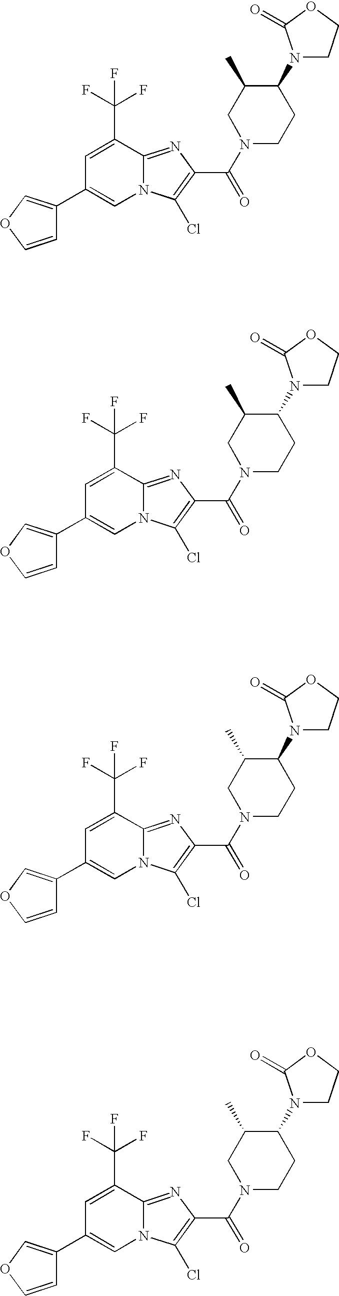

### Step A: (±)-1,1-dimethylethyl 4-[(2-hydroxyethyl)amino]-3-methyl-1-piperidinecarboxylate

A solution of 1,1-dimethylethyl 3-methyl-4-oxo-1-piperidinecarboxylate (1.85 g, 8.67 mmol, ARK PHARMA), ethanolamine (1.574 mL, 26.0 mmol), and AcOH (1.490 mL, 26.0 mmol) in methanol (40 mL) was stirred for 1 hour and then treated in one portion with sodium cyanoborohydride (1.635 g, 26.0 mmol). The mixture was stirred overnight and then poured into dichloromethane and washed with saturated sodium bicarbonate. The organic layer was dried over sodium sulfate, filtered, and taken to a residue under reduced pressure to afford 1,1-dimethylethyl 4-[(2-hydroxyethyl)amino]-3-methyl-1-piperidinecarboxylate (2.15 g, 8.32 mmol, 96% yield) as a yellow oil.

### Step B: (±)-cis&trans-1,1-dimethylethyl 3-methyl-4-(2-oxo-1,3-oxazolidin-3-yl)-1-piperidinecarboxylate

A solution of 1,1-dimethylethyl 4-[(2-hydroxyethyl)amino]-3-methyl-1-piperidinecarboxylate (2.15 g, 8.32 mmol) and CDI (2.70 g, 16.64 mmol) in benzene (75 mL) was stirred at room temperature for 1 hour and then warmed to reflux and maintained with rapid stirring for 16 hours. The mixture was cooled, poured into ethyl acetate, and washed with saturated ammonium chloride (aq) and saturated sodium chloride (aq). The organic layer was dried over sodium sulfate, filtered, taken to a residue under reduced pressure, and purified by column chromatography to afford 1,1-dimethylethyl 3-methyl-4-(2-oxo-1,3-oxazolidin-3-yl)-1-piperidinecarboxylate (2.200 g, 7.74 mmol, 93% yield) as a clear oil.

### Step C: (±)-cis&trans-3-(3-methyl-4-piperidinyl)-1,3-oxazolidin-2-one

A solution of 1,1-dimethylethyl 3-methyl-4-(2-oxo-1,3-oxazolidin-3-yl)-1-piperidinecarboxylate (2.20 g, 7.74 mmol) was dissolved in dichloromethane (50 mL), treated with TFA (8.94 mL, 116 mmol), and maintained with stirring for 3 hours. The mixture was concentrated under reduced pressure to afford 3-(3-methyl-4-piperidinyl)-1,3-oxazolidin-2-one (3.97 g, 6.66 mmol, 86% yield) as a yellow oil.

Step D:

3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 1)

3(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 2)

3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 3)

3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 4)

A solution of 3-chloro-6-(3-furanyl)-8-(trifluoromethyl)imidazo[1,2-a]pyridine-2-carboxylic acid (750 mg, 2.268 mmol), (±)-3-(3-methyl-4-piperidinyl)-1,3-oxazolidin-2-one (1691 mg, 2.84 mmol, mixture of cis and trans), HATU (1035 mg, 2.72 mmol), and DIPEA (1.981 mL, 11.34 mmol) in N,N-dimethylformamide (15 mL) was maintained with stirring for 2 hours. The mixture was poured into ethyl acetate and washed three times with 5% LiCl (aq) and once with saturated sodium chloride (aq). The organic layer was dried over sodium sulfate, filtered, taken to a residue under reduced pressure, and purified by column chromatography. Separation of the four constituent enantiomers (which were in roughly a 3:3:1:1 ratio of diasteromers) was accomplished using a Berger SFC with a ChiralPak OJ 60A 5u; 4.6×250 mm column with an isochratic 15% MeOH mobile phase to afford a first eluting isomer (15 mg, >99% ee, 91% chemical purity, LCMS (m/z, ES⁺)=497 (M+H)), a second eluting isomer (enantiomer of first eluting isomer, 11 mg, 97% ee, >99% chemical purity, LCMS (m/z, ES⁺)=497 (M+H)), a third eluting isomer (42 mg, >99% ee, 97% chemical purity, LCMS (m/z, ES⁺)=497 (M+H)), and a fourth eluting isomer (27 mg, enantiomer of the third eluting isomer, 99% ee, >99% chemical purity, LCMS (m/z, ES⁺)=497 (M+H)) all as white solids. Absolute and relative stereochemical assignments could not be made.

### Example 110

### 3-(1-{[3-chloro-6-(3-furanyl)-8-(trifluoromethyl)imidazo[1,2-a]pyridin-2-yl]carbonyl}-2,2-dimethyl-4-piperidinyl)-1,3-oxazolidin-2-one (Compound 211)

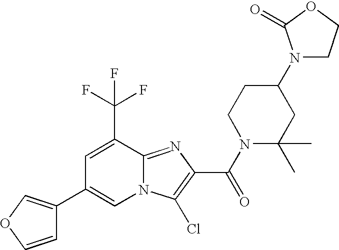

### Step A: 2-{[2,2-dimethyl-1-(phenylmethyl)-4-piperidinyl]amino}ethanol

To a solution of 2,2-dimethyl-1-(phenylmethyl)-4-piperidinone (1.08 g, 4.98 mmol, see: Blanco-Pillado, Maria-Jesus; Benesh, Dana Rae; Filla, Sandra Ann; Hudziak, Kevin John; Mathes, Brian Michael; Kohlman, Daniel Timothy; Ying, Bai-Ping; Zhang, Deyi; Xu, Yao-Chang. Preparation of N-[(piperidinyloxy)phenyl]-, N-[(piperidinyloxy)pyridinyl]-, N-[(piperidinylsulfanyl)phenyl]-, and N-[(piperidinylsulfanyl)pyridinyl]amides as 5-HT1F agonists for treatment of migraine. PCT Int. Appl. (2004), 186 pp. WO 2004094380), and ethanolamine (0.46 g, 7.47 mmol) in methanol (10 mL) was added acetic acid (0.45 g, 7.47 mmol). The mixture was stirred at room temperature for 3 hours and then sodium triacetoxyborohyride (1.58 g, 7.67 mmol) was added and the mixture was maintained with stirring overnight. Saturated aqueous potassium carbonate was added and the mixture was diluted with ethyl acetate. The organic layer was separated, dried, filtered, and concentrated to afford 2-{[2,2-dimethyl-1-(phenylmethyl)-4-piperidinyl]amino}ethanol (1.3 g, 4.98 mmol) with purity sufficient for direct use in the subsequent transformation.

### Step B: 3-[2,2-dimethyl-1-(phenylmethyl)-4-piperidinyl]-1,3-oxazolidin-2-one

A solution of 2-{[2,2-dimethyl-1-(phenylmethyl)-4-piperidinyl]amino}ethanol (1.1g, 4.2 mmol) and sodium methoxide (0.11 g, 2.1 mmol) in methanol (10 mL) was treated with dimethyl carbonate (0.4 g, 4.41 mmol) and the mixture was heated to reflux overnight. The mixture was diluted with water and extracted with dichloromethane. The combined extracts were washed with water, dried, concentrated, and purified by column chromatography to afford 3-[2,2-dimethyl-1-(phenylmethyl)-4-piperidinyl]-1,3-oxazolidin-2-one (1.2 g, 0.2 mmol) as a white solid.

### Step C: 3-(2,2-dimethyl-4-piperidinyl)-1,3-oxazolidin-2-one

A mixture of 3-[2,2-dimethyl-1-(phenylmethyl)-4-piperidinyl]-1,3-oxazolidin-2-one (0.670 mg, 1.6 mmol) and 10% Pd/C (0.5 g) in ethanol (10 mL) was maintained at 70° C. under an atmosphere of hydrogen overnight. The mixture was filtered, concentrated, suspended in ethyl acetate, and treated with HCl. Filtration afforded 3-(2,2-dimethyl-4-piperidinyl)-1,3-oxazolidin-2-one as a white solid as the hydrochloride salt.

### Step D: 3-(1-{[3-chloro-6-(3-furanyl)-8-(trifluoromethyl)imidazo[1,2-a]pyridin-2-yl]carbonyl}-2,2-dimethyl-4-piperidinyl)-1,3-oxazolidin-2-one

A solution of 3-chloro-6-(3-furanyl)-8-(trifluoromethyl)imidazo[1,2-a]pyridine-2-carboxylic acid (20 mg, 0.060 mmol), 3-(2,2-dimethyl-4-piperidinyl)-1,3-oxazolidin-2-one HCl (15.62 mg, 0.067 mmol), HATU (27.6 mg, 0.073 mmol), and DIPEA (0.042 mL, 0.242 mmol) in N,N-dimethylformamide (5 mL) was maintained with stirring at room temperature for 1 hour. The mixture was loaded directly into a reverse phase hplc and pure fractions were concentrated to afford 3-(1-{[3-chloro-6-(3-furanyl)-8-(trifluoromethyl)imidazo[1,2-a]pyridin-2-yl]-2,2-dimethyl-4-piperidinyl)-1,3-oxazolidin-2-one (11 mg, 0.022 mmol, 35.6% yield) as a yellow solid. LCMS (m/z, ES⁺)=511 (M+H).

### Example 111

### (±)-trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one (Compound 212)

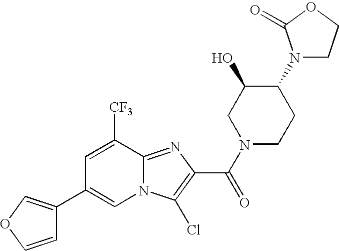

### Step A: 1,1-dimethylethyl-4-({[(2-chloroethyl)oxy]carbonyl}amino)-3-hydroxy-1-piperidinecarboxylate

A solution of 1,1-dimethylethyl-4-amino-3-hydroxy-1-piperidinecarboxylate (2.040 g, 9.43 mmol, 4:1 mixture of regioisomers—SEE: Boyd, Michael J.; Crane, Sheldon N.; Robichaud, Joel; Scheigetz, John; Black, W. Cameron; Chauret, Nathalie; Wang, Qingping; Masse, Frederic; Oballa, Renata M. Investigation of ketone warheads as alternatives to the nitrile for preparation of potent and selective cathepsin K inhibitors. Bioorganic & Medicinal Chemistry Letters (2009), 19(3), 675-679.), 2-chloroethyl chloridocarbonate (1.073 ml, 10.38 mmol), and DIPEA (4.12 ml, 23.58 mmol) in dichloromethane was maintained at 0° C. with stirring for 2 hours. The mixture was poured into saturated sodium bicarbonate (aq) and diluted with additional dichloromethane. The organic layer was dried over sodium sulfate, filtered, and concentrated under reduced pressure to afford 1,1-dimethylethyl-4-({[(2-chloroethyl)oxy]carbonyl}amino)-3-hydroxy-1-piperidinecarboxylate (3.77 g, 8.76 mmol, 93% yield) as a mixture of regioisomers (roughly 4:1).

### Step B: (±)-trans-1,1-dimethylethyl-3-hydroxy-4-(2-oxo-1,3-oxazolidin-3-yl)-1-piperidinecarboxylate

A solution of 1,1-dimethylethyl-4-({[(2-chloroethyl)oxy]carbonyl}amino)-3-hydroxy-1-piperidinecarboxylate (3.77 g, 11.68 mmol) from above in N,N-dimethylformamide (50 mL) was maintained with stirring at room temperature and treated portionwise with NaH (0.561 g, 23.36 mmol) over 15 minutes. The mixture was stirred until bubbling had ceased and then the reaction was quenched via careful addition of saturated ammonium chloride (aq) under nitrogen and diluted with ethyl acetate. The organic layer was dried over sodium sulfate, filtered, taken to a residue under reduced pressure, and purified by column chromatography to afford (±)-trans-1,1-dimethylethyl-3-hydroxy-4-(2-oxo-1,3-oxazolidin-3-yl)-1-piperidinecarboxylate (690 mg, 2.410 mmol, 20.63% yield) as a white solid and single regioisomer (minor regioisomer discarded during chromatography).

### Step C: (±)-trans-3-[3-hydroxy-4-piperidinyl]-1,3-oxazolidin-2-one

A solution of (±)-trans-1,1-dimethylethyl-3-hydroxy-4-(2-oxo-1,3-oxazolidin-3-yl)-1-piperidinecarboxylate (150 mg, 0.524 mmol) in dichloromethane (DCM) (5 mL) was treated with TFA (0.404 mL, 5.24 mmol) and maintained with stirring at room temperature for 1 hour. All volatiles were removed under reduced pressure to afford (±)-trans-3-[3-hydroxy-4-piperidinyl]-1,3-oxazolidin-2-one (150 mg, 0.500 mmol, 95% yield) as a yellow oil.

### Step D: (±)-trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one

A solution of 3-chloro-6-(3-furanyl)-8-(trifluoromethyl)imidazo[1,2-a]pyridine-2-carboxylic acid (135 mg, 0.408 mmol), (±)-trans-3-[3-hydroxy-4-piperidinyl]-1,3-oxazolidin-2-one (147 mg, 0.490 mmol), HATU (186 mg, 0.490 mmol), and DIPEA (0.285 mL, 1.633 mmol) in N,N-dimethylformamide (5 mL) was maintained with stirring at room temperature for 45 minutes. The mixture was diluted with ethyl acetate and poured into 5% LiCl (aq). The organic layer was separated, dried over sodium sulfate, taken to a residue under reduced pressure, and purified by column chromatography to afford (±)-trans-3-((1-{[3-chloro-6-(3-furanyl)-8-(trifluoromethyl)imidazo[1,2-a]pyridin-2-yl]carbonyl}-3-hydroxy-4-piperidinyl)-1,3-oxazolidin one (111 mg, 0.223 mmol, 54.5% yield) as a white solid. LCMS (m/z, ES⁺)=499 (M+H).

### Example 112

### (±)-cis-3-(1-{[3-chloro-6-(3-furanyl)-8-(trifluoromethyl)imidazo[1,2-a]pyridin-2-yl]carbonyl}-3-fluoro-4-piperidinyl)-1,3-oxazolidin-2-one (Compound 213)

### Example 113

### (±)-trans-3-(1-{[3-chloro-6-(3-furanyl)-8-(trifluoromethyl)imidazo[1,2-a]pyridin-2-yl]carbonyl}-3-fluoro-4-piperidinyl)-1,3-oxazolidin-2-one (Compound 214)

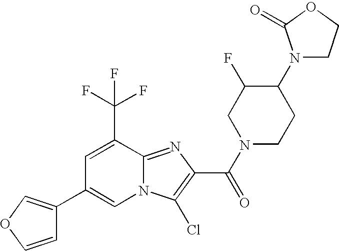

A solution of (±)-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one (40 mg, 0.080 mmol) in dichloromethane (5 mL) was treated with DAST (0.159 mL, 1.203 mmol) and maintained with stirring at room temperature for 45 minutes, at which time all solids had been observed to dissolve. The mixture was quenched with saturated sodium bicarbonate and diluted with additional dichloromethane. The organic layer was dried over sodium sulfate, filtered, taken to a residue under reduced pressure, passed through a short silica plug, and purified by reverse phase chromatography to afford both isomers of 3-(1-{[3-chloro-6-(3-furanyl)-8-(trifluoromethyl)imidazo[1,2-a]pyridin-2-yl]carbonyl}-3-fluoro-4-piperidinyl)-1,3-oxazolidin-2-one as an early eluting isomer (6 mg, LCMS (m/z, ES⁺)=501 (M+H)), and a second eluting isomer (5 mg, LCMS (m/z, ES⁺)=501 (M+H)).

### Example 114

### (±) trans-3-(1-{[3-chloro-6-(2-methylpropyl)-8-(trifluoromethyl)imidazo[1,2-a]pyridin-2-yl]carbonyl}-3-hydroxy-4-piperidinyl)-1,3-oxazolidin-2-one

### (Compound 215)

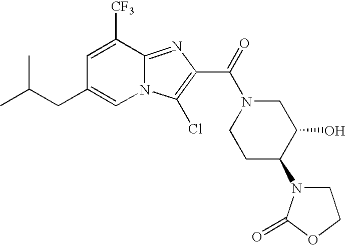

### Step A: (±) trans-3-[3-hydroxy-4-piperidinyl]-1,3-oxazolidin-2-one trifluoroacetamide

A solution of (±) trans-1,1-dimethylethyl 3-hydroxy-4-(2-methylidene-1,3-oxazolidin-3-yl)-1-piperidinecarboxylate (0.420 g, 1.47 mmol) in DCM (7.33 ml) was treated with TFA (1.13 ml, 14.67 mmol). The reaction was stirred at room temperature for 3 hours. The reaction was concentrated under reduced pressure to give (±) trans-3-[3-hydroxy-4-piperidinyl]-1,3-oxazolidin-2-one TFA salt as a oil, assumed quantitative. ES-LCMS: 187.0 (M+1).

### Step B: (±) trans-3-(1-{[3-chloro-6-(2-methylpropyl)-8-(trifluoromethyl)imidazo[1,2-a]pyridin-2-yl]carbonyl}-3-hydroxy-4-piperidinyl)-1,3-oxazolidin-2-one

A solution of (±) trans-3-[3-hydroxy-4-piperidinyl]-1,3-oxazolidin-2-one TFA (0.11 g, 0.366 mmol) and 3-chloro-6-(2-methylpropyl)-8-(trifluoromethyl)imidazo[1,2-a]pyridine-2-carboxylic acid (0.129 g, 0.40 mmol) in DMF (1.83 ml) was treated with DIEA (0.192 ml, 1.10 mmol) and HATU (0.160 g, 0.42 mmol). The reaction was stirred at room temperature for 2.5 hours. The reaction was then diluted with EtOAc and washed with 5% LiCl (×2) and then brine. The organics were dried MgSO₄, filtered, and concentrated. The crude residue (200 mg) was purified by silica gel chromatography (0-10% MeOH/DCM+1% (2M NH₄ in MeOH)) to give (±) trans-3-(1-{[3-chloro-6-(2-methylpropyl)-8-(trifluoromethyl)imidazo[1,2-a]pyridin-2-yl]carbonyl}-3-hydroxy-4-piperidinyl)-1,3-oxazolidin-2-one (0.054 g, 30%). ES-LCMS: 489.2 (M+1).

### Example 115

### (±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (compound 216)

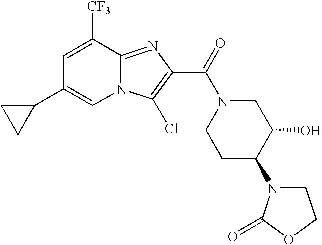

A solution of (±) trans-3-[3-hydroxy-4-piperidinyl]-1,3-oxazolidin-2-one TFA salt (0.111 g, 0.37 mmol) and 3-chloro-6-cyclopropyl-8-(trifluoromethyl)imidazo[1,2-a]pyridine-2-carboxylic acid (0.124 g, 0.41 mmol) in DMF (1.849 ml) was treated with DIEA (0.194 ml, 1.11 mmol) and HATU (0.162 g, 0.46 mmol). The reaction was stirred at room temperature for 2.5 hours. The reaction was then diluted with EtOAc and washed with 5% LiCl (×2) and then brine. The organics were dried MgSO₄, filtered, and concentrated. The crude residue (195 mg) was purified by silica gel chromatography (0-10% MeOH/DCM+1% (2M NH₄ in MeOH)) to give (±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (0.129 g, 74%). ES-LCMS: 473.2 (M+1).

### Example 116

### 3-((trans)-1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one (enantiomer 1) (Compound 217)

### Example 117

### 3-((trans)-1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one (enantiomer 2) (Compound 218)

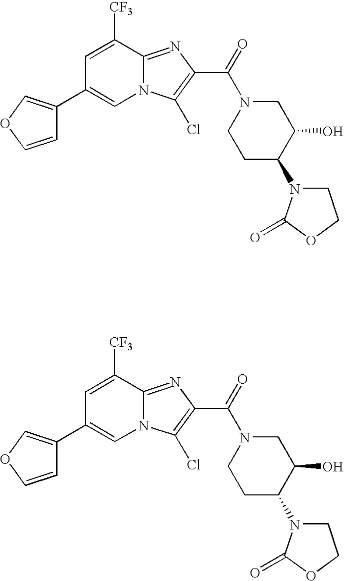

Separation of (±)-trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one was performed on ChiralPak ASH column (250×10 mm i.d., 5 um; ChiralTechnologies, West Chester, Pa.) under supercritical conditions maintained at 40° C., 140 bar, with methanol modified CO₂ (25%MeOH, 75% CO₂) delivered at a combined flow rate of 10 ml/min on a Berger Minigram SFC system (Berger Instruments, Inc.; Neward, Del.). Triggered collections were made using a Knauer selectable wavelength UV-Vis detector at 265 nm to afford a first eluting isomer (24.9 mg, >99% ee, ES-LCMS: 499.1 (M+1)) and a second eluting isomer (27 mg, >99% ee, ES-LCMS: 499.2 (M+1)). Absolute stereochemical assignments were not made.

### Example 118

### (±) trans-3-(1-{[6-bromo-3-chloro-8-(trifluoromethyl)imidazo[1,2-a]pyridin-2-yl]carbonyl}-3-hydroxy-4-piperidinyl)-1,3-oxazolidin-2-one (compound 219)

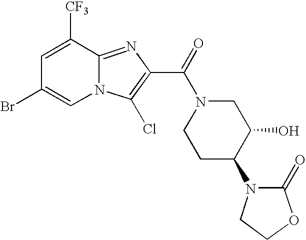

A solution of 6-bromo-3-chloro-8-(trifluoromethyl)imidazo[1,2-a]pyridine-2-carboxylic acid (0.132 g, 0.385 mmol) and (±) trans-3-[3-hydroxy-4-piperidinyl]-1,3-oxazolidin-2-one TFA (0.105 g, 0.35 mmol) in DMF (1.749 ml) was treated by the addition of DIEA (0.183 ml, 1.05 mmol) and HATU (0.146 g, 0.39 mmol). The reaction was stirred at room temperature for 2 hours and the reaction was diluted with EtOAc and washed with 5% LiCl solution (2×), then brine. The reaction was dried MgSO₄, filtered, and concentrated. The crude residue was purified by silica gel chromatography (1-5% MeOH/DCM) to give the product and an impurity. The residue was repurified by silica gel chromatography (1-3% MeOH/DCM) to give (±) trans-3-(1-{[6-bromo-3-chloro-8-(trifluoromethyl)imidazo[1,2-a]pyridin-2-yl]carbonyl}-3-hydroxy-4-piperidinyl)-1,3-oxazolidin-2-one (0.070 g, 39%). ES-LCMS: 513.0 (M+1).

### Example 119

### 3-((trans)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 1) (Compound 220)

### Example 120

### 3-((trans)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 2) (Compound 2)

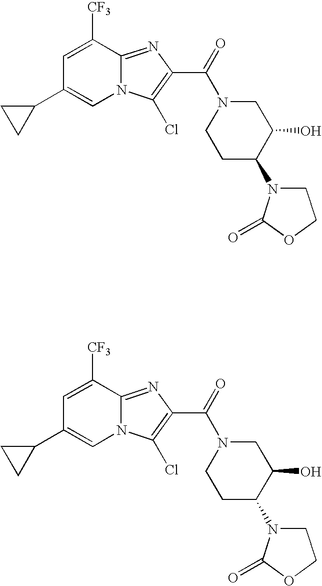

Separation of (±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one was performed on ChiralPak ASH column (250×10 mm i.d., 5 um; ChiralTechnologies, West Chester, Pa.) under supercritical conditions maintained at 40° C., 140 bar, with methanol modified CO₂ (20% MeOH, 80% CO₂) delivered at a combined flow rate of 10 ml/min on a Thar Discovery Series SFC system (Thar Instruments, Inc.; Pittsburgh, Pa). Triggered collections were made using a Gilson selectable wavelength 151 UV-Vis detector at 280 nm to afford a first eluting isomer (9.1 mg, >99% ee, ES-LCMS: 473.4 (M+1)) and a second eluting isomer (11 mg, >99% ee, ES-LCMS: 473.3 (M+1)). Absolute stereochemical assignments were not made.

### Example 121

### (±) trans-3-(1-{[6-bromo-3-chloro-8-(trifluoromethyl)imidazo[1,2-a]pyridin-2-yl]carbonyl}-3-hydroxy-4-piperidinyl)-1,3-oxazolidine-2,4-dione (Compound 222)

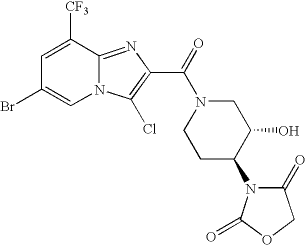

### Step A: (±) trans-1,1-dimethylethyl 4-azido-3-{[(1,1-dimethylethyl)(dimethyl)silyl]oxy}-1-piperidinecarboxylate

A solution of (±) trans-1,1-dimethylethyl 4-azido-3-hydroxy-1-piperidinecarboxylate (3.0 g, 12.38 mmol) in DMF (61.9 ml) was treated with imidazole (1.26 g, 18.57 mmol) followed by the addition of TBSCl (2.05 g, 13.62 mmol) in DMF (61.9 ml). The reaction was stirred at room temperature overnight and then the reaction was diluted with EtOAc and washed with 5% LiCl (×2), brine, dried MgSO₄, filtered, and concentrated. The crude residue was purified by silica gel chromatography (0-30% EtOAc/Hexane) to give (±) trans-1,1-dimethylethyl 4-azido-3-{[(1,1-dimethylethyl)(dimethyl)silyl]oxy}-1-piperidinecarboxylate.

### Step B: (±) trans-1,1-dimethylethyl 4-amino-3-{[(1,1-dimethylethyl)(dimethyl)silyl]oxy}-1-piperidinecarboxylate

A solution of (±) trans-1,1-dimethylethyl 4-azido-3-{[(1,1-dimethylethyl)(dimethyl)silyl]oxy}-1-piperidinecarboxylate (3.29 g, 9.23 mmol) in MeOH (46.1 ml) was treated with 10% Pd/C (0.98 g, 0.92 mmol) and hydrogenated at 40 psi overnight. The catalyst was then filtered through GF/F and concentrated under reduced pressure to give (±) trans-1,1-dimethylethyl 4-amino-3-{[(1,1-dimethylethyl)(dimethyl)silyl]oxy}-1-piperidinecarboxylate (2.88 g, 94%). ES-LCMS: 331.5 (M+1).

### Step C: (±) trans-1,1-dimethylethyl 3-{[(1,1-dimethylethyl)(dimethyl)silyl]oxy}-4-[(hydroxyacetyl)amino]-1-piperidinecarboxylate

A solution of (±) trans-1,1-dimethylethyl 4-amino-3-{[(1,1-dimethylethyl)(dimethyl)silyl]oxy}-1-piperidinecarboxylate (1.4 g, 4.24 mmol) and hydroxyacetic acid (0.354 g, 4.66 mmol) in DMF (21.18 ml) was treated with DIEA (1.11 ml, 6.35 mmol) and HATU (1.77 g, 4.66 mmol). The reaction was stirred at room temperature for 3 hours. The reaction was diluted with EtOAc and washed with 5% LiCl (2×), saturated NaHCO₃, brine, dried MgSO₄, filtered, and concentrated to give (±) trans-1,1-dimethylethyl 3-{[(1,1-dimethylethyl)(dimethyl)silyl]oxy}-4-[(hydroxyacetyl)amino]-1-piperidinecarboxylate (1.61 g, 98%) which was used without further purification. ES-LCMS: 389.3 (M+1).

### Step D: (±) trans-1,1-dimethylethyl 3-{[(1,1-dimethylethyl)(dimethyl)silyl]oxy}-4-(2,4-dioxo-1,3-oxazolidin-3-yl)-1-piperidinecarboxylate

A solution of (±) trans-1,1-dimethylethyl 3-{[(1,1-dimethylethyl)(dimethyl)silyl]oxy}-4-[(hydroxyacetyl)amino]-1-piperidinecarboxylate (1.61 g, 4.14 mmol) in DMF (20.72 ml) was treated with CDI (0.74 g, 4.56 mmol) was stirred at room temperature for 1.5 hours. The reaction was then heated to 80° C. overnight. The reaction was concentrated and the crude residue was purified silica gel chromatography (0-30% EtOAc/Hexanes) to give (±) trans-1,1-dimethylethyl 3-{[(1,1-dimethylethyl)(dimethyl)silyl]oxy}-4-(2,4-dioxo-1,3-oxazolidin-3-yl)-1-piperidinecarboxylate (0.892 g, 52%). ES-LCMS: 415.4 (M+1).

### Step E: (±) trans-3-(3-{[(1,1-dimethylethyl)(dimethyl)silyl]oxy}-4-piperidinyl)-1,3-oxazolidine-2,4-dione TFA

A solution of (±) trans-1,1-dimethylethyl 3-{[(1,1-dimethylethyl)(dimethyl)silyl]oxy}-4-(2,4-dioxo-1,3-oxazolidin-3-yl)-1-piperidinecarboxylate (0.892 g, 2.15 mmol) in DCM (10.76 ml) was treated with TFA (1.66 ml, 21.52 mmol). The reaction was stirred at room temperature for 5 hours. The solvents were removed under reduced pressure to give (±) trans-3-(3-{[(1,1-dimethylethyl)(dimethyl)silyl]oxy}-4-piperidinyl)-1,3-oxazolidine-2,4-dione TFA (0.968 g, 105%). ES-LCMS: 316.4 (M+1).

### Step F: (±) trans-3-(-1-{[6-bromo-3-chloro-8-(trifluoromethyl)imidazo[1,2-a]pyridin-2-yl]carbonyl}-3-{[(1,1-dimethylethyl)(dimethyl)silyl]oxy}-4-piperidinyl)-1,3-oxazolidine-2,4-dione

A solution of (±) trans-3-(3-{[(1,1-dimethylethyl)(dimethyl)silyl]oxy}-4-piperidinyl)-1,3-oxazolidine-2,4-dione TFA (0.170 g, 0.40 mmol) and DIEA (0.173 ml, 0.99 mmol) in DMF (1.95 ml) was treated by the addition of 6-bromo-3-chloro-8-(trifluoromethyl)imidazo[1,2-a]pyridine-2-carboxylic acid (0.150 g, 0.44 mmol) and HATU (0.166 g, 0.44 mmol). The reaction was stirred at room temperature for 3 hours and then diluted with EtOAc. The organics were washed with 5% LiCl (2×), saturated NaHCO₃, brine, dried MgSO₄, filtered, and concentrate to give (±) trans-3-(1-{[6-bromo-3-chloro-8-(trifluoromethyl)imidazo[1,2-a]pyridin-2-yl]carbonyl}-3-{[(1,1-dimethylethyl)(dimethyl)silyl]oxy}-4-piperidinyl)-1,3-oxazolidine-2,4-dione (0.282 g, 111%) which was used without further purification. ES-LCMS: 641.2 (M+1).

### Step G: (±) trans-3-(1-{[6-bromo-3-chloro-8-(trifluoromethyl)imidazo[1,2-a]pyridin-2-yl]carbonyl}-3-hydroxy-4-piperidinyl)-1,3-oxazolidine-2,4-dione

A solution (±) trans-3-(1-{[6-bromo-3-chloro-8-(trifluoromethyl)imidazo[1,2-a]pyridin-2-yl]carbonyl}-3-{[(1,1-dimethylethyl)(dimethyl)silyl]oxy}-4-piperidinyl)-1,3-oxazolidine-2,4-dione (0.282 g, 0.441 mmol) in THF (2.20 ml) was treated with 1M in THF TBAF (0.66 ml, 0.66 mmol). The reaction was stirred at room temperature for 1.5 hours. The reaction was concentrated and the residue was taken up in EtOAc and washed with water, brine, dried MgSO₄, filtered, and concentrated. The crude residue was purified by silica gel chromatography (0-6% MeOH/DCM) to give (±) trans-3-(1-{[6-bromo-3-chloro-8-(trifluoromethylimidazo[1,2-a]pyridin-2-yl]carbonyl}-3-hydroxy-4-piperidinyl)-1,3-oxazolidine-2,4-dione (0.033 g, 14%). ES-LCMS: 527.0 (M+1).

### Example 122

### (±) trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidine-2,4-dione (Compound 223)

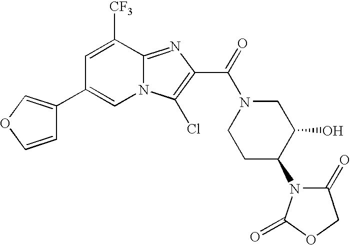

Step A: (±) trans-3-(1-{[3-chloro-6-(3-furanyl)-8-(trifluoromethyl)imidazo[1,2-a]pyridin-2-yl]carbonyl}-3-{[(1,1-dimethylethyl)(dimethyl)silyl]oxy}-4-piperidinyl)-1,3-oxazolidine-2,4-dione

A solution of (±) trans-3-(3-{[(1,1-dimethylethyl)(dimethyl)silyl]oxy}-4-piperidinyl)-1,3-oxazolidine-2,4-dione TFA (0.170 g, 0.40 mmol) and DIEA (0.173 ml, 0.99 mmol) in DMF (1.98 ml) was treated by the addition of 3-chloro-6-(3-furanyl)-8-(trifluoromethyl)imidazo[1,2-a]pyridine-2-carboxylic acid (0.144 g, 0.44 mmol) and HATU (0.166 g, 0.44 mmol). The reaction was stirred at room temperature for 3 hours and then diluted with EtOAc. The organics were washed with 5% LiCl (2×), saturated NaHCO₃, brine, dried MgSO₄, filtered, and concentrated to give (±) trans-3-(1-{[3-chloro-6-(3-furanyl)-8-(trifluoromethyl)imidazo[1,2-a]pyridin-2-yl]carbonyl}-3-{[(1,1-dimethylethyl)(dimethyl)silyl]oxy}-4-piperidinyl)-1,3-oxazolidine-2,4-dione (0.274 g, 110%) which was used without further purification. LCMS: 627.2 (M+1).

### Step B: (±) trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidine-2,4-dione

A solution (±) trans-3-(1-{[3-chloro-6-(3-furanyl)-8-(trifluoromethyl)imidazo[1,2-a]pyridin-2-yl]carbonyl}-3-{[(1,1-dimethylethyl)(dimethyl)silyl]oxy}-4-piperidinyl)-1,3-oxazolidine-2,4-dione (0.274 g, 0.44 mmol) in THF (2.19 ml) was treated with 1M in THF TBAF (0.655 ml, 0.66 mmol). The reaction was stirred at room temperature for 1.5 hours. The reaction was concentrated and the residue was taken up in EtOAc and washed with water, brine, dried MgSO₄, filtered, and concentrated. The crude residue was purified by silica gel chromatography (0-6% MeOH/DCM) to give (±) trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidine-2,4-dione (0.081 g, 36%). ES-LCMS: 513.2 (M+1).

### Example123

### (±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidine-2,4-dione (Compound 224)

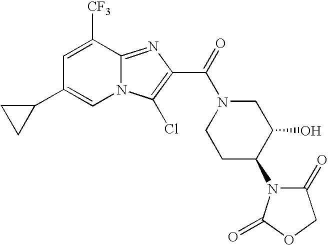

### Step A: (±) trans-3-(1-{[3-chloro-6-cyclopropyl-8-(trifluoromethyl)imidazo[1,2-a]pyridin-2-yl]carbonyl}-3-{[(1,1-dimethylethyl)(dimethyl)silyl]oxy}-4-piperidinyl)-1,3-oxazolidine-2,4-dione

A solution of (±) trans-3-(3-{[(1,1-dimethylethyl)(dimethyl)silyl]oxy}-4-piperidinyl)-1,3-oxazolidine-2,4-dione TFA (0.170 g, 0.40 mmol) and DIEA (0.173 ml, 0.99 mmol) in DMF (1.984 ml) was treated by the addition of 3-chloro-6-cyclopropyl-8-(trifluoromethyl)imidazo[1,2-a]pyridine-2-carboxylic acid (0.133 g, 0.44 mmol) and HATU (0.166 g, 0.44 mmol). The reaction was stirred at room temperature for 3 hours and then diluted with EtOAc. The organics were washed with 5% LiCl (2×), saturated NaHCO₃, brine, dried MgSO₄, filtered, and concentrated to give (±) trans-3-(1-{[3-chloro-6-cyclopropyl-8-(trifluoromethyl)imidazo[1,2-a]pyridin-2-yl]carbonyl}-3-{[(1,1-dimethylethyl)(dimethyl)silyl]oxy}-4-piperidinyl)-1,3-oxazolidine-2,4-dione (0.290 g, 122%) which was used without further purification. ES-LCMS: 601.3 (M+1).

### Step B: (±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidine-2,4-dione

A solution (±) trans-3-(1-{[3-chloro-6-cyclopropyl-8-(trifluoromethyl)imidazo[1,2-a]pyridin-2-yl]carbonyl}-3-{[(1,1-dimethylethyl)(dimethyl)silyl]oxy}-4-piperidinyl)-1,3-oxazolidine-2,4-dione (0.290 g, 0.48 mmol) in THF (2.41 ml) was treated with 1M in THF TBAF (0.724 ml, 0.72 mmol). The reaction was stirred at room temperature for 1.5 hours. The reaction was concentrated and the residue was taken up in EtOAc and washed with water, brine, dried MgSO₄, filtered, and concentrated. The crude residue was purified by silica gel chromatography (0-6% MeOH/DCM) to give (±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidine-2,4-dione (0.009 g, 4%). ES-LCMS: 487.2 (M+1).

### Example124

### (±) trans-1-(1-{[3-chloro-6-cyclopropyl-8-(trifluoromethyl)imidazo[1,2-a]pyridin-2-yl]carbonyl}-3-hydroxy-4-piperidinyl)-2-pyrrolidinone (Compound 225)

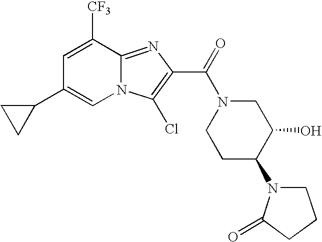

### Step A: (±) trans-1,1-dimethylethyl 4-azido-3-{[(1,1-dimethylethyl)(dimethyl)silyl]oxy}-1-piperidinecarboxylate

A solution of (±) trans-1,1-dimethylethyl 4-azido-3-hydroxy-1-piperidinecarboxylate (3.0 g, 12.38 mmol) in DMF (61.9 ml) was treated with imidazole (1.26 g, 18.57 mmol) followed by the addition of TBSCl (2.05 g, 13.62 mmol) in DMF (61.9 ml). The reaction was stirred at room temperature overnight and then the reaction was diluted with EtOAc and washed with 5% LiCl (×2), brine, dried MgSO₄, filtered, and concentrated. The crude residue was purified by silica gel chromatography (0-30% EtOAc/Hexane) to give (±) trans-1,1-dimethylethyl 4-azido-3-{[(1,1-dimethylethyl)(dimethyl)silyl]oxy}-1-piperidinecarboxylate (3.29 g, 75%).

### Step B: (±) trans-1,1-dimethylethyl 4-amino-3-{[(1,1-dimethylethyl)(dimethyl)silyl]oxy}-1-piperidinecarboxylate

A solution of (±) trans-1,1-dimethylethyl 4-azido-3-{[(1,1-dimethylethyl)(dimethyl)silyl]oxy}-1-piperidinecarboxylate (3.29 g, 9.23 mmol) in MeOH (46.1 ml) was treated with 10% Pd/C (0.982 g, 0.92 mmol) and hydrogenated at 40 psi overnight. The catalyst was then filtered through GF/F and concentrated under reduced pressure to give (±) trans-1,1-dimethylethyl 4-amino-3-{[(1,1-dimethylethyl)(dimethyl)silyl]oxy}-1-piperidinecarboxylate which was used without further purification (2.88 g, 94%). ES-LCMS: 331.5 (M+1).

### Step C: (±) trans-1,1-dimethylethyl 4-[(4-chlorobutanoyl)amino]-3-{[(1,1-dimethylethyl)(dimethyl)silyl]oxy}-1-piperidinecarboxylate

A solution of (±) trans-1,1-dimethylethyl 4-amino-3-{[(1,1-dimethylethyl)(dimethyl)silyl]oxy}-1-piperidinecarboxylate (1.4 g, 4.24 mmol) and TEA (1.48 ml, 10.59 mmol) in DCM (21.18 ml) was treated by the drop wise addition of 4-chlorobutanoyl chloride (0.499 ml, 4.45 mmol). The mixture was stirred at room temperature for 2 hours. The reaction was diluted with DCM and washed with brine, dried MgSO₄, filtered, and concentrated to give (±) trans-1,1-dimethylethyl 4-[(4-chlorobutanoyl)amino]-3-{[(1,1-dimethylethyl)(dimethyl)silyl]oxy}-1-piperidinecarboxylate which was used without further purification (1.86 g, 100%). ES-LCMS: 435.4 (M+1).

### Step D: (±) trans-1,1-dimethylethyl 3-{[(1,1-dimethylethyl)(dimethyl)silyl]oxy}-4-(2-oxo-1-pyrrolidinyl)-1-piperidinecarboxylate

A solution of (±) trans-1,1-dimethylethyl 4-[(4-chlorobutanoyl)amino]-3-{[(1,1-dimethylethyl)(dimethyl)silyl]oxy}-1-piperidinecarboxylate (1.86 g, 4.28 mmol) in THF (21.38 ml) was cooled to 0° C. and treated by the portion wise addition of NaH (0.342 g, 8.55 mmol). The mixture was allowed to warm to room temperature and stirred for 1 hour. The mixture was then quenched by the addition of saturated NH₄Cl, diluted with water and extracted with EtOAc. The combined organics were washed with saturated NaHCO₃, brine, dried MgSO₄, filtered, and concentrated to give (±) trans-1,1-dimethylethyl 3-{[(1,1-dimethylethyl)(dimethyl)silyl]oxy}-4-(2-oxo-1-pyrrolidinyl)-1-piperidinecarboxylate which was used without further purification (1.66 g, 97%). ES-LCMS: 399.4 (M+1).

### Step E: (±) trans-1-(3-{[(1,1-dimethylethyl)(dimethyl)silyl]oxy}-4-piperidinyl)-2-pyrrolidinone TFA

A solution of (±) trans-1,1-dimethylethyl 3-{[(1,1-dimethylethyl)(dimethyl)silyl]oxy}-4-(2-oxo-1-pyrrolidinyl)-1-piperidinecarboxylate (0.200 g, 0.50 mmol) in DCM (2.51 ml) was treated by the addition of TFA (0.387 ml, 5.02 mmol). The reaction was stirred at room temperature for 2 hours. The reaction was concentrated to give (±) trans-1-(3-{[(1,1-dimethylethyl)(dimethyl)silyl]oxy}-4-piperidinyl)-2-pyrrolidinone TFA (0.200 g, 97%) which is used without further purification. ES-LCMS: 300.4 (M+1). Upon sitting at the bench (±) trans-1-(3-{[(1,1-dimethylethyl)(dimethyl)silyl]oxy}-4-piperidinyl)-2-pyrrolidinone TFA was converted to (±) trans-1-[3-hydroxy-4-piperidinyl]-2-pyrrolidinone TFA.

### Step F: (±) trans-1-(1-{[3-chloro-6-cyclopropyl-8-(trifluoromethyl)imidazo[1,2-a]pyridin-2-yl]carbonyl}-3-hydroxy-4-piperidinyl)-2-pyrrolidinone

A solution of (±) trans-1-[3-hydroxy-4-piperidinyl]-2-pyrrolidinone TFA (0.200 g, 0.485 mmol) and DIEA (0.212 ml, 1.21 mmol) in DMF (2.42 ml) was treated by the addition of 3-chloro-6-cyclopropyl-8-(trifluoromethyl)imidazo[1,2-a]pyridine-2-carboxylic acid (0.162 g, 0.53 mmol) and HATU (0.203 g, 0.53 mmol). The reaction was stirred at room temperature overnight and then diluted with EtOAc. The organics were washed with 5% LiCl (2×), saturated NaHCO₃, brine, dried MgSO₄, filtered, and concentrated. The crude residue was purified by silica gel chromatography (0-6% MeOH/DCM) to yield (±) trans-1-(1-{[3-chloro-6-cyclopropyl-8-(trifluoromethyl)imidazo[1,2-a]pyridin-2-yl]carbonyl}-3-hydroxy-4-piperidinyl)-2-pyrrolidinone (0.120 g, 52%). ES-LCMS: 471.4 (M+1).

### Example125

### (±) trans-1-(1-{[6-bromo-3-chloro-8-(trifluoromethyl)imidazo[1,2-a]pyridin-2-yl]carbonyl}-3-hydroxy-4-piperidinyl)-2-pyrrolidinone (Compound 226)

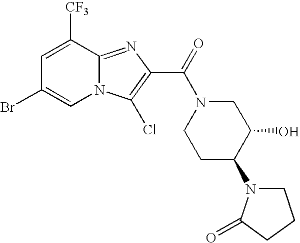

Step A: (±) trans-1-[3-hydroxy-4-piperidinyl]-2-pyrrolidinone TFA

A solution of (±) trans-1,1-dimethylethyl 3-{[(1,1-dimethylethyl)(dimethyl)silyl]oxy}-4-(2-oxo-1-pyrrolidinyl)-1-piperidinecarboxylate (1.32 g, 3.31 mmol) in DCM (16.56 ml) was treated by the addition of TFA (2.55 ml, 33.1 mmol) and stirred at room temperature for 3 hours. The reaction was concentrated however the reaction was not complete. The crude mixture was dissolved in DCE (17 mL) and treated with TFA (2.5 mL) and heated to 40° C. for 1 hour. The reaction was then concentrated to give (±) trans-143-hydroxy-4-piperidinyl]-2-pyrrolidinone TFA which was used without further purification. ES-LCMS: 185.2 (M+1).

### Step B: (±) trans-1-(1-{[6-bromo-3-chloro-8-(trifluoromethyl)imidazo[1,2-a]pyridin-2-yl]carbonyl}-3-hydroxy-4-piperidinyl)-2-pyrrolidinone

A solution of (±) trans-1-[3-hydroxy-4-piperidinyl]-2-pyrrolidinone TFA (0.160 g, 0.30 mmol) and DIEA (0.133 ml, 0.76 mmol) in DMF (1.52 ml) was treated by the addition of 6-bromo-3-chloro-8-(trifluoromethyl)imidazo[1,2-a]pyridine-2-carboxylic acid (0.115 g, 0.33 mmol) and HATU (0.127 g, 0.33 mmol). The reaction was stirred at room temperature overnight and then diluted with EtOAc. The organics were washed with 5% LiCl (2×), saturated NaHCO₃, brine, dried MgSO₄, filtered, and concentrated. The crude residue was purified by silica gel chromatography (0-6% MeOH/DCM) to give (±) trans-1-(1-{[6-bromo-3-chloro-8-(trifluoromethyl)imidazo[1,2-a]pyridin-2-yl]carbonyl}-3-hydroxy-4-piperidinyl)-2-pyrrolidinone (0.046 g, 30%). ES-LCMS: 511.2 (M+1).

### Example 126

### (±) trans-1-(1-{[3-chloro-6-(3-furanyl)-8-(trifluoromethyl)imidazo[1,2-a]pyridin-2-yl]carbonyl}-3-hydroxy-4-piperidinyl)-2-pyrrolidinone (compound 227)

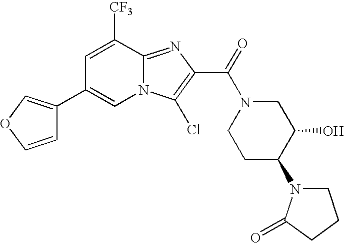

A solution of (±) trans-1-[3-hydroxy-4-piperidinyl]-2-pyrrolidinone TFA (0.160 g, 0.30 mmol) and DIEA (0.133 ml, 0.76 mmol) in DMF (1.52 ml) was treated by the addition of 3-chloro-6-(3-furanyl)-8-(trifluoromethyl)imidazo[1,2-a]pyridine-2-carboxylic acid (0.111 g, 0.33 mmol) and HATU (0.127 g, 0.33 mmol). The reaction was stirred at room temperature overnight and then diluted with EtOAc. The organics were washed with 5% LiCl (2×), saturated NaHC0₃, brine, dried MgSO₄, filtered, and concentrated. The crude residue was purified by silica gel chromatography (0-6% MeOH/DCM) to give (±) trans-1-(1-{[3-chloro-6-(3-furanyl)-8-(trifluoromethyl)imidazo[1,2-a]pyridin-2-yl]carbonyl}-3-hydroxy-4-piperidinyl)-2-pyrrolidinone (0.059 g, 39%). ES-LCMS: 497.2 (M+1).

### Example 127

### (±) cis-1-(1-{[3-chloro-6-cyclopropyl-8-(trifluoromethyl)imidazo[1,2-a]pyridin-2-yl]carbonyl}-3-hydroxy-4-piperidinyl)-2-pyrrolidinone (Compound 228)

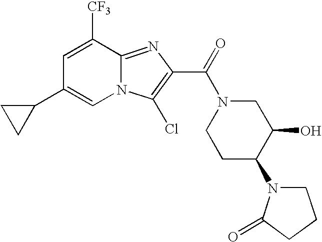

### Step A: (±) cis-4-chloro-N-[3-hydroxy-1-(phenylmethyl)-4-piperidinyl]butanamide

A solution of (±) cis-4-amino-1-(phenylmethyl)-3-piperidinol 2HCl (1.0 g, 3.58 mmol) and TEA (1.747 ml, 12.54 mmol) in DCM (17.91 ml) was treated by the drop wise addition of 4-chlorobutanoyl chloride (0.442 ml, 3.94 mmol). The mixture was stirred at room temperature for 2 hours. The reaction was diluted with DCM and washed with saturated NaHCO₃, brine, dried MgSO₄, filtered, and concentrated to give 4-chloro-N-[(3R,4S)-3-hydroxy-1-(phenylmethyl)-4-piperidinyl]butanamide (1.23 g, 110%) which was used without further purification. ES-LCMS: 313.3 (M+1).

### Step B: (±) cis-4-chloro-N-[3-{[(1,1-dimethylethyl)(dimethyl)silyl]oxy}-1-(phenylmethyl)-4-piperidinyl]butanamide

A solution of (±) cis-4-chloro-N-[3-{[(1,1-dimethylethyl)(dimethyl)silyl]oxy}-1-(phenylmethyl)-4-piperidinyl]butanamide (1.86 g, 4.38 mmol) in DMF (19.79 ml) was treated with imidazole (0.404 g, 5.94 mmol) followed by the addition of TBSCl (0.746 g, 4.95 mmol). The reaction was stirred at room temperature overnight and then the reaction was diluted with EtOAc and washed with 5% LiCl (×2), brine, dried MgSO₄, filtered, and concentrated to give (±) cis-4-chloro-N-[3-{[(1,1-dimethylethyl)(dimethyl)silyl]oxy}-1-(phenylmethyl)-4-piperidinyl]butanamide (1.86 g, 111%) which was used without further purification. ES-LCMS: 426.8 (M+1).

### Step C: (±) cis-1-[3-{[(1,1-dimethylethyl)(dimethyl)silyl]oxy}-1-(phenylmethyl)-4-piperidinyl]-2-pyrrolidinone

A solution of (±) cis-4-chloro-N-[3-{[(1,1-dimethylethyl)(dimethyl)silyl]oxy}-1-(phenylmethyl)-4-piperidinyl]butanamide (1.86 g, 4.38 mmol) in DMF (21.88 ml) was treated by the portion wise addition of 60% NaH (0.350 g, 8.75 mmol). The reaction was stirred at room temperature for 1 hour. The reaction was then quenched by the addition of saturated NH₄Cl and stirred over the weekend for convenience. The mixture was diluted with EtOAc and separated. The organic layer was washed with 5% LiCl (2×) and brine, dried MgSO₄, filtered, and concentrated to give (±) cis-1-[3-{[(1,1-dimethylethyl)(dimethyl)silyl]oxy}-1-(phenylmethyl)-4-piperidinyl]-2-pyrrolidinone (1.46g, 86%) which was used without further purification. ES-LCMS: 390.7 (M+1).

### Step D: (±) cis-1-(3-{[(1,1-dimethylethyl)(dimethyl)silyl]oxy}-4-piperidinyl)-2-pyrrolidinone

A solution of (±) cis-1-[3-{[(1,1-dimethylethyl)(dimethyl)silyl]oxy}-1-(phenylmethyl)-4-piperidinyl]-2-pyrrolidinone (1.58 g, 4.07 mmol) in EtOH (20.33 ml) was treated with 20% PdOH₂ (0.114 g, 0.163 mmol) and hydrogenated under 40 psi overnight. The reaction was purged with nitrogen and the catalyst was filtered using GF/F and the solvents were removed under reduced pressure to give (±) cis-1-(3-{[(1,1-dimethylethyl)(dimethyl)silyl]oxy}-4-piperidinyl)-2-pyrrolidinone (1.15 g, 95%) which is used without further purification. ES-LCMS: 301.3 (M+1).

### Step E: (±) cis-1-(3-{[(1,1-dimethylethyl)(dimethyl)silyl]oxy}-4-piperidinyl)-2-pyrrolidine TFA

A solution of (±) cis-1-(3-{[(1,1-dimethylethyl)(dimethyl)silyl]oxy}-4-piperidinyl)-2-pyrrolidinone (1.15 g, 3.85 mmol) in DCE (19.26 ml) was treated with TFA (2.97 ml, 38.5 mmol) and the reaction was heated to 40° C. for 3 hours. No reaction. The reaction was treated with MeOH (3 mL) and heated at 40° C. for 1 hour. No reaction. The mixture was concentrated to give (±) cis-1-(3-{[(1,1-dimethylethyl)(dimethyl)silyl]oxy}-4-piperidinyl)-2-pyrrolidinone TFA salt (1.849 g, 100%) which had a purple color and was used without further purification. ES-LCMS: 300.5 (M+1).

### Step F: (±) cis-1-(1-{[3-chloro-6-cyclopropyl-8-(trifluoromethyl)imidazo[1,2-a]pyridin-2-yl]carbonyl}-3-{[(1,1-dimethylethyl)(dimethyl)silyl]oxy}-4-piperidinyl)-2-pyrrolidinone

A solution of (±) cis-1-(3-{[(1,1-dimethylethyl)(dimethyl)silyl]oxy}-4-piperidinyl)-2-pyrrolidinone TFA (0.200 g, 0.34 mmol) in DMF (1.714 ml) was treated by the addition of DIEA (0.180 ml, 1.03 mmol), 3-chloro-6-cyclopropyl-8-(trifluoromethyl)imidazo[1,2-a]pyridine-2-carboxylic acid (0.115 g, 0.38 mmol), and HATU (0.143 g, 0.38 mmol). The reaction was stirred at room temperature overnight. The mixture was diluted with EtOAc, washed with 5% LiCl (2×), saturated NaHCO₃, brine, dried MgSO₄, filtered, and concentrated to give (±) cis-1-(1-{[3-chloro-6-cyclopropyl-8-(trifluoromethyl)imidazo[1,2-a]pyridin-2-yl]carbonyl}-3-{[(1,1-dimethylethyl)(dimethyl)silyl]oxy}-4-piperidinyl)-2-pyrrolidinone (0.204 g, 100%) which was used without further purification. ES-LCMS: 585.3 (M+1).

### Step G: (±) cis-1-(1-{[3-chloro-6-cyclopropyl-8-(trifluoromethyl)imidazo[1,2-a]pyridin-2-yl]carbonyl}-3-hydroxy-4-piperidinyl)-2-pyrrolidinone

A solution of (±) cis-1-(1-{[3-chloro-6-cyclopropyl-8-(trifluoromethyl)imidazo[1,2-a]pyridin-2-yl]carbonyl}-3-{[(1,1-dimethylethyl)(dimethyl)silyl]oxy}-4-piperidinyl)-2-pyrrolidinone (0.154 g, 0.26 mmol) in THF (1.32 ml) was treated with 1M in THF TBAF (0.40 ml, 0.40 mmol). The reaction was stirred at room temperature for 4 hours and concentrated under reduced pressure. The crude residue was purified by column chromatography (1-10% MeOH/DCM) to give (±) cis-1-(1-{[3-chloro-6-cyclopropyl-8-(trifluoromethyl)imidazo[1,2-a]pyridin-2-yl]carbonyl}-3-hydroxy-4-piperidinyl)-2-pyrrolidinone. ES-LCMS: 471.3 (M+1).

### Example 128

### (±) cis-1-(1-{[6-bromo-3-chloro-8-(trifluoromethyl)imidazo[1,2-a]pyridin-2-yl]carbonyl}-3-hydroxy-4-piperidinyl)-2-pyrrolidinone (Compound 229)

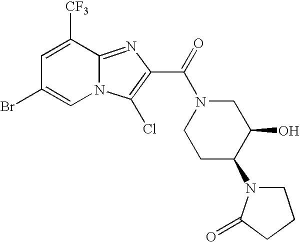

### Step A: (±) cis-1-(1-{[6-bromo-3-chloro-8-(trifluoromethyl)imidazo[1,2-a]pyridin-2-yl]carbonyl}-3-{[(1,1-dimethylethyl)(dimethyl)silyl]oxy}-4-piperidinyl)-2-pyrrolidinone

A solution of (±) cis-1-(3-{[(1,1-dimethylethyl)(dimethyl)silyl]oxy}-4-piperidinyl)-2-pyrrolidinone TFA (0.200 g, 0.34 mmol) in DMF (1.71 ml) was treated by the addition of DIEA (0.180 ml, 1.03 mmol), 6-bromo-3-chloro-8-(trifluoromethyl)imidazo[1,2-a]pyridine-2-carboxylic acid (0.129 g, 0.38 mmol), and HATU (0.143 g, 0.38 mmol). The reaction was stirred at room temperature overnight. The mixture was diluted with EtOAc, washed with 5% LiCl (2×), saturated NaHCO₃, brine, dried MgSO₄, filtered, and concentrated to give (±) cis-1-(1-{[6-bromo-3-chloro-8-(trifluoromethyl)imidazo[1,2-a]pyridin-2-yl]carbonyl}-3-{[(1,1-dimethylethyl)(dimethyl)silyl]oxy}-4-piperidinyl)-2-pyrrolidinone (0.205 g, 96%) which was used without further purification. ES-LCMS: 625.2 (M+1).

### Step B: (±) cis-1-(1-{[6-bromo-3-chloro-8-(trifluoromethyl)imidazo[1,2-a]pyridin-2-yl]carbonyl}-3-hydroxy-4-piperidinyl)-2-pyrrolidinone

A solution of (±) cis-1-(1-{[6-bromo-3-chloro-8-(trifluoromethyl)imidazo[1,2-a]pyridin-2-yl]carbonyl}-3-{[(1,1-dimethylethyl)(dimethyl)silyl]oxy}-4-piperidinyl)-2-pyrrolidinone (0.205 g, 0.33 mmol) in THF (1.64 ml) was treated with 1M in THF TBAF (0.49 ml, 0.49 mmol). The reaction was stirred at room temperature for 1 hour and concentrated under reduced pressure. The crude residue was purified by silica gel chromatography (1-10% MeOH/DCM) to give (±) cis-1-(1-{[6-bromo-3-chloro-8-(trifluoromethylimidazo[1,2-a]pyridin-2-yl]carbonyl}-3-hydroxy-4-piperidinyl)-2-pyrrolidinone (0.035 g, 21%). ES-LCMS: 511.1 (M+1).

### Example 129

### (±) cis-1-(1-{[3-chloro-6-(3-furanyl)-8-(trifluoromethyl)imidazo[1,2-a]pyridin-2-yl]carbonyl}-3-hydroxy-4-piperidinyl)-2-pyrrolidinone (Compound 230)

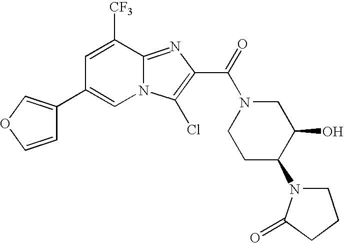

### Step A: (±) cis-1-[3-hydroxy-4-piperidinyl]-2-pyrrolidinone TFA

A solution of (±) cis-1-(3-{[(1,1-dimethylethyl)(dimethyl)silyl]oxy}-4-piperidinyl)-2-pyrrolidinone (1.15 g, 3.85 mmol) in DCE (19.26 ml) was treated with TFA (2.97 ml, 38.5 mmol) and the reaction was heated to 40° C. for 3 hours. No reaction, so the reaction was treated with MeOH (3 mL) and heated at 40° C. for 1 hour. The reaction was concentrated to give (±) cis-1-[3-hydroxy-4-piperidinyl]-2-pyrrolidinone TFA (1.849 g, 100%) which had a purple color and was used without further purification. ES-LCMS: 300.5 (M+1).

### Step B: (±) cis-1-(1-{[3-chloro-6-(3-furanyl)-8-(trifluoromethyl)imidazo[1,2-a]pyridin-2-yl]carbonyl}-3-{[(1,1-dimethylethyl)(dimethyl)silyl]oxy}-4-piperidinyl)-2-pyrrolidinone

A solution of (±) cis-1-(3-{[(1,1-dimethylethyl)(dimethyl)silyl]oxy}-4-piperidinyl)-2-pyrrolidinone TFA (0.200 g, 0.34 mmol) in DMF (1.71 ml) was treated by the addition of DIEA (0.180 ml, 1.03 mmol), 3-chloro-6-(3-furanyl)-8-(trifluoromethyl)imidazo[1,2-a]pyridine-2-carboxylic acid (0.125 g, 0.38 mmol), and HATU (0.143 g, 0.38 mmol). The reaction was stirred at room temperature overnight. The mixture was diluted with EtOAc, washed with 5% LiCl (2×), saturated NaHCO₃, brine, dried MgSO₄, filtered, and concentrated to give (±) cis-1-(1-{[3-chloro-6-(3-furanyl)-8-(trifluoromethyl)imidazo[1,2-a]pyridin-2-yl]carbonyl}-3-{[(1,1-dimethylethyl)(dimethyl)silyl]oxy}-4-piperidinyl)-2-pyrrolidinone (0.204 g, 97%) which was used without further purification. ES-LCMS: 611.3 (M+1).

### Step C: (±) cis-1-(1-{[3-chloro-6-(3-furanyl)-8-(trifluoromethyl)imidazo[1,2-a]pyridin-2-yl]carbonyl}-3-hydroxy-4-piperidinyl)-2-pyrrolidinone

A solution of (±) cis-1-(1-{[3-chloro-6-(3-furanyl)-8-(trifluoromethyl)imidazo[1,2-a]pyridin-2-yl]carbonyl}-3-{[(1,1-dimethylethyl)(dimethyl)silyl]oxy}-4-piperidinyl)-2-pyrrolidinone (0.204 g 0.334 mmol) in THF (1.67 ml) treated with 1M in THF TBAF (0.51 ml, 0.50 mmol). The reaction was stirred at room temperature for 5 hour and concentrated under reduced pressure. The crude residue was purified by silica gel chromatography to give (±) cis-1-(1-{[3-chloro-6-(3-furanyl)-8-(trifluoromethyl)imidazo[1,2-a]pyridin-2-yl]carbonyl}-3-hydroxy-4-piperidinyl)-pyrrolidinone (0.058 g, 35%). ES-LCMS: 497.2 (M+1).

### Example 130

### (±) cis-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Compound 231)

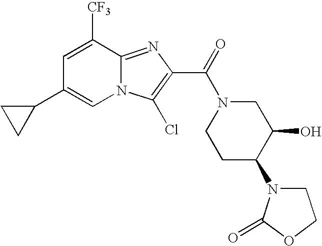

### Step A: (±) cis-2-chloroethyl [3-hydroxy-1-(phenylmethyl)-4-piperidinyl]carbamate

A solution of (±) cis-4-amino-1-(phenylmethyl)-3-piperidinol 2HCl (.700 g, 2.507 mmol) and DIEA (1.53 ml, 8.77 mmol) in DCM (12.54 ml) was cooled to 0° C. and treated by the drop wise addition of 2-chloroethyl chloridocarbonate (0.285 ml, 2.76 mmol). The mixture was stirred at 0° C. for 2 hours. The reaction was diluted with DCM and washed with saturated NaHCO₃, brine, dried MgSO₄, filtered, and concentrated to give (±) cis-2-chloroethyl [3-hydroxy-1-(phenylmethyl)-4-piperidinyl]carbamate (0.847 g, 111%).

### Step B: (±) cis-2-chloroethyl [3-{[(1,1-dimethylethyl)(dimethyl)silyl]oxy}-1-(phenylmethyl)-4-piperidinyl]carbamate

A solution of (±) cis-2-chloroethyl [3-hydroxy-1-(phenylmethyl)-4-piperidinyl]carbamate (0.874 g, 2.79 mmol) in DMF (13.97 ml) was treated with imidazole (0.285 g, 4.19 mmol) followed by the addition of TBSCl (0.526 g, 3.49 mmol). The reaction was stirred at room temperature overnight. The reaction was treated by the addition of 0.25 equivalents TBSCl and 0.30 equivalents imidazole and the reaction was stirred overnight. The reaction was diluted with EtOAc and washed with 5% LiCl (×2), brine, dried MgSO₄, filtered, and concentrated to give (±) cis-2-chloroethyl [3-{[(1,1-dimethylethyl)(dimethyl)silyl]oxy}-1-(phenylmethyl)-4-piperidinyl]carbamate (0.947 g, 79%) which was used without further purification. ES-LCMS: 428.7 (M+1).

### Step C: (±) cis-3-[3-{[(1,1-dimethylethyl)(dimethyl)silyl]oxy}-1-(phenylmethyl)-4-piperidinyl]-1,3-oxazolidin-2-one

A solution of (±) cis-2-chloroethyl [3-{[(1,1-dimethylethyl)(dimethyl)silyl]oxy}-1-(phenylmethyl)-4-piperidinyl]carbamate (0.947 g, 2.218 mmol) in DMF (11.09 ml) was stirred at room temperature and treated by the portion wise addition of NaH (0.177 g, 4.44 mmol). The mixture was stirred at room temperature for 2 hours and then quenched by the addition of saturated NH₄Cl. The reaction was extracted with EtOAc, washed with 5% LiCl (2×), brine, dried MgSO₄, filtered, and concentrated to give (±) cis-3-[3-{[(1,1-dimethylethyl)(dimethyl)silyl]oxy}-1-(phenylmethyl)-4-piperidinyl]-1,3-oxazolidin-2-one (0.605 g, 70%) which was used without further purification. ES-LCMS: 392.6 (M+1).

### Step D: (±) cis-3-(3-{[(1,1-dimethylethyl)(dimethyl)silyl]oxy}-4-piperidinyl)-1,3-oxazolidin-2-one

A solution of (±) cis-3-[3-{[(1,1-dimethylethyl)(dimethyl)silyl]oxy}-1-(phenylmethyl)-4-piperidinyl]-1,3-oxazolidin-2-one (0.300 g, 0.77 mmol) in EtOH (3.84 ml) was treated with 20% PdOH₂ (0.114 g, 0.16 mmol) and hydrogenated under 40 psi overnight. The reaction was purged with nitrogen and the catalyst was filtered using GF/F and the solvents were removed under reduced pressure to give (±) cis-3-(3-{[(1,1-dimethylethyl)(dimethyl)silyl]oxy}-4-piperidinyl)-1,3-oxazolidin-2-one (0.250 g, 100%). ES-LCMS: 302.5 (M+1).

### Step E: (±) cis-3-[3-hydroxy-4-piperidinyl]-1,3-oxazolidin-2-one HCl

A solution of (±) cis-3-(3-{[(1,1-dimethylethyl)(dimethyl)silyl]oxy}-4-piperidinyl)-1,3-oxazolidin-2-one (0.125 g, 0.416 mmol) in DCM (2 mL) was treated by the addition of 4N HCl in dioxane (0.5 mL). The reaction was stirred at room temperature for 6 hours and concentrated. The reaction was not complete and was treated with DCM (2 mL) and 4N HCl in dioxanes (0.5 mL) and stirred at room temperature overnight. The reaction is concentrated to give (±) cis-3-[3-hydroxy-4-piperidinyl]-1,3-oxazolidin-2-one HCl (0.106 g, 114%) which was used without further purification.

### Step F: (±) cis-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one

A solution of (±) cis-3-[3-hydroxy-4-piperidinyl]-1,3-oxazolidin-2-one HCl (0.041 g, 0.184 mmol) in DMF (0.92 ml) was treated by the addition of DIEA (0.080 ml, 0.46 mmol), followed by 3-chloro-6-cyclopropyl-8-(trifluoromethyl)imidazo[1,2-a]pyridine-2-carboxylic acid (0.062 g, 0.20 mmol), and HATU (0.077 g, 0.20 mmol). The reaction was stirred at room temperature for 3 hours. The reaction was diluted with EtOAc, washed with 5% LiCl, brine, dried MgSO₄, filtered, and concentrated. The crude residue was purified by silica gel chromatography (1-10% MeOH/DCM) to give compound with unacceptable purity. The residue was repurified by silica gel chromatography (1-5% MeOH/DCM) to give (±) cis-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one. ES-LCMS: 473.2 (M+1).

### Example 131

### 1-{[3-chloro-6-cyclopropyl-8-(trifluoromethyl)imidazo[1,2-a]pyridin-2-yl]carbonyl}-4-(2-oxo-1,3-oxazolidin-3-yl)-3-piperidinone (compound 232)

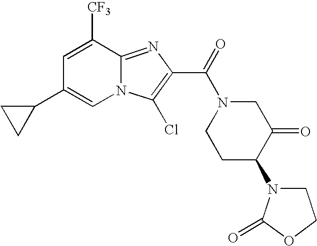

### Step A: (±) trans-1,1-dimethylethyl 4-amino-3-hydroxy-1-piperidinecarboxylate

A solution of (±) trans-1,1-dimethylethyl 4-azido-3-hydroxy-1-piperidinecarboxylate (4.0 g, 16.51 mmol) in methanol (83 ml) was treated with 10% Pd/C (1.757 g, 1.651 mmol) and hydrogenated at 40 psi H2 overnight. The catalyst was filtered through celite then GF/F, washing with MeOH. The solvent was removed under reduced pressure to yield pure (±) trans-1,1-dimethylethyl 4-amino-3-hydroxy-1-piperidinecarboxylate.

### Step B: (±) trans-1,1-dimethylethyl 4-({[(2-chloroethyl)oxy]carbonyl}amino)-3-hydroxy-1-piperidinecarboxylate

A solution of (±) trans-1,1-dimethylethyl 4-amino-3-hydroxy-1-piperidinecarboxylate (2.0 g, 9.25 mmol) and DIEA (4.04 ml, 23.12 mmol) in DCM (46.2 ml) in was cooled to 0° C. The reaction was treated with 2-chloroethyl chloridocarbonate (1.052 ml, 10.17 mmol) and stirred at 0° C. for 2 hours. The mixture was diluted with DCM and washed with saturated NaHCO₃. The combined extracts were washed with brine, dried Na₂SO₄, filtered, and concentrated to give (±) trans-1,1-dimethylethyl 4-({[(2-chloroethyl)oxy]carbonyllamino)-3-hydroxy-1-piperidinecarboxylate (3.6 g. 121%). ES-LCMS: 323.2 (M+1-BOC).

### Step C: (±) trans-1,1-dimethylethyl 3-hydroxy-4-(2-oxo-1,3-oxazolidin-3-yl)-1-piperidinecarboxylate

A solution of (±) trans-1,1-dimethylethyl 4-({[(2-chloroethyl)oxy]carbonyl}amino)-3-hydroxy-1-piperidinecarboxylate (2.98 g, 9.23 mmol) in DMF (46.2 ml) was stirred at room temperature and treated by the portion wise addition of NaH (0.739 g, 18.46 mmol). The mixture was stirred at room temperature for 2 hours and then quenched by the addition of saturated NH₄Cl. The reaction was extracted with EtOAc, washed with brine, dried Na₂SO₄, filtered, and concentrated to give (±) trans-1,1-dimethylethyl 3-hydroxy-4-(2-oxo-1,3-oxazolidin-3-yl)-1-piperidinecarboxylate. ES-LCMS: 287.2 (M+1-BOC).

### Step D: 1,1-dimethylethyl 3-oxo-4-(2-oxo-1,3-oxazolidin-3-yl)-1-piperidinecarboxylate

A −78° C. solution of 2M in DCM oxalyl chloride (0.699 ml, 1.397 mmol) in DCM (2.328 ml) was treated by the addition of DMSO (0.173 ml, 2.445 mmol). After stirring for 10 min, a solution of (±) trans-1,1-dimethylethyl 3-hydroxy-4-(2-oxo-1,3-oxazolidin-3-yl)-1-piperidinecarboxylate (0.200 g, 0.699 mmol) in DCM (1.164 ml) was added drop wise and the reaction mixture was stirred at −78° C. for 30 minutes. The reaction was then treated by the addition of TEA (0.487 ml, 3.49 mmol) and then allowed to warm to room temperature and stirred overnight. The reaction was then diluted with water and extracted with DCM. The combined organics were washed with brine, dried MgSO4, filtered, and concentrated to give 1,1-dimethylethyl 3-oxo-4-(2-oxo-1,3-oxazolidin-3-yl)-1-piperidinecarboxylate (0.187 g, 94%). ES-LCMS: 185.2 (M+1-BOC).

### Step E: 4-(2-oxo-1,3-oxazolidin-3-yl)-3-piperidinone TFA

A solution of 1,1-dimethylethyl 3-oxo-4-(2-oxo-1,3-oxazolidin-3-yl)-1-piperidinecarboxylate (0.187 g, 0.658 mmol) in DCM (3.29 ml) was treated by the addition of TFA (0.507 ml, 6.58 mmol). The reaction was stirred at room temperature for 3 hours and the reaction was concentrated to give 4-(2-oxo-1,3-oxazolidin-3-yl)-3-piperidinone TFA (0.196 g, 100%). ES-LCMS: 185.1 (M+1).

### Step F: 1-{[3-chloro-6-cyclopropyl-8-(trifluoromethyl)imidazo[1,2-a]pyridin-2-yl]carbonyl}-4-(2-oxo-1,3-oxazolidin-3-yl)-3-piperidinone

A solution of 4-(2-oxo-1,3-oxazolidin-3-yl)-3-piperidinone TFA (0.196 g, 0.657 mmol) and 3-chloro-6-cyclopropyl-8-(trifluoromethyl)imidazo[1,2-a]pyridine-2-carboxylic acid (0.220 g, 0.723 mmol) in DMF (3.29 ml) was treated by the addition of DIEA (0.287 ml, 1.643 mmol) and HATU (0.275 g, 0.723 mmol). The reaction was stirred at room temperature overnight. The mixture was diluted with EtOAc and washed with 5% LiCl, saturated NaHCO₃, brine, dried MgSO₄, filtered, and concentrated. The crude residue was purified by silica gel chromatography (0-5% MeOH/DCM) to give the desired product (0.033 g). The residue was repurified by reverse phase chromatography to give 1-{[3-chloro-6-cyclopropyl-8-(trifluoromethyl)imidazo[1,2-a]pyridin-2-yl]carbonyl}-4-(2-oxo-1,3-oxazolidin-3-yl)-3-piperidinone (0.006 g, 2%). 471.3 (M+1).

### Example 132

### (±) cis-3-(1-{[6-bromo-3-chloro-8-(trifluoromethyl)imidazo[1,2-a]pyridin-2-yl]carbonyl}-3-hydroxy-4-piperidinyl)-1,3-oxazolidin-2-one (compound 233)

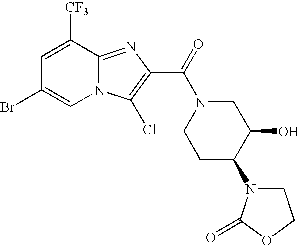

A solution of (±) cis-3-[3-hydroxy-4-piperidinyl]-1,3-oxazolidin-2-one HCl (0.041 g, 0.18 mmol), 6-bromo-3-chloro-8-(trifluoromethyl)imidazo[1,2-a]pyridine-2-carboxylic acid (0.070 g, 0.20 mmol), HATU (0.077 g, 0.20 mmol), and DIEA (0.096 ml, 0.55 mmol) in DMF (0.921 ml) was stirred at room temperature for 2 hours. The reaction was diluted with EtOAc, washed with 5% LiCl, saturated NaHCO₃, brine, dried MgSO₄, filtered, and concentrated. The crude residue was purified by silica gel chromatography (0-5% MeOH/DCM) to give (±) cis-3-(1-{[6-bromo-3-chloro-8-(trifluoromethyl)imidazo[1,2-a]pyridin-2-yl]carbonyl}-3-hydroxy-4-piperidinyl)-1,3-oxazo one (0.037 g, 39%). ES-LCMS: 513.1 (M+1).

### Example 133

### 3-((cis)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 1) (compound 234)

### Example 134

### 3-((cis)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 2) (compound 235)

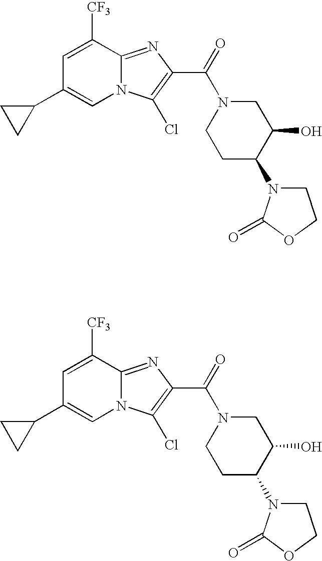

Separation of (±) cis-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one was performed on 30×250 mm column from ES Industries at 40 mL/min using 50% EtOH in hexanes. UV was monitored at 254 nm to afford a first eluting isomer (35 mg, >99% ee, ES-LCMS: 473.2 (M+1)) and a second eluting isomer (30 mg, >99% ee, ES-LCMS: 473.3 (M+1)). Absolute stereochemical assignments were not made.

### Example 135

### (±) trans-3-[1-{[3-chloro-6-(3-furanyl)-8-(trifluoromethyl)imidazo[1,2-a]pyridin-2-yl]carbonyl}-3-(methyloxy)-4-piperidinyl]-1,3-oxazolidin-2-one (compound 236)

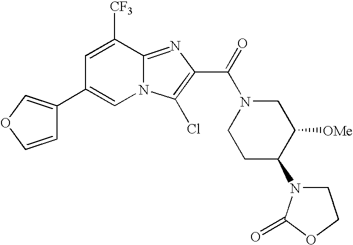

### Step A: (±) trans-1,1-dimethylethyl 3-(methyloxy)-4-(2-oxo-1,3-oxazolidin-3-yl)-1-piperidinecarboxylate

A solution of (±) trans-1,1-dimethylethyl 3-hydroxy-4-(2-oxo-1,3-oxazolidin-3-yl)-1-piperidinecarboxylate (0.150 g, 0.524 mmol) and Mel (0.098 ml, 1.57 mmol) in THF (2.62 ml) under N₂ was treated by the portion wise addition of NaH (0.025 g, 0.629 mmol). The reaction was stirred at room temperature for 1.5 hours. The reaction was quenched by the addition of water and the aqueous solution was extracted with EtOAc. The combined organics were washed with brine, dried MgSO₄, filtered, and concentrated to give (±) trans-1,1-dimethylethyl 3-(methyloxy)-4-(2-oxo-1,3-oxazolidin-3-yl)-1-piperidinecarboxylate (0.134 g, 85%) which was used without further purification. ES-LCMS: 301.3 (M+1).

### Step B: (±) trans-3-[3-(methyloxy)-4-piperidinyl]-1,3-oxazolidin-2-one TFA

A solution of (±) trans-1,1-dimethylethyl 3-(methyloxy)-4-(2-oxo-1,3-oxazolidin-3-yl)-1-piperidinecarboxylate (0.134 g, 0.45 mmol) in DCM (2.23 ml) was treated with TFA (0.344 ml, 4.46 mmol). The reaction was stirred at room temperature for 2 hours and concentrated under reduced pressure to give (±) trans-3-[3-(methyloxy)-4-piperidinyl]-1,3-oxazolidin-2-one TFA (0.186 g, 100%). ES-LCMS: 201.4 (M+1).

### Step C: (±) trans-3-1-{[3-chloro-6-(3-furanyl)-8-(trifluoromethyl)imidazo[1,2-a]pyridin-2-yl]carbonyl}-3-(methyloxy)-4-piperidinyl]-1,3-oxazolidin-2-one

A solution of (±) trans-3[3-(methyloxy)-4-piperidinyl]-1,3-oxazolidin-2-one TFA (0.140 g, 0.46 mmol) and 3-chloro-6-(3-furanyl)-8-(trifluoromethyl)imidazo[1,2-a]pyridine-2-carboxylic acid (0.162 g, 0.49 mmol) in DMF (2.23 ml) was treated by the addition of DIEA (0.233 ml, 1.34 mmol) and HATU (0.186 g, 0.49 mmol). The reaction was stirred at room temperature for 2 hours and diluted with EtOAc and washed with 5% LiCl (2×), brine, dried MgSO₄, filtered, and concentrated. The crude residue was purified by silica gel chromatography (1-5% MeOH/DCM) to give (±) trans-3-[1-{[3-chloro-6-(3-furanyl)-8-(trifluoromethyl)imidazo[1,2-a]pyridin-2-yl]carbonyl}-3-(methyloxy)-4-piperidinyl]-1,3-oxazolidin-2-one (0.093 g, 41%). ES-LCMS: 513.2 (M+1).

### Example 136

### (±) trans-3-[1-{[6-bromo-3-chloro-8-(trifluoromethyl)imidazo[1,2-a]pyridin-2-yl]carbonyl}-3-(methyloxy)-4-piperidinyl]-1,3-oxazolidin-2-one (Compound 137)

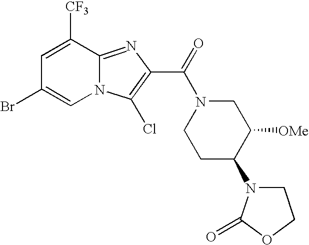

A solution of (±) trans-3-[3-(methyloxy)-4-piperidinyl]-1,3-oxazolidin-2-one TFA (0.138 g, 0.44 mmol) and 6-bromo-3-chloro-8-(trifluoromethyl)imidazo[1,2-a]pyridine-2-carboxylic acid (0.166 g, 0.48 mmol) in DMF (2.20 ml) was treated by the addition of DIEA (0.230 ml, 1.32 mmol) and HATU (0.184 g, 0.48 mmol). The reaction was stirred at room temperature for 2 hours and diluted with EtOAc and washed with 5% LiCl (2×), brine, dried MgSO₄, filtered, and concentrated. The crude residue was purified by silica gel chromatography (1-4% MeOH/DCM) to give (±) trans-3-[1-{[6-bromo-3-chloro-8-(trifluoromethylimidazo[1,2-a]pyridin-2-yl]carbonyl}-3-(methyloxy)-4-piperidinyl]-1,3-oxazolidin-2-one (0.08 g, 35%). ES-LCMS: 527.1 (M+1).

### Example 137

### (±) trans-3-[1-{[3-chloro-6-cyclopropyl-8-(trifluoromethyl)imidazo[1,2-a]pyridin-2-yl]carbonyl}-3-(methyloxy)-4-piperidinyl]-1,3-oxazolidin-2-one (Compound 238)

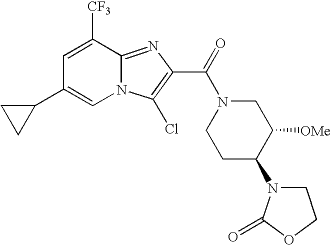

A solution of (±) trans-3-[3-(methyloxy)-4-piperidinyl]-1,3-oxazolidin-2-one TFA (0.138 g, 0.44 mmol) and 3-chloro-6-cyclopropyl-8-(trifluoromethyl)imidazo[1,2-a]pyridine-2-carboxylic acid (0.147 g, 0.48 mmol) in DMF (2.20 ml) was treated by the addition of DIEA (0.230 ml, 1.32 mmol) and HATU (0.184 g, 0.48 mmol). The reaction was stirred at room temperature for 2 hours and diluted with EtOAc and washed with 5% LiCl (2×), brine, dried MgSO₄, filtered, and concentrated. The crude residue was purified by silica gel chromatography (1-4% MeOH/DCM) to give (±) trans-341-{[3-chloro-6-cyclopropyl-8-(trifluoromethyl)imidazo[1,2-a]pyridin-2-yl]carbonyl}-3-(methyloxy)-4-piperidinyl]-1,3-oxazolidin-2-one (0.082 g, 38%). ES-LCMS: 487.5 (M+1).

### Example 138

### 3-chloro-2-{[4-(2-oxo-1,3-oxazolidin-3-yl)-1-piperidinyl]carbonyl}-8-(trifluoromethyl)imidazo[1,2-a]pyridine-6-carbonitrile (Compound 239)

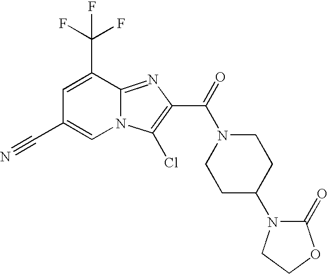

A solution of 3-(1-{[6-bromo-3-chloro-8-(trifluoromethyl)imidazo[1,2-a]pyridin-2-yl]carbonyl}-4-piperidinyl)-1,3-oxazolidin-2-one (217 mg, 0.438 mmol), zinc cyanide (27.7 mg, 0.263 mmol) and Pd(Ph₃P)₄ (25.3 mg, 0.022 mmol) in DMF (3 mL) was irradiated in a personal microwave reactor for 30 minutes at 160° C. The solvent was removed and the crude material was purified via reverse phase HPLC to give 3-chloro-2-{[4-(2-oxo-1,3-oxazolidin-3-yl)-1-piperidinyl]carbonyl}-8-(trifluoromethyl)imidazo[1,2-a]pyridine-6-carbonitrile (17 mg, 0.035 mmol, 8% yield). ES-LCMS: 442.2 (M+1).

### Biological Examples

### Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture was disclosed in U.S. Pat. No. 5,738,985 to Miles et al. In vitro assays have been reported in Ferrari et al. Jnl. of Vir., 73:1649-1654, (1999); Ishii et al., Hepatology, 29:1227-1235, (1999); Lohmann et al., J. Biol. Chem., 274:10807-10815, (1999); and Yamashita et al., J. Biol. Chem., 273:15479-15486, (1998).

### Example 2

### Replicon Assay

Compounds were assayed for activity against HCV using the genotype 1a and 1b subgenomic replicon model systems. Stable cell lines bearing the genotype 1a and 1b replicons were used for screening of compounds. Both replicons are bicistonic and contain the firefly luciferase gene. The ET cell line is stably transfected with RNA transcripts harboring a I₃₈₉luc-ubi-neo/NS3-3′/ET replicon with firefly luciferase-ubiquitin-neomycin phosphotransferase fusion protein and EMCV-IRES driven NS3-5B polyprotein containing the cell culture adaptive mutations (E1202G; T12801; K1846T) (Krieger at al, 2001 and unpublished). The genotype 1a replicon is a stable cell line licensed from Apath LLC, modified to contain the firefly luciferase gene. The cells were grown in DMEM, supplemented with 10% fetal calf serum, 2 mM Glutamine, Penicillin (100 IU/mL)/Streptomycin (100 μg/mL), 1× nonessential amino acids, and 250-500 μg/mL G418 (“Geneticin”). They were all available through Life Technologies (Bethesda, Md.). The cells were plated at 0.5×10⁴ cells/well in 384 well plates containing compounds. The final concentration of compounds ranged between 0.03 μM to 50 μm and the final DMSO concentration of 0.5-1%.

Luciferase activity was measured 48 hours later by adding a Steady glo (Promega, Madison, Wis.). Percent inhibition of replication data was plotted relative to no compound control. Under the same condition, cytotoxicity of the compounds was determined using cell titer glo (Promega, Madison, Wis). IC50s were determined from a 10 point dose response curve using 3-4-fold serial dilution for each compound, which spans a concentration range >1000 fold. BioAssay determines the level of inhibition for each compound by normalizing cross-talk corrected plate values against the negative (low or background, cells with no compound present) and positive (high DMSO, no cells) controls to determine Percent Inhibition:

$$\frac{100 * ( 1 - ( \begin{matrix}Cross \text{-} talk corrected value - \\ Compound Positive Control Mean\end{matrix} ) )}{\begin{matrix}DMSO Negative Control Mean - \\ Compound Positive Control Mean\end{matrix}}$$

These normalized values are exported to EC₅₀ where they are plotted against the molar compound concentrations using the standard four parameter logistic equation:

$$y = A + \frac{B - A}{1 + {[ \frac{{10}^{X}}{{10}^{c}} ]}^{D}}$$

Where:

A=minimum y

B=maximum y

C=log₁₀EC₅₀

D=slope factor

x=log₁₀ compound concentration [M]

As shown below, the tested compounds tested were found to inhibit the activity of the replicon with EC₅₀ values of about 10,000 nM or less. Preferably, the compounds will exhibit EC₅₀ values of about 500 nM or less, in some embodiments about 100 or less, and in some embodiments, about 40 or less, and in some embodiments 10 or less, and in some embodiments 5 or less, and in some embodiments, 1 or less. Further, compounds of the present disclosure, which were tested against more than one genotype of HCV replicon, were found to have similar inhibitory properties.

Percent inhibition values at a specific concentration, 10 μM for example, can also be derived from the equation above. In some aspects, the compounds of Formula (I) will exhibit a % inhibition of at least 80% at 10 μM. In other aspects, the % inhibition is at least 50% at 10 μM. In other aspects, the % inhibition is at least 10% at 10 μM.

The compounds of Formula (I) are expected to have improved activity and/or solubility due to the cyclic 5-membered oxo substituted moiety attached the the pyrrolidinyl ring.

When tested, certain compounds of Table 1 were found to have EC₅₀ values listed in Table 2 and Table 3 below. Some EC₅₀ measurements were repeated a second time and for both 1A and 1B genotypes after those reported in Table 2 for compounds 101-108 and the repeated data was reported in Table 3.

TABLE 2

| Compound No. | EC₅₀ (μM) |
|---|---|
| 101 | 0.057 |
| 102 | 0.683 |
| 103 | 0.026 |
| 104 | 0.283 |
| 105 | 0.071 |
| 106 | 0.005 |
| 107 | 0.016 |
| 108 | 0.213 |

TABLE 3

| | HCV Genotype | HCV Genotype |
|---|---|---|
| Compound | 1A | 1B |
| Number | Replicon | Replicon |
| (From Table 1) | EC₅₀ (μM) | EC₅₀ (μM) |
| 101 | — | — |
| 102 | — | — |
| 103 | 0.01 | 0.02 |
| 104 | — | — |
| 105 | 0.03 | 0.04 |
| 106 | 0.01 | 0.02 |
| 107 | — | — |
| 108 | 0.02 | 0.13 |
| 109 | 38.02 | 28.18 |
| 110 | 1.74 | 0.80 |
| 111 | 5.01 | 11.75 |
| 112 | 0.13 | 0.17 |
| 113 | 0.74 | 9.66 |
| 114 | 0.03 | 0.31 |
| 115 | 0.06 | 3.69 |
| 116 | 0.36 | 7.41 |
| 117 | 0.47 | 2.54 |
| 118 | 0.15 | 17.58 |
| 119 | 0.01 | 0.05 |
| 120 | 0.29 | 36.03 |
| 121 | 0.44 | 2.85 |
| 122 | 2.34 | 15.85 |
| 123 | 0.03 | 0.38 |
| 124 | 0.03 | 3.47 |
| 125 | 0.09 | 13.34 |
| 126 | 13.65 | 50.12 |
| 127 | 0.01 | 0.17 |
| 128 | 0.01 | 0.03 |
| 129 | 0.02 | 0.12 |
| 130 | 0.02 | 0.05 |
| 131 | 0.01 | 0.70 |
| 132 | 0.13 | 0.21 |
| 133 | 0.11 | 3.72 |
| 134 | 0.32 | 2.19 |
| 135 | 0.07 | 0.22 |
| 136 | 0.32 | 1.84 |
| 137 | 0.31 | 1.27 |
| 138 | 0.28 | 0.42 |
| 139 | 2.66 | 5.01 |
| 140 | 0.05 | 0.15 |
| 141 | 0.16 | 0.55 |
| 142 | 0.07 | 0.07 |
| 143 | 0.48 | 0.20 |
| 144 | 0.02 | 0.05 |
| 145 | 0.05 | 0.05 |
| 146 | 0.02 | 0.03 |
| 147 | 0.02 | 0.02 |
| 148 | 0.22 | 0.54 |
| 149 | 2.25 | 0.08 |
| 150 | 0.23 | 0.45 |
| 151 | 0.65 | 0.50 |
| 152 | 0.05 | 0.07 |
| 153 | 0.08 | 0.07 |
| 154 | 0.02 | 0.02 |
| 155 | 0.63 | 1.08 |
| 156 | 0.01 | 0.01 |
| 157 | 0.02 | 0.15 |
| 158 | 0.01 | 0.04 |
| 159 | 0.03 | 0.03 |
| 160 | 0.01 | 0.01 |
| 161 | 0.03 | 0.03 |
| 162 | 0.01 | 0.01 |
| 163 | 0.38 | 0.08 |
| 164 | 0.03 | 0.10 |
| 165 | 5.01 | 5.01 |
| 166 | 0.92 | 2.00 |
| 167 | 0.63 | 0.52 |
| 168 | 0.10 | 0.07 |
| 169 | 1.88 | 0.16 |
| 170 | 8.81 | 1.32 |
| 171 | 0.81 | 0.58 |
| 172 | 3.21 | 3.76 |
| 173 | 0.75 | 0.48 |
| 174 | 25.12 | 2.29 |
| 175 | 2.57 | 0.14 |
| 176 | 50.12 | 50.12 |
| 177 | 50.12 | 50.12 |
| 178 | 0.02 | 0.03 |
| 179 | 0.06 | 0.36 |
| 180 | 1.62 | 2.85 |
| 181 | 6.53 | 7.08 |
| 182 | 0.13 | 0.66 |
| 183 | 5.01 | 4.47 |
| 184 | 0.22 | 0.08 |
| 185 | 1.48 | 1.55 |
| 186 | 0.01 | 0.02 |
| 187 | 4.68 | 1.62 |
| 188 | 0.19 | 0.06 |
| 189 | 0.22 | 0.03 |
| 190 | 1.50 | 0.07 |
| 191 | 0.21 | 0.04 |
| 192 | 0.28 | 0.02 |
| 193 | 0.77 | 0.09 |
| 194 | 0.03 | 0.03 |
| 195 | 0.05 | 0.03 |
| 196 | 1.19 | 0.96 |
| 197 | 0.01 | 0.01 |
| 198 | 5.01 | 5.01 |
| 199 | 4.79 | 5.37 |
| 200 | 0.17 | 0.03 |
| 201 | 0.10 | 0.03 |
| 202 | 0.02 | 0.01 |
| 203 | 0.32 | 2.34 |
| 204 | 0.04 | 0.30 |
| 205 | 1.07 | 1.76 |
| 206 | 0.07 | 0.14 |
| 207 | 0.01 | 0.09 |
| 208 | 0.62 | 2.82 |
| 209 | 0.02 | 0.09 |
| 210 | 0.09 | 0.53 |
| 211 | 0.58 | 0.34 |
| 212 | 0.05 | 0.08 |
| 213 | 0.08 | 0.60 |
| 214 | 0.01 | 0.03 |
| 215 | 0.09 | 0.35 |
| 216 | 0.05 | 0.05 |
| 217 | 0.01 | 0.02 |
| 218 | 4.37 | 5.01 |
| 219 | 0.15 | 0.58 |
| 220 | 0.02 | 0.03 |
| 221 | 2.54 | 0.12 |
| 222 | 0.94 | 0.02 |
| 223 | 1.58 | 0.81 |
| 224 | 1.43 | 0.21 |
| 225 | 0.48 | 0.15 |
| 226 | 2.26 | 1.45 |
| 227 | 0.69 | 0.34 |
| 228 | 0.25 | 0.09 |
| 229 | 0.96 | 0.35 |
| 230 | 0.42 | 0.18 |
| 231 | 0.02 | 0.03 |
| 232 | 0.06 | 0.08 |
| 233 | 0.11 | 0.18 |
| 234 | 0.06 | 0.05 |
| 235 | 0.02 | 0.03 |
| 236 | 5.01 | 5.01 |
| 237 | 5.01 | 5.01 |
| 238 | 5.01 | 5.01 |
| 239 | 0.80 | 7.94 |

### Formulation Examples

The following are representative pharmaceutical formulations containing a compound of Formula (I) or a pharmaceutically acceptable salt thereof.

### Formulation Example 1

### Tablet Formulation

The following ingredients are mixed intimately and pressed into single scored tablets.

| Ingredient | Quantity per tablet (mg) |
|---|---|
| compound | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| Ingredient | Quantity per capsule (mg) |
|---|---|
| compound | 200 |
| Lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |
| distilled water | q.s. (quantity sufficient) to 100 | mL |

### Formulation Example 4

### Injectable Formulation

The following ingredients are mixed to form an injectable formulation.

| Ingredient | Amount |
|---|---|
| compound | 0.2 mg-20 mg |
| sodium acetate buffer solution, | 0.4 M 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

What is claimed is:

1. A compound of Formula (I): 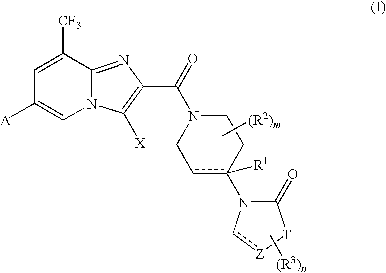
or a pharmaceutically acceptable salt thereof, wherein:
Z and T are independently selected from the group consisting of O, N, NR⁴, CH, CH₂, CH═CH, and CH₂═CH₂, optionally substituted with one to two R³;
R¹ is absent or is selected from the group consisting of hydrogen, halo, cyano, hydroxy, and alkyl;
R² and R³ are independently selected from the group consisting of hydrogen, deuterium, halo, cyano, oxo, —OR⁶, —NR⁶R⁶, —NR⁶S(O)₂(alkyl), —NR⁶C(O)OR⁶, —NR⁶C(O)R⁶, —C(O)NR⁶R⁶, —C(O)OR⁶, —C(O)R⁶, alkyl, and alkyl substituted with one to three R⁵;
R⁴ is hydrogen or alkyl;
R⁵ is independently selected from the group consisting of halo, cyano, —OR⁶, —C(O)OR⁶, —NR⁶R⁶, and alkoxy substituted with one to three R^{5a} wherein R^{5a} independently selected from the group consisting of halo, hydroxy, cyano, —OR⁶, —C(O)OR⁶, and —NR⁶R⁶;
m is 0, 1, 2, 3, 4, 5, or 6;
n is 0, 1, 2, 3, or 4; [symbol]
independently represents a single bond or a double bond, provided that when [symbol] is a double bond, then R¹ is absent;
X is selected from the group consisting of hydrogen, cyano, halo, —CH₂OH, hydroxy, alkyl, and cycloalkyl;
A is selected from the group consisting of hydrogen, halo, cyano, alkyl, alkoxy, alkenyl, alkynyl, cycloalkyl, aryl, heteroaryl, or heterocyclic, —OR⁶, —NR⁶R⁶, —NR⁶C(O)NR⁶R⁶, —NR⁶C(S)NR⁶R⁶, —NR⁶S(O)₂R⁶ —NR⁶C(O)OR⁶, and —NR⁶C(O)R⁶; wherein said alkyl, alkoxy, alkenyl, alkynyl, cycloalkyl, aryl, heteroaryl, or heterocyclic is optionally substituted with one to three substituents independently selected from alkyl, alkenyl, alkynyl, halo, hydroxy, cyano, —OR⁶, —SR⁶, —S(O)R⁶, —S(O)₂R⁶, —S(O)₂NR⁶R⁶, —NR⁶R⁶, —NR⁶C(O)NR⁶R⁶, —NR⁶C(S)NR⁶R⁶, —NR⁶S(O)₂R⁶ —NR⁶C(O)OR⁶, —NR⁶C(O)R⁶, —C(NR⁶)NR⁶R⁶, —C(O)NR⁶R⁶, —C(O)OR⁶, —CN, —C(O)R⁶, cycloalkyl, aryl, heteroaryl, and heterocyclic; and
R⁶ is independently hydrogen or alkyl.

2. A compound of claim 1 wherein T is CH₂.

3. A compound of claim 1 wherein T is O.

4. A compound of claim 1 wherein R¹ is hydrogen

5. A compound of claim 1 wherein X is halo.

6. A compound of claim 5 wherein X is chloro.

7. A compound of claim 1 wherein X is cyano.

8. A compound of claim 6 wherein T is CH₂ or O.

9. A compound of claim 1 wherein R¹ is hydrogen.

10. A compound of claim 1 wherein m is 0.

11. A compound of claim 1 wherein n is 0.

12. A compound of claim 1 wherein [symbol] represents a single bond.

13. A compound of claim 1 wherein A is selected from the group consisting of halo, cyano, alkenyl, cycloalkyl, aryl, heteroaryl, and heterocyclic.

14. A compound of claim 13 wherein A is heteroaryl.

15. A compound of claim 14 wherein A is selected from furanyl and pyrazolyl.

16. A compound of claim 13 wherein A is aryl.

17. A compound of claim 16, wherein A is phenyl.

18. A compound of claim 13, wherein A is cycloalkyl.

19. A compound of claim 18, wherein A is cyclopentenyl.

20. A compound of claim 13, wherein A is halo.

21. A compound of claim 20, wherein A is bromo.

22. A compound of claim 13, wherein A is alkenyl.

23. A compound of claim 22, wherein A is isopropenyl.

24. A compound of claim 13, wherein A is cyano.

25. A compound selected from the group consisting of
1-(1-{[3-chloro-6-(3-furanyl)-8-(trifluoromethyl)imidazo[1,2-a]pyridin-2-yl]carbonyl}-4-piperidinyl)-2-pyrrolidinone,
1-(1-{[3-chloro-6-(1H-pyrazol-4-yl)-8-(trifluoromethyl)imidazo[1,2-a]pyridin-2-yl]carbonyl}-4-piperidinyl)-2-pyrrolidinone,
3-(1-{[3-chloro-6-(3-furanyl)-8-(trifluoromethyl)imidazo[1,2-a]pyridin-2yl]carbonyl}-4-piperidinyl)-1,3-oxazolidin-2-one,
3-(1-{[3-chloro-6-phenyl-8-(trifluoromethyl)imidazo[1,2-a]pyridin-2-yl]carbonyl}-4-piperidinyl)-1,3-oxazolidin-2-one,
3-(1-{[6-bromo-3-chloro-8-(trifluoromethyl)imidazo[1,2-a]pyridin-2-yl]carbonyl}-4-piperidinyl)-1,3-oxazolidin-2-one,
3-(1-{[3-chloro-6-(1-methylethenyl)-8-(trifluoromethyl)imidazo[1,2-a]pyridin-2-yl]carbonyl}-4-piperidinyl)-1,3-oxazolidin-2-one,
3-(1-{[3-chloro-6-(1-cyclopenten-1-yl)-8-(trifluoromethyl)imidazo[1,2-a]pyridin-2-yl]carbonyl}-4-piperidinyl)-1,3-oxazolidin-2-one,
3-(1-{[3-chloro-6-(1H-pyrazol-4-yl)-8-(trifluoromethyl)imidazo[1,2-a]pyridin-2-yl]carbonyl}-4-piperidinyl)-1,3-oxazolidin-2-one,
1-(1-{[3-chloro-8-(trifluoromethyl)imidazo[1,2-a]pyridin-2-yl]carbonyl}-4-piperidinyl)-2-pyrrolidinone,
1-(1-{[3,6-dichloro-8-(trifluoromethyl)imidazo[1,2-a]pyridin-2-yl]carbonyl}-4-piperidinyl)-2-pyrrolidinone,
1-(1-{[3-chloro-8-(trifluoromethyl)imidazo[1,2-a]pyridin-2-yl]carbonyl}-4-piperidinyl)-2-pyrrolidinone,
3-(1-{[3,6-dichloro-8-(trifluoromethyl)imidazo[1,2-a]pyridin-2-yl]carbonyl}-4-piperidinyl)-1,3-oxazolidin-2-one,
3-(1-{[3-chloro-6-(4-pyridinyl)-8-(trifluoromethyl)imidazo[1,2-a]pyridin-2-yl]carbonyl}-4-piperidinyl)-1,3-oxazolidin-2-one,
3-(1-{[3-chloro-6-(3-fluorophenyl)-8-(trifluoromethyl)imidazo[1,2-a]pyridin-2-yl]carbonyl}-4-piperidinyl)-1,3-oxazolidin-2-one,
3-(1-{[3-chloro-6-(4-fluorophenyl)-8-(trifluoromethyl)imidazo[1,2-a]pyridin-2-yl]carbonyl}-4-piperidinyl)-1,3-oxazolidin-2-one,
3-(1-{[3-chloro-6-[2-(methyloxy)phenyl]-8-(trifluoromethyl)imidazo[1,2-a]pyridin-2-yl]carbonyl}-4-piperidinyl)-1,3-oxazolidin-2-one,
3-(1-{[3-chloro-6-[3-(methyloxy)phenyl]-8-(trifluoromethyl)imidazo[1,2-a]pyridin-2-yl]carbonyl}-4-piperidinyl)-1,3-oxazolidin-2-one,
3-(1-{[3-chloro-6-[4-(methyloxy)phenyl]-8-(trifluoromethyl)imidazo[1,2-a]pyridin-2-yl]carbonyl}-4-piperidinyl)-1,3-oxazolidin-2-one,
3-(1-{[3-chloro-6-(3-thienyl)-8-(trifluoromethyl)imidazo[1,2-a]pyridin-2-yl]carbonyl}-4-piperidinyl)-1,3-oxazolidin-2-one,
3-(1-{[3-chloro-6-(8-quinolinyl)-8-(trifluoromethyl)imidazo[1,2-a]pyridin-2-yl]carbonyl}-4-piperidinyl)-1,3-oxazolidin-2-one,
3-(1-{[3-chloro-6-(2-hydroxyphenyl)-8-(trifluoromethyl)imidazo[1,2-a]pyridin-2-yl]carbonyl}-4-piperidinyl)-1,3-oxazolidin-2-one,
3-(1-{[3-chloro-6-(3-hydroxyphenyl)-8-(trifluoromethyl)imidazo[1,2-a]pyridin-2-yl]carbonyl}-4-piperidinyl)-1,3-oxazolidin-2-one,
3-(1-{[3-chloro-6-(4-hydroxyphenyl)-8-(trifluoromethyl)imidazo[1,2-a]pyridin-2-yl]carbonyl}-4-piperidinyl)-1,3-oxazolidin-2-one,
3-(1-{[3-chloro-6-(1-methyl-1H-pyrazol-4-yl)-8-(trifluoromethyl)imidazo[1,2-a]pyridin-2-yl]carbonyl}-4-piperidinyl)-1,3-oxazolidin-2-one,
3-(1-{[3-chloro-6-(1-ethyl-1H-pyrazol-4-yl)-8-(trifluoromethyl)imidazo[1,2-a]pyridin-2-yl]carbonyl}-4-piperidinyl)-1,3-oxazolidin-2-one,
3-(1-{[3-chloro-6-[1-(2-methylpropyl)-1H-pyrazol-4-yl]-8-(trifluoromethyl)imidazo[1,2-a]pyridin-2-yl]carbonyl}-4-piperidinyl)-1,3-oxazolidin-2-one,
3-(1-{[3-chloro-6-cyclopentyl-8-(trifluoromethyl)imidazo[1,2-a]pyridin-2-yl]carbonyl}-4-piperidinyl)-1,3-oxazolidin-2-one,
3-(1-{[3-chloro-6-cyclobutyl-8-(trifluoromethyl)imidazo[1,2-a]pyridin-2-yl]carbonyl}-4-piperidinyl)-1,3-oxazolidin-2-one,
3-(1-{[3-chloro-6-propyl-8-(trifluoromethyl)imidazo[1,2-a]pyridin-2-yl]carbonyl}-4-piperidinyl)-1,3-oxazolidin-2-one,
3-(1-{[3-chloro-6-(2-methylpropyl)-8-(trifluoromethyl)imidazo[1,2-a]pyridin-2-yl]carbonyl}-4-piperidinyl)-1,3-oxazolidin-2-one,
3-(1-{[6-butyl-3-chloro-8-(trifluoromethyl)imidazo[1,2-a]pyridin-2-yl]carbonyl}-4-piperidinyl)-1,3-oxazolidin-2-one,
3-(1-{[3-chloro-6-(methyloxy)-8-(trifluoromethyl)imidazo[1,2-a]pyridin-2-yl]carbonyl}-4-piperidinyl)-1,3-oxazolidin-2-one,
3-(1-{[3-chloro-6-(5-methyl-2-furanyl)-8-(trifluoromethyl)imidazo[1,2-a]pyridin-2-yl]carbonyl}-4-piperidinyl)-1,3-oxazolidin-2-one,
3-(1-{[3-chloro-6-(tetrahydro-2-furanyl)-8-(trifluoromethyl)imidazo[1,2-a]pyridin-2-yl]carbonyl}-4-piperidinyl)-1,3-oxazolidin-2-one,
3-(1-{[3-chloro-6-(ethyloxy)-8-(trifluoromethyl)imidazo[1,2-a]pyridin-2-yl]carbonyl}-4-piperidinyl)-1,3-oxazolidin-2-one,
3-(1-{[3-chloro-6-(3-pyridinyl)-8-(trifluoromethyl)imidazo[1,2-a]pyridin-2-yl]carbonyl}-4-piperidinyl)-1,3-oxazolidin-2-one,
3-(1-{[3-chloro-6-(tetrahydro-3-furanyl)-8-(trifluoromethyl)imidazo[1,2-a]pyridin-2-yl]carbonyl}-4-piperidinyl)-1,3-oxazolidin-2-one,
3-(1-{[3-chloro-6-[(1-methylethyl)oxy]-8-(trifluoromethyl)imidazo[1,2-a]pyridin-2-yl]carbonyl}-4-piperidinyl)-1,3-oxazolidin-2-one,
3-(1-{[3-chloro-6-(1H-imidazol-1-yl)-8-(trifluoromethyl)imidazo[1,2-a]pyridin-2-yl]carbonyl}-4-piperidinyl)-1,3-oxazolidin-2-one,
3-(1-{[3-chloro-6-(2-methylpropyl)-8-(trifluoromethyl)imidazo[1,2-a]pyridin-2-yl]carbonyl}-4-piperidinyl)-1,3-oxazolidine-2,4-dione,
3-(1-{[3-chloro-6-(1-methylpropyl)-8-(trifluoromethyl)imidazo[1,2-a]pyridin-2-yl]carbonyl}-4-piperidinyl)-1,3-oxazolidin-2-one,
3-(1-{[6-bromo-3-chloro-8-(trifluoromethyl)imidazo[1,2-a]pyridin-2-yl]carbonyl}-4-piperidinyl)-1,3-oxazolidine-2,4-dione,
1-(1-{[6-bromo-3-chloro-8-(trifluoromethyl)imidazo[1,2-a]pyridin-2-yl]carbonyl}-4-piperidinyl)-2-pyrrolidinone,
3-(1-{[3-chloro-6-[(difluoromethyl)oxy]-8-(trifluoromethyl)imidazo[1,2-a]pyridin-2-yl]carbonyl}-4-piperidinyl)-1,3-oxazolidin-2-one,
3-(1-{[3-chloro-6-[(difluoromethyl)oxy]-8-(trifluoromethyl)imidazo[1,2-a]pyridin-2-yl]carbonyl}-4-piperidinyl)-1,3-oxazolidine-2,4-dione,
3-(1-{[3-chloro-6-(2-furanyl)-8-(trifluoromethyl)imidazo[1,2-a]pyridin-2-yl]carbonyl}-4-piperidinyl)-1,3-oxazolidin-2-one,
3-(1-{[3-chloro-6-(1H-pyrrol-3-yl)-8-(trifluoromethyl)imidazo[1,2-a]pyridin-2-yl]carbonyl}-4-piperidinyl)-1,3-oxazolidin-2-one,
3-(1-{[3-chloro-6-(2H-1,2,3-triazol-4-yl)-8-(trifluoromethyl)imidazo[1,2-a]pyridin-2-yl]carbonyl}-4-piperidinyl)-1,3-oxazolidin-2-one,
3-(1-{[3-chloro-6-(1H-imidazol-2-yl)-8-(trifluoromethyl)imidazo[1,2-a]pyridin-2-yl]carbonyl}-4-piperidinyl)-1,3-oxazolidin-2-one,
3-(1-{[3-chloro-6-(1H-pyrrol-2-yl)-8-(trifluoromethyl)imidazo[1,2-a]pyridin-2-yl]carbonyl}-4-piperidinyl)-1,3-oxazolidin-2-one,
3-(1-{[3-chloro-6-methyl-8-(trifluoromethyl)imidazo[1,2-a]pyridin-2-yl]carbonyl}-4-piperidinyl)-1,3-oxazolidin-2-one,
3-(1-{[3-chloro-6-ethyl-8-(trifluoromethyl)imidazo[1,2-a]pyridin-2-yl]carbonyl}-4-piperidinyl)-1,3-oxazolidin-2-one,
3-(1-{[3-chloro-6-ethenyl-8-(trifluoromethyl)imidazo[1,2-a]pyridin-2-yl]carbonyl}-4-piperidinyl)-1,3-oxazolidin-2-one,
3-(1-{[3-chloro-6-(1-methylethyl)-8-(trifluoromethyl)imidazo[1,2-a]pyridin-2-yl]carbonyl}-4-piperidinyl)-1,3-oxazolidin-2-one,
3-(1-{[3-chloro-6-(1-hydroxyethyl)-8-(trifluoromethyl)imidazo[1,2-a]pyridin-2-yl]carbonyl}-4-piperidinyl)-1,3-oxazolidin-2-one,
3-(1-{[3-chloro-6-cyclopropyl-8-(trifluoromethyl)imidazo[1,2-a]pyridin-2-yl]carbonyl}-4-piperidinyl)-1,3-oxazolidin-2-one,
3-(1-{[3-chloro-6-(difluoromethyl)-8-(trifluoromethyl)imidazo[1,2-a]pyridin-2-yl]carbonyl}-4-piperidinyl)-1,3-oxazolidin-2-one,
3-(1-{[3-bromo-6-(3-furanyl)-8-(trifluoromethyl)imidazo[1,2-a]pyridin-2-yl]carbonyl}-4-piperidinyl)-1,3-oxazolidin-2-one,
3-(1-{[3-bromo-6-(3-furanyl)-8-(trifluoromethyl)imidazo[1,2-a]pyridin-2-yl]carbonyl}-4-piperidinyl)-1,3-oxazolidine-2,4-dione,
6-(3-furanyl)-2-{[4-(2-oxo-1,3-oxazolidin-3-yl)-1-piperidinyl]carbonyl}-8-(trifluoromethyl)imidazo[1,2-a]pyridine-3-carbonitrile,
2-{[4-(2,4-dioxo-1,3-oxazolidin-3-yl)-1-piperidinyl]carbonyl}-6-(3-furanyl)-8-(trifluoromethyl)imidazo[1,2-a]pyridine-3-carbonitrile,
6-(3-furanyl)-2-{[4-(2-oxo-1,3-oxazolidin-3-yl)-1-piperidinyl]carbonyl}-8-(trifluoromethyl)imidazo[1,2-a]pyridine-3-carbonitrile,
1-(1-{[3-chloro-6-cyclopropyl-8-(trifluoromethyl)imidazo[1,2-a]pyridin-2-yl]carbonyl}-4-piperidinyl)-4-hydroxy-2-pyrrolidinone,
3-(1-{[6-(3-furanyl)-3-methyl-8-(trifluoromethyl)imidazo[1,2-a]pyridin-2-yl]carbonyl}-4-piperidinyl)-1,3-oxazolidin-2-one,
3-(1-{[6-(3-furanyl)-3-(1-methylethyl)-8-(trifluoromethyl)imidazo[1,2-a]pyridin-2-yl]carbonyl}-4-piperidinyl)-1,3-oxazolidin-2-one,
3-(1-{[6-(3-furanyl)-3-(hydroxymethyl)-8-(trifluoromethyl)imidazo[1,2-a]pyridin-2-yl]carbonyl}-4-piperidinyl)-1,3-oxazolidin-2-one,
1-(1-{[3-chloro-6-(1H-pyrazol-4-yl)-8-(trifluoromethyl)imidazo[1,2-a]pyridin-2-yl]carbonyl}-4-piperidinyl)-2,5-pyrrolidinedione,
1-(1-{[3-chloro-6-(3-furanyl)-8-(trifluoromethyl)imidazo[1,2-a]pyridin-2-yl]carbonyl}-4-piperidinyl)-2,5-pyrrolidinedione,
1-(1-{[3-chloro-6-(3-furanyl)-8-(trifluoromethyl)imidazo[1,2-a]pyridin-2-yl]carbonyl}-4-piperidinyl)-2-imidazolidinone,
1-(1-{[3-chloro-6-(1H-pyrazol-4-yl)-8-(trifluoromethyl)imidazo[1,2-a]pyridin-2-yl]carbonyl}-4-piperidinyl)-2-imidazolidinone,
1-(1-{[3-chloro-6-(3-furanyl)-8-(trifluoromethyl)imidazo[1,2-a]pyridin-2-yl]carbonyl}-4-piperidinyl)-2,4-imidazolidinedione,
1-(1-{[3-chloro-6-(1H-pyrazol-4-yl)-8-(trifluoromethyl)imidazo[1,2-a]pyridin-2-yl]carbonyl}-4-piperidinyl)-2,4-imidazolidinedione,
1-(1-{[3-chloro-6-(3-furanyl)-8-(trifluoromethyl)imidazo[1,2-a]pyridin-2-yl]carbonyl}-4-piperidinyl)-3-methyl-2-imidazolidinone,
3-(1-{[3-chloro-6-(1H-pyrazol-4-yl)-8-(trifluoromethyl)imidazo[1,2-a]pyridin-2-yl]carbonyl}-4-piperidinyl)-2,4-imidazolidinedione,
3-(1-{[3-chloro-6-(3-furanyl)-8-(trifluoromethyl)imidazo[1,2-a]pyridin-2-yl]carbonyl}-4-piperidinyl)-2,4-imidazolidinedione,
3-(1-{[3-chloro-6-(3-furanyl)-8-(trifluoromethyl)imidazo[1,2-a]pyridin-2-yl]carbonyl}-4-piperidinyl)-4,4-dimethyl-1,3-oxazolidin-2-one,
3-(1-{[3-chloro-6-(1H-pyrazol-4-yl)-8-(trifluoromethyl)imidazo[1,2-a]pyridin-2-yl]carbonyl}-4-piperidinyl)-4,4-dimethyl-1,3-oxazolidin-2-one,
3-(1-{[3-chloro-6-(3-furanyl)-8-(trifluoromethyl)imidazo[1,2-a]pyridin-2-yl]carbonyl}-4-piperidinyl)-1,3-oxazolidine-2,4-dione,
3-(1-{[3-chloro-6-(1H-pyrazol-4-yl)-8-(trifluoromethyl)imidazo[1,2-a]pyridin-2-yl]carbonyl}-4-piperidinyl)-1,3-oxazolidine-2,4-dione,
(4S)-3-(1-{[3-chloro-6-(3-furanyl)-8-(trifluoromethyl)imidazo[1,2-a]pyridin-2-yl]carbonyl}-4-piperidinyl)-4-methyl-1,3-oxazolidin-2-one,
3-(1-{[3-chloro-6-(3-furanyl)-8-(trifluoromethyl)imidazo[1,2-a]pyridin-2-yl]carbonyl}-4-piperidinyl)-5,5-dimethyl-1,3-oxazolidin-2-one,
(4R)-3-(1-{[3-chloro-6-(3-furanyl)-8-(trifluoromethyl)imidazo[1,2-a]pyridin-2-yl]carbonyl}-4-piperidinyl)-4-methyl-1,3-oxazolidin-2-one,
3-(1-{[3-chloro-6-(3-furanyl)-8-(trifluoromethyl)imidazo[1,2-a]pyridin-2-yl]carbonyl}-4-methyl-4-piperidinyl)-1,3-oxazolidin-2-one,
(5S)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidin-2-one,
(5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidin-2-one,
3-(1-{[3-chloro-6-(3-furanyl)-8-(trifluoromethyl)imidazo[1,2-a]pyridin-2-yl]carbonyl}-4-piperidinyl)-1,3-oxazol-2(3H)-one,
3-(1-{[6-amino-3-chloro-8-(trifluoromethylimidazo[1,2-a]pyridin-2-yl]carbonyl}-4-piperidinyl)-1,3-oxazolidin-2-one,
(5S)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione,
(5S)-3-(1-{[3-chloro-6-cyclopropyl-8-(trifluoromethyl)imidazo[1,2-a]pyridin-2-yl]carbonyl}-4-piperidinyl)-5-methyl-1,3-oxazolidine-2,4-dione,
(5S)-3-(1-{[6-bromo-3-chloro-8-(trifluoromethyl)imidazo[1,2-a]pyridin-2-yl]carbonyl}-4-piperidinyl)-5-methyl-1,3-oxazolidine-2,4-dione,
(5R)-3-(1-{[3-chloro-6-(3-furanyl)-8-(trifluoromethyl)imidazo[1,2-a]pyridin-2-yl]carbonyl}-4-piperidinyl)-5-methyl-1,3-oxazolidine-2,4-dione,
(5R)-3-(1-{[3-chloro-6-cyclopropyl-8-(trifluoromethyl)imidazo[1,2-a]pyridin-2-yl]carbonyl}-4-piperidinyl)-5-methyl-1,3-oxazolidine-2,4-dione,
(5R)-3-(1-{[6-bromo-3-chloro-8-(trifluoromethyl)imidazo[1,2-a]pyridin-2-yl]carbonyl}-4-piperidinyl)-5-methyl-1,3-oxazolidine-2,4-dione,
3-(1-{[3-bromo-6-iodo-8-(trifluoromethyl)imidazo[1,2-a]pyridin-2-yl]carbonyl}-4-piperidinyl)-1,3-oxazolidin-2-one,
3-(1-{[3-bromo-6-iodo-8-(trifluoromethyl)imidazo[1,2-a]pyridin-2-yl]carbonyl}-4-piperidinyl)-1,3-oxazolidine-2,4-dione,
3-(1-{[6-(1-methylethyl)-8-(trifluoromethyl)imidazo[1,2-a]pyridin-2-yl]carbonyl}-4-piperidinyl)-1,3-oxazolidin-2-one,
3-(1-{[3-bromo-6-cyclopropyl-8-(trifluoromethyl)imidazo[1,2-a]pyridin-2-yl]carbonyl}-4-piperidinyl)-1,3-oxazolidin-2-one,
N-[3-chloro-2-{[4-(2-oxo-1,3-oxazolidin-3-yl)-1-piperidinyl]carbonyl}-8-(trifluoromethyl)imidazo[1,2-a]pyridin-6-yl]acetamide,
4-(1-{[3-chloro-6-(3-furanyl)-8-(trifluoromethyl)imidazo[1,2-a]pyridin-2-yl]carbonyl}-4-piperidinyl)-2,4-dihydro-3H-1,2,4-triazol-3-one,
1-(1-{[3-chloro-6-cyclopropyl-8-(trifluoromethyl)imidazo[1,2-a]pyridin-2-yl]carbonyl}-4-piperidinyl)-2,5-pyrrolidinedione,
1-(1-{[3-chloro-6-cyclopropyl-8-(trifluoromethyl)imidazo[1,2-a]pyridin-2-yl]carbonyl}-4-piperidinyl)-2-pyrrolidinone,
3-(1-{[3-chloro-6-cyclopropyl-8-(trifluoromethyl)imidazo[1,2-a]pyridin-2-yl]carbonyl}-4-piperidinyl)-1,3-oxazolidine-2,4-dione,
3-(1-{[6-(3-furanyl)-8-(trifluoromethyl)imidazo[1,2-a]pyridin-2-yl]carbonyl}-4-piperidinyl)-1,3-oxazolidin-2-one,
3-(1-{[3-chloro-6-(3-furanyl)-8-(trifluoromethyl)imidazo[1,2-a]pyridin-2-yl]carbonyl}-3,3-dimethyl-4-piperidinyl)-1,3-oxazolidin-2-one,
(±) cis-3-(1-{[3-chloro-6-(3-furanyl)-8-(trifluoromethyl)imidazo[1,2-a]pyridin-2-yl]carbonyl}-2-methyl-4-piperidinyl)-1,3-oxazolidin-2-one,
(±) trans-3-(1-{[3-chloro-6-(3-furanyl)-8-(trifluoromethyl)imidazo[1,2-a]pyridin-2-yl]carbonyl}-2-methyl-4-piperidinyl)-1,3-oxazolidin-2-one,
3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 1),
3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 2),
3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 3),
3-(1-{[3-chloro-6-(3-furanyl)-8-(trifluoromethyl)imidazo[1,2-a]pyridin-2-yl]carbonyl}-3-methyl-4-piperidinyl)-1,3-oxazolidin-2-one (Isomer 4),
3-(1-{[3-chloro-6-(3-furanyl)-8-(trifluoromethyl)imidazo[1,2-a]pyridin-2-yl]carbonyl}-2,2-dimethyl-4-piperidinyl)-1,3-oxazolidin-2-one,
(±)-trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one,
(±)-cis-3-(1-{[3-chloro-6-(3-furanyl)-8-(trifluoromethyl)imidazo[1,2-a]pyridin-2-yl]carbonyl}-3-fluoro-4-piperidinyl)-1,3-oxazolidin-2-one,
(±)-trans-3-(1-{[3-chloro-6-(3-furanyl)-8-(trifluoromethyl)imidazo[1,2-a]pyridin-2-yl]carbonyl}-3-fluoro-4-piperidinyl)-1,3-oxazolidin-2-one,
(±) trans-3-(1-{[3-chloro-6-(2-methylpropyl)-8-(trifluoromethyl)imidazo[1,2-a]pyridin-2-yl]carbonyl}-3-hydroxy-4-piperidinyl)-1,3-oxazolidin-2-one,
(±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one,
3-((trans)-1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one (enantiomer 1),
3-((trans)-1-{[3-chloro-6-(3-furanyl)-8-(trifluoromethyl)imidazo[1,2-a]pyridin-2-yl]carbonyl}-3-hydroxy-4-piperidinyl)-1,3-oxazolidin-2-one (enantiomer 2),
(±) trans-3-(1-{[6-bromo-3-chloro-8-(trifluoromethyl)imidazo[1,2-a]pyridin-2-yl]carbonyl}-3-hydroxy-4-piperidinyl)-1,3-oxazolidin-2-one,
3-((trans)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 1),
3-((trans)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 2),
(±) trans-3-(1-{[6-bromo-3-chloro-8-(trifluoromethyl)imidazo[1,2-a]pyridin-2-yl]carbonyl}-3-hydroxy-4-piperidinyl)-1,3-oxazolidine-2,4-dione,
(±) trans-3-(1-{[3-chloro-6-(3-furanyl)-8-(trifluoromethyl)imidazo[1,2-a]pyridin-2-yl]carbonyl}-3-hydroxy-4-piperidinyl)-1,3-oxazolidine-2,4-dione,
(±) trans-3-(1-{[3-chloro-6-cyclopropyl-8-(trifluoromethyl)imidazo[1,2-a]pyridin-2-yl]carbonyl}-3-hydroxy-4-piperidinyl)-1,3-oxazolidine-2,4-dione,
(±) trans-1-(1-{[3-chloro-6-cyclopropyl-8-(trifluoromethyl)imidazo[1,2-a]pyridin-2-yl]carbonyl}-3-hydroxy-4-piperidinyl)-2-pyrrolidinone,
(±) trans-1-(1-{[6-bromo-3-chloro-8-(trifluoromethyl)imidazo[1,2-a]pyridin-2-yl]carbonyl}-3-hydroxy-4-piperidinyl)-2-pyrrolidinone,
(±) trans-1-(1-{[3-chloro-6-(3-furanyl)-8-(trifluoromethyl)imidazo[1,2-a]pyridin-2-yl]carbonyl}-3-hydroxy-4-piperidinyl)-2-pyrrolidinone piperidinyl,
(±) cis-1-(1-{[3-chloro-6-cyclopropyl-8-(trifluoromethyl)imidazo[1,2-a]pyridin-2-yl]carbonyl}-3-hydroxy-4-)-2-pyrrolidinone,
(±) cis-1-(1-{[6-bromo-3-chloro-8-(trifluoromethyl)imidazo[1,2-a]pyridin-2-yl]carbonyl}-3-hydroxy-4-piperidinyl)-2-pyrrolidinone,
(±) cis-1-(1-{[3-chloro-6-(3-furanyl)-84trifluoromethyl)imidazo[1,2-a]pyridin-2-yl]carbonyl}-3-hydroxy-4-piperidinyl)-2-pyrrolidinone,
(±) cis-3-(1-{[3-chloro-6-cyclopropyl-84trifluoromethyl)imidazo[1,2-a]pyridin-2-yl]carbonyl}-3-hydroxy-4-piperidinyl)-1,3-oxazolidin-2-one,
1-{[3-chloro-6-cyclopropyl-8-(trifluoromethyl)imidazo[1,2-a]pyridin-2-yl]carbonyl}-4-(2-oxo-1,3-oxazolidin-3-yl)-3-piperidinone,
(±) cis-3-(1-{[6-bromo-3-chloro-8-(trifluoromethyl)imidazo[1,2-a]pyridin-2-yl]carbonyl}-3-hydroxy-4-piperidinyl)-1,3-oxazolidin-2-one,
3-((cis)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 1),
3-((cis)-1-{[3-chloro-6-cyclopropyl-8-(trifluoromethyl)imidazo[1,2-a]pyridin-2-yl]carbonyl}-3-hydroxy-4-piperidinyl)-1,3-oxazolidin-2-one (Enantiomer 2),
(±) trans-3-[1-{[3-chloro-6-(3-furanyl)-8-(trifluoromethyl)imidazo[1,2-a]pyridin-2-yl]carbonyl}-3-(methyloxy)-4-piperidinyl]-1,3-oxazolidin-2-one,
(±) trans-3-[1-{[6-bromo-3-chloro-8-(trifluoromethyl)imidazo[1,2-a]pyridin-2-yl]carbonyl}-3-(methyloxy)-4-piperidinyl]-1,3-oxazolidin-2-one,
(±) trans-341-{[3-chloro-6-cyclopropyl-8-(trifluoromethyl)imidazo[1,2-a]pyridin-2-yl]carbonyl}-3-(methyloxy)-4-piperidinyl]-1,3-oxazolidin-2-one, and
3-chloro-2-{[4-(2-oxo-1,3-oxazolidin-3-yl)-1-piperidinyl]carbonyl}-8-(trifluoromethyl)imidazo[1,2-a]pyridine-6-carbonitrile, or a pharmaceutically acceptable salt thereof.

26. A pharmaceutical composition comprising a pharmaceutically acceptable diluent and a therapeutically effective amount of a compound according to claim 1.

27. A method for treating a viral infection in a mammal mediated at least in part by a virus in the Flaviviridae family of viruses which method comprises administering to a mammal, that has been diagnosed with said viral infection or is at risk of developing said viral infection a compound according to claim 1.

28. The method of claim 27, wherein said virus is hepatitis C virus.

29. The method of claim 28, further comprising administration of a therapeutically effective amount of one or more agents active against hepatitis C virus.

30. The method of claim 29, wherein said agent active against hepatitis C virus is an inhibitor of HCV protease, HCV polymerase, HCV helicase, HCV NS4B protein, HCV entry, HCV assembly, HCV egress, HCV replicase, HCV NS5A protein, or inosine 5′-monophosphate dehydrogenase.

31. The method of claim 28, wherein said agent active against hepatitis C virus is interferon.
